SEQUENCE LISTING (32 sequences)

SEQ ID NO: 1 - protein, 584 aa, synthetic construct
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDDKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 2 - protein, 584 aa, synthetic construct
MKAIIVLLMVVTSNADRICTGITSSNSPHVVKTATQGEVXVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDDKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLXKSKPYYTGKHAKAIGNCPIWVKTPLKLAXGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCXQTCLDRIAAGTFNAGEFSLPTFDSLXITAASLNDDGLDXHTILLYYSTAASSLAVTLMLAIFIVYMVSRDNVSCSICL

SEQ ID NO: 3 - protein, 584 aa, synthetic construct
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDDKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLQKSKPYYTGKHAKAIGNCPIWVKTPLKLAQGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCQQTCLDRIAAGTFNAGEFSLPTFDSLQITAASLNDDGLDQHTILLYYSTAASSLAVTLMLAIFIVYMVSRDNVSCSICL

SEQ ID NO: 4 - protein, 566 aa, synthetic construct
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIAARPKVRDRAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 5 - protein, 566 aa, synthetic construct
MKAILVVMLYTFTTANADTLCIGYHAXXSTDTVDTVLEKXVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIAARPKVRDRAGRMNYYWTLVEPGDKITFEATGNLVAPRYAFTMEKEAGSGIIISDTPVHDCXATCQTPEGAIXTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKXGTYDYPKYSEEAKLNREKIDGVKLDSTRIYQILAIYSTVASSLVLVVSLGAISFWMCSNGSLQCRICI

SEQ ID NO: 6 - protein, 566 aa, synthetic construct
MKAILVVMLYTFTTANADTLCIGYHANNSTDTVDTVLEKQVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIAARPKVRDRAGRMNYYWTLVEPGDKITFEATGNLVAPRYAFTMEKEAGSGIIISDTPVHDCQATCQTPEGAIQTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKQGTYDYPKYSEEAKLNREKIDGVKLDSTRIYQILAIYSTVASSLVLVVSLGAISFWMCSNGSLQCRICI

SEQ ID NO: 7 - protein, 566 aa, synthetic construct
MKAIIALSNILCLVFAQKIPGNDNSTATLCLGHHAVPNGTIVKTITNDRIEVTNATELVQNSSIGKICNSPHQILDGGNCTLIDALLGDPQCDGFQNKEWDLFVERSRANSSCYPYDVPDYASLRSLVASSGTLEFKNESFNWTGVKQNGTSSACKRGSSSSFFSRLNWLTSLNNIYPAQNVTMPNKEQFDKLYIWGVHHPDTDKNQFSLFAQSSGRITVSTKRSQQAVIPNIGSRPRVRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 8 - protein, 566 aa, synthetic construct
MKAIIALSNILCLVFAQKIPGNDXSTATLCLGHHAVPXGTIVKTITNDRIEVTNATELVQNSSIGKICNSPHQILDGGNCTLIDALLGDPQCDGFQNKEWDLFVERSRANSSCYPYDVPDYASLRSLVASSGTLEFKNESFNWTGVKQNGTSSACKRGSSSSFFSRLNWLTSLNNIYPAQNVTMPNKEQFDKLYIWGVHHPDTDKNQFSLFAQSSGRITVSTKRSQQAVIPNIGSRPRVRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRXETYDHNVYRDEALNNRFQIKGVELKSGYKDWILWISFAMSCFLLCIALLGFIMWACQKGNIRCNICI

SEQ ID NO: 9 - protein, 566 aa, synthetic construct
MKAIIALSNILCLVFAQKIPGNDNSTATLCLGHHAVPQGTIVKTITNDRIEVTNATELVQNSSIGKICNSPHQILDGGNCTLIDALLGDPQCDGFQNKEWDLFVERSRANSSCYPYDVPDYASLRSLVASSGTLEFKNESFNWTGVKQNGTSSACKRGSSSSFFSRLNWLTSLNNIYPAQNVTMPNKEQFDKLYIWGVHHPDTDKNQFSLFAQSSGRITVSTKRSQQAVIPNIGSRPRVRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRQETYDHNVYRDEALNNRFQIKGVELKSGYKDWILWISFAMSCFLLCIALLGFIMWACQKGNIRCNICI

SEQ ID NO: 10 - protein, 566 aa, synthetic construct
MKAIIALSNILCLVFAQKIPGNDQSTATLCLGHHAVPQGTIVKTITNDRIEVTQATELVQQSSIGKICNSPHQILDGGQCTLIDALLGDPQCDGFQNKEWDLFVERSRANSSCYPYDVPDYASLRSLVASSGTLEFKNESFNWTGVKQNGTSSACKRGSSSSFFSRLNWLTSLNNIYPAQNVTMPNKEQFDKLYIWGVHHPDTDKNQFSLFAQSSGRITVSTKRSQQAVIPNIGSRPRVRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRQETYDHNVYRDEALNNRFQIKGVELKSGYKDWILWISFAMSCFLLCIALLGFIMWACQKGNIRCNICI

SEQ ID NO: 11 - protein, 566 aa, synthetic construct
MKAIIALSNILCLVFAQKIPGNDQSTATLCLGHHAVPQGTIVKTITNDRIEVTQATELVQQSSIGKICNSPHQILDGGQCTLIDALLGDPQCDGFQNKEWDLFVERSRANSSCYPYDVPDYASLRSLVASSGTLEFKNESFNWTGVKQNGTSSACKRGSSSSFFSRLNWLTSLNNIYPAQNVTMPNKEQFDKLYIWGVHHPDTDKNQFSLFAQSSGRITVSTKRSQQAVIPNIGSRPRVRDIPSRISIYWTIVKPGDILLINSTGNLIAPRGYFKIRSGKSSIMRSDAPIGKCKSECITPQGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRQETYDHNVYRDEALNNRFQIKGVELKSGYKDWILWISFAMSCFLLCIALLGFIMWACQKGNIRCNICI

SEQ ID NO: 12 - protein, 566 aa, synthetic construct
MKAIIALSNILCLVFAQKIPGNDQSTATLCLGHHAVPQGTIVKTITNDRIEVTNATELVQQSSIGKICNSPHQILDGGNCTLIDALLGDPQCDGFQNKEWDLFVERSRANSSCYPYDVPDYASLRSLVASSGTLEFKNESFNWTGVKQNGTSSACKRGSSSSFFSRLNWLTSLNNIYPAQNVTMPNKEQFDKLYIWGVHHPDTDKNQFSLFAQSSGRITVSTKRSQQAVIPNIGSRPRVRDIPSRISIYWTIVKPGDILLINSTGNLIAPRGYFKIRSGKSSIMRSDAPIGKCKSECITPQGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRQETYDHNVYRDEALNNRFQIKGVELKSGYKDWILWISFAMSCFLLCIALLGFIMWACQKGNIRCNICI

SEQ ID NO: 13 - protein, 566 aa, synthetic construct
MKAIIALSNILCLVFAQKIPGNDQSTATLCLGHHAVPQGTIVKTITNDRIEVTNATELVQNSSIGKICNSPHQILDGGNCTLIDALLGDPQCDGFQNKEWDLFVERSRANSSCYPYDVPDYASLRSLVASSGTLEFKNESFNWTGVKQNGTSSACKRGSSSSFFSRLNWLTSLNNIYPAQNVTMPNKEQFDKLYIWGVHHPDTDKNQFSLFAQSSGRITVSTKRSQQAVIPNIGSRPRVRDIPSRISIYWTIVKPGDILLIQSTGNLIAPRGYFKIRSGKSSIMRSDAPIGKCKSECITPQGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRQETYDHNVYRDEALNNRFQIKGVELKSGYKDWILWISFAMSCFLLCIALLGFIMWACQKGNIRCNICI

SEQ ID NO: 14 - protein, 582 aa, Synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 15 - protein, 582 aa, synthetic construct
MKAIIVLLMVVTSNADRICTGITSSNSPHVVKTATQGEVXVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLXKSKPYYTGEHAKAIGNCPIWVKTPLKLAXGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCXQTCLDRIAAGTFDAGEFSLPTFDSLXITAASLNDDGLDXHTILLYYSTAASSLAVTLMIAIFVVYMVSRDNVSCSICL

SEQ ID NO: 16 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLQKSKPYYTGEHAKAIGNCPIWVKTPLKLAQGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCQQTCLDRIAAGTFDAGEFSLPTFDSLQITAASLNDDGLDQHTILLYYSTAASSLAVTLMIAIFVVYMVSRDNVSCSICL

SEQ ID NO: 17 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLAQGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCQQTCLDRIAAGTFDAGEFSLPTFDSLQITAASLNDDGLDQHTILLYYSTAASSLAVTLMIAIFVVYMVSRDNVSCSICL

SEQ ID NO: 18 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCQQTCLDRIAAGTFDAGEFSLPTFDSLQITAASLNDDGLDQHTILLYYSTAASSLAVTLMIAIFVVYMVSRDNVSCSICL

SEQ ID NO: 19 - protein, 565 aa, synthetic construct
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 20 - protein, 1761 aa, synthetic construct
ATGAAGGCCATCATCGTGCTGCTGATGGTGGTGACCTCCAACGCCGATAGAATCTGCACCGGCATCACATCCTCCAATTCCCCTCACGTGGTGAAGACCGCCACACAGGGCGAGGTGAACGTGACAGGCGTGATCCCTCTGACCACAACACCTACCAAGTCCTACTTCGCCAACCTGAAGGGCACCAGGACAAGGGGCAAGCTGTGCCCTGATTGTCTGAACTGTACCGACCTGGATGTGGCCCTGGGCAGGCCTATGTGTGTGGGCACCACACCTAGCGCCAAGGCCAGCATCCTGCACGAGGTGAGGCCTGTGACAAGCGGCTGCTTTCCCATCATGCACGATAGAACAAAGATCAGACAGCTGCCCAATCTGCTGAGGGGCTACGAGAAGATCAGGCTGTCCACCCAGAACGTGATCGATGCCGAGAAGGCCCCCGGCGGCCCTTACAGACTGGGCACAAGCGGCAGCTGCCCCAACGCCACCAGCAAGATCGGCTTTTTCGCCACCATGGCCTGGGCCGTGCCTAAGGATAACTACAAGAATGCCACCAACCCCCTGACAGTGGAGGTGCCCTACATCTGTACCGAGGGCGAGGATCAGATCACAGTGTGGGGCTTTCACTCCGATGATAAGACACAGATGAAGTCCCTGTACGGCGACTCCAATCCTCAGAAGTTCACATCCTCCGCCAATGGCGTGACAACCCACTACGTGAGCCAGATCGGCGACTTTCCTGACCAGACCGAGGACGGCGGCCTGCCACAGAGCGGAAGGATCGTGGTGGACTACATGATGCAGAAGCCTGGCAAGACAGGCACAATCGTGTACCAGAGGGGCGTGCTGCTGCCCCAGAAGGTGTGGTGCGCCTCCGGCAGGTCCAAGGTCATTAAGGGCAGCCTGCCCCTGATCGGCGAGGCCGATTGTCTGCACGAGGAGTACGGCGGCCTGAATAAGTCCAAGCCCTACTACACCGGCAAGCACGCCAAGGCCATCGGCAATTGTCCTATCTGGGTGAAGACCCCTCTGAAGCTGGCCAACGGCACCAAGTACAGGCCTCCTGCCAAGCTGCTGAAGGAGAGGGGCTTCTTTGGCGCCATCGCCGGCTTTCTGGAGGGCGGATGGGAGGGCATGATCGCCGGCTGGCACGGCTACACCAGCCACGGAGCCCACGGCGTGGCCGTTGCTGCTGATCTGAAGAGCACACAGGAGGCCATCAACAAGATCACAAAGAACCTGAACTCCCTGTCCGAGCTGGAGGTGAAGAACCTGCAGAGGCTGAGCGGCGCCATGGACGAGCTGCACAATGAGATCCTGGAGCTGGATGAGAAGGTGGACGACCTGAGAGCCGATACAATCTCCTCCCAGATCGAGCTGGCCGTGCTGCTGTCCAATGAGGGCATCATCAACAGCGAGGACGAGCACCTGCTGGCCCTGGAGAGGAAGCTGAAGAAGATGCTGGGCCCCAGCGCCGTGGACATCGGCAATGGCTGCTTCGAGACAAAGCACAAGTGCAACCAGACATGCCTGGACAGAATCGCCGCCGGCACCTTCAATGCCGGCGAGTTCTCCCTGCCCACATTTGACTCCCTGAACATCACCGCCGCCTCCCTGAACGACGATGGCCTGGATAACCACACCATCCTGCTGTACTACAGCACAGCCGCCAGCAGCCTGGCCGTGACCCTGATGCTGGCCATCTTTATCGTGTACATGGTGTCCAGGGATAATGTGAGCTGCTCCATCTGTCTGTGATAGTAA

SEQ ID NO: 21 - protein, 1755 aa, synthetic construct
ATGAAGGCCATCATCGTGCTGCTGATGGTGGTGACATCCAATGCCGACAGAATCTGCACAGGCATCACCAGCTCCAACAGCCCCCACGTGGTGAAGACAGCCACACAGGGCGAGGTGAACGTGACCGGCGTGATCCCTCTGACAACCACACCTACCAAGTCCCACTTCGCCAATCTGAAGGGCACCGAGACAAGGGGCAAGCTGTGCCCTAAGTGTCTGAACTGTACCGATCTGGATGTGGCCCTGGGCAGGCCTAAGTGTACCGGCAAGATCCCTAGCGCCAGGGTGAGCATCCTGCACGAGGTGAGACCTGTGACAAGCGGCTGCTTCCCCATCATGCACGACAGAACCAAGATCAGGCAGCTGCCCAATCTGCTGAGAGGCTACGAGCACGTGAGACTGTCCACCCACAACGTGATCAATACCGAGGATGCCCCTGGCGGCCCTTACGAGATCGGCACCAGCGGCTCCTGTCTGAATATCACAAATGGCAAGGGCTTCTTTGCCACCATGGCCTGGGCCGTGCCTAAGAACAAGACCGCCACCAACCCTCTGACCATCGAGGTGCCCTACATCTGTACAGAGGAGGAGGATCAGATCACCGTGTGGGGCTTTCACTCCGATGACGAGACCCAGATGGCCAGACTGTACGGCGACAGCAAGCCCCAGAAGTTCACCTCCTCCGCCAACGGCGTGACCACACACTACGTGTCCCAGATCGGCGGCTTCCCTAACCAGACCGAGGACGGCGGCCTGCCTCAGAGCGGAAGGATCGTGGTGGATTACATGGTGCAGAAGTCCGGCAAGACAGGCACCATCACATACCAGAGGGGCATCCTGCTGCCTCAGAAGGTGTGGTGCGCCTCCGGCAAGAGCAAGGTCATTAAGGGCAGCCTGCCTCTGATCGGCGAGGCCGACTGCCTGCACGAGAAGTACGGCGGCCTGAATAAGAGCAAGCCCTACTACACCGGCGAGCACGCCAAGGCCATCGGCAACTGTCCCATCTGGGTGAAGACACCTCTGAAGCTGGCCAATGGCACCAAGTACAGACCCCCCGCCAAGCTGCTGAAGGAGAGAGGCTTTTTCGGCGCCATCGCCGGCTTCCTGGAGGGCGGATGGGAGGGAATGATCGCCGGCTGGCACGGCTACACCAGCCACGGAGCCCACGGCGTGGCCGTTGCTGCTGACCTGAAGAGCACCCAGGAGGCCATCAACAAGATCACCAAGAATCTGAATAGCCTGTCCGAGCTGGAGGTGAAGAATCTGCAGAGACTGTCCGGCGCCATGGATGAGCTGCACAATGAGATCCTGGAGCTGGATGAGAAGGTGGACGACCTGAGAGCCGACACAATCTCCAGCCAGATCGAGCTGGCCGTGCTGCTGAGCAACGAGGGCATCATCAATAGCGAGGATGAGCACCTGCTGGCCCTGGAGAGAAAGCTGAAGAAGATGCTGGGCCCTTCCGCCGTGGAGATCGGCAACGGCTGTTTTGAGACCAAGCACAAGTGCAACCAGACATGTCTGGATAGGATCGCCGCCGGCACCTTTGACGCCGGCGAGTTCTCCCTGCCCACATTCGATAGCCTGAATATCACCGCCGCCTCCCTGAACGATGACGGCCTGGATAATCACACCATCCTGCTGTACTACAGCACCGCCGCCAGCAGCCTGGCCGTGACACTGATGATCGCCATCTTCGTGGTGTACATGGTGAGCAGGGATAATGTGTCCTGTTCCATCTGTCTGTGATAGTAA

SEQ ID NO: 22 - protein, 1707 aa, synthetic construct
ATGAAGACAATCATCGCCCTGAGCAACATCCTGTGCCTGGTGTTCGCCCAGAAGATCCCTGGCAATGATAACAGCACCGCCACCCTGTGTCTGGGCCACCACGCCGTGCCTCAGGGCACAATCGTGAAGACAATCACAAACGACAGAATCGAGGTGACAAACGCCACCGAGCTGGTGCAGAATTCCTCCATCGGCGAGATCTGTGACAGCCCCCACCAGATCCTGGACGGCGGCAATTGTACCCTGATCGACGCCCTGCTGGGCGACCCCCAGTGTGATGGCTTCCAGAACAAGGAGTGGGATCTGTTTGTGGAGAGGTCCAGAGCCAACTCCAACTGCTACCCTTACGATGTGCCTGACTACGCCAGCCTGAGGAGCCTGGTGGCCAGCTCCGGCACCCTGGAGTTTAAGAACGAGTCCTTCAACTGGACCGGCGTGAAGCAGAATGGCACCTCCAGCGCCTGTATCAGAGGCAGCTCCAGCAGCTTCTTTAGCAGGCTGAACTGGCTGACAAGCCTGAATAACATCTACCCTGCCCAGAACGTGACCATGCCCAACAAGGAGCAGTTTGATAAGCTGTACATCTGGGGCGTGCACCACCCCAACACCGATAAGAATCAGATCTCCCTGTTCGCCCAGTCCTCCGGCAGAATCACAGTGAGCACAAAGAGAAGCCAGCAGGCCGTGATCCCCAACATCGGCTCCAGACCCAGAATCAGAGGCATCCCTAGCAGAATCTCCATCTACTGGACCATCGTGAAGCCTGGCGATATCCTGCTGATCAACTCCACCGGCAACCTGATCGCCCCTAGGGGCTACTTCAAGATCAGATCCGGCAAGAGCAGCATCATGAGGAGCGACGCCCCTATCGGCAAGTGTAAGTCCGAGTGCATCACACCCAATGGCTCCATCCCTAATGATAAGCCCTTTCAGAATGTGAACAGAATCACCTACGGCGCCTGCCCCAGGTACGTGAAGCAGAGCACCCTGAAGCTGGCCACAGGCATGAGAAATGTGCCCGAGAAGCAGACCAGGGGCATCTTCGGCGCCATCGCCGGCTTCATCGAGAATGGCTGGGAGGGCATGGTGGATGGCTGGTACGGCTTCAGACACCAGAACTCCGAGGGCAGGGGCCAGGCCGCTGATCTGAAGAGCACCCAGGCCGCCATCGACCAGATCAACGGCAAGCTGAACAGGCTGATCGGCAAGACAAATGAGAAGTTCCACCAGATCGAGAAGGAGTTCTCCGAGGTGGAGGGCAGAGTGCAGGACCTGGAGAAGTACGTGGAGGATACCAAGATCGACCTGTGGAGCTACAACGCCGAGCTGCTGGTGGCCCTGGAGAATCAGCACACCATCGACCTGACCGATTCCGAGATGAATAAGCTGTTCGAGAAGACCAAGAAGCAGCTGAGGGAGAATGCCGAGGATATGGGCAATGGCTGCTTTAAGATCTACCACAAGTGCGATAACGCCTGCATCGGCAGCATCAGAAATGAGACCTACGACCACCAGGTGTACAGAGATGAGGCCCTGAATAATAGGTTTCAGATCAAGGGCGTGGAGCTGAAGAGCGGCTACAAGGACTGGATCCTGTGGATCTCCTTTGCCATGTCCTGCTTTCTGCTGTGCATCGCCCTGCTGGGATTCATTATGTGGGCCTGCCAGAAGGGCAATATCAGATGTAACATCTGTATCTGATAGTAA

SEQ ID NO: 23 - protein, 1707 aa, synthetic construct
ATGAAGGCCATCATCGCCCTGAGCAACATCCTGTGTCTGGTGTTCGCCCAGAAGATCCCTGGCAACGATCAAAGCACCGCCACCCTGTGTCTGGGCCACCACGCCGTGCCCCAAGGCACAATCGTGAAGACCATCACCAATGACAGGATCGAGGTGACACAAGCCACAGAGCTGGTGCAGCAATCCAGCATCGGCAAGATCTGCAATAGCCCCCACCAGATCCTGGACGGCGGCCAATGCACCCTGATCGACGCCCTGCTGGGCGATCCCCAGTGCGATGGCTTTCAGAATAAGGAGTGGGATCTGTTCGTGGAGAGGTCCAGAGCCAATTCCTCCTGCTACCCCTACGACGTGCCTGACTACGCCTCCCTGAGAAGCCTGGTGGCCAGCAGCGGCACCCTGGAGTTTAAGAATGAGAGCTTCAACTGGACAGGCGTGAAGCAGAACGGCACATCCAGCGCCTGTAAGAGGGGCAGCTCCAGCAGCTTCTTCTCCAGACTGAATTGGCTGACCTCCCTGAACAACATCTACCCCGCCCAGAATGTGACAATGCCCAACAAGGAGCAGTTCGACAAGCTGTACATCTGGGGCGTGCACCACCCCGATACAGACAAGAACCAGTTCTCCCTGTTTGCCCAGTCCAGCGGCAGAATCACAGTGTCCACCAAGAGATCCCAGCAGGCCGTGATCCCCAACATCGGCAGCAGACCCAGAGTGAGGGATATCCCCAGCAGGATCTCCATCTACTGGACAATCGTGAAACCTGGCGACATCCTGCTGATCAACAGCACCGGCAATCTGATCGCCCCCAGGGGCTACTTTAAGATCAGGAGCGGCAAGAGCTCCATCATGAGGAGCGACGCCCCCATCGGCAAGTGCAAGTCCGAGTGTATCACCCCTAACGGCAGCATCCCCAACGATAAGCCTTTTCAGAATGTGAATAGGATCACCTACGGCGCCTGTCCTAGATACGTGAAGCAGAGCACCCTGAAGCTGGCCACCGGCATGAGAAATGTGCCTGAGAAGCAGACAAGAGGCATCTTCGGCGCCATCGCCGGCTTTATCGAGAACGGCTGGGAGGGCATGGTGGATGGCTGGTACGGCTTCAGGCACCAGAATTCCGAGGGCAGAGGCCAGGCCGCCGATCTGAAGAGCACCCAGGCCGCCATCGATCAGATCTCCGGCAAGCTGAACAGACTGATCGGCAAGACCAACGAGAAGTTCCACCAGATCGAGAAGGAGTTCAGCGAGGTGGAGGGCAGGGTGCAGGATCTGGAGAAGTACGTGGAGGACACAAAGATCGACCTGTGGAGCTACAATGCCGAGCTGCTGGTGGCCCTGGAGAACCAGCACACAATCGACCTGACAGATAGCGAGATGAATAAGCTGTTTGAGAAGACAAAGAAGCAGCTGAGGGAGAACGCCGAGGATATGGGCAATGGCTGTTTTAAGATCTACCACAAGTGCGACAATGCCTGTATCGGCAGCATCAGACAGGAGACATACGACCACAACGTGTACAGGGATGAGGCCCTGAACAATAGATTCCAGATCAAGGGCGTGGAGCTGAAGTCCGGCTACAAGGACTGGATCCTGTGGATCAGCTTTGCCATGTCCTGTTTCCTGCTGTGCATCGCCCTGCTGGGATTTATCATGTGGGCCTGTCAGAAGGGCAATATCAGATGCAACATCTGTATCTGATAGTAA

SEQ ID NO: 24 - protein, 1755 aa, synthetic construct
ATGAAGGCCATCATCGTGCTGCTGATGGTGGTGACCAGCAATGCCGATAGGATCTGCACAGGCATCACCTCCAGCAATAGCCCCCACGTGGTGAAGACAGCCACCCAGGGCGAGGTGAACGTGACCGGCGTGATCCCCCTGACAACCACACCTACAAAGTCCCACTTCGCCAACCTGAAGGGCACAGAGACAAGAGGCAAGCTGTGTCCCAAGTGCCTGAACTGCACAGACCTGGATGTGGCCCTGGGCAGACCCAAGTGTACCGGCAAGATCCCCAGCGCCAGGGTGTCCATCCTGCACGAGGTGAGACCTGTGACATCCGGCTGTTTCCCCATCATGCACGATAGGACCAAGATCAGGCAGCTGCCTAACCTGCTGAGAGGCTACGAGCACGTGAGGCTGAGCACACACAACGTGATCAATACAGAGGACGCCCCCGGCGGCCCCTACGAAATCGGAACAAGCGGCAGCTGTCTGAACATCACCAACGGCAAGGGCTTTTTCGCCACCATGGCCTGGGCCGTGCCTAAGAATAAGACCGCCACCAATCCCCTGACCATCGAGGTGCCCTACATCTGCACCGAGGAGGAGGATCAGATCACCGTGTGGGGCTTTCACAGCGACGATGAGACCCAGATGGCCAGGCTGTACGGCGACTCCAAGCCTCAGAAGTTTACAAGCTCCGCCAACGGCGTGACCACCCACTACGTGTCCCAGATCGGCGGCTTTCCCAATCAGACAGAGGACGGCGGCCTGCCTCAGTCCGGCAGAATCGTGGTGGATTACATGGTGCAGAAGTCCGGCAAGACAGGCACCATCACCTACCAGAGAGGCATCCTGCTGCCCCAGAAGGTGTGGTGCGCCAGCGGCAAGTCCAAGGTCATTAAGGGCTCCCTGCCCCTGATCGGCGAGGCCGATTGCCTGCACGAGAAGTACGGCGGCCTGCAGAAGAGCAAGCCTTACTACACCGGCGAGCACGCCAAGGCCATCGGCAACTGTCCCATCTGGGTGAAGACACCTCTGAAGCTGGCCCAGGGCACAAAGTACAGACCTCCCGCCAAGCTGCTGAAGGAGAGAGGCTTTTTCGGCGCCATCGCCGGCTTTCTGGAGGGCGGATGGGAGGGCATGATCGCCGGCTGGCACGGCTACACCAGCCACGGAGCCCACGGCGTGGCCGTTGCTGCTGATCTGAAGAGCACCCAGGAGGCCATCAATAAGATCACAAAGAACCTGAACTCCCTGAGCGAGCTGGAGGTGAAGAATCTGCAGAGGCTGAGCGGCGCCATGGATGAGCTGCACAACGAGATCCTGGAGCTGGACGAGAAGGTGGACGACCTGAGGGCCGACACAATCAGCAGCCAGATCGAGCTGGCCGTGCTGCTGAGCAACGAGGGCATCATCAATAGCGAGGATGAGCACCTGCTGGCCCTGGAGAGGAAGCTGAAGAAGATGCTGGGCCCCAGCGCCGTGGAGATCGGCAACGGATGTTTTGAGACAAAGCACAAGTGTCAGCAGACATGCCTGGACAGGATCGCCGCCGGCACATTTGATGCCGGCGAGTTTTCCCTGCCCACATTCGATAGCCTGCAGATCACCGCCGCCAGCCTGAACGATGACGGCCTGGATCAGCACACAATCCTGCTGTACTACTCCACCGCCGCCTCCAGCCTGGCCGTGACACTGATGATCGCCATCTTCGTGGTGTACATGGTGTCCAGGGATAATGTGAGCTGTTCCATCTGCCTGTGATAGTAA

SEQ ID NO: 25 - protein, 1755 aa, synthetic construct
ATGAAGGCCATCATCGTGCTGCTGATGGTGGTGACATCCAATGCCGACAGAATCTGCACAGGCATCACCAGCTCCAACAGCCCCCACGTGGTGAAGACAGCCACACAGGGCGAGGTGAACGTGACCGGCGTGATCCCTCTGACAACCACACCTACCAAGTCCCACTTCGCCAATCTGAAGGGCACCGAGACAAGGGGCAAGCTGTGCCCTAAGTGTCTGAACTGTACCGATCTGGATGTGGCCCTGGGCAGGCCTAAGTGTACCGGCAAGATCCCTAGCGCCAGGGTGAGCATCCTGCACGAGGTGAGACCTGTGACAAGCGGCTGCTTCCCCATCATGCACGACAGAACCAAGATCAGGCAGCTGCCCAATCTGCTGAGAGGCTACGAGCACGTGAGACTGTCCACCCACAACGTGATCAATACCGAGGATGCCCCTGGCGGCCCTTACGAGATCGGCACCAGCGGCTCCTGTCTGAATATCACAAATGGCAAGGGCTTCTTTGCCACCATGGCCTGGGCCGTGCCTAAGAACAAGACCGCCACCAACCCTCTGACCATCGAGGTGCCCTACATCTGTACAGAGGAGGAGGATCAGATCACCGTGTGGGGCTTTCACTCCGATGACGAGACCCAGATGGCCAGACTGTACGGCGACAGCAAGCCCCAGAAGTTCACCTCCTCCGCCAACGGCGTGACCACACACTACGTGTCCCAGATCGGCGGCTTCCCTAACCAGACCGAGGACGGCGGCCTGCCTCAGAGCGGAAGGATCGTGGTGGATTACATGGTGCAGAAGTCCGGCAAGACAGGCACCATCACATACCAGAGGGGCATCCTGCTGCCTCAGAAGGTGTGGTGCGCCTCCGGCAAGAGCAAGGTCATTAAGGGCAGCCTGCCTCTGATCGGCGAGGCCGACTGCCTGCACGAGAAGTACGGCGGCCTGAATAAGAGCAAGCCCTACTACACCGGCGAGCACGCCAAGGCCATCGGCAACTGTCCCATCTGGGTGAAGACACCTCTGAAGCTGGCCAATGGCACCAAGTACAGACCCCCCGCCAAGCTGCTGAAGGAGAGAGGCTTTTTCGGCGCCATCGCCGGCTTCCTGGAGGGCGGATGGGAGGGAATGATCGCCGGCTGGCACGGCTACACCAGCCACGGAGCCCACGGCGTGGCCGTTGCTGCTGACCTGAAGAGCACCCAGGAGGCCATCAACAAGATCACCAAGAATCTGAATAGCCTGTCCGAGCTGGAGGTGAAGAATCTGCAGAGACTGTCCGGCGCCATGGATGAGCTGCACAATGAGATCCTGGAGCTGGATGAGAAGGTGGACGACCTGAGAGCCGACACAATCTCCAGCCAGATCGAGCTGGCCGTGCTGCTGAGCAACGAGGGCATCATCAATAGCGAGGATGAGCACCTGCTGGCCCTGGAGAGAAAGCTGAAGAAGATGCTGGGCCCTTCCGCCGTGGAGATCGGCAACGGCTGTTTTGAGACCAAGCACAAGTGCCAACAGACATGTCTGGATAGGATCGCCGCCGGCACCTTTGACGCCGGCGAGTTCTCCCTGCCCACATTCGATAGCCTGCAAATCACCGCCGCCTCCCTGAACGATGACGGCCTGGATCAACACACCATCCTGCTGTACTACAGCACCGCCGCCAGCAGCCTGGCCGTGACACTGATGATCGCCATCTTCGTGGTGTACATGGTGAGCAGGGATAATGTGTCCTGTTCCATCTGTCTGTGATAGTAA

SEQ ID NO: 26 - protein, 4 aa, synthetic construct
QSRG

SEQ ID NO: 27 - protein, 4 aa, synthetic construct
QTRG

SEQ ID NO: 28 - protein, 8 aa, synthetic construct
RRKKRGLF

SEQ ID NO: 29 - protein, 4 aa, synthetic construct
KERG

SEQ ID NO: 30 - protein, 14 aa, synthetic construct
GLFGAIAGFIEGGW

SEQ ID NO: 31 - protein, 14 aa, synthetic construct
GIFGAIAGFIEGGW

SEQ ID NO: 32 - protein, 17 aa, synthetic construct
GLFGAIAGFIEGGWTGM

(19) United States
(12) Patent Application Publication
(10) Pub. No.: US 2026/0158130 A1
(43) Pub. Date: Jun. 11, 2026

(54) LOW-SUGAR INFLUENZA VACCINE AND METHODS THEREOF

(71) Applicant: Rock BioMedical, Inc., Taipei City (TW)

(72) Inventors: Chung-Yi WU, Taipei City (TW); Chi-Huey Wong, Rancho Santa Fe, CA (US); Jeng-Shin LEE, Lincoln, MA (US)

(21) Appl. No.: 19/409,259

(22) Filed: Dec. 4, 2025

Related U.S. Application Data
(60) Provisional application No. 63/727,790, filed on Dec. 4, 2024.

(51) Int. Cl. A61K 39/145; A61K 39/00; A61P 37/04; C07K 14/005

(52) U.S. Cl. CPC A61K 39/145; A61P 37/04; C07K 14/005; A61K 2039/53; A61K 2039/6018; C12N 2760/16121; C12N 2760/16134; C12N 2760/16171; C12N 2760/16222; C12N 2760/16234; C12N 2760/16271

(57) ABSTRACT

The present disclosure relates to a low glycosylated influenza hemagglutinin (HA) protein and a vaccine designed to express the HA protein in vivo. The present disclosure also teaches a method for generating an immune response by utilizing the low glycosylated HA protein, which provides a broader protection across different influenza strains or lineages.

### CROSS-REFERENCE TO RELATED APPLICATIONS

This application claims the benefit of priority of U.S. Provisional Patent Applications No. 63/727,790, filed on Dec. 4, 2024. The entirety of the aforementioned application is incorporated herein by reference.

### SEQUENCE LISTING

The instant disclosure contains a Sequence Listing which is submitted electronically in .xml format and is hereby incorporated by reference in its entirety. The .xml copy, created on Dec. 4, 2025, is named “A1000-01500US_20251204_SeqListing.xml” and is 49,777 bytes in size.

### FIELD

The instant disclosure relates to vaccines for preventing influenza virus infection, especially to an mRNA vaccine configured to express hemagglutinins in vivo to prevent influenza virus infection.

### BACKGROUND

Seasonal influenza (“flu”) is an acute respiratory infection caused by influenza viruses that circulate globally. These viruses belong to the Orthomyxoviridae family, which includes four species: influenza A virus (IAV), influenza B virus (IBV), influenza C virus (ICV), and influenza D virus (IDV). Among these, IAV and IBV are primarily responsible for annual epidemics. IAV exhibits extensive genetic diversity and cross-species transmission, driven by antigenic drift and shift across its 18 hemagglutinin (HA) subtypes and 11 neuraminidase (NA) subtypes. In contrast, IBV consists of a single virus type with two antigenically and genetically distinct lineages, represented by the prototype strains B/Victoria/2/1987 (Victoria lineage) and B/Yamagata/16/1988 (Yamagata lineage).

Seasonal influenza epidemics cause up to 650,000 deaths worldwide each year. Moreover, the rapid evolution and cross-species spillover of highly pathogenic influenza viruses have heightened global concern that another influenza pandemic is inevitable. Accordingly, there is an increasing need for vaccines with improved efficacy.

### BRIEF SUMMARY

One aspect of the present disclosure is an isolated nucleic acid, which encodes a recombinant influenza hemagglutinin (HA) comprising a signal peptide, an HA1 subunit, and an HA2 subunit, wherein the influenza HA comprises at least one glycosylation site, and the HA2 subunit is devoid of a glycosylation site.

Another aspect of the present disclosure is an isolated nucleic acid, encoding a recombinant influenza hemagglutinin (HA), wherein the influenza hemagglutinin (HA) comprises an amino acid sequence at least 80%, 85%, 90%, 91%, 92%, 93%, 94%, 95%, 96%, 97%, 98%, or 99%, or 100% identical to SEQ ID NO: 3, SEQ ID NO: 6, SEQ ID NO: 9, SEQ ID NO: 11, SEQ ID NO: 16, or SEQ ID NO: 18.

Another aspect of the present disclosure is an expression vector, comprising the isolated nucleic acid of the present disclosure.

Another aspect of the present disclosure is an immunogenic composition comprising the expression vector of the present disclosure.

Another aspect of the present disclosure is a multiple valent immunogenic composition comprising a first isolated nucleic acid, encoding a first recombinant influenza hemagglutinin (HA), which comprises an amino acid sequence at least 80%, 85%, 90%, 91%, 92%, 93%, 94%, 95%, 96%, 97%, 98%, or 99%, or 100% identical to SEQ ID NO: 6, provided that each of the 40^{th}, the 293^{rd}, the 304^{th}, and the 498^{th} amino acids thereof does not form a glycosylation site, and the 27^{th} amino acid thereof forms a glycosylation site; a second isolated nucleic acid, encoding a second recombinant influenza hemagglutinin (HA), which comprises an amino acid sequence at least 80%, 85%, 90%, 91%, 92%, 93%, 94%, 95%, 96%, 97%, 98%, or 99%, or 100% identical to SEQ ID NO: X5, provided that each of the 24^{th}, the 38^{th}, the 54^{th}, the 61^{st}, the 79^{th}, the 301^{st}, and the 499^{th} amino acids thereof does not form a glycosylation site; a third isolated nucleic acid, encoding a third recombinant influenza hemagglutinin (HA), which comprises an amino acid sequence at least 80%, 85%, 90%, 91%, 92%, 93%, 94%, 95%, 96%, 97%, 98%, or 99%, or 100% identical to SEQ ID NO: 3, provided that each of the 318^{th}, the 347^{th}, the 506^{th}, the 532^{nd}, and the 545^{th} amino acids does not form a glycosylation site, and the 40^{th} amino acid forms a glycosylation site; and a fourth isolated nucleic acid, encoding a fourth recombinant influenza hemagglutinin (HA), which comprises an amino acid sequence at least 80%, 85%, 90%, 91%, 92%, 93%, 94%, 95%, 96%, 97%, 98%, or 99%, or 100% identical to SEQ ID NO: Y4, provided that each of the 504^{th}, the 530^{th}, and the 543^{rd} amino acid does not form a glycosylation site, and each of the 40^{th}, the 316^{th}, and the 345^{th}, amino acids forms a glycosylation site.

Another aspect of the present disclosure is a method for generating an immune response against influenza virus infection, comprising administering the isolated nucleic acid of the present disclosure to a subject in need at an effective amount.

Another aspect of the present disclosure is a recombinant influenza hemagglutinin, comprising an amino acid sequence at least 80%, 85%, 90%, 91%, 92%, 93%, 94%, 95%, 96%, 97%, 98%, or 99%, or 100% identical to SEQ ID NO: 3, SEQ ID NO: 6, SEQ ID NO: 9, SEQ ID NO: 11, SEQ ID NO: 16, or SEQ ID NO: 18.

### BRIEF DESCRIPTION OF THE DRAWINGS

FIG. 1 provides a graphical representation of the 3-dimensional structure of an influenza A hemagglutinin (HA) obtained from the Protein Data Bank (PDB). The five glycosylation sites near the conserved surface epitopes (circled) of the stem region are labeled with the Asn residues for glycosylation indicated.

FIG. 2 provides a graphical representation showing the IgG titers induced by the exemplary mRNA vaccines against a B/Phuket/3073/2013 strain. The tested vaccines include (1) a wild-type vaccine encoding an influenza HA of the vaccine strain (B/Phuket/3073/2013; “WT”), (2) a deglycosylation vaccine encoding a recombinant influenza HA of the vaccine strain with all glycosylation site in the stem region removed (“deg-stem”), and (3) a deglycosylation vaccine encoding a recombinant influenza HA of the vaccine strain with all glycosylation site in the stem region removed except for the N40 site (“deg-stem+N40”). PBS serves as a negative control. The IgG titer was reported as the OD values at 450 nm.

FIG. 3A provides a graphical representation showing cross-binding IgG titers induced by the exemplary mRNA vaccines against a B/Austria/1359417/2021 strain. The tested vaccines include the same exemplary vaccines as described in FIG. 2. The IgG titer was presented as the OD values detected at 450 nm.

FIG. 3B provides a graphical representation showing cross-binding IgG titers induced by the exemplary mRNA vaccines against a B/Maryland/1959strain. The tested vaccines include the same exemplary vaccines as described in FIG. 2. The IgG titer was presented as the OD values detected at 450 nm.

FIG. 4 provides a graphical representation showing the IgG titers induced by the exemplary mRNA vaccines against a A/Victoria/4897/2022 strain. The tested vaccines include (1) a wild-type vaccine encoding an influenza HA of the vaccine strain (A/Victoria/4897/2022 strain; “WT”), (2) a deglycosylation vaccine encoding a recombinant influenza HA of the vaccine strain with all glycosylation site in the stem region removed (“deg-stem”), and (3) a deglycosylation vaccine encoding a recombinant influenza HA of the vaccine strain with all glycosylation site in the stem region removed except for the N27 site (“deg-stem+N27/28”). PBS serves as a negative control. The IgG titer was reported as the OD at 450 nm.

FIG. 5A provides a graphical representation showing the cross-binding IgG titers induced by the exemplary mRNA vaccines against a A/California/062009 strain. The tested vaccines include the same exemplary vaccines, as described in FIG. 4. IgG titer was reported as OD values at 450 nm.

FIG. 5B provides a graphical representation showing the cross-binding IgG titers induced by the exemplary mRNA vaccines against a A/Pavia/65/2016 strain. The tested vaccines include the same exemplary vaccines, as described in FIG. 4. IgG titer was reported as OD values at 450 nm.

FIG. 5C provides a graphical representation showing the cross-binding IgG titers induced by the exemplary mRNA vaccines against a A/Cambodia/NPH230032/2023 (H5N1) strain. The tested vaccines include the same exemplary vaccines as described in FIG. 4. The IgG titer was reported as OD values at 450 nm.

FIG. 6 provides a graphical representation showing the T cell response induced by the exemplary mRNA vaccines against A/Victoria/4897/2022 (H1N1) strain. The tested vaccines include the same exemplary vaccines as described in FIG. 4.

FIG. 7 provides a graphical representation showing the T cell response induced by the exemplary mRNA vaccines against a A/Califoria/07/2009 strain. The tested vaccines include the same exemplary vaccines as described in FIG. 4.

FIG. 8 provides a graphical representation showing the T cell response induced by the exemplary mRNA vaccines against A/Cambodia/NPH230032/2023 strain. The tested vaccines include the same exemplary vaccines as described in FIG. 4.

FIG. 9 provides a graphical representation showing the IgG titers induced by the exemplary mRNA vaccines against a A/Thailand/8/2022 (H3N2) strain. The tested vaccines include (1) a wild-type vaccine encoding an influenza HA of the vaccine strain (A/Thailand/8/2022 strain; “WT”), (2) a deglycosylation vaccine encoding a recombinant influenza HA of the vaccine strain with all glycosylation site in the stem region removed (“deg-stem”), and (3) a deglycosylation vaccine encoding a recombinant influenza HA of the vaccine strain with all glycosylation site in the stem region removed except for the N24 site (“deg-stem+N24”). PBS serves as a negative control. The IgG titer was reported as the OD at 450 nm.

FIG. 10 provides a graphical representation showing the cross-binding IgG titers induced by the exemplary mRNA vaccines against a A/Victoria/361/2001 strain. The tested vaccines include the same exemplary vaccines as described in FIG. 9. The IgG titer was presented as the OD values detected at 450 nm.

FIG. 11 provides a graphical representation showing the cross-binding IgG titers induced by the exemplary mRNA vaccines against a A/swine/Colorado/A01203748/2012 strain. The tested vaccines include the same exemplary vaccines as described in FIG. 9. The IgG titer was presented as the OD values detected at 450 nm.

FIG. 12 provides a graphical representation of the cross-binding IgG titers induced by the exemplary mRNA vaccines against a A/Shanghai/2/2013 strain. The tested vaccines include the same exemplary vaccines as described in FIG. 9. The IgG titer was presented as the OD values detected at 450 nm.

FIG. 13 provides a graphical representation showing the T cell response induced by the exemplary mRNA vaccines against a A/Thailand/8/2022 strain The tested vaccines include the same exemplary vaccines as described in FIG. 9.

FIG. 14 provides a graphical representation showing the T cell response induced by the exemplary mRNA vaccines against A/Victoria/361/2001 strain. The tested vaccines include the same exemplary vaccines as described in FIG. 9.

FIG. 15 provides a graphical representation showing the T cell response induced by the exemplary mRNA vaccines against A/swine/Colorado/A01203748/2012 strain. The tested vaccines include the same exemplary vaccines as described in FIG. 9. The IgG titer was presented as the OD values detected at 450 nm.

FIG. 16 provides a graphical representation showing the IgG titers induced by the exemplary mRNA vaccines against a A/Thailand/8/2022 (H3N2) strain. The tested vaccines include (1) a wild-type vaccine encoding an influenza HA of the vaccine strain (A/Thailand/8/2022 strain; “WT”), (2) a deglycosylation vaccine encoding a recombinant influenza HA of the vaccine strain with all glycosylation site in the stem region removed and with additional glycosylation sites at N54, N61, and N79 removed (“deg-stem-N54-61-79”), (3) a deglycosylation vaccine encoding a recombinant influenza HA of the vaccine strain with all glycosylation site in the stem region removed and with additional glycosylation sites at N54, N61, N79, N262, and N301 removed (“deg-stem-N54-61-79-262-301”), (4) a deglycosylation vaccine encoding a recombinant influenza HA of the vaccine strain with all glycosylation site in the stem region removed and with additional glycosylation sites at N54, N61, N79, and N301 removed (“deg-stem-N54-61-79-301”), (5) a deglycosylation vaccine encoding a recombinant influenza HA of the vaccine strain with all glycosylation site in the stem region removed and with additional glycosylation sites at N61 and N301 removed (“deg-stem-N61-301”), and (6) a deglycosylation vaccine encoding a recombinant influenza HA of the vaccine strain with all glycosylation site in the stem region removed and with additional glycosylation sites at N262 and N301 removed (“deg-stem-N262-301”). PBS serves as a negative control. The IgG titer was reported as the OD at 450 nm.

FIG. 17 provides a graphical representation showing the cross-binding IgG titers induced by the exemplary mRNA vaccines against a A/Shanghai/2/2013 (H7N9) strain. The tested vaccines include the same exemplary vaccines as described in FIG. 16. The IgG titer was presented as the OD values detected at 450 nm.

FIG. 18 provides a graphical representation showing the percentage of responsive IgG in all IgG induced by the exemplary mRNA vaccines against a A/Shanghai/2/2013 (H7N9) strain. The tested vaccines include the same exemplary vaccines as described in FIG. 16. The IgG titer was presented as the OD values detected at 450 nm.

FIG. 19 provides a graphical representation showing the level of cross-binding IgG induced by the WT vaccine, deg-stem vaccine, and the deg-stem+N40 vaccine. The Y-axis indicates the endpoint IgG showing cross-activities against a B/Colorado/06/2017 strain.

FIG. 20 provides a graphical representation showing the T cell response against a B/Phuket/3073/2013 strain induced by the exemplary mRNA vaccines of the present disclosure. The test vaccines include: (1) a wild-type (“WT”) vaccine encoding the Yamagata B/Phuket/3073/2013 HA, and (2) two deglycosylation vaccines. One deglycosylation vaccine encodes the same HA with all glycosylation sites in the stem region removed (“deg-stem”), while the other encodes the HA with all stem-region glycosylation sites removed except for the N40 site (“deg-stem+40”). PBS served as a negative control. The IgG titer was reported as the OD at 450 nm.

FIG. 21 provides a graphical representation showing the T cell response against a B/Colorado/06/2017 strain induced by the exemplary mRNA vaccines of the present disclosure. The tested vaccines include the same exemplary vaccines as described in FIG. 20. The IgG titer was presented as the OD values detected at 450 nm.

FIG. 22 provides a graphical representation showing the T cell response against a B/Maryland/1959 strain induced by the exemplary mRNA vaccines of the present disclosure. The tested vaccines include the same exemplary vaccines as described in FIG. 20. The titer was presented as the OD values detected at 450 nm.

FIG. 23 provides a graphical representation showing the IgG titers against a B/Austria/1359417/2021 strain induced by the exemplary mRNA vaccines of the present disclosure. The test vaccines include: (1) a wild-type (“WT”) vaccine encoding the IBV Victoria B/Austria/1359417/2021 HA strain, (2) a deglycosylation vaccine encoding a B/Austria/1359417/2021 HA with all glycosylation sites in the HA2 subunit removed (“deg-HA2”), (3) a deglycosylation vaccine encoding a B/Austria/1359417/2021 HA with all stem-region glycosylation sites removed except for the N40 and N316 sites (“deg-stem+N40,316”). PBS served as a negative control. The IgG titer was reported as the OD at 450 nm.

FIG. 24 provides a graphical representation showing the IgG titers against a B/Maryland/1959 strain induced by the exemplary mRNA vaccines of the present disclosure. The tested vaccines include the same exemplary vaccines as described in FIG. 23. The IgG titer was presented as the OD values detected at 450 nm.

FIG. 25A to FIG. 25B provide a graphical representation of the FACS analysis results demonstrating the efficacy of the exemplary inventive embodiments. The experiments demonstrated the uptake of the novel nano-delivery formulations targeting BDMCs, according to embodiments of the present disclosure, compared to traditional LNPs. The FITC+ values shown in the figures are fluorescent intensities in arbitrary units (A.U.). FIG. 25A shows the BDMC cells uptake of a negative control group. FIG. 25B shows the non-BDMC cells uptake of a negative control group. FIG. 25C shows the BDMC cells uptake of a positive control group treated with FITC-labeled LNP. FIG. 25D shows the non-BDMC cells uptake of a positive control group treated with FITC-labeled LNP. FIG. 25E shows the BDMC cells uptake of an experiment group treated with FITC-labeled Compound 24-LNP according to an example of the present disclosure. FIG. 25F shows the non-BDMC cells uptake of a positive control group treated with FITC-labeled Compound 24-LNP according to an example of the present disclosure. FIG. 25G shows the BDMC cells uptake of an experiment group treated with FITC-labeled Compound 25-LNP according to an example of the present disclosure. FIG. 25H shows the non-BDMC cells uptake of a positive control group treated with FITC-labeled Compound 25-LNP according to an example of the present disclosure.

FIG. 26A to FIG. 26L provide a graphical representation of the FACS analysis results demonstrating the efficacy of the exemplary novel dendritic cell-targeting formulations of the present disclosure. The FITC+ values shown in the figures are fluorescent intensities in arbitrary units (A.U.). FIG. 26A shows the results of non-LNP treated negative control group of dendritic cells (DC). FIG. 26B shows the results of non-LNP treated negative control group of B cells. FIG. 26C shows the results of non-LNP treated negative control group of T cells. FIG. 26D shows the results of a positive control group of dendritic cells (DC) treated with FITC-labelled LNPs. FIG. 26E shows the results of a positive control group of B cells treated with FITC-labelled LNPs. FIG. 26F shows the results of a positive control group of T cells treated with FITC-labelled LNPs. FIG. 26G shows dendritic cell (DC) uptake of the exemplary novel dendritic targeting formulations of Compound 24-LNPs according to embodiments of the present disclosure. FIG. 26H shows B cell uptake of the exemplary novel dendritic targeting formulations of Compound 24-LNPs according to embodiments of the present disclosure. FIG. 26I T cell uptake of the exemplary novel dendritic targeting formulations of Compound 24-LNPs according to embodiments of the present disclosure. FIG. 26J shows dendritic cell (DC) uptake of the exemplary novel dendritic targeting formulations of Compound 25-LNPs according to embodiments of the present disclosure. FIG. 26K B cell uptake of the exemplary novel dendritic targeting formulations of Compound 25-LNPs according to embodiments of the present disclosure. FIG. 26L T cell uptake of the exemplary novel dendritic targeting formulations of Compound 25-LNPs according to embodiments of the present disclosure.

FIG. 27A to FIG. 27G provide a graphical representation showing the FACS analysis results demonstrating the efficacy of the exemplary novel dendritic targeting formulations of the present disclosure. The experiments showed the uptake of the dendritic cell-targeting formulations according to embodiments of the present disclosure targeting BDMCs compared with conventional LNPs. The FITC+ values shown in the figures are fluorescent intensities in arbitrary units (A.U.). 22-LNP represents the targeting formulation made using compound 22 of the present disclosure, and the percentage in parentheses indicates the molar ratio of compound 22. Likewise, 23-LNP represents the targeting formulation made using compound 23 of the present disclosure, and the percentage in parentheses indicates the molar ratio of compound 23. The negative control was an LNP without using the present disclosure's novel targeting compound/formulation (i.e., “traditional LNP” as described herein). FIG. 27A shows negative control. FIG. 27B shows the experimental group treated with Compound 22-LNPs (22-LNP) comprising 5 mol % Compound 22. FIG. 27C shows the experimental group treated with Compound 22-LNPs (22-LNP) comprising 10 mol % Compound 22. FIG. 27D shows the experimental group treated with Compound 22-LNPs (22-LNP) comprising 20 mol % Compound 22. FIG. 27E shows the experimental group treated with Compound 23-LNPs (23-LNP) comprising 5 mol % Compound 23. FIG. 27F shows the experimental group treated with Compound 23-LNPs (23-LNP) comprising 10 mol % Compound 23. FIG. 27G shows the experimental group treated with Compound 23-LNPs (23-LNP) comprising 20 mol % Compound 23.

FIG. 28A to FIG. 28G provide a graphical representation showing the FACS analysis results demonstrating the efficacy of the exemplary novel dendritic cell-targeting formulations of the present disclosure. The experiments showed the transfection of the targeting formulations BDMCs compared with LNPs without the compound of the present disclosure. The FITC+ values shown in the figures are fluorescent intensities in arbitrary units (A.U.). 22-LNP represents the targeting formulation made using compound 22 of the present disclosure, and the percentage in parentheses indicates the molar ratio of compound 22. Likewise, 23-LNP represents the targeting formulation made using compound 23 of the present disclosure, and the percentage in parentheses indicates the molar ratio of compound 23. The negative control was an LNP formed without using the present disclosure's novel targeting compound/formulation (i.e., “traditional LNP” as described herein). FIG. 28A shows negative control. FIG. 28B shows the experimental group treated with Compound 22-LNPs (22-LNP) comprising 5 mol % Compound 22. FIG. 28C shows the experimental group treated with Compound 22-LNPs (22-LNP) comprising 10 mol % Compound 22. FIG. 28D shows the experimental group treated with Compound 22-LNPs (22-LNP) comprising 20 mol % Compound 22. FIG. 28E shows the experimental group treated with Compound 23-LNPs (23-LNP) comprising 5 mol % Compound 23. FIG. 28F shows the experimental group treated with Compound 23-LNPs (23-LNP) comprising 10 mol % Compound 23. FIG. 28G shows the experimental group treated with Compound 23-LNPs (23-LNP) comprising 20 mol % Compound 23.

FIG. 29A to FIG. 29C provide a graphical representation demonstrating the targeting efficacy and specificity of the exemplary formulation based on the distribution of the targeting LNPs in an animal model. The LNPs carried an mRNA configured to encode a luciferase in the targeted cells of the tested animals. The assay would generate detectable luminescence if the LNPs successfully transfect cells and the cells express the luciferase. The results clearly showed tissue-specific targeting of spleen and lymph tissue by the exemplary targeting formulation, thereby providing supporting evidence of immune cell (e.g., dendritic cell) specificity. FIG. 29A shows the result of a positive control LNP. FIG. 29B shows the result of the Compound 22-LNP. FIG. 29C shows the result of the Compound 12-LNP.

FIG. 30 shows the ¹H NMR spectrum of compound 12 of the present disclosure.

FIG. 31 shows the ¹³C NMR spectrum of compound 12 of the present disclosure.

FIG. 32A and FIG. 32B present bar charts showing the IFNγ (FIG. 32A) and IL-4 (FIG. 32B) induction by the exemplary targeting LNPs formulation according to an embodiment of the present disclosure in vivo. The sera were collected from experimental animals 2 hours, 24 hours, and 48 hours after administration.

FIG. 33 shows a graphical representation demonstrating the neutralization inhibitory effects of the exemplary LNPs of the present disclosure compared to control LNPs. The neutralization inhibition was evaluated against different dilution factors to show the difference between samples.

FIG. 34 provides a bar chart demonstrating that the exemplary LNPs, according to the present disclosure, carrying mRNA encoding the wild-type spike protein, were able to invoke IgG production in vivo against the wild-type virus and the Delta and Omicron strains thereof.

FIG. 35A and FIG. 35B show a comparative bar chart demonstrating the IgG titer (FIG. 35A) and neutralization ability (FIG. 35B) induced respectively by commercially available LNPs vs. LNP formulation formulated based on, and/or constructed with, exemplary compounds according to embodiments of the present disclosure. Both LNPs carried mRNA encoding a wild-type spike protein.

FIG. 36 shows the LC-MS spectrum of compound 21 of the present disclosure.

### DETAILED DESCRIPTION

Influenza hemagglutinin (HA) is a homotrimeric glycoprotein expressed on the viral surface and is essential for viral infectivity. Each monomer of the trimer comprises a signal peptide and two subunits, HA1 and HA2. The HA1 subunit forms the membrane-distal globular head, which contains the receptor-binding site (RBS) and the highly variable immunodominant regions surrounding the RBS. During host-pathogen interaction, HA1 binds to sialylated receptors on the epithelial surface of host cells, triggering endocytosis and facilitating viral entry. In contrast, the HA2 subunit, together with the N- and C-terminal regions of HA1, forms the highly conserved membrane-proximal stem. Because HA is the major surface protein of the influenza virus and is indispensable for viral infectivity, it remains the primary target for antiviral therapeutics and vaccine development.

The persistent threat of seasonal influenza outbreaks and the ongoing risk of influenza pandemics highlight the need for vaccines capable of providing protection across a broad range of influenza strains. Messenger RNA (mRNA) vaccines have demonstrated strong potential for rapid adaptation to emerging viral variants and for eliciting robust immune responses. Consequently, mRNA vaccines designed to express recombinant HA proteins in vivo appear well suited to meet this demand. However, conventional vaccines generally do not provide sufficiently broad-spectrum protection.

### Broad Spectrum Vaccines of Low-Sugar Hemagglutinin

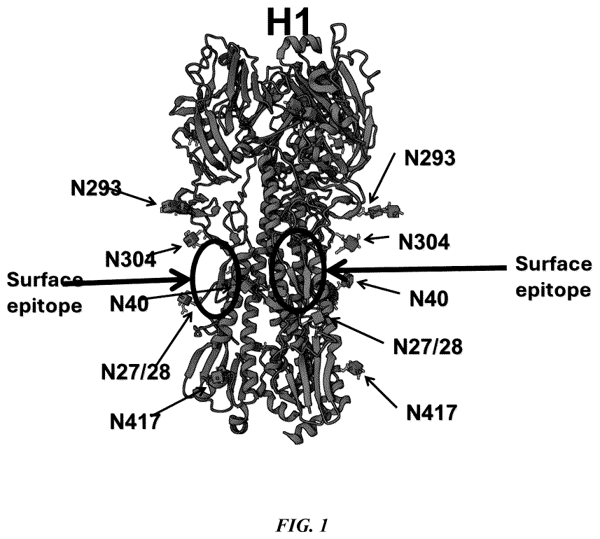

To develop a influenza, flu, vaccine with a broader spectrum of protection, the present disclosure compares the hemagglutinins of A/Victoria/4897/2022 (H1N1), A/Thailand/8/2022 (H3N2), B/Austria/1359417/2021 (B/Victoria lineage), and B/Phuket/3073/2013 (B/Yamagata lineage), based on the 2024-2025 Northern Hemisphere influenza vaccine composition recommended by the World Health Organization (WHO). The present disclosure finds that, although the hemagglutinin (HA) sequences of influenza A viruses and influenza B viruses exhibit limited overall similarity, the surface epitopes located in the stem region of both IAV and IBV display notable structural similarity. The region containing the conserved surface epitopes is encircled by a few glycosylation sites, e.g., five glycosylation sites in the H1 subtype (FIG. 1). Without wishing to be bound by theory, the present disclosure believes that the glycans conjugated to those glycosylation sites shield the conversed surface epitopes. The present disclosure demonstrates that removing those glycosylation sites reveals the conserved surface epitopes, thereby increasing the chances for the host immune system to recognize the flu viruses more effectively and broadly. Therefore, a vaccine using the deglycosylated version of Ha as an immunogen or encoding the deglycosylated version of HA is believed to generate a broader spectrum of protection.

Nevertheless, glycans are believed to contribute, and some might be essential, to the structural integrity and proper folding of the hemagglutinin (HA). Removing glycosylation sites randomly poses a risk of affecting the overall structure of the HA. Consequently, the deglycosylated HA might not fold into a proper structure to induce an immune response that can recognize the wild-type, without deglycosylation, viruses in the natural environment. The present disclosure demonstrates that certain glycosylation sites show essential role as removing those sites resulting in a deglycosylated HA that does not trigger a proper IgG response, nor does it initiate a proper T cell response. Furthermore, according to the data of mRNA vaccines encoding the deglycosylation HAs of the present disclosure, keeping some essential glycosylation sites not only ensures the structural integrity of the HA but also increase cross-activities of the vaccines in recognizing virus variants other than the vaccine strain.

### HA2 Deglycosylation and Stem Region Deglycosylation

In the first aspect of the present disclosure, an isolated nucleic acid is provided. The isolated nucleic acid encodes a recombinant influenza hemagglutinin (HA) comprising a signal peptide, an HA1 subunit, and an HA2 subunit, wherein the influenza HA comprises at least one glycosylation site, and the HA2 subunit is devoid of a glycosylation site. As used herein, a “nucleic acid” can be DNA or RNA (e.g., messenger RNA).

In some embodiments, the recombinant influenza HA has at least one glycosylation site, provided that the HA2 subunit is devoid of a glycosylation site. For example, in some embodiments, the HA1 subunit comprises a glycosylation site. In some embodiments, the stem region of the recombinant influenza HA is devoid of a glycosylation site, while other parts of the recombinant influenza HA can or does have a glycosylation site. Yet in some embodiments, a HA1 N-terminal loop motif, which is considered as part of the stem region, comprises a glycosylation site, but the rest of the stem region is devoid of a glycosylation site.

The present disclosure discovered that the glycan shield covering the influenza HA plays a critical role in host-pathogen recognition and surprisedly discovered that the absence of the glycan shield (i.e., “low-sugar” HA), especially the HA2 subunit or the stem region of the HA, generates an immune response that provides broader cross-activities against influenza viruses of different strains/lineages. Without wishing to be bound by theories, the absence of the glycan shield increases the exposure of the HA surface structures and conserved surface regions thereof to the host immune systems. Particularly, it is believed that the absence of at least one of the glycosylation sites in the HA2 subunit or the stem region better exposes the hydrophobic pockets formed by those conserved surface epitopes to the host immune systems.

As a result, the immune response induced by such a low-sugar HA gains better cross-activities against various strains/lineages. For example, a flu vaccine comprising or encoding a low-sugar influenza B-Yamagata virus HA would be able to trigger a host immune response against both Yamagata lineage and Victoria lineage and even influenza A viruses; a flu vaccine comprising or encoding a low-sugar influenza A H1 virus HA would be able to trigger a host immune response against a homologous H1 virus, a heterologous H1 virus, a heterosubtypic Group 1 virus (including H2, H5, H6, H8, H9, H11, H12, H13, H16, H17, and H18 virus), and/or a heterosubtypic Group 2 virus (including H3, H4, H7, H10, H14, and H15 virus). Accordingly, a low-sugar HA or a nucleic acid encoding a low-sugar HA is believed to be an effective tool to control and prevent a potential flu pandemic.

As used herein, the phrase “devoid of a glycosylation site” describes that the subunit, domain, motif, or a portion of a bigger peptide or protein that the phrase is referring to does not have a glycosylation site. In some cases, the subunit, domain, motif, or the portion used to have a glycosylation site, but it is removed or disrupted so a glycan cannot be conjugated to the site anymore. In some cases, the subunit, domain, motif, or the portion does not have the glycosylation site, compared with a reference influenza HA. That said, at least one of the glycosylation sites that used to be present on the reference influenza HA is absent on the recombinant influenza HA of the present disclosure.

Glycosylation sequon and Deglycosylation sequon. A glycosylation site normally comprises a glycosylation sequon: N-Xa-S/T, wherein Xa in the sequon is any amino acid residue except proline, and S/T denotes a serine or threonine residue. The glycosylation site can be removed by disrupting the sequon, for example by replacing the Asn (N) residue and/or the Ser(S)/Thr (T) residue with other amino acids. In some specific embodiments, the Asn (N) residue is commonly replaced with a Gln (Q) residue to disrupt the sequon, thereby removing the glycosylation site. Consequently, if a glycosylation is disrupted by replacing the Asn (N) residue with other amino acids, the deglycosylation will leave a deglycosylation sequon of Z-Xₐ-S/T, wherein Z denotes any amino acid residue except for asparagine (N; Asn), S denotes a serine (S; Ser) residue, T denotes a threonine (T; Thr) residue, and Xₐ is any amino acid residue except for proline (P).

HA1 subunit and HA2 subunit. Influenza hemagglutinin (HA) is a homotrimeric glycoprotein, and each monomer of the trimer comprises a signal peptide and two subunits, HA1 and HA2. The HA is synthesized in a host cell as a single polypeptide, which is subsequently cleaved into the HA1 subunit and the HA2 subunit. Therefore, the HA1 subunit and the HA2 subunit are defined by a proteolytic cleavage site, conserved across most influenza virus, where upstream (N-terminal) the proteolytic cleavage site is the HA1 subunit, and downstream (C-terminal) the proteolytic cleavage site is the HA2 subunit.

In some embodiments, the conserved proteolytic cleavage site comprises a X_{b}-X_{b}—R-G peptide, wherein G denotes a glycine (G) residue, R denotes an arginine (R) residue, and X_{b} denotes any amino acid. A protease usually cuts in between the R residue and the G residue, thereby leaving the amino acid G as the first amino acid of the HA2 subunit. Most influenza A viruses (H1 and H3 strains) use trypsin-like proteases, and the conserved proteolytic cleavage site might have a QSRG (SEQ ID NO: 26) or QTRG (SEQ ID NO: 27) sequence where it is cut in between the R and G residues. A H5 strain normally uses furin-like proteases, therefore, the conserved proteolytic cleavage site might have multiple R or K residues, such as a RRKKRGLF (SEQ ID NO: 28) sequence. On the other hand, a typical IBV proteolytic cleavage site comprises a KERG (SEQ ID NO: 29) sequence, but other variations also exist.

Alternatively, a HA2 subunit is known to start with a fusion peptide. The fusion peptide is conserved in length, hydrophobicity, and key residues across influenza viruses and can be used to distinguish the HA2 domain from the HA1 domain. An influenza A virus fusion peptide normally comprises a sequence of GLFGAIAGFIEGGW (SEQ ID NO: 30) GIFGAIAGFIEGGW (SEQ ID NO: 31). In influenza B virus, the fusion peptide normally comprises a sequence of GLFGAIAGFIEGGWTGM (SEQ ID NO: 32). Note that the first three amino acids of the fusion peptide is the conserved proteolytic cleavage site described above.

Stem Region. A stem region of the influenza HA is a membrane-proximal, α-helical bundle of the HA trimer that supports the globular head. The stem region is composed mostly of the HA2 subunit (the entire HA2 subunit is within the stem region) and parts of the HA1 subunit, including a HA1 N-terminal loop motif, a HA1 lower helix motif, and a HA1 C-terminal beta-strands motif.

There are not, however, universal definitions of the HA1 N-terminal loop motif, the HA1 lower helix motif, and the HA1 C-terminal beta-strands motif. It is believed that HA1 N-terminal loop motif normally comprises the first 10 to 40 amino acids, the first 15 to 40 amino acids, or the first 20 to 40 amino acids immediately C-terminal to the signal peptide. The HA1 C-terminal beta-strands motif usually comprises the last 10 to 30 amino acids of the HA1 domainHA1 subunit, and the HA1 lower helix motif usually comprises the 5 to 15 amino acids immediately upstream of the HA1 C-terminal beta-strands motif.

In some embodiments, a N-terminal of the HA1 subunit that forms part of the stem region comprises the first 30 to 40 amino acids of the HA1 subunit. In certain embodiments, the N-terminal of the HA1 subunit that forms part of the stem region comprises the first 35 to 40 amino acids, the first 30 to 38 amino acids, or the first 35 to 37 amino acids of the HA1 subunit. In some embodiments, the N-terminal of the HA1 subunit that forms part of the stem region comprises the 12^{th} to the 60^{th}, the 15^{th} to the 55^{th}, the 16^{th} to the 52^{nd}, or 18^{th} to the 52^{nd} amino acid residues of the HA.

It is known that glycosylation also plays a role in protein folding. Removal or absence of glycosylation might cause the recombinant influenza HA misfolding or improper folding. Without wishing to be bound by theories, the present disclosure shows that the glycosylation on the N-terminal region of the HA1 subunit (e.g. HA1 N-terminal loop motif) impacts the protein folding more significantly than other regions that form the stem region, while in some variants, the glycosylation or not in the N-terminal region of HA1 subunit does not significantly affect the folding of the recombinant influenza HA.

### Reference Influenza HA

As used herein, the phrase “compared with a reference influenza HA” considers the recombinant influenza HA is modified or derived from the reference influenza HA to demonstrate or emphasize the feature of the recombinant influenza HA. In some embodiments, the reference influenza HA is a wild-type influenza HA. As viral genomes exhibit a versatile nature, a wild-type virus, in this context, is defined as a naturally occurring virus strain whose HA shares high similarity (at least 70%) with the recombinant influenza HA, with differences mainly in glycosylation sites. In some embodiments, the recombinant influenza HA is derived from the reference influenza HA. As used herein, “derived from the reference influenza HA” only describes the homology of the recombinant influenza HA and the reference influenza HA and does not limit whether the recombinant influenza HA is actually modified from the reference influenza HA. In some other embodiments, the recombinant influenza HA is modified from the reference influenza HA using genetic engineering approaches.

In some embodiments, the reference influenza HA can be a HA of, but not limited to, a A/Victoria/4897/2022 (H1N1) strain (SEQ ID NO: 4), a A/Puerto Rico/8/1934 (H1N1) strain (SEQ ID NO: 19), a Thailand/8/2022 (H3N2), a Darwin/9/2021 (H3N2), a Phuket/3073/2013 (Yamagata) (SEQ ID NO: 1), A/Thailand/8/2022 (H3N2) (SEQ ID NO: 7), or B/Austria/1359417/2021 (IBV Victoria) (SEQ ID NO: 14).

On top of that, given the versatile nature of viral genomes, the corresponding amino acids of the reference influenza HA and the recombinant influenza HA might be numbered differently. For example, in the embodiments that the reference influenza HA comprises an amino acid sequence as set forth in SEQ ID NO: 1, the stem region of the reference influenza HA comprises the 16^{th} to 52^{nd} amino acids and the 318^{th} to 584^{th} amino acids of SEQ ID NO: 1. The stem region of the recombinant influenza HA comprises corresponding amino acids, but those amino acids might be or not be the 16^{th} to 52^{nd} amino acids and the 318^{th} to 584^{th} amino acids of the recombinant influenza HA. Nevertheless, those corresponding amino acids can be identified by, for example, the Basic Local Alignment Search Tool (BLAST) of the National Institute of Health, RSCB of Patent Data Bank (PDB), Expasy of the Swiss Bioinformatics Resource Portal, and/or other bioinformatics tools available in the field for sequence alignment or structure prediction and comparison.

### Exemplary Influenza B Virus (IBV), Yamagata.

In some embodiments, the reference influenza HA is an influenza B Yamagata lineage wild-type B/Phuket/3073/2013 strain, comprising an amino acid sequence of at least 80%, 85%, 90%, 91%, 92%, 93%, 94%, 95%, 96%, 97%, 98%, or 99%, or 100% identical to SEQ ID NO: 1. The stem region of the recombinant influenza HA comprises amino acids corresponding to the 16^{th} to 52^{nd} amino acids and the 318^{th} to 597^{th} amino acids of SEQ ID NO: 1 (Table 1). In such embodiments, the reference influenza HA comprises six glycosylation sites in the stem region, including respectively the N40 residue, the N318 residue, the N347 residue, the N506 residue, the N532 residue, and the N545 residue of SEQ ID NO: 1, and, in comparison with the reference influenza HA, the recombinant influenza HA has at least one, two, three, four, or all six glycosylation sites absent.

TABLE 1
SEQ ID NO: 1 analysis
MKAIIVLLMV VTSNADRICT GITSSNSPHV VKTATQGEVN VTGVIPLTTT
PTKSYFANLK GTRTRGKLCP DCLNCTDLDV ALGRPMCVGT TPSAKASILH
EVRPVTSGCF PIMHDRTKIR QLPNLLRGYE KIRLSTQNVI DAEKAPGGPY
RLGTSGSCPN ATSKIGFFAT MAWAVPKDNY KNATNPLTVE VPYICTEGED
QITVWGFHSD DKTQMKSLYG DSNPQKFTSS ANGVTTHYVS QIGDFPDQTE
DGGLPQSGRI VVDYMMQKPG KTGTIVYQRG VLLPQKVWCA SGRSKVIKGS
LPLIGEADCL HEEYGGLNKS KPYYTGKHAK AIGNCPIWVK TPLKLANGTK

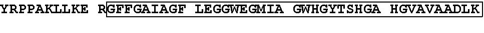

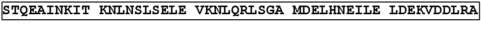

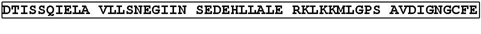

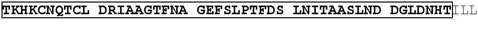

YYSTAASSLA VTLMLAIFIV YMVSRDNVSC SICL
Signal Peptide: the 1^{st} to 15^{th} residues.
HA1 subunit: the 16^{th} to 361^{st} residues.
HA2 subunit: the 362^{nd} to 597^{th} residues.
Stem region: 16^{th} to 52^{nd} and 318^{th} to 597^{th} residues.
HA1 N-terminal loop motif: 16^{th} to 52^{nd} residues.
the HA1 C-terminal beta-strands motif + the HA1 lower
helix motif: the 318^{th} to 361^{st} residues.

More specifically, in some embodiments, the recombinant influenza HA has residues corresponding to the N40 residue, the N318 residue, the N347 residue, the N506 residue, the N532 residue, and the N545 of the reference influenza HA. While the six residues above of the reference influenza HA respectively form a glycosylation site (e.g., as the N residue of the N-Xₐ-S/T sequon), at least one of the corresponding residues of the recombinant influenza HA does not form a glycosylation site. For example, at least one of the corresponding residues is replaced with a non-Asn amino acid (e.g., a Gln (Q) residue), thereby disrupting the sequon. Instead, at least one of the corresponding residues form a deglycosylation sequon, as described herein.

It is important to note that, as discussed above, the corresponding residues of the recombinant influenza HA might not be located as the 40^{th}, 318^{th}, 347^{th}, 506^{th}, 532^{nd}, and 545^{th} amino acid of the recombinant influenza HA. Nevertheless, using bioinformatics tools, those corresponding amino acids can be identified in reference to SEQ ID NO: 1. As used herein and throughout this disclosure, “in reference to” describes using the SEQ ID NO: 1, its sequences and 3-dimensional structure, as a reference to compare with a query sequence (e.g., given recombinant influenza HA) to determine corresponding amino acids on the query sequence.

Without wishing to be bound by theories, the amino acid of the recombinant influenza HA that corresponds to the N40 residue of SEQ ID NO: 1 forms a glycosylation site in the HA1 N-terminal loop motif, which is considered to have a greater impact on the folding of the recombinant influenza HA. Therefore, in some embodiments, the glycosylation site comprising N40 residue is kept.

In some embodiments, the recombinant influenza HA comprises an amino acid sequence of at least 80%, 85%, 90%, 91%, 92%, 93%, 94%, 95%, 96%, 97%, 98%, or 99%, or 100% identical to SEQ ID NO: 2 below:

MKAIIVLLMV VISNADRICT GITSSNSPHV VKTATQGEVN
VTGVIPLITT PTKSYFANLK GTRTRGKLCP DCLNCTDLDV
ALGRPMCVGT TPSAKASILH EVRPVTSGCF PIMHDRTKIR
QLPNLLRGYE KIRLSTQNVI DAEKAPGGPY RLGTSGSCPN
ATSKIGFFAT MAWAVPKDNY KNATNPLTVE VPYICTEGED
QITVWGFHSD DKTQMKSLYG DSNPQKFTSS ANGVTTHYVS
QIGDEPDQTE DGGLPQSGRI VVDYMMQKPG KTGTIVYQRG
VLLPQKVWCA SGRSKVIKGS LPLIGEADCL HEEYGGLXKS
KPYYTGKHAK AIGNCPIWVK TPLKLAXGTK YRPPAKLLKE
GFFGAIAGF LEGGWEGMIA GWHGYTSHGA HGVAVAADLK
STQEAINKIT KNLNSLSELE VKNLQRLSGA MDELHNEILE
LDEKVDDLRA DTISSQIELA VLLSNEGIIN SEDEHLLALE
RKLKKMLGPS AVDIGNGCFE TKHKCXQTCL DRIAAGTFNA
GEFSLPTFDS LXITAASLND DGLDXHTILL YYSTAASSLA
VILMLAIFIV YMVSRDNVSC SICL;

and wherein X denotes any amino acid, provided that (1) X318, X347, X506, X532, and X545 are not asparagine (N; Asn), and N40 X40 is Asn or (2) X40, X318, X347, X506, X532, and X545 are not Asn.

In certain embodiments, all X318, X347, X506, X532, and X545 are Gln (Q), and the recombinant influenza HA comprises an amino acid sequence of at least 80%, 85%, 90%, 91%, 92%, 93%, 94%, 95%, 96%, 97%, 98%, or 99%, or 100% identical to SEQ ID NO: 3. In some embodiments, the nucleic acid encoding the recombinant influenza HA might comprise a nucleotide sequence of at least 80%, 85%, 90%, 91%, 92%, 93%, 94%, 95%, 96%, 97%, 98%, or 99%, or 100% identical to SEQ ID NO: 20.

### Exemplary Influenza B Virus (IBV), Victoria.

In some embodiments, the reference influenza HA is an influenza B Victoria lineage wild-type B/Austria/1359417/2021 strain, comprising comprises an amino acid sequence of at least 80%, 85%, 90%, 91%, 92%, 93%, 94%, 95%, 96%, 97%, 98%, or 99%, or 100% identical to SEQ ID NO: 14. The stem region of the recombinant influenza HA comprises amino acids corresponding to the 16^{th} to 52^{nd} amino acids and the 317^{th} to 545^{th} amino acids of SEQ ID NO: 14 (Table 2). In such embodiments, the reference influenza HA comprises six glycosylation sites in the stem region, including respectively the N40 residue, the N316* residue, the N345 residue, the N504 residue, the N530 residue, and the N543 residue of SEQ ID NO: 14, and, in comparison with the reference influenza HA, the recombinant influenza HA has at least one, two, three, four, or all six glycosylation sites absent. It is important to note that, the 316^{th} residue is not part of the stem region, but, in this embodiment, the glycosyation site comprising that reside is considered part of the stem region.

TABLE 2
SEQ ID NO: 14 analysis
MKAIIVLLMV VTSNADRICT GITSSNSPHV VKTATQGEVN VTGVIPLTTT
PTKSHFANLK GTETRGKLCP KCLNCTDLDV ALGRPKCTGK IPSARVSILH
EVRPVTSGCF PIMHDRTKIR QLPNLLRGYE HVRLSTHNVI NTEDAPGGPY
EIGTSGSCLN ITNGKGFFAT MAWAVPKNKT ATNPLTIEVP YICTEEEDQI
TVWGFHSDDE TQMARLYGDS KPQKFTSSAN GVTTHYVSQI GGFPNQTEDG
GLPQSGRIVV DYMVQKSGKT GTITYQRGIL LPQKVWCASG KSKVIKGSLP
LIGEADCLHE KYGGLNKSKP YYTGEHAKAI GNCPIWVKTP LKLANGTKYR

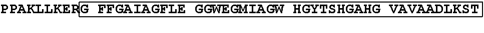

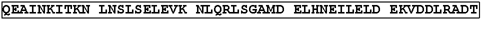

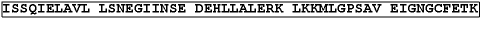

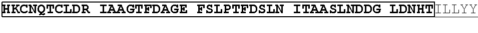

STAASSLAVT LMIAIFVVYM VSRDNVSCSI CL
Signal Peptide: the 1^{st} to 15^{th} residues.
HA1 subunit: the 16^{th} to 359^{st} residues.
HA2 subunit: the 360^{th} to 545^{th} residues.
Stem region: 16^{th} to 52^{nd} and 317^{th} to 545^{th} residues.
HA1 N-terminal loop motif: 16^{th} to 52^{nd} residues.
the HA1 C-terminal beta-strands motif + the HA1 lower
helix motif: the 317^{th} to 359^{st} residues.

More specifically, in some embodiments, the recombinant influenza HA has residues corresponding to the N40 residue, the N316 residue, the N345 residue, the N504 residue, the N530 residue, and the N543 residue of the reference influenza HA. While those residues respectively form a glycosylation site (e.g., as the N residue of the N-Xₐ-S/T sequon) for the reference influenza HA, at least one of the corresponding residues of the recombinant influenza HA does not form a glycosylation site. For example, at least one of the corresponding residues is replaced with a non-Asn amino acid (e.g., a Gln (Q) residue), thereby disrupting the sequon. Instead, at least one of the corresponding residues form a deglycosylation sequon, as described herein.

It is important to note that, as discussed above, the corresponding residues of the recombinant influenza HA might not be located as the 40^{th}, 316^{th}, 345^{rd}, 504^{th}, 530^{th}, and 543^{rd} amino acid of the recombinant influenza HA. Nevertheless, using bioinformatics tools, those corresponding amino acids can be identified in reference to SEQ ID NO: 14. As used herein and throughout this disclosure, “in reference to” describes using the SEQ ID NO: 14, its sequences and 3-dimensional structure, as a reference to compare with a query sequence (e.g., given recombinant influenza HA) to determine corresponding amino acids on the query sequence.

Without wishing to be bound by theories, the amino acid of the recombinant influenza HA that corresponds to the N40 residue of SEQ ID NO: 14 forms a glycosylation site in the HA1 N-terminal loop motif, which is considered to have a greater impact on the folding of the recombinant influenza HA. Therefore, in some embodiments, the glycosylation site comprising N40 residue is kept.

Furthermore, in some embodiments, the present disclosure demonstrated that the stem region's glycosylation sites, which are within the HA1 subunit, can be kept, and only the HA2 subunit is deglycosylated. A vaccine encoding such a recombinant influenza HA showed promising immunogenicity.

In some embodiments, the recombinant influenza HA comprises an amino acid sequence of at least 80%, 85%, 90%, 91%, 92%, 93%, 94%, 95%, 96%, 97%, 98%, or 99%, or 100% identical to SEQ ID NO: 15 below:

MKAIIVLLMV VTSNADRICT GITSSNSPHV VKTATQGEVX VTGVIPLTTT
PTKSHFANLK GTETRGKLCP KCLNCTDLDV ALGRPKCTGK IPSARVSILH
EVRPVTSGCF PIMHDRTKIR QLPNLLRGYE HVRLSTHNVI NTEDAPGGPY
EIGTSGSCLN ITNGKGFFAT MAWAVPKNKT ATNPLTIEVP YICTEEEDQI
TVWGFHSDDE TQMARLYGDS KPQKFTSSAN GVTTHYVSQI GGFPNQTEDG
GLPQSGRIVV DYMVQKSGKT GTITYQRGIL LPQKVWCASG KSKVIKGSLP
LIGEADCLHE KYGGLXKSKP YYTGEHAKAI GNCPIWVKTP LKLAXGTKYR

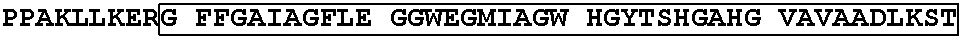

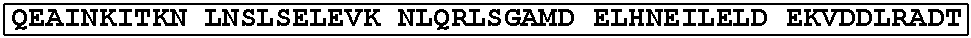

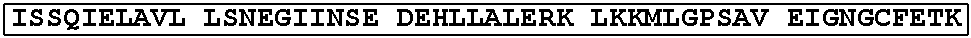

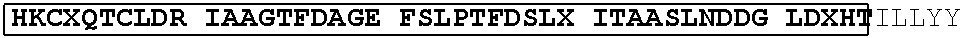

STAASSLAVT LMIAIFVVYM VSRDNVSCSI CL;
and

wherein X denotes any amino acid, provided that (1) X316, X345, X504, X530, and X543 are not asparagine (N; Asn), and X40 is Asn; or (2) X504, X530, and X543 are not asparagine (N; Asn), and X40, X316, and X345 are Asn; or (3) X40, X316, X345, X504, X530, and X543 are not Asn.

In certain embodiments, all 316, X345, X504, X530, and X543 are Gln (Q), and X40 is Asn (N), and the recombinant influenza HA comprises an amino acid sequence of at least 80%, 85%, 90%, 91%, 92%, 93%, 94%, 95%, 96%, 97%, 98%, or 99%, or 100% identical to SEQ ID NO: 16. In some embodiments, the nucleic acid encoding the recombinant influenza HA might comprise a nucleotide sequence of at least 80%, 85%, 90%, 91%, 92%, 93%, 94%, 95%, 96%, 97%, 98%, or 99%, or 100% identical to SEQ ID NO: 24.

In another embodiment, 504, X530, and X543 are Gln (Q), and X40, X316, and X345 are Asn (N), and the recombinant influenza HA comprises an amino acid sequence of at least 80%, 85%, 90%, 91%, 92%, 93%, 94%, 95%, 96%, 97%, 98%, or 99%, or 100% identical to SEQ ID NO: 18. In some embodiments, the nucleic acid encoding the recombinant influenza HA might comprise a nucleotide sequence of at least 80%, 85%, 90%, 91%, 92%, 93%, 94%, 95%, 96%, 97%, 98%, or 99%, or 100% identical to SEQ ID NO: 25.

### Exemplary Influenza A Virus (IAV), H1N1.

In some embodiments, the reference influenza HA is an influenza A H1N1 wild-type A/Victoria/4897/2022 strain, comprising an amino acid sequence of at least 80%, 85%, 90%, 91%, 92%, 93%, 94%, 95%, 96%, 97%, 98%, or 99%, or 100% identical to the amino acid sequence set forth in SEQ ID NO: 4. The stem region of the recombinant influenza HA comprises amino acids corresponding to the 18^{th} to 52^{nd} amino acids and the 268^{th} to 566^{th} amino acids of SEQ ID NO: 4 (Table 3). In such embodiments, the reference influenza HA comprises five glycosylation sites in the stem region, including the N27 residue, the N40 residue, the N293 residue, the N304 residue, and the N498 residue respectively, and, in comparison with the reference influenza HA, the recombinant influenza HA has at least one, two, three, four or all five glycosylation sites absent. In some embodiments, the 28^{th} residue, as part of the N-Xₐ-S/T sequon of N27, is also not a N residue, while in some embodiments, the 27^{th} residue remains as a N residue, but the 28^{th} residue is not a N residue.

TABLE 3
SEQ ID NO: 4 analysis

MKAILVVMLY TFTTANADTL CIGYHANNST DTVDTVLEKN VTVTHSVNLL
EDKHNGKLCK LRGVAPLHLG QCNIAGWILG NPECESLSTA RSWSYIVETS
NSDNGTCYPG DFINYEELRE QLSSVSSFER FEIFPKTSSW PNHDSDNGVT
AACSHAGARS FYKNLIWLVK KGKSYPKINQ TYINDKGKEV LVLWGIHHPP
TITDQESLYQ NADAYVFVGT SRYSKKFKPE IAARPKVRDR AGRMNYYWTL
VEPGDKITFE ATGNLVAPRY AFTMEKEAGS GIIISDTPVH DCNATCQTPE

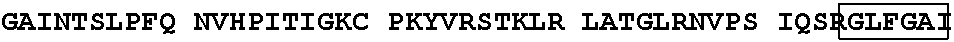

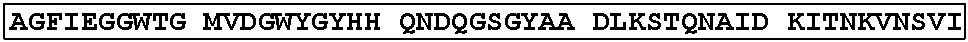

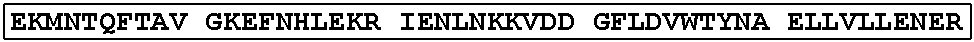

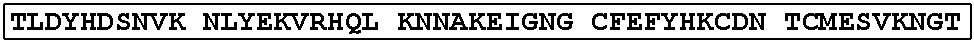

SFWMCSNGSL QCRICI
Signal Peptide: the 1^{st} to 17^{th} residues.
HA1 subunit: the 18^{th} to 344^{th} residues.
HA2 subunit: the 345^{th} to 526^{th} residues.
Stem region: 18^{th} to 52^{nd} and 268^{th} to 566^{th} residues.
HA1 N-terminal loop motif: 18^{th} to 52^{nd} residues.
the HA1 C-terminal beta-strands motif + the HA1 lower helix
motif: the 268^{th} to 344^{th} residues.

More specifically, in some embodiments, the recombinant influenza HA has residues corresponding to the N27 residue, the N40 residue, the N293 residue, the N304 residue, and the N498 residue of the reference influenza HA. While those residues respectively form a glycosylation site (e.g., as the N residue of the N-Xₐ-S/T sequon) for the reference influenza HA, at least one of the corresponding residues of the recombinant influenza HA does not form a glycosylation site. For example, at least one of the corresponding residues is replaced with a non-Asn amino acid (e.g., a Gln (Q) residue), thereby disrupting the sequon. Instead, at least one of the corresponding residues form a deglycosylation sequon, as described herein.

It is important to note that, as discussed above, the corresponding residues of the recombinant influenza HA might not be located as the 27^{th}, 40^{th}, 293^{rd}, 304^{th}, and 498^{th} amino acid of the recombinant influenza HA. Nevertheless, using bioinformatics tools, those corresponding amino acids can be identified in reference to SEQ ID NO: 4. As used herein and throughout this disclosure, “in reference to” describes using the SEQ ID NO: 4, its sequences and 3-dimensional structure, as a reference to compare with a query sequence (e.g., given recombinant influenza HA) to determine corresponding amino acids on the query sequence. Without wishing to be bound by theories, the amino acid of the recombinant influenza HA that corresponds to the N27 residue of SEQ ID NO: 4 forms a glycosylation site in the HA1 N-terminal loop motif, which is considered to have a greater impact on the folding of the recombinant influenza HA. Therefore, in some embodiments, the glycosylation site comprising N27 residue is kept.

In some embodiments, the recombinant influenza HA comprises an amino acid sequence of at least 80%, 85%, 90%, 91%, 92%, 93%, 94%, 95%, 96%, 97%, 98%, or 99%, or 100% identical to SEQ ID NO: 5 below:

MKAILVVMLY TFTTANADTL CIGYHANNST DTVDTVLEKX
VTVTHSVNLL EDKHNGKLCK LRGVAPLHLG QCNIAGWILG
NPECESLSTA RSWSYIVETS NSDNGTCYPG DFINYEELRE
QLSSVSSFER FEIFPKTSSW PNHDSDNGVT AACSHAGARS
FYKNLIWLVK KGKSYPKINQ TYINDKGKEV LVLWGIHHPP
TITDQESLYQ NADAYVFVGT SRYSKKFKPE IAARPKVRDR
AGRMNYYWTL VEPGDKITFE ATGNLVAPRY AFTMEKEAGS
GIIISDTPVH DCXATCQTPE GAIXTSLPFQ NVHPITIGKC
PKYVRSTKLR LATGLRNVPS IQSRGLFGAI AGFIEGGWTG
MVDGWYGYHH QNDQGSGYAA DLKSTQNAID KITNKVNSVI
EKMNTQFTAV GKEFNHLEKR IENLNKKVDD GFLDVWTYNA
ELLVLLENER TLDYHDSNVK NLYEKVRHQL KNNAKEIGNG
CFEFYHKCDN TCMESVKXGT YDYPKYSEEA KLNREKIDGV
KLDSTRIYQI LAIYSTVASS LVLVVSLGAI SFWMCSNGSL
QCRICI;

and wherein X denotes any amino acid, provided that (1) X40, X293, X304, and X498 are not asparagine (N; Asn), and both X27 and X28 are Asn; or (2) X27, X40, X293, X304, and X498 are not asparagine (N; Asn).

In certain embodiments, all the X40, X293, X304, and X498 are Q residues, and the recombinant influenza HA comprises an amino acid sequence of at least 80%, 85%, 90%, 91%, 92%, 93%, 94%, 95%, 96%, 97%, 98%, or 99%, or 100% identical to SEQ ID NO: 6. In some embodiments, the nucleic acid encoding the recombinant influenza HA might comprise a nucleotide sequence of at least 80%, 85%, 90%, 91%, 92%, 93%, 94%, 95%, 96%, 97%, 98%, or 99%, or 100% identical to SEQ ID NO: 21.

### Exemplary Influenza a Virus (IAV), H3N2.

In some embodiments, the reference influenza HA is an influenza A H1N1 wild-type A/Thailand/8/2022 strain, comprising an amino acid sequence of at least 80%, 85%, 90%, 91%, 92%, 93%, 94%, 95%, 96%, 97%, 98%, or 99%, or 100% identical to the amino acid sequence set forth in SEQ ID NO: 7. The stem region of the recombinant influenza HA comprises amino acids corresponding to the 17^{th} to 43^{rd} amino acids and the 310^{th} to 528^{th} amino acids of SEQ ID NO: 7 (Table 4). In such embodiments, the reference influenza HA comprises three glycosylation sites in the stem region, including the N24 residue, the N38 residue, and the N499 residue, and, in comparison with the reference influenza HA, the recombinant influenza HA has at least one, two, or all three glycosylation sites absent.

TABLE 4
SEQ ID NO: 7 analysis

MKAIIALSNI LCLVFAQKIP GNDNSTATLC LGHHAVPNGT IVKTITNDRI
EVTNATELVQ NSSIGKICNS PHQILDGGNC TLIDALLGDP QCDGFQNKEW
DLFVERSRAN SSCYPYDVPD YASLRSLVAS SGTLEFKNES FNWTGVKQNG
TSSACKRGSS SSFFSRLNWL TSLNNIYPAQ NVTMPNKEQF DKLYIWGVHH
PDTDKNQFSL FAQSSGRITV STKRSQQAVI PNIGSRPRVR DIPSRISIYW
TIVKPGDILL INSTGNLIAP RGYFKIRSGK SSIMRSDAPI GKCKSECITP

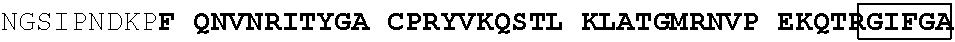

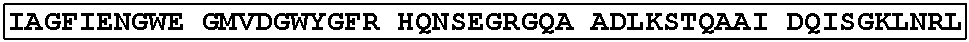

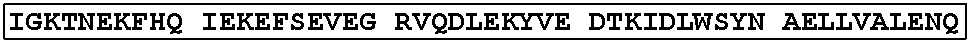

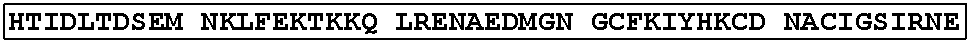

IMWACQKGNI RCNICI
Signal Peptide: the 1^{st} to 16^{th} residues.
HA1 subunit: the 17^{th} to 345^{th} residues.
HA2 subunit: the 346^{th} to 528^{th} residues.
Stem region: 17^{th} to 43^{rd} and 310^{th} to 528^{th} residues.
HA1 N-terminal loop motif: 17^{th} to 43^{rd} residues.
the HA1 C-terminal beta-strands motif + the HA1 lower helix
motif: the 310^{th} to 345^{th} residues.

More specifically, in some embodiments, the recombinant influenza HA has residues corresponding to the N24 residue, the N38 residue, the N499 residue of the reference influenza HA. While those residues respectively form a glycosylation site (e.g., as the N residue of the N-Xₐ-S/T sequon) for the reference influenza HA, at least one of the corresponding residues of the recombinant influenza HA does not form a glycosylation site. For example, at least one of the corresponding residues is replaced with a non-Asn amino acid (e.g., a Gln (Q) residue), thereby disrupting the sequon. Instead, at least one of the corresponding residues form a deglycosylation sequon, as described herein.

It is important to note that, as discussed above, the corresponding residues of the recombinant influenza HA might not be located as the 24^{th}, 38^{th}, and 499^{th} amino acid of the recombinant influenza HA. Nevertheless, using bioinformatics tools, those corresponding amino acids can be identified in reference to SEQ ID NO: 7. As used herein and throughout this disclosure, “in reference to” describes using the SEQ ID NO: 7, its sequences and 3-dimensional structure, as a reference to compare with a query sequence (e.g., given recombinant influenza HA) to determine corresponding amino acids on the query sequence.

Without wishing to be bound by theories, the amino acid of the recombinant influenza HA that corresponds to the N24 residue of SEQ ID NO: 7 forms a glycosylation site in the HA1 N-terminal loop motif, which is considered to have a greater impact on the folding of the recombinant influenza HA. Therefore, in some embodiments, the glycosylation site comprising N24 residue is kept.

In some embodiments, the recombinant influenza HA comprises an amino acid sequence of at least 80%, 85%, 90%, 91%, 92%, 93%, 94%, 95%, 96%, 97%, 98%, or 99%, or 100% identical to SEQ ID NO: 8 below:

MKAIIALSNI LCLVFAQKIP GNDXSTATLC LGHHAVPXGT IVKTITNDRI
EVTNATELVQ NSSIGKICNS PHQILDGGNC TLIDALLGDP QCDGFQNKEW
DLFVERSRAN SSCYPYDVPD YASLRSLVAS SGTLEFKNES FNWTGVKQNG
TSSACKRGSS SSFFSRLNWL TSLNNIYPAQ NVTMPNKEQF DKLYIWGVHH
PDTDKNQFSL FAQSSGRITV STKRSQQAVI PNIGSRPRVR DIPSRISIYW
TIVKPGDILL INSTGNLIAP RGYFKIRSGK SSIMRSDAPI GKCKSECITP

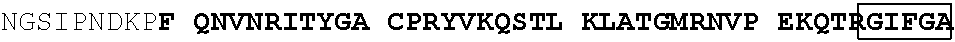

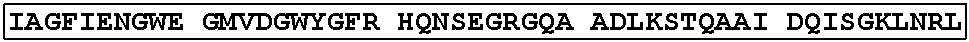

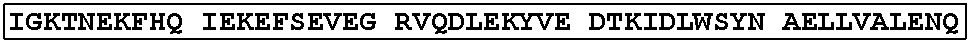

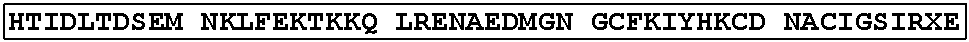

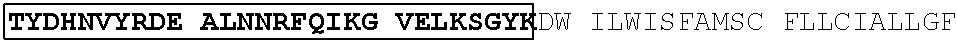

IMWACQKGNI RCNICI;

and wherein X denotes any amino acid, provided that (1) X38 and X499 are not asparagine (N; Asn), and N24 is Asn, or (2) X24, X38 and X499 are not asparagine (N; Asn).

In certain embodiments, both X38 and X499 are Gln (Q), and the recombinant influenza HA comprises an amino acid sequence of at least 80%, 85%, 90%, 91%, 92%, 93%, 94%, 95%, 96%, 97%, 98%, or 99%, or 100% identical to SEQ ID NO: 9. In some embodiments, the nucleic acid comprises a nucleotide sequence of at least 80%, 85%, 90%, 91%, 92%, 93%, 94%, 95%, 96%, 97%, 98%, or 99%, or 100% identical to SEQ ID NO: 22.

Furthermore, in some embodiments, X24, X38, and X499 are all Gln (Q), and the recombinant influenza HA comprises additional deglycosylations in the 54^{th}, 61^{st}, 79^{th}, and 301^{st} residues. In some embodiments, the recombinant influenza HA might comprise an amino acid sequence of at least 80%, 85%, 90%, 91%, 92%, 93%, 94%, 95%, 96%, 97%, 98%, or 99%, or 100% identical to SEQ ID NO: 11, and the nucleic acid encoding the recombinant influenza HA might comprise a nucleotide sequence of at least 80%, 85%, 90%, 91%, 92%, 93%, 94%, 95%, 96%, 97%, 98%, or 99%, or 100% identical to SEQ ID NO: 23.

### Vector and Immunogenic Composition

In one aspect, the present disclosure provides an expression vector comprising an expression cassette, which comprises a nucleic acid of the present disclosure. In some embodiments, the expression vector is a lipid nanoparticle (LNP), a liposome, a polymersome, a viral particle, a plasmid, or a bead. In some embodiments, the nucleic acid is DNA. In some embodiments, the nucleic acid is RNA, such as a messenger RNA (mRNA) designed to encode the recombinant influenza HA according to an exemplary embodiment of the present disclosure.

In the embodiments that the nucleic acid is a mRNA designed to encode the recombinant influenza HA in vivo, the nucleic acid can be further modified to improve its stability and translation capacity in a host cell. For example, in some embodiments, the expression cassette further comprises a promoter, which can be recognized effectively by the ribosome of the host cells. In some embodiments, the nucleic acid further comprises a 5′ untranslated region (UTR), a 3′ UTR, or both to increase the stability and regulate the translation of the mRNA. In some embodiments, the nucleic acid further comprises a poly-A tail, which helps regulate the stability of the mRNA. In yet some embodiments, the nucleic acid further comprises a 5′ cap, which is important for recruiting translation initiation factors. In some embodiments, the promoter, the 5′ untranslated region (5′UTR), the 3′ untranslated region (3′UTR), the 5′ cap, and/or the poly-A tail are operably linked to the isolated nucleic acid of the present disclosure.

Furthermore, except for the additional elements described above, the mRNA configured to encode the recombinant influenza HA according to an exemplary embodiment of the present disclosure can have its sequences modified. For example, in some embodiments, by codon optimization, the mRNA can be modified to use frequent codons, which enhances stability and translation. In some embodiments, codon optimization can be performed to modify the secondary structure of the mRNA. For example, the uridines of the mRNA might be replaced with 1-methyl-pseudouridine, which can effectively minimize the innate immune response to foreign mRNA, thereby enhancing the stability and translation of the mRNA in host cells.

### Synthesis of the Nucleic Acid

The nucleic acid of the present disclosure can be prepared using in vitro translation following conventional methods in the field. In some embodiments, a gene encoding a wild-type HA can be cloned into a conventional plasmid. Plasmids are used in the synthesis because they are easy to replicate and can reliably contain the target gene sequence. Genetic engineering approaches can be performed to modify the wild-type gene so that the N residue of the sequon N-X-S/T is substituted or to replace certain nucleotides for codon optimization. In some embodiments, the modified gene (i.e., a nucleic acid according to one exemplary embodiment of the present disclosure) can be cloned to an in vitro transcription (IVT) plasmid and flanked by a 5′UTR and a 3′UTR followed by a poly-A tail. The IVT plasmid can be reacted with a polymerase and treated with DNases to remove linear DNA. The product of the reaction can then, in some embodiments, react with capping enzymes (such as Faustovirus Capping Enzyme (FCE), Vaccinia Capping Enzyme (VCE), or mRNA cap 2′-O-methyltransferase) to obtain mRNA molecules ready for use. However, the present disclosure is not limited to the general synthesis methods described above or exemplified herein.

### Lipid Nanoparticle

In some embodiments, the expression vector is a lipid nanoparticle (LNP). LNPs are the leading delivery system used for mRNA vaccines. Conventional LNPs usually have four major components: a neutral phospholipid, cholesterol, a polyethylene-glycol (PEG)-lipid, and an ionizable cationic lipid. An exemplary LNP suitable for the present disclosure consists of SM-102 (heptadecan-9-yl 8-((2-hydroxyethyl) (6-oxo-6-(undecyloxy) hexyl) amino) octanoate), PEG2000-DMG (1-monomethoxypolyethyleneglycol-2,3-dimyristylglycerol with polyethylene glycol of average molecular weight 2000), 1,2-Distearoyl-sn-glycero-3 phosphocholine (DSPC), and cholesterol at a 50:10:38.5:1.5 ratio. Another exemplary LNP suitable for the present disclosure consists of ALC-0315 ((4-hydroxybutyl) azanediyl)bis (hexane-6,1-diyl)bis(2-hexyldecanoate)), ALC-0159 (2-[(polyethylene glycol)-2000]—N,N ditetradecylacetamide), 1,2-Distearoyl-sn-glycero-3-phosphocholine (DSPC), and cholesterol at a 46.3:9.4:42.7:1.6 ratio.

In some embodiments, the LNP further comprises a compound as described in PCT Application No. PCT/US24/23590, filed on Apr. 8, 2024, titled “METHODS AND COMPOSITIONS FOR DENDRITIC CELL TARGETING NANO-DELIVERY,” which is hereby incorporated by reference in its entirety. The compound might comprise:

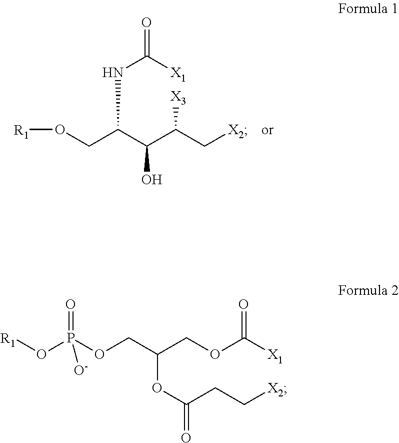

wherein R₁ comprises a substituted or non-substituted glycosyl group; wherein X₁ and X₂ are each independently hydrogen, alkyl, alkenyl, alkynyl, aryloxy, or a substituted version thereof, or —(CH₂)nX₄, n is 0 to 50, and X4 is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N; and wherein X₃ is hydrogen, C₁₋₆ alkyl, or hydroxyl.

### R₁ Group.

Targeting Functionality. In certain embodiments, the R₁ group is configured to provide selective delivery or targeted delivery functionality for the exemplary LNP formulation formed by the component of the present disclosure. In some embodiments, the R₁ group is configured to target an antigen-presenting cell (e.g., a dendritic cell). In some embodiments, the target cell can be other types of immune cells. In yet some other embodiments, the target can be any biological cells where the payload is designed. In certain embodiments, the R₁ group is designed to have a targeting moiety, which can be a ligand of a receipt on a target cell. For example, the R₁ group might be configured to target the DC-SIGN of a dendritic cell.

Without wishing to be bound by theory, it is believed that mannoside and fucoside can bind a dendritic cell (e.g., via binding to DC-SIGN) with specificity. Therefore, in some embodiments, the R₁ group comprises a mannoside, fucoside, or both as the targeting moiety. The mannoside and/or the fucoside can be a terminal mannose or a terminal fucoside of the R₁ group, which might provide better chances to interact with a dendritic cell.

In some other embodiments, the R₁ group is configured to target Siglec-1, so the glycosyl group can comprise 9-N-(4H-thieno[3,2-c]chromene-2-carbamoyl)-Neu5Ac-α2,3-Gal-GlcNAc. In some embodiments, the R₁ group is configured to target Siglec-2, and the glycosyl group can comprise 9-Biphenyl Neu5Ac-α2,6-Gal-GlcNAc. In some embodiments, the R₁ group is configured to target Siglec-5/E, and the glycosyl group can comprise Neu5Ac-α2,3-Gal-GlcNAc.

In some embodiments, the R₁ group comprises a formula of R₂—R_{A}—, wherein R₂ is the substituted or non-substituted glycosyl group, and R_{A} is an attachment group, and wherein the attachment group is an aryl, an alkyl, an amide, an alkyl amide, a combination thereof, or a covalent bond. In some embodiments, the aryl comprises 0 to 3 substituents (e.g., 1 to 3 substituents), wherein the substituent of the aryl is C₁₋₆ alkyl, halide, or C₁₋₆ alkyl halide. In some embodiments, the attachment group is configured to provide structural flexibilities and/or facilitate the binding between the targeting moiety and the target. In certain embodiments, R₂ is conjugated covalently to R_{A} at a carbon of the glycosyl group, resulting in an O-glycosylation.

Binding in acidic conditions. In some embodiments, the binding between the glycosyl group of R₁ and a target is Ca²⁺-correlated, and the calcium coordination might decrease at a low pH environment, resulting in lower binding affinity. Therefore, to provide a better binding affinity under acidic conditions, the attachment group can comprise an aryl group. Without wishing to be bound by any theories, the aryl group may engage in the CH-π and hydrophobic interactions that enhance the binding under acidic conditions. The aryl group can be an unsubstituted benzene or a benzene substituted with a halide or an alkyl halide (e.g., a CF₃). In some embodiments, the aryl group is coupled with the targeting moiety. For example, the R₁ group can comprise an O-aryl mannoside.

Spacer. In some embodiments, the attachment group of R₁ comprises a spacer. The spacer is configured to provide structural flexibility to R₁. Without wishing to be bound by theories, the flexibility allows the glycosyl group of R₁ to move during the interaction between the targeting moiety and the target, thereby facilitating the binding between them.

In certain embodiments, a preferred spacer is biocompatible. In some embodiments, the initiator spacer comprises a saturated carbon moiety, a polyethylene glycol (PEG) moiety, or a combination thereof. For example, the spacer can be a polyethylene glycol (PEG) moiety, formed by 1, 2, 3, 4, 5, 6, 7, 8, 9, 10, 12, 15, 18, 20, 24, 30, 36, 40, 48, 50, 55, 60, 65, or 72 (OCH₂CH₂) subunits, or any ranges defined by the foregoing endpoints, such as 2 to 72, 2 to 60, 2 to 48, 2 to 36, 2 to 24, 2 to 18, 2 to 15, 2 to 10, 4 to 72, 4 to 60, 4 to 48, 4 to 36, 4 to 24, 4 to 18, 4 to 15, 4 to 10, 8 to 72, 8 to 60, 8 to 48, 8 to 36, 8 to 24, 8 to 18, 8 to 15, or 8 to 10 (OCH₂CH₂) subunits. In some embodiments, the PEG moiety can be a linear, branched, or star structure.

Structural configuration. In certain embodiments, the glycosyl group can be a linear structure or a branched structure. In some embodiments, the glycosyl group might have a plurality of targeting moieties, for example, 2, 3, 4, 5, 6, 7, 8, 9, or 10 targeting moieties. The plurality of targeting moieties can be arranged in a linear, branched, or star configuration. For example, the glycosyl group might comprise a mono-mannoside, a di-mannoside, or a tri-mannoside, and when the glycosyl group comprises a tri-mannoside, the tri-mannoside can be a linear form or a branched structure, such as a α-1,3-α-1,6-trimannoside. In certain embodiments, it is noticed that a branched configuration (e.g., a tri-mannoside glycan head) shows superior binding affinity to its target receptor.

In some embodiments, the R₁ group is a substituted glycosyl group. The glycosyl group might comprise 1 to 6 substituents, and each substituent can be C₁₋₆ alkyl, C₁₋₆ alkenyl, halogen, C₁₋₆ alkyl halide, C₁₋₆ alkoxy, amine, nitro, C₁₋₆ alkyl amine, amide, azido, aryl, cycloalkyl, heterocycloalkyl, sulfite, or a substituted version thereof, or a combination thereof. In certain embodiments, the substituent is conjugated to a carbon of the glycosyl group directly or is conjugated to the carbon via an O-yl conjugation (e.g., by replacing the hydrogen of the hydroxyl group on the carbon).

In certain embodiments, the substituent of the glycosyl group is selected from the group consisting of aryl, 5-membered cycloalkyl, 6-membered cycloalkyl, 5-membered heterocycloalkyl, and 6-membered heterocycloalkyl, and a substituted version thereof, which comprises 1 to 6 substituents selected from the group consisting of C₁₋₆ alkyl, halogen, C₁₋₆ alkyl halide, C₁₋₆ alkoxy, amine, nitro, C₁₋₆ alkyl amine, azido, amide, carboxyl, hydroxyl, aryl, cycloalkyl, heterocycloalkyl, or a substituted version thereof, or a combination thereof. In some embodiments, the heterocycloalkyl comprises 1 to 3 heteroatoms, selected from the group consisting of O, S, and N.

In some embodiments, the substituent of the glycosyl group is a substituted or non-substituted aryl, for example, a substituted or non-substituted phenyl group. In certain embodiments, the aryl is substituted with 1 to 6 substituents, each is independently selected from the group consisting of C₁₋₆ alkyl, halogen, C₁₋₆ alkyl halide, C₁₋₆ alkoxy, amine, nitro, C₁₋₆ alkyl amine, azido, amide, carboxyl, hydroxyl, aryl, cycloalkyl, heterocycloalkyl, or a substituted version thereof, or a combination thereof. In certain embodiments, the substituent of the glycosyl group is a phenyl (benzene ring) substituted with OH, CH₃, NH₂, CF₃, OCH₃, F, Br, Cl, NO₂, N₃, or a combination thereof. For example, the substituted benzene ring can be a phenol group.

In some embodiments, the R₁ group is a mono-mannoside substituted with 1 to 6 substituents, and each substituent can be C₁₋₆ alkyl, C₁₋₆ alkenyl, halogen, C₁₋₆ alkyl halide, amine, C₁₋₆ alkyl amine, amide, aryl, cycloalkyl, heterocycloalkyl, sulfite, or a substituted version thereof, or a combination thereof. In certain embodiments, the R₁ group is a mono-mannoside substituted with a first substitute and a second substitute; each of the first substitute and the second substitute is independently selected from a group consisting of C₁₋₆ alkyl, C₁₋₆ alkenyl, halogen, C₁₋₆ alkyl halide, amine, C₁₋₆ alkyl amine, amide, aryl, cycloalkyl, heterocycloalkyl, and sulfite.

In some embodiments, the R₁ group comprises a first mannoside and a second mannoside. Each of the first mannoside and the second mannoside is independently substituted with 1 to 6 substituents, and each substituent can be C₁₋₆ alkyl, C₁₋₆ alkenyl, halogen, C₁₋₆ alkyl halide, amine, C₁₋₆ alkyl amine, amide, aryl, cycloalkyl, heterocycloalkyl, sulfite, or a substituted version thereof, or a combination thereof.

Binding affinity. In some embodiments, the binding affinity between the glycosyl group of R₁ and a target can be defined by a dissociation constant (K_{D}). In some embodiments, the K_{D} at pH 7.4 can be 5, 10, 15, 20, 30, 40, 50, 75, 100, 125, 150, 200, 250, 300, 350, 400, 450, 500, 600, 700, 800, 900, 1000, 1250, 1500, 1750, 2000, 2250, 2500, 3000, 3250, 3500, 3750, 4000, 4250, 4500, 4750, 5000, 5250, 5500, 5750, 6000, 6250, 6500, 6750, 7000, 7250, 7500, 7750, or 8000 nM, or any range defined by the foregoing endpoints, such as, 5 to 8000, 5 to 7000, 5 to 6000, 5 to 5000, 5 to 4000, 5 to 3000, 5 to 2500, 5 to 2000, 5 to 1500, 5 to 1250, 5 to 1000, 5 to 900, 5 to 800, 5 to 700, 5 to 600, 5 to 500, 5 to 400, 5 to 300, 5 to 200, 5 to 150, 5 to 100, 5 to 75, 5 to 50, 5 to 30, 5 to 20, 10 to 8000, 10 to 7000, 10 to 6000, 10 to 5000, 10 to 4000, 10 to 3000, 10 to 2500, 10 to 2000, 10 to 1500, 10 to 1250, 10 to 1000, 10 to 900, 10 to 800, 10 to 700, 10 to 600, 10 to 500, 10 to 400, 10 to 300, 10 to 200, 10 to 150, 10 to 100, 10 to 75, 10 to 50, 10 to 30, or 10 to 20 nM.

In some other embodiments, the K_{D} at pH 5 can be 1, 2, 3, 4, 5, 10, 15, 20, 30, 40, 50, 75, 100, 125, 150, 200, 250, 300, 350, 400, 450, 500, 550, 600, 650, 700, 750, 800, 850, 900, 1000, 1250, 1500, 1750, or 2000 nM, or any range defined by the foregoing endpoints, such as, 1 to 2000, 1 to 1500, 1 to 1000, 1 to 900, 1 to 800, 1 to 750, 1 to 700, 1 to 650, 1 to 600, 1 to 550, 1 to 500, 1 to 450, 1 to 400, 1 to 350, 1 to 300, 1 to 250, 1 to 200, 1 to 150, 1 to 100, 1 to 75, 1 to 50, 1 to 40, 1 to 30, 1 to 20, 1 to 10, or to 5, 5 to 2000, 5 to 1500, 5 to 1000, 5 to 900, 5 to 800, 5 to 750, 5 to 700, 5 to 650, 5 to 600, 5 to 550, 5 to 500, 5 to 450, 5 to 400, 5 to 350, 5 to 300, 5 to 250, 5 to 200, 5 to 150, 5 to 100, 5 to 75, 5 to 50, 5 to 40, 5 to 30, 5 to 20, 5 to 10 nM.

Examples. In some embodiments, the R₁ group is selected from the group consisting of (each structure shown below is independent from one another despite whether it is separated using a semicolon with an adjacent structure):

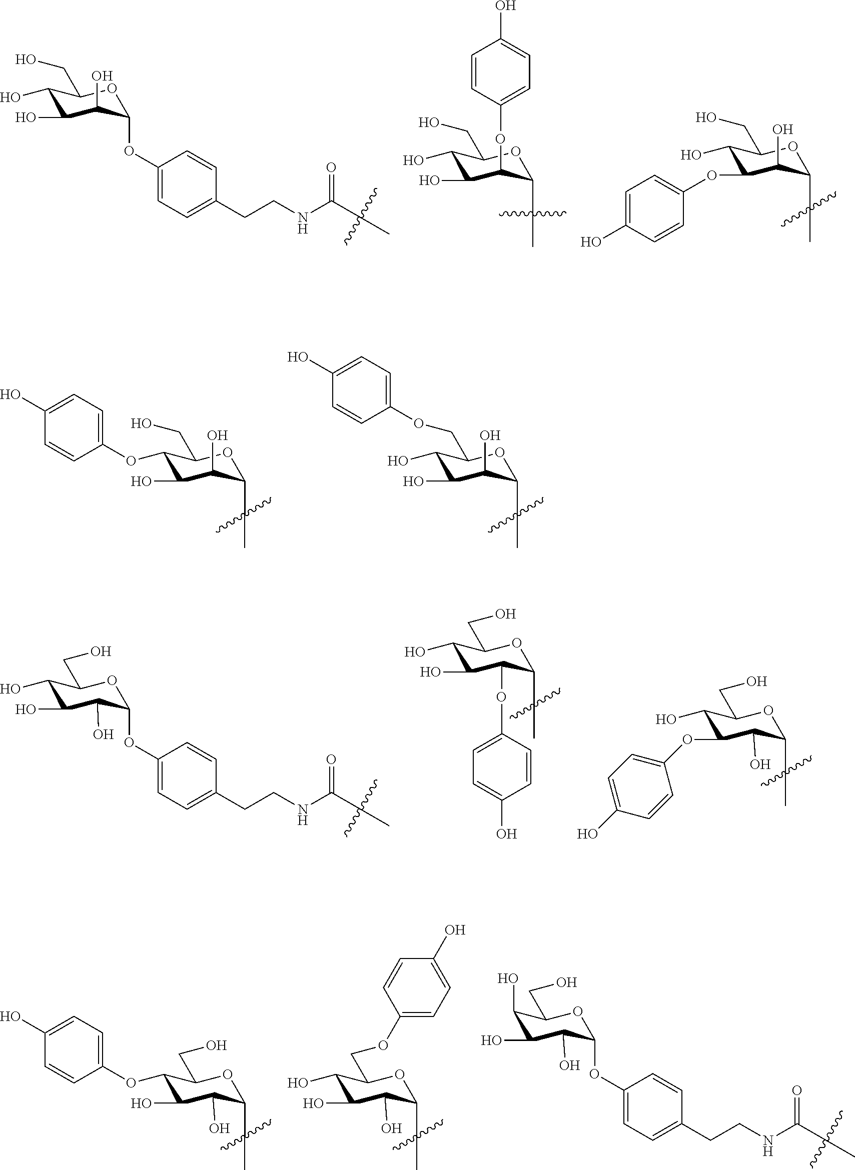

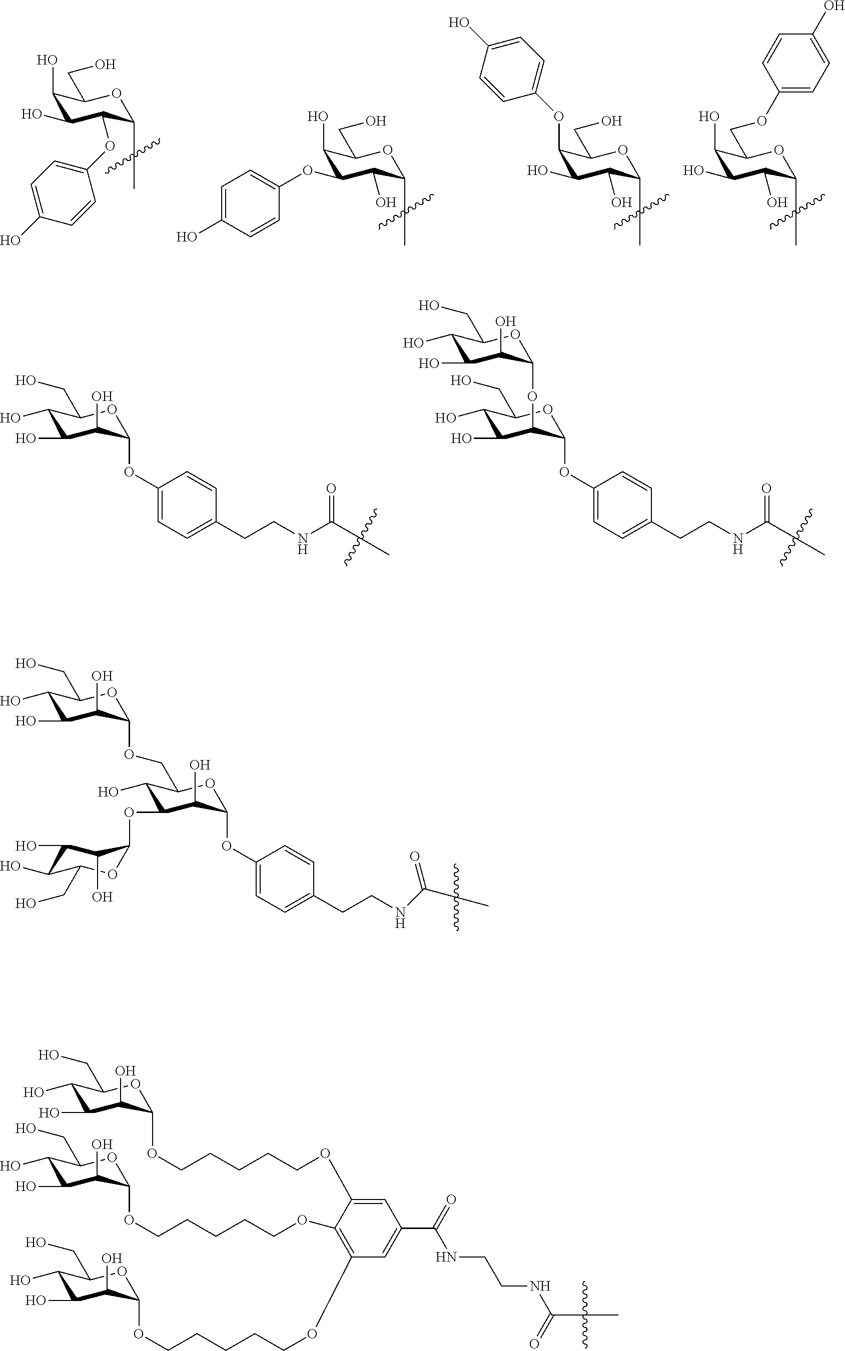

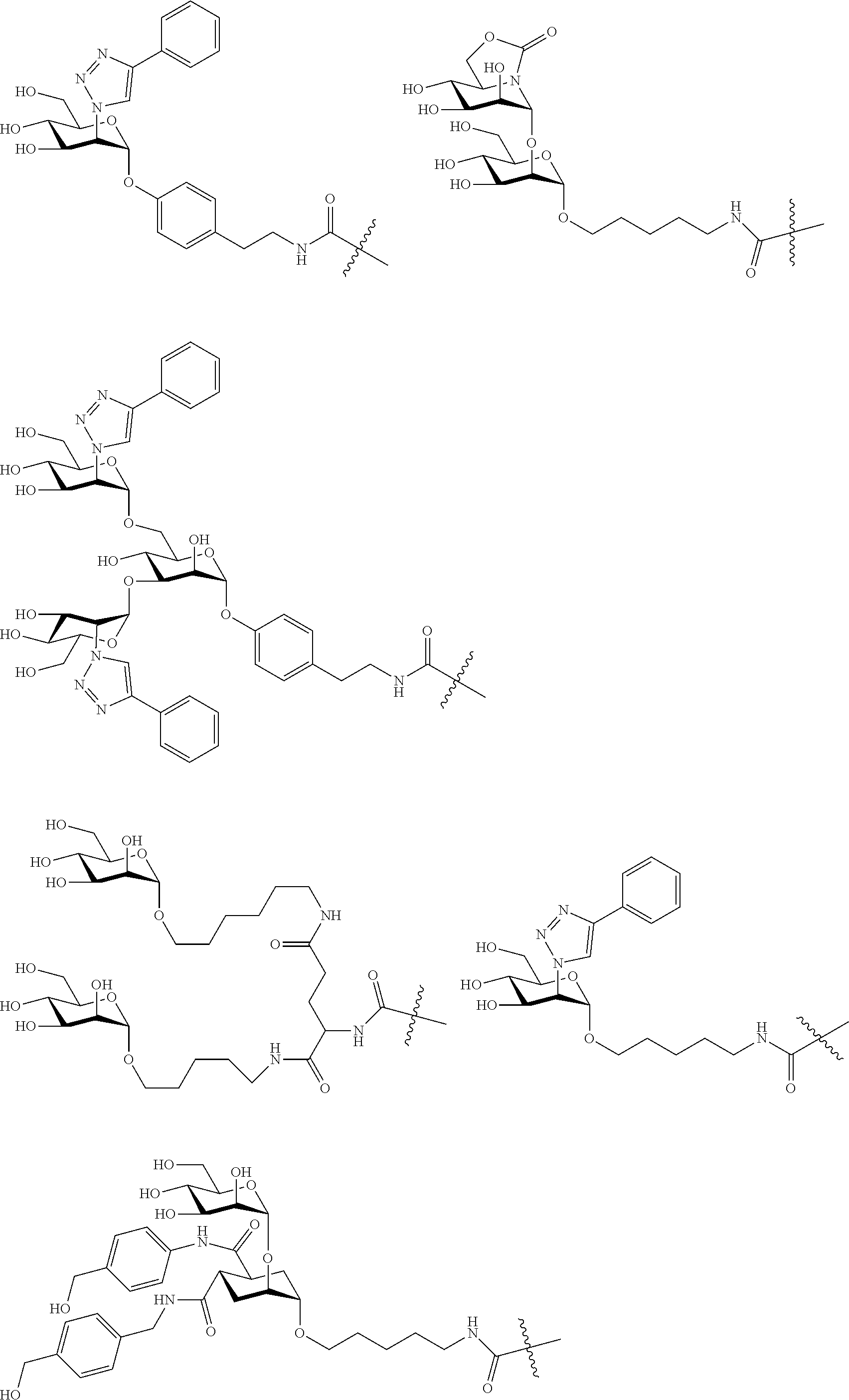

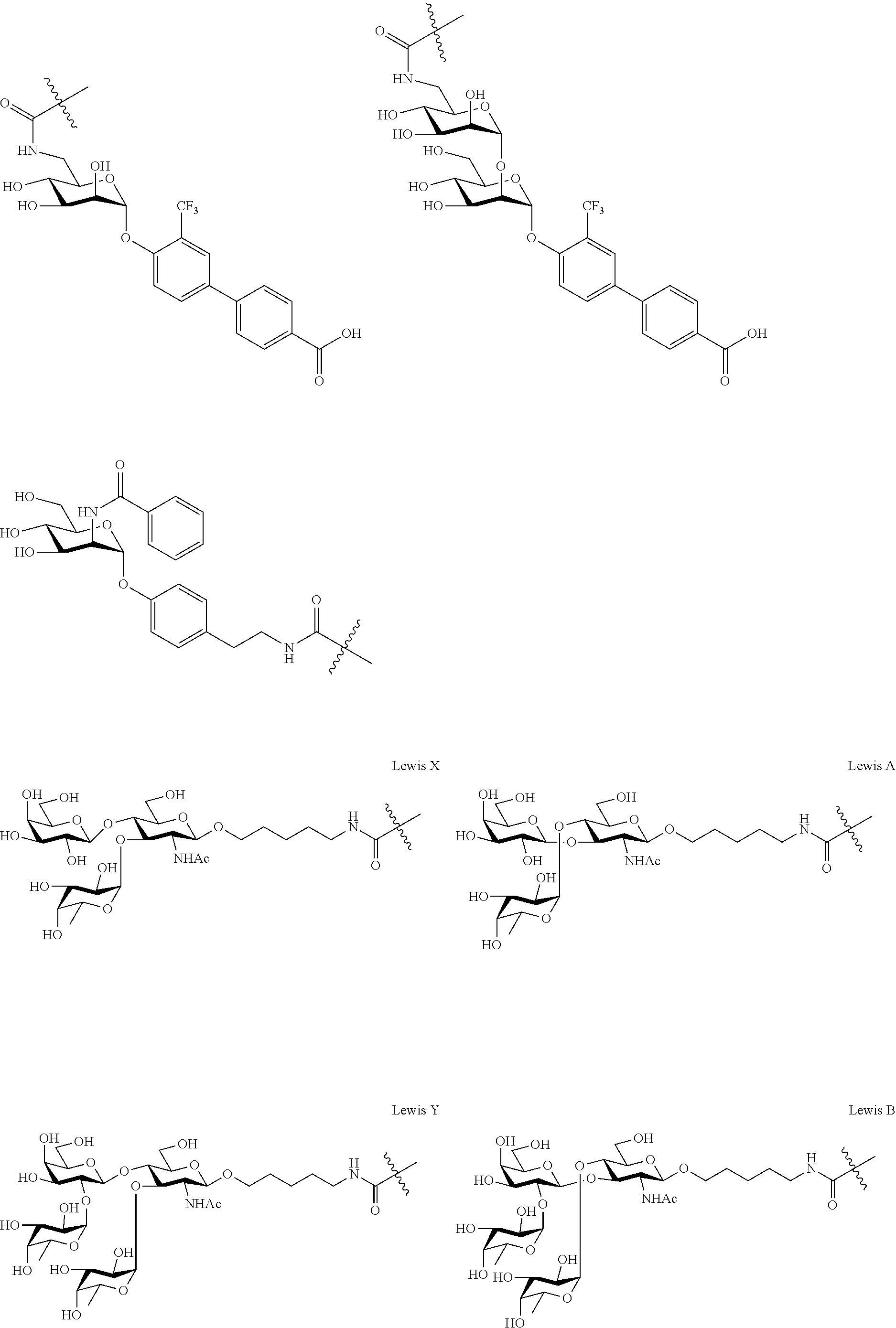

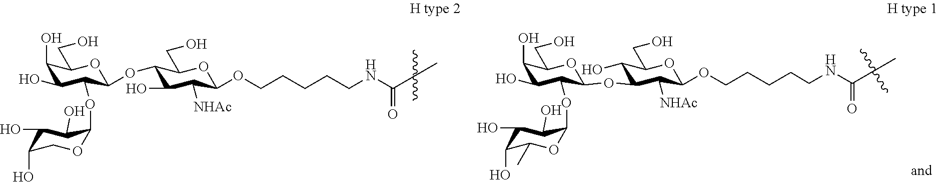

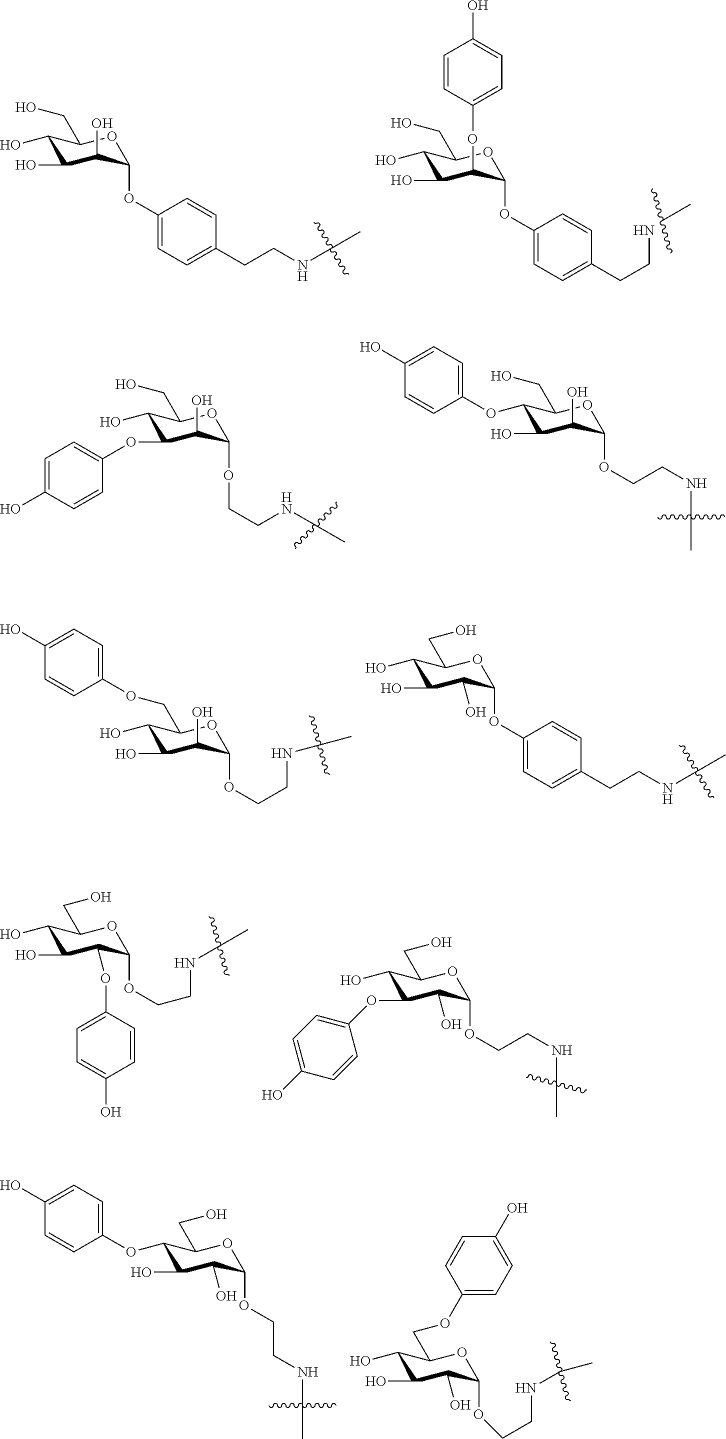

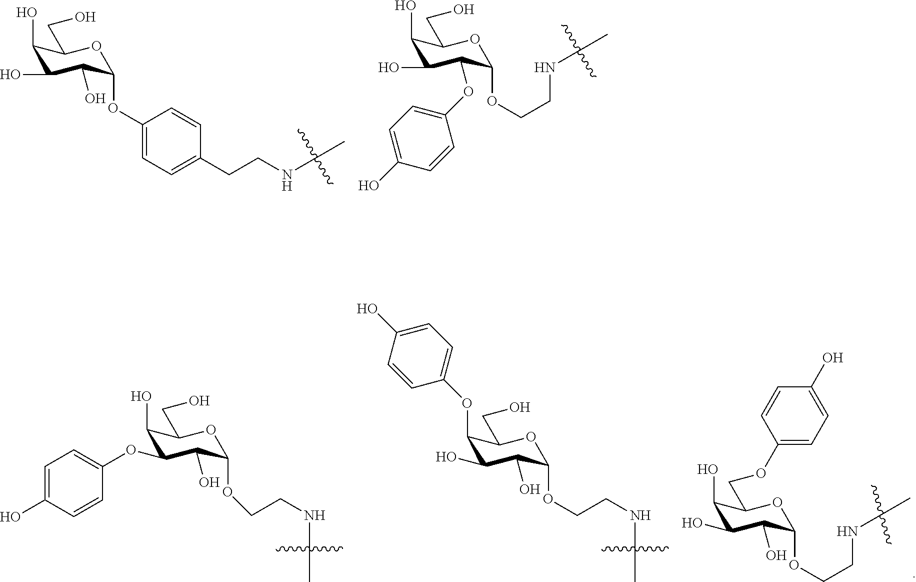

In some embodiments, the compound of the present disclosure has the structure shown in Formula 3:

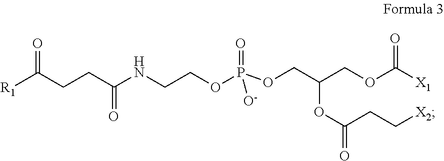

and
wherein the R₁ group is selected from the group consisting of (each structure shown below is independent from one another despite whether it is separated using a semicolon with an adjacent structure):

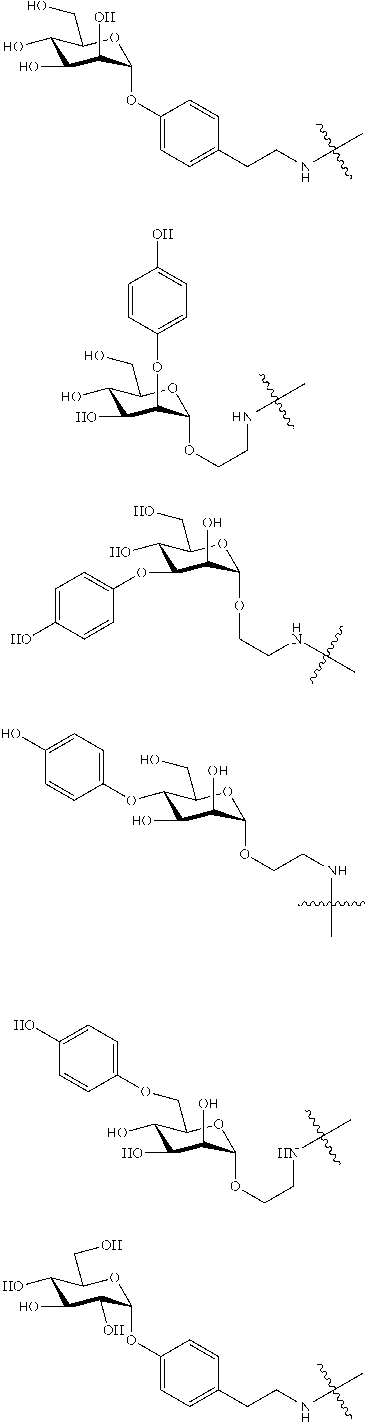

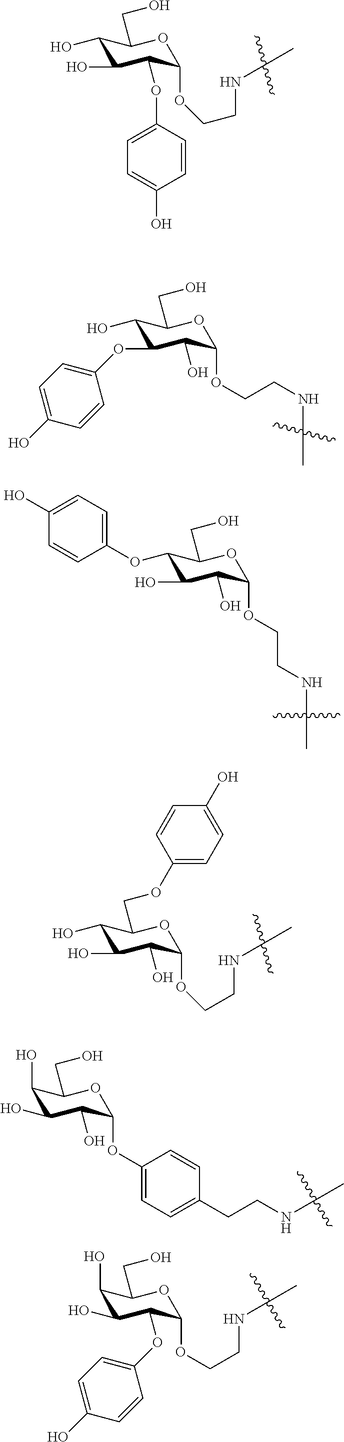

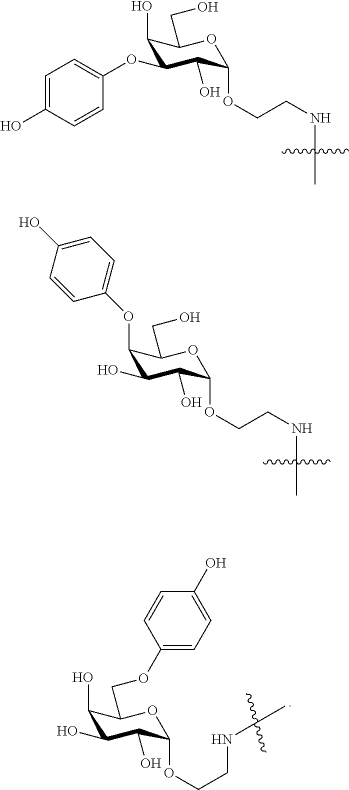

X₁ and X₂

The X₁ and X₂ are each independently hydrogen, C₁₋₃₀ alkyl, C₁₋₃₀ alkenyl, C₁₋₃₀ alkynyl, aryl, aryloxy, or a substituted version thereof, or —(CH₂)nX₄, n is 0 to 30, and X4 is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof. Without wishing to be bound by theories, at least one of the X₁ and X₂ groups is designed to provide the compound of the present disclosure with desired hydrophobicity.

In some embodiments, at least one of the X₁ and X₂ comprises a saturated hydrocarbon chain, which comprises at least 2, 3, 4, 5, 6, 7, 8, 9, 10, 11, 12, 13, 14, 15, 16, 17, 18, 19, 20, 22, 24, 26, 28, or 30 carbons, or any range of carbons defined by the foregoing endpoints, such as 2 to 30, 2 to 28, 2 to 26, 2 to 24, 2 to 20, 2 to 18, 2 to 15, 2 to 12, 2 to 10, 2 to 8, 2 to 6, 2 to 4, 3 to 30, 3 to 28, 3 to 26, 3 to 24, 3 to 20, 3 to 18, 3 to 15, 3 to 14, 3 to 13, 3 to 12, 3 to 11, 3 to 10, 3 to 9, 3 to 8, 3 to 7, 3 to 6, 3 to 5, 4 to 30, 4 to 28, 4 to 26, 4 to 24, 4 to 20, 4 to 18, 4 to 15, 4 to 14, 4 to 13, 4 to 12, 4 to 11, 4 to 10, 4 to 9, 4 to 8, 4 to 7, 4 to 6, 6 to 15, 6 to 14, 6 to 13, 6 to 12, 6 to 11, 6 to 10, 6 to 9, 6 to 8, 10 to 30, 10 to 20, 15 to 30, 15 to 28, 15 to 26, or 15 to 20 carbons.

In some embodiments, X₁ and X₂ are each independently hydrogen, C₄₋₃₀ alkyl, C₄₋₃₀ alkenyl, C₄₋₃₀ alkynyl, aryl, aryloxy, or a substituted version thereof, or —(CH₂)nX₄, n is 4 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof.

In some embodiments, X₁ and X₂ are each independently hydrogen, C₈₋₃₀ alkyl, C₈₋₃₀ alkenyl, C₈₋₃₀ alkynyl, aryl, aryloxy, or a substituted version thereof, or —(CH₂)nX₄, n is 8 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof.

In some embodiments, when one of X₁ and X₂ is hydrogen, the other one is not hydrogen. In some embodiments, when one of X₁ and X₂ is hydrogen, the other one comprises a saturated hydrocarbon chain, comprising at least 2, 3, 4, 5, 6, 7, 8, 9, 10, 11, 12, 13, 14, 15, 16, 17, 18, 19, 20, 22, 24, 26, 28, or 30 carbons, or any range of carbons defined by the foregoing endpoints, such as 2 to 30, 2 to 28, 2 to 26, 2 to 24, 2 to 20, 2 to 18, 2 to 15, 2 to 12, 2 to 10, 2 to 8, 2 to 6, 2 to 4, 3 to 30, 3 to 28, 3 to 26, 3 to 24, 3 to 20, 3 to 18, 3 to 15, 3 to 14, 3 to 13, 3 to 12, 3 to 11, 3 to 10, 3 to 9, 3 to 8, 3 to 7, 3 to 6, 3 to 5, 4 to 30, 4 to 28, 4 to 26, 4 to 24, 4 to 20, 4 to 18, 4 to 15, 4 to 14, 4 to 13, 4 to 12, 4 to 11, 4 to 10, 4 to 9, 4 to 8, 4 to 7, 4 to 6, 6 to 15, 6 to 14, 6 to 13, 6 to 12, 6 to 11, 6 to 10, 6 to 9, 6 to 8, 10 to 30, 10 to 20, 15 to 30, 15 to 28, 15 to 26, or 15 to 20 carbons. In some embodiments, one of X₁ and X₂ is C₁₅₋₃₀ alkyl, and the other is —(CH₂)nX₄, as defined above.

In some embodiments, X₄ is an aryl, aryloxy, heterocyclic group, cycloalkyl, heterocycloalkyl, or a combination thereof, and wherein X₄ comprises 0 to 6 substituents, selected from the group consisting of C₁₋₆ alkyl, halogen, C₁₋₆ alkyl halogen, and C₁₋₆ alkoxy. In certain embodiments, X₄ comprises 1 to 3 substituents. The substituent can be, but is not limited to, CH₃, CF₃, F, or OCH₃.

In some embodiments, X₄ is —R₃—O—R₄, wherein R₃ and R₄ are each independently aryl, heterocyclic group, cycloalkyl, heterocycloalkyl, each comprising 0 to 6 substituents selected from the group consisting of C₁₋₆ alkyl, halogen, C₁₋₆ alkyl halogen, and C₁₋₆ alkoxy.

In certain embodiments, X₄ is selected from the group consisting of:

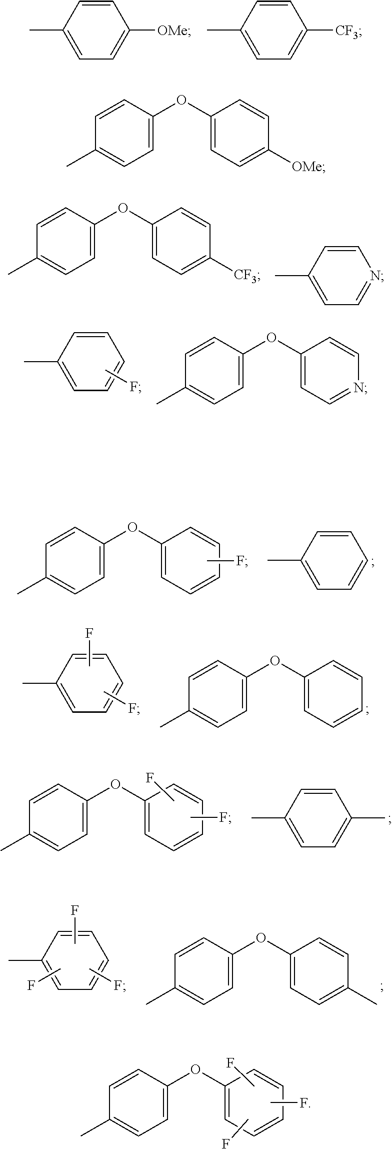

### Exemplary Compound of the Present Disclosure

This section lists some exemplary structures of the compound of the present disclosure. However, the present disclosure is not limited to the exemplary structures listed below or in the specification. In some embodiments, the compound of the present disclosure does not comprise glycolipid C34 or α-galactosylceramide (α-GalCer).

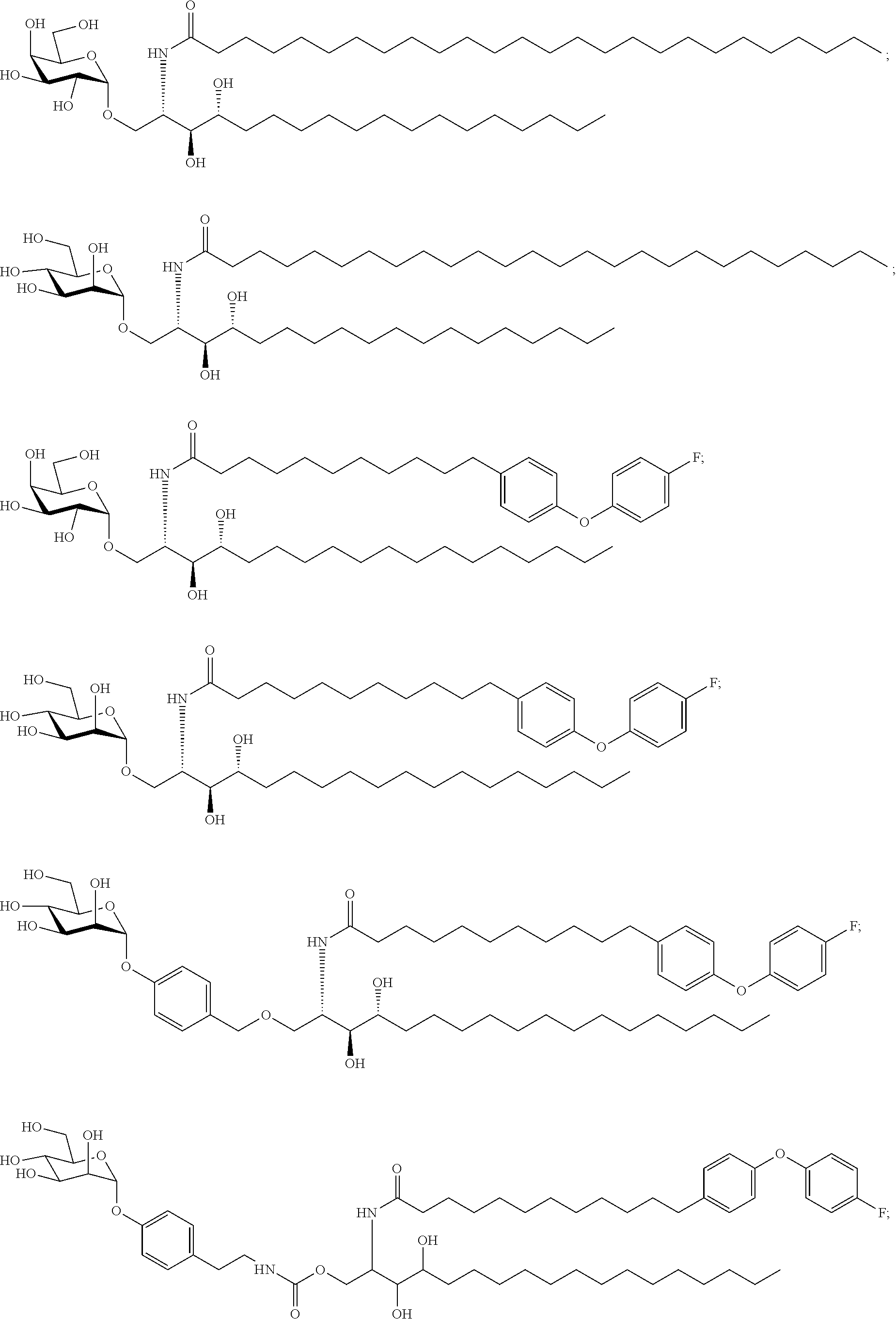

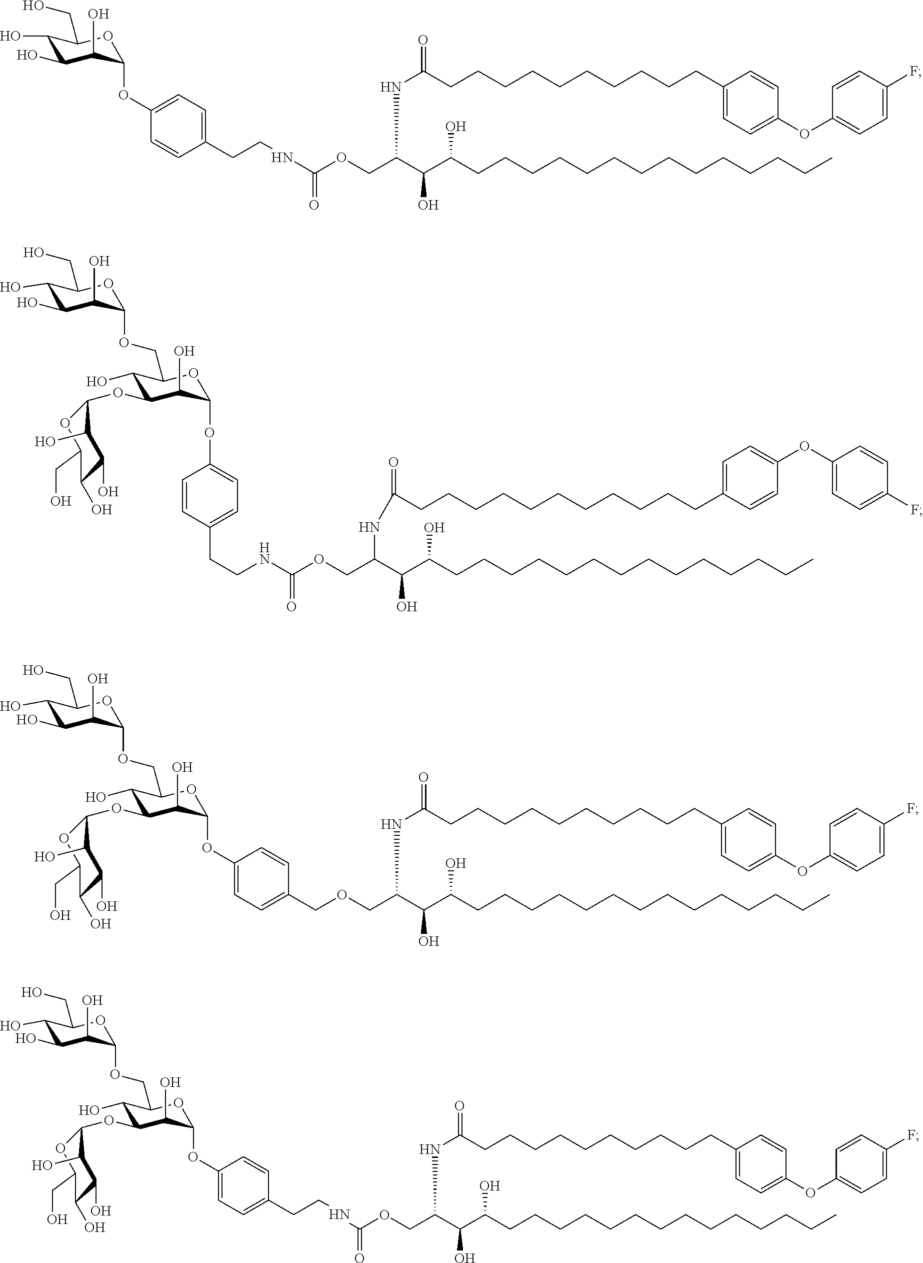

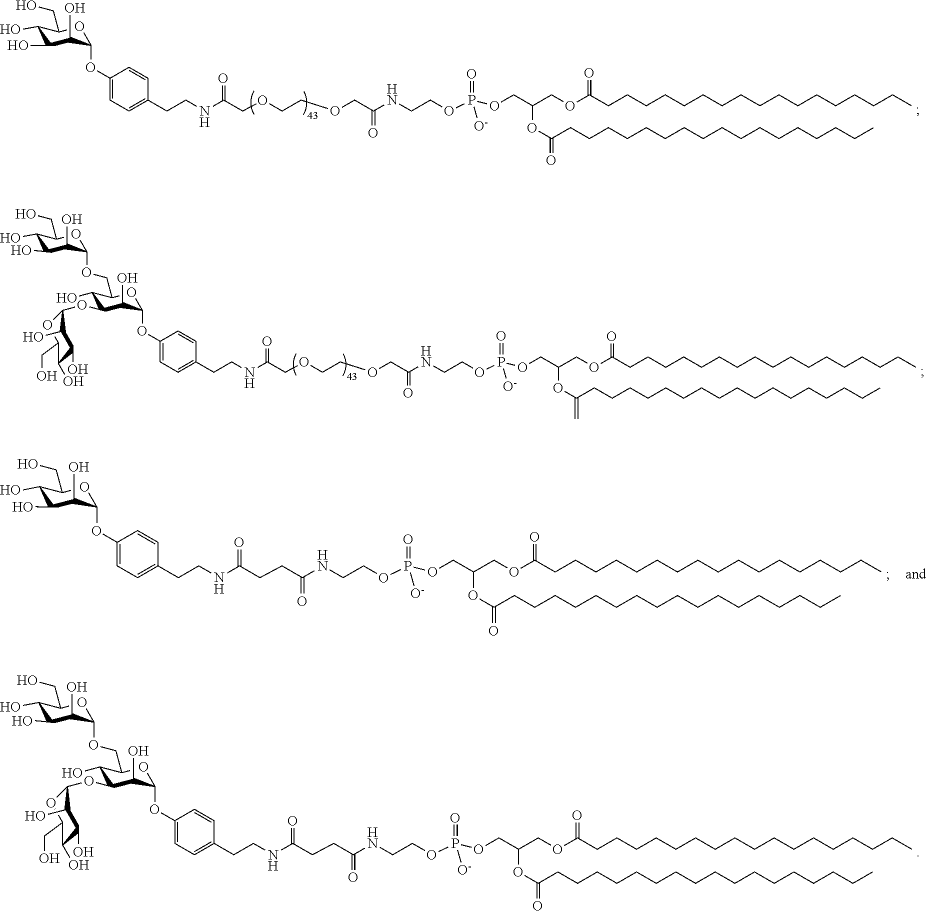

### Composition and Immunogenic Composition

In one aspect, the present disclosure provides a composition comprising the nucleic acid according to an embodiment of the present disclosure. In some embodiments, the nucleic acid is encapsulated or carried by a vector as described above according to an exemplary vector of the present disclosure. The composition can be an immunogenic composition that is designed to deliver the nucleic acid according to an embodiment of the present disclosure using a vector (e.g., as described herein) to a host cell, thereby inducing an immune response against the HA. In some embodiments, the immune response induced has cross-activities across various influenza virus strains or lineages.

In some embodiments, the composition comprises at least about 0.01, 0.05, 0.1, 0.5, 1, 2, 3, 4, 5, 10, 15, 20, 25, 30, 40, 50, 60, 70, 80, 90, 95% (w/w) the vector of the present disclosure, which encapsulates or carries an exemplary nucleic acid of the present disclosure, or any range defined by the foregoing endpoints, such as, included or excluded, 0.01% to 95% (w/w), 0.01% to 90% (w/w), 0.01% to 80% (w/w), 0.01% to 70% (w/w), 0.01% to 60% (w/w), 0.01% to 50% (w/w), 0.01% to 40% (w/w), 0.01% to 30% (w/w), 0.01% to 20% (w/w), 0.01% to 10% (w/w), 0.01% to 5% (w/w), 0.01% to 1% (w/w), 0.01% to 0.1% (w/w), 0.1% to 95% (w/w), 0.1% to 90% (w/w), 0.1% to 80% (w/w), 0.1% to 70% (w/w), 0.1% to 60% (w/w), 0.1% to 50% (w/w), 0.1% to 40% (w/w), 0.1% to 30% (w/w), 0.1% to 20% (w/w), 0.1% to 10% (w/w), 0.1% to 5% (w/w), 0.1% to 1% (w/w), 1% to 95% (w/w), 1% to 90% (w/w), 1% to 80% (w/w), 1% to 70% (w/w), 1% to 60% (w/w), 1% to 50% (w/w), 1% to 40% (w/w), 1% to 30% (w/w), 1% to 20% (w/w), 1% to 10% (w/w), 1% to 5% (w/w), 5% to 95% (w/w), 5% to 90% (w/w), 5% to 80% (w/w), 5% to 70% (w/w), 5% to 60% (w/w), 5% to 50% (w/w), 5% to 40% (w/w), 5% to 30% (w/w), 5% to 20% (w/w), or 5% to 10% (w/w). The rest of the percentages of the composition can be an excipient as described herein.

### Multivalent Composition.

In some embodiments, the immunogenic composition of the present disclosure can be a monovalent vaccine, a multivalent vaccine, or a combined vaccine. A multivalent vaccine refers to an immunogenic composition comprising immunogenic substances against more than one strain/lineage of the same organisms. A combined vaccine refers to an immunogenic composition comprising immunogenic substances against more than one organism.

In some embodiments, the immunogenic composition of the present disclosure can comprise a first isolated nucleic acid and a second isolated nucleic acid, each encodes a deglycosylated recombinant influenza HA as described herein. In some embodiments, the first isolated nucleic acid and the second isolated nucleic acid can be selected from a group consisting of the four nucleic acids below. In certain embodiments, the immunogenic composition of the present disclosure can comprise all four nucleic acid below.
(1) an isolated nucleic acid encoding a recombinant influenza HA, which comprises an amino acid sequence at least 80%, 85%, 90%, 91%, 92%, 93%, 94%, 95%, 96%, 97%, 98%, or 99%, or 100% identical to SEQ ID NO: 6, provided that each of the 40^{th}, the 293^{rd}, the 304^{th}, and the 498^{th} amino acids thereof does not form a glycosylation site, and the 27^{th} amino acid thereof forms a glycosylation site;
(2) an isolated nucleic acid encoding a recombinant influenza HA, which comprises an amino acid sequence at least 80%, 85%, 90%, 91%, 92%, 93%, 94%, 95%, 96%, 97%, 98%, or 99%, or 100% identical to SEQ ID NO: 11, provided that each of the 24^{th}, the 38^{th}, the 54^{th}, the 61^{st}, the 79^{th}, the 301^{st}, and the 499^{th} amino acids thereof does not form a glycosylation site;
(3) an isolated nucleic acid encoding a recombinant influenza HA, which comprises an amino acid sequence at least 80%, 85%, 90%, 91%, 92%, 93%, 94%, 95%, 96%, 97%, 98%, or 99%, or 100% identical to SEQ ID NO: 3, provided that each of the 318^{th}, the 347^{th}, the 506^{th}, the 532^{nd}, and the 545^{th} amino acids does not form a glycosylation site, and the 40^{th} amino acid forms a glycosylation site; and
(4) an isolated nucleic acid encoding a recombinant influenza HA, which comprises an amino acid sequence at least 80%, 85%, 90%, 91%, 92%, 93%, 94%, 95%, 96%, 97%, 98%, or 99%, or 100% identical to SEQ ID NO: 18, provided that each of the 504^{th}, the 530^{th}, and the 543^{rd} amino acid does not form a glycosylation site, and each of the 40^{th}, the 316^{th}, and the 345^{th}, amino acids forms a glycosylation site.

### Formulation

In some embodiments, the composition is a pharmaceutical composition or pharmaceutical formulation. In such embodiments, the composition can further comprise a pharmaceutically acceptable excipient, adjuvant, or a combination thereof. The pharmaceutically acceptable excipient might comprise a solvent, dispersion media, diluent, dispersion, suspension aid, surface active agent, isotonic agent, thickening or emulsifying agent, preservative, polymer, peptide, protein, cell, hyaluronidase, or mixtures thereof. Various excipients for formulating pharmaceutical compositions and techniques for preparing the composition are known in the art (see Remington: The Science and Practice of Pharmacy, 22nd Edition, Edited by Allen, Loyd V., Jr, Pharmaceutical Press). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Formulation of standard pharmaceutically acceptable excipients may be carried out using routine methods in the pharmaceutical art (See Remington's Pharmaceutical Sciences, 19^{th} Edition, Mack Publishing Company, Eastern Pennsylvania, USA.).

In certain embodiments, the adjuvant can be but is not limited to C34, Gluco-C34, 7DW8-5, C17, C23, C30, α-galactosylceramide (α-GalCer), Aluminum salt (e.g., aluminum hydroxide, aluminum phosphate, alum (potassium aluminum sulfate), mixed aluminum salts), Squalene, MF59, QS-21, Freund's complete adjuvant, Freund's incomplete adjuvant, AS03 (GlaxoSmithKline), MF59 (Seqirus), CpG 1018 (Dynavax), or a combination thereof.

### Methods of Use

In one aspect, the present disclosure provides a method for generating an immune response against influenza virus infection, comprising administering a nucleic acid of the present disclosure to a subject in need at an effective amount. In some embodiment, the immune response can be characterized by an increased immunoglobin titer (e.g., an IgG titer) in the subject (e.g., in serum collected from the subject), and the immune response can be considered as being generated if the titer is higher than a benchmark level measured before the administration. In certain embodiments, the immunoglobin titer is higher than the benchmark by about 1, 2, 3, 4, 5, 6, 7, 8, 9, or 10 logs, or any range defined by the foregoing endpoints, such as, included or excluded 1 to 10 logs, 1 to 8 logs, 1 to 6 logs, 1 to 4 logs, 2 to 9 logs, 2 to 7 logs, 2 to 5 logs, 3 to 10 logs, 3 to 8 logs, 3 to 5 logs, or 4 to 6 logs. In yet some embodiments, the immunoglobin titer is higher than the benchmark by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200%, or any range defined by the foregoing endpoints, such as, included or excluded, 5 to 200%, 5 to 150%, 5 to 100%, 5 to 75%, 5 to 50%, 5 to 25%, 10 to 200%, 10 to 175%, 10 to 125%, 10 to 100%, 10 to 75%, 10 to 50%, 10 to 25%, 25 to 200%, 25 to 150%, 25 to 100%, 25 to 75%, 25 to 50%, 50 to 200%, 50 to 175%, 50 to 125%, 50 to 100%, or 50 to 75%. In some embodiments, the measurement can be conducted using an Enzyme-linked immunosorbent assay (ELISA).

In some embodiments, generating an immune response comprises preventing the subject from being infected by the influenza virus, but the method is not so limited. As described herein, preventing the subject from being infected by the influenza virus does not necessarily mean the subject would not be infected at all but alleviates the symptoms of influenza infections if the subject has been or will be infected by the influenza virus.

In some embodiments, the influenza infection is caused by an influenza A virus, an influenza B virus, or both. In some embodiments, the influenza A virus comprises a Group 1 IAV, comprising a H2, H5, H6, H8, H9, H11, H12, H13, H16, H17, or H18 virus, and/or a Group 2 IAV virus, comprising a H3, H4, H7, H10, H14, or H15 virus. In some embodiments, the influenza B virus comprises an IBV Victoria lineage and/or an IBV Yamagata lineage.

In some embodiments, the nucleic acid is delivered by a vector. In certain embodiments, the nucleic acid is configured as an expression vector according to an embodiment of the present disclosure. In some embodiments, the nucleic acid and/or the expression vector is formulated as a composition according to an embodiment of the present disclosure.

### Administration

Regarding the methods of the present disclosure, in some embodiments, the subject is administered with a single dose of the nucleic acid of the present disclosure. Yet in some embodiments, the subject is administered with an initial dose followed by at least one booster dose, e.g., one, two, three, four, five, six, seven, eight, nine, ten, eleven, twelve, thirteen, fourteen, fifteen, sixteen, seventeen, eighteen, nineteen, twenty, or more follow-up doses, with an interval of each dose in about, 1, 2, 3, 4, 5, 6, 7 days, about 1, 2, 3, 4, 5, 6, 7, 8, 9, or 10 weeks, or about 1, 2, 3, 4, 5, 6, 7, 8, 9, 10, 11, or 12 months, or any range defined by the foregoing endpoints, such as, included or excluded, 1 to 7 days, 1 to 5 days, 1 to 3 days, 1 to 10 weeks, 1 to 8 weeks, 1 to 6 weeks, 1 to 4 weeks, 1 to 2 weeks, 1 to 12 months, 1 to 8 months, 1 to 6 months, 1 to 4 months, 1 to 2 months, or 6 to 12 months. In certain embodiments, the nucleic acid of the present disclosure encapsulating is administered twice at the same or different doses, and the two administrations are separated by 1 day, 3 days, 5 days, 1 week, 2 weeks, 1 month, 2 months, 3 months, 6 months, 1 year, 1 to 5 days, 1 to 2 weeks, 1 to 3 months, 1 to 6 months, 1 month to 1 year, 3 months to 1 year, or 6 months to 1 year.

Administration route. The nucleic acid, as described herein, may be administered (as an expression vector or a composition according to an embodiment of the present disclosure) by any route. Suitable routes include, but are not limited to, oral, nasal, mucosal, submucosal, intravenous, intramuscular, intraperitoneal, subcutaneous, intradermal, transdermal, and buccal routes. Other possible routes of administration are by spray, aerosol, or powder application through inhalation via the respiratory tract.

Effective amount of administration. The effective amount described herein refers to the amount of the nucleic acid, the expression vector comprising the nucleic acid, or the composition comprising the expression vector according to an embodiment of the present disclosure that is sufficient to provide a desired effect. In the embodiments where the purpose of administering the nucleic acid of the present disclosure is to treat or alleviate an existing infection, the effective amount refers to a therapeutically effective amount, while in some other embodiments where the purpose is to prevent infection, the effective amount refers to a prophylactically effective amount.

The effective amount of the methods of the present disclosure can be determined based on several factors, including but not limited to the conditions of the subjects (age, gender, species, body weight, health status, etc.), the progress of the disease to be treated, the administration route, the dosage and interval of the administration, and the nature of the nucleic acid (such as the stability and/or translation capacity thereof). Accordingly, the effective amount of the methods of the present disclosure is about 5, 10, 15, 20, 25, 30, 35, 40, 45, 50, 55, 60, 65, 70, 75, 80, 90, 100, 125, 150, 175, 200, 250, 300, 350, 400, 450, 500, 550, 600, 650, 700, 750, 800, 850, 900, 950 or 1000 micrograms (μg or ug), or any range defined by the foregoing endpoints, such as, include or exclude, 5 micrograms to 1000 micrograms, 5 micrograms to 900 micrograms, 5 micrograms to 800 micrograms, 5 micrograms to 700 micrograms, 5 micrograms to 600 micrograms, 5 micrograms to 500 micrograms, 5 micrograms to 400 micrograms, 5 micrograms to 300 micrograms, 5 micrograms to 200 micrograms, 5 micrograms to 175 micrograms, 5 micrograms to 150 micrograms, 5 micrograms to 125 micrograms, 5 micrograms to 100 micrograms, 5 micrograms to 90 micrograms, 5 micrograms to 80 micrograms, 5 micrograms to 70 micrograms, 5 micrograms to 60 micrograms, 5 micrograms to 50 micrograms, 5 micrograms to 40 micrograms, 5 micrograms to 30 micrograms, 5 micrograms to 20 micrograms, 5 micrograms to 10 micrograms, 10 micrograms to 1000 micrograms, 10 micrograms to 900 micrograms, 10 micrograms to 800 micrograms, 10 micrograms to 700 micrograms, 10 micrograms to 600 micrograms, 10 micrograms to 500 micrograms, 10 micrograms to 400 micrograms, 10 micrograms to 300 micrograms, 10 micrograms to 200 micrograms, 10 micrograms to 175 micrograms, 10 micrograms to 150 micrograms, 10 micrograms to 125 micrograms, 10 micrograms to 100 micrograms, 10 micrograms to 90 micrograms, 10 micrograms to 80 micrograms, 10 micrograms to 70 micrograms, 10 micrograms to 60 micrograms, 10 micrograms to 50 micrograms, 10 micrograms to 40 micrograms, 10 micrograms to 30 micrograms, 10 micrograms to 20 micrograms, 50 micrograms to 1000 micrograms, 50 micrograms to 900 micrograms, 50 micrograms to 800 micrograms, 50 micrograms to 700 micrograms, 50 micrograms to 600 micrograms, 50 micrograms to 500 micrograms, 50 micrograms to 400 micrograms, 50 micrograms to 300 micrograms, 50 micrograms to 200 micrograms, 50 micrograms to 175 micrograms, 50 micrograms to 150 micrograms, 50 micrograms to 125 micrograms, 50 micrograms to 100 micrograms, 50 micrograms to 90 micrograms, 50 micrograms to 80 micrograms, 50 micrograms to 70 micrograms, or 50 micrograms to 60 micrograms. 100 micrograms to 1000 micrograms, 100 micrograms to 900 micrograms, 100 micrograms to 800 micrograms, 100 micrograms to 700 micrograms, 100 micrograms to 600 micrograms, 100 micrograms to 500 micrograms, 100 micrograms to 400 micrograms, 100 micrograms to 300 micrograms, 100 micrograms to 200 micrograms, 100 micrograms to 175 micrograms, 100 micrograms to 150 micrograms, 300 micrograms to 1000 micrograms, 300 micrograms to 900 micrograms, 300 micrograms to 800 micrograms, 300 micrograms to 700 micrograms, 300 micrograms to 600 micrograms, 300 micrograms to 500 micrograms, 300 micrograms to 400 micrograms, 500 micrograms to 1000 micrograms, 500 micrograms to 900 micrograms, 500 micrograms to 800 micrograms, 500 micrograms to 700 micrograms, 500 micrograms to 600 micrograms, 600 micrograms to 800 micrograms, or 700 micrograms to 900 micrograms.

In some embodiments, the nucleic acid, the expression vector, or the composition comprising the expression vector is administered at a dosage level from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, per subject body weight per day, one or more times a day, to obtain the desired in vivo effect.

### Other Definition

Unless specifically defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The practice of the present disclosure will employ, unless otherwise indicated, conventional techniques of microbiology, tissue culture, molecular biology, chemistry, biochemistry, and recombinant DNA technology, which are within the skill of the art. The materials, methods, and examples are illustrative only and not limiting. The following is presented by way of illustration and is not intended to limit the scope of the disclosure.

Numbers expressing quantities of ingredients, properties such as molecular weight, reaction conditions and results, and so forth, used to describe and claim certain embodiments of the present disclosure are to be understood as being modified in some instances by the term “about.” A skilled artisan in the field would understand the meaning of the term “about” in the context of the value that it qualifies. The numerical values presented in some embodiments of the present disclosure may contain certain errors resulting from the standard deviation in their respective testing measurements. For example, the term “about,” as used herein, refers to a measurable value such as an amount, a temporal duration, and the like and is meant to encompass variations of ±20%, ±10%, ±5%, ±1%, or ±0.1% from the specified value, as such variations are appropriate.

As used herein, “substantially” means sufficient to work for the intended purpose. The term “substantially” thus allows for minor, insignificant variations from an absolute or perfect state, dimension, measurement, result, or the like, such as expected by a person of ordinary skill in the field, but that does not appreciably affect overall performance. When used with respect to numerical values or parameters or characteristics expressed as numerical values, “substantially” means within ten percent.

As used herein, “treat,” “treatment,” and “treating” refer to an approach for obtaining beneficial or desired results, for example, clinical results. For the purposes of this disclosure, beneficial or desired results may include inhibiting or suppressing the initiation or progression of an infection or a disease, ameliorating or reducing the development of symptoms of an infection or disease, or a combination thereof.

As used herein, “preventing” and “prevention” are used interchangeably with “prophylaxis” and can mean complete prevention of infection or prevention of the development of symptoms of that infection, a delay in the onset of a disease or its symptoms, or a decrease in the severity of a subsequently developed infection or its symptoms.

An “effective amount” of a composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutically effective amount” may be used in reference to treatment, whereas “prophylactically effective amount” may be used to describe administering an effective amount to a subject that is susceptible to or at risk of developing a disease or disease-state (e.g., recurrence) as a beneficial and/or protective course of reducing (e.g., in a statistically significant manner relative to an untreated state) the likelihood of occurrence and/or severity of the disease or disease-state.

As described herein, “isolated” means that a subject protein or polypeptide (1) is free of at least some other proteins or polypeptides with which it would typically be found in nature, (2) is essentially free of other proteins or polypeptides from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a protein or polypeptide with which the “isolated protein” or “isolated polypeptide” may be associated in nature, (6) is operably associated (by covalent or noncovalent interaction) with a polypeptide with which it is not associated in nature, or (7) does not occur in nature. Such an isolated protein or polypeptide can be encoded by genomic DNA, cDNA, RNA or other RNA, of may be of synthetic origin according to any of a number of well-known chemistries for artificial peptide and protein synthesis, or any combination thereof. In certain embodiments, the isolated protein or polypeptide is substantially free from proteins or polypeptides or other contaminants that are found in its natural environment that would interfere with its use (therapeutic, diagnostic, prophylactic, research or otherwise).

The term “isolated nucleic acid” as used herein shall mean a polynucleotide of genomic, cDNA, or synthetic origin or some combination thereof, wherein by virtue of its origin the isolated nucleic acid (1) is not associated with all or a portion of a polynucleotide in which the isolated nucleic acid is found in nature, (2) is linked to a polynucleotide to which it is not linked in nature, or (3) does not occur in nature as part of a larger sequence. Reference to a nucleotide sequence as set out herein encompasses a DNA molecule with the specified sequence, and an RNA molecule with the specified sequence in which U is substituted for T, unless context requires otherwise.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein-coding sequence is achieved under conditions compatible with the transcriptional activity of the control sequences.

3. The term “control sequence,” as used herein, refers to polynucleotide sequences that can affect the expression, processing, or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences, and polyadenylation sequences. In certain embodiments, “control sequences” can include leader sequences and/or fusion partner sequences. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

As used herein, “recombinant” modifying a protein describes that the protein is designed to be produced by introducing an engineered nucleic acid into a host organism, like bacteria, yeast, or mammalian cells, using laboratory or industrial processes.

As described herein, percent identity between two polypeptides or nucleic acid sequences is determined in various ways that are within the skill in the art, for instance, using publicly available computer software such as Smith Waterman Alignment (Smith and Waterman (1981) J Mol Biol 147:195-7); “BestFit” (Smith and Waterman, Advances in Applied Mathematics, (1981) 482-489) as incorporated into GeneMatcher Plus™, Schwarz and Dayhof, Atlas of Protein Sequence and Structure, Dayhof, M. O., Ed (1979) 353-358; BLAST program (Basic Local Alignment Search Tool; (Altschul et al. (1990) J Mol Biol 215:403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the full length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Two sequences are said to be “identical” if they contain the same residues at corresponding positions when aligned for maximum correspondence, as described herein. Comparisons between two sequences are typically performed by aligning the sequences to identify regions of optimal local or global similarity. A “comparison region” or “comparison window,” as used herein, refers to a segment of at least about 20 contiguous positions, typically about 30 to about 75, or about 40 to about 50 positions, in which one sequence is aligned to a reference sequence of the same number of contiguous positions after optimal alignment has been achieved. When the sequence being compared is shorter than the stated comparison region, the comparison is made over the entire length of the sequence.

In certain embodiments, “percent identity” is determined by aligning two sequences to obtain the highest degree of correspondence using a suitable sequence alignment algorithm (for example, the Needleman-Wunsch or Smith-Waterman algorithm). The “percent identity” is calculated as the number of identical residues (nucleotides or amino acids) shared between the two sequences within the aligned region divided by the total number of aligned positions, and multiplied by 100. Insertions or deletions (gaps) may be introduced in one or both sequences to optimize alignment; however, such gaps are generally minimized by use of appropriate gap penalties. Unless otherwise specified, the reference sequence is the longer or full-length sequence being compared.

In some embodiments, the modification described herein is also contemplated according to certain embodiments of the present disclosure. The modification can include conservative and non-conservative amino acid substitutions. A “conservative substitution” refers to amino acid substitutions that do not significantly affect or alter a particular characteristic (e.g., a binding activity such as a specific binding activity) of a particular protein. Generally, conservative substitutions are ones in which a substituted amino acid residue is replaced with an amino acid residue having a similar side chain. Exemplary conservative substitutions include a substitution found in one of the following groups:

| | |
|---|---|
| Group 1 | Alanine (Ala or A), Glycine (Gly or G), Serine (Ser or S), Threonine (Thr or T) |
| Group 2 | Aspartic acid (Asp or D), Glutamic acid (Glu or Z) |
| Group 3 | Asparagine (Asn or N), Glutamine (Gln or Q) |
| Group 4 | Arginine (Arg or R), Lysine (Lys or K), Histidine (His or H) |
| Group 5 | Isoleucine (Ile or I), Leucine (Leu or L), Methionine (Met or M), Valine (Val or V) |
| Group 6 | Phenylalanine (Phe or F), Tyrosine (Tyr or Y), Tryptophan (Trp or W) |

Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitution groups include sulfur-containing: Met and Cysteine (Cys or C); acidic: Asp, Glu, Asn, and Gin; small aliphatic, nonpolar or slightly polar residues: Ala, Ser, Thr, Pro, and Gly; polar, negatively charged residues and their amides: Asp, Asn, Glu, and Gin; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

Amino acid sequence variants may be prepared, for instance, by introducing appropriate nucleotide changes into a nucleic acid that encodes the desired peptide or by peptide synthesis. Such modifications include, for example, deletions from, and/or insertions into, and/or substitutions of, residues within the amino acid sequence of a protein or polypeptide of interest. Any combination of deletion, insertion, and substitution may be made to arrive at the final product, provided that the final product exhibits the similar and desirable characteristics.

As described elsewhere herein, determination of the three-dimensional structures of the presently disclosed antibodies or antigen-binding fragments thereof may be made through routine methodologies such that substitution, addition, deletion, or insertion of one or more amino acids with selected natural or non-natural amino acids can be virtually modeled for purposes of determining whether a so derived structural variant retains the space-filling properties of presently disclosed species. A variety of computer programs are known to the skilled artisan for determining appropriate amino acid substitutions (or appropriate nucleic acids encoding the amino acid sequence).

As used herein, “glycan” or “glycosyl group refers to a polysaccharide, oligosaccharide, or monosaccharide. Glycans can be monomers or polymers of sugar residues and can be linear or branched. A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′ sulfo N-acetylglucosamine, etc.).

As used herein, “alkyl” refers to a hydrocarbon chain that may be a straight chain or branched chain, saturated or unsaturated, containing the indicated number of carbon atoms. For example, C₁₋₆ indicates that the group may have from 1 to 6 (inclusive) carbon atoms in it. Non-limiting examples include methyl, ethyl, iso-propyl, tert-butyl, n-hexyl. A “heteroalkyl” group is an alkyl group in which at least one carbon of the chain has been replaced by a heteroatom. In some embodiments, the heteroalkyl group has 1 to 20 carbon atoms. The term “alkoxy” is intended to mean the moiety-OR, where R is alkyl. The term “aryloxy” is intended to mean the moiety-OR, where R is aryl.

As used herein, “alkenyl” refers to a hydrocarbon chain including at least one double bond, which may be a straight chain or branched chain, and containing the indicated number of carbon atoms. For example, C₂₋₆ indicates that the group may have from 2 to 6 (inclusive) carbon atoms in it. Non-limiting examples include ethenyl and prop-1-en-2-yl.

As used herein, “alkynyl” refers to a hydrocarbon chain including at least one triple bond, which may be a straight chain or branched chain, and containing the indicated number of carbon atoms. For example, C₂₋₆ indicates that the group may have from 2 to 6 (inclusive) carbon atoms in it. Non-limiting examples include ethynyl and 3,3-dimethylbut-1-yn-1-yl.

As used herein, “cycloalkyl” refers to a nonaromatic cyclic, bicyclic, fused, or spiro hydrocarbon radical having 3 to 10 carbons, such as 3 to 8 carbons, such as 3 to 7 carbons, wherein the cycloalkyl group, which may be optionally substituted. Examples of cycloalkyls include five-membered, six-membered, and seven-membered rings. A cycloalkyl can include one or more elements of unsaturation; a cycloalkyl that includes an element of unsaturation is herein also referred to as a “cycloalkenyl”. Examples include cyclopropyl, cyclobutyl, cyclopentyl, cyclopentenyl, cyclohexyl, cyclohexenyl, cycloheptyl, and cyclooctyl.

As used herein, “heterocycloalkyl” refers to a nonaromatic 5-8 membered monocyclic, 8-12 membered bicyclic, or 11-14 membered tricyclic ring fused or spiro system radical having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Heterocycloalkyls can also include oxidized ring members, such as —N(O)—, —S(O)—, and —S(O)₂—. Examples of heterocycloalkyls include five-membered, six-membered, and seven-membered heterocyclic rings. Examples include piperazinyl, pyrrolidinyl, dioxanyl, morpholinyl, tetrahydrofuranyl, and the like.

As used herein, “aryl” or “aryl group” refers to a moiety formed by the removal of one or more hydrogen (“H”) or deuterium (“D”) from an aromatic compound. The aryl group may be a single ring (monocyclic) or have multiple rings (bicyclic, or more) fused together or linked covalently. A “carbocyclic aryl” has only carbon atoms in the aromatic ring(s). A “heteroaryl” is intended to mean an aromatic ring system containing 5 to 14 aromatic ring atoms that may be a single ring, two fused rings or three fused rings wherein at least one aromatic ring atom is a heteroatom selected from, but not limited to, the group consisting of O, S and N. Heteroaryls can also include oxidized ring members, such as —N(O)—, —S(O)—, and —S(O)₂—. Examples include furanyl, thienyl, pyrrolyl, imidazolyl, oxazolyl, thiazolyl, isoxazolyl, pyrazolyl, isothiazolyl, oxadiazolyl, triazolyl, thiadiazolyl, pyridinyl, pyrazinyl, pyrimidinyl, pyridazinyl, triazinyl and the like. Examples also include carbazolyl, quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, isobenzofuran and the like.

As used herein, “amine” refers to a compound that contains a basic nitrogen atom with a lone pair. The term “amino” refers to the functional group or moiety —NH₂, —NHR, or —NR₂, where R is the same or different at each occurrence and can be an alkyl group or an aryl group.

As used herein, “halogen” or “halo” refers to fluorine, bromine, chlorine, or iodine. In particular, it typically refers to fluorine or chlorine when attached to an alkyl group and further includes bromine or iodine when on an aryl or heteroaryl group.

As used herein, the term “haloalkyl” refers to an alkyl as defined herein, which is substituted by one or more halo groups. The haloalkyl can be monohaloalkyl, dihaloalkyl, trihaloalkyl, or polyhaloalkyl, including perhaloalkyl. A monohaloalkyl can have one chloro or fluoro within the alkyl group. Chloro and fluoro are commonly present as substituents on alkyl or cycloalkyl groups; fluoro, chloro, and bromo are often present on aryl or heteroaryl groups. Dihaloalkyl and polyhaloalkyl groups can have two or more of the same halo atoms or a combination of different halo groups on the alkyl. Typically, the polyhaloalkyl contains up to 12, or 10, or 8, or 6, or 4, or 3, or 2 halo groups. Non-limiting examples of haloalkyl include fluoromethyl, difluoromethyl, trifluoromethyl, chloromethyl, dichloromethyl, trichloromethyl, 2,2,2-trifluoroethyl, pentafluoroethyl, heptafluoropropyl, difluorochloromethyl, dichlorofluoromethyl, difluoroethyl, difluoropropyl, dichloroethyl and dichloropropyl. A perhalo-alkyl refers to an alkyl having all hydrogen atoms replaced with halo atoms, e.g., trifluoromethyl.

As used herein, unless otherwise specified, the term “heteroatom” refers to a nitrogen (N), oxygen (O), or sulfur(S) atom.

### EXAMPLE

### Example A1: Low-Sugar IBV Yamagata mRNA Vaccine

This experiment prepared an exemplary RNA vaccine comprising a nucleic acid encoding a low-sugar IBV HA and tested its cross activities. The IBA HA tested in this experiment was designed to have no glycosylation site in the stem region or only one glycosylation site at the N40 residue. The IBA HA was derived from a Yamagata B/Phuket/3073/2013, and the exemplary mRNA vaccine encoding the Phuket IBA HA was tested for its cross-activities in an IBV Austria/1359417/2021 strain, an IBV Maryland/1959 strain, and an IBV Colorado/06/2017 strain.

mRNA synthesis of WT and de-glycosylated HA. The codon-optimized gene of HA variants was synthesized by BIOTOOLS (BIOTOOLS Co., Ltd., Taipei, Taiwan) and cloned into pcDNA3.1. To delete the N-glycosites, the putative sequon N-Xa-S/T was changed to Q-X-S/T in the plasmid synthesized. After confirming the protein expression in the HEK293 cell line, the genes were cloned to an in vitro transcription (IVT) plasmid (the Taiwan Bio-manufacturing Corporation) flanked by the 5′ and 3′ UTR followed by a 120-nucleotide poly(A) tail. For mRNA production, each DNA plasmid was digested by SwaI at 25° C. overnight and then checked with a 1% agarose gel. The mRNA synthesis was performed through a two-step enzymatic method. First, the IVT reactions were carried out with T7 polymerase and DNA template at 37° C. for 3 hours. The sample was treated with DNase I at 37° C. for 30 minutes and then purified with LiCl to remove linear DNA. Second, the capping reactions were carried out with two capping enzymes. Samples were heated with a reaction mixture at 65° C. for 5 minutes, and the enzymatic reaction was performed at 37° C. for 3 hours. All mRNA products were then stored in the DEPC water. As a result, exemplary mRNA vaccines encoding the following HAs were prepared.

TABLE
Exemplary mRNA vaccines of Example A1

| Name | HA |
|---|---|
| Yamagata Wild-type | Yamagata B/Phuket/3073/2013 HA (Control) |
| deg-stem IBV | N40Q/N318Q/N347Q/N506Q/N532Q/N545Q variant HA |
| deg-stem + N40 IBV | N318Q/N347Q/N506Q/N532Q/N545Q variant HA (SEQ ID NO: 3) |

mRNA-LNP preparation. The LNPs (MODERNA®) were prepared by mixing an aqueous phase (containing mRNA) and an organic phase (containing lipid mixtures) into a microfluidic cartridge equipped with Precision Nanoassemblr® Ignite™. The mRNA was diluted with 25 mM sodium acetic buffer at pH 5, while the lipid mixture was dissolved in ethanol using a molar ratio of 50:10:38.5:1.5, SM-102, DSPC, cholesterol, and PEG-lipid, respectively. Microfluidic mixing was performed at a flow rate ratio (aqueous to ethanol) of 3:1 (vol/vol) and a total flow rate of 12 mL/min. The N/P ratio was maintained at 6:1. All instrument parameters were the same as the above parameters. The resulting mRNA-LNPs solution was immediately diluted with 20 mM Tris buffer (pH 7.4) containing 8% sucrose and concentrated in an Amicon® Ultra centrifugal filter. Then, mRNA-LNPs were filtered through a 0.22-μm PES syringe filter for sterilization and stored at −80° C. in a refrigerator for further use.

Animals and Immunization. BALB/c mice (aged 6 to 8 weeks) were randomly grouped into three groups, each with 5 mice (n=5). Each mouse was immunized intramuscularly with 20 μg of mRNA-LNPs as prepared above in phosphate-buffered saline (PBS) with 300 mM sucrose. Animals were immunized at Week 0 and boosted with a second vaccination at Week 2, and serum samples were collected from each mouse 1 and 2 weeks (Week 3 and Week 4) after the booster immunization.

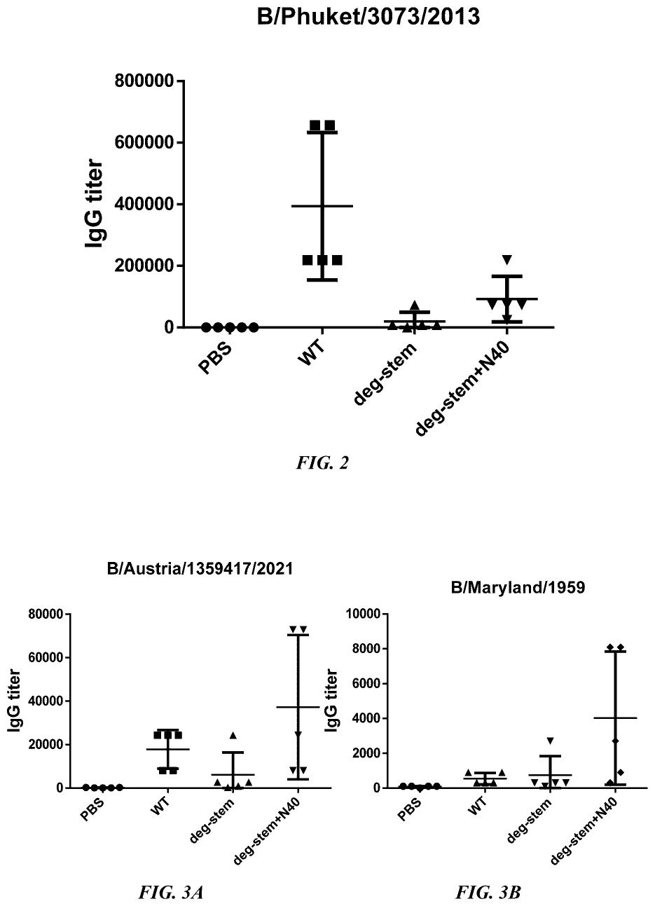

ELISA. The collected serum samples were then examined for their IgG titers against wild-type HA of the same or different lineages. Briefly, 0.1 μg of wild-type HA proteins were added to each well of 96-well plates and incubated overnight at 4° C. After the PBST wash, the serum samples obtained from immunized mice were serially diluted three times (starting from 100× dilution) and respectively added onto plates and incubated for 1 hour at room temperature (around 25 to 28° C.). The plates were then washed three times with PBST, and a reagent containing horseradish peroxidase-labeled anti-mouse IgGs was added to each well, followed by another 1-hour incubation at room temperature. All samples were performed in duplicates. The results of the ELISA assay are shown in FIG. 2.

T cell response. Splenocytes obtained from BALB/c mice immunized with either indicated influenza mRNA vaccine or PBS were collected and restimulated with strain-specific HA peptide pools, including H1N1 (A/Victoria/4897/2022, A/California/07/2009) and H5N1 (A/Cambodia/NPH230032/2023) for 48 hours. Protein transport inhibitor (Brefeldin A) was added at the last 4 hours of the culture. Subsequently, the cells were harvested, fixed, permeabilized and analyzed for T cell surface markers (CD3 and CD8) and intracellular granzyme B (GrzB) staining by flow.

Results. FIG. 2 shows the IgG titers (presented as OD values) of the serum samples against a wild-type IBV Yamagata lineage. It was observed that the mice immunized with the mRNA-LNPs encoding wild-type IBV Yamagata HA generated the highest IgG titers among the groups. The mice immunized with the mRNA-LNPs encoding mutated IBV Yamagata HA showed lower IgG titer against the wild-type IBV Yamagata HA, suggesting that the mutation affected the protein folding and, as a result, the immune response against the wild-type HA. It was also noticed that the mRNA-LNP encoding the N40Q/N318Q/N347Q/N506Q/N532Q/N545Q variant IBV Yamagata HA (“LNP-deg-stem IBV”) induced lower IgG titer than the mRNA-LNP encoding the N318Q/N347Q/N506Q/N532Q/N545Q variant IBV Yamagata HA (“LNP-deg-stem+N40 IBV”). This observation implies that N40, located in the N-terminal of the HA1 subunit, impacts the folding at a greater level than other glycosylation sites in the stem region.

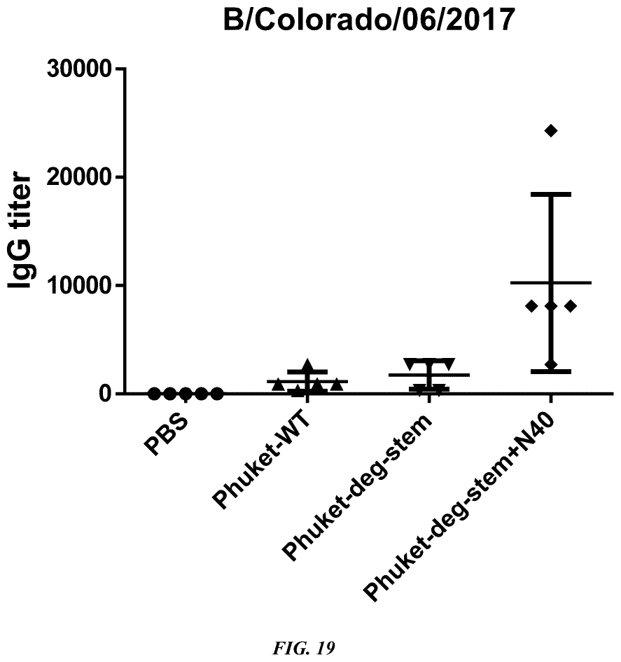

Surprisedly, while the deg-stem vaccine induced a slightly higher IgG titer against IBV Victoria strains, IBV Austria/1359417/2021 (FIG. 3A), IBV Maryland/1959 strain (FIG. 3B), and IBV Colorado/06/2017 (FIG. 19), the deg-stem+N40 vaccine demonstrated an even stronger IgG titer against the three Victoria strains.

See FIG. 3A. Most importantly, the IgGs with cross-activities comprised about 40% (37260/92340*100%=40.3%) of the total IgGs induced by the deg-stem+N40 IBV Yamagata HA, which was over ten times higher than that of the IgGs induced by the wild-type Yamagata HA (14580/393600*100%=3.7%). Although removing N40 affected the HA folding, the LNP-deg-stem IBV group still displayed a higher percentage (about two-fold) of IgG having the cross-activities against the Victoria lineage than the wild-type group (1500/24480*100%=6.1%). The incremental in IgG titer was much more significant in IBV Maryland/1959 strain (FIG. 3B) and IBV Colorado/06/2017 (FIG. 19). The IgG titers induced by the deg-stem+N40 vaccine were 8 times and 9 times higher than the wild-type vaccine respectively. As shown more clearly in the table below, the LNP-deg-stem+N40 IBV group induced a higher IgG titer against the B/Austria/1359417/2021 (Victoria) strain, compared with that of the wild-type group (37260 v. 14580).

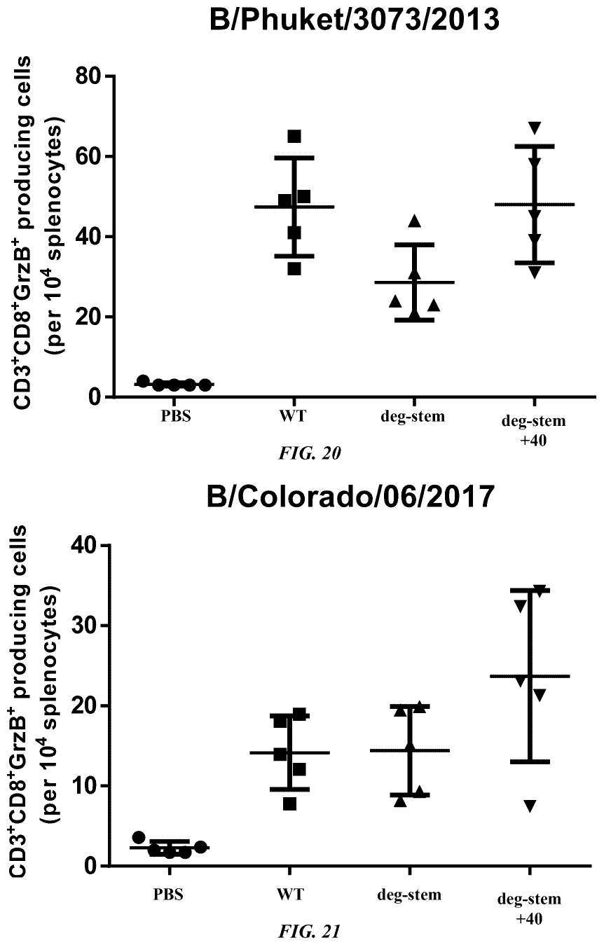

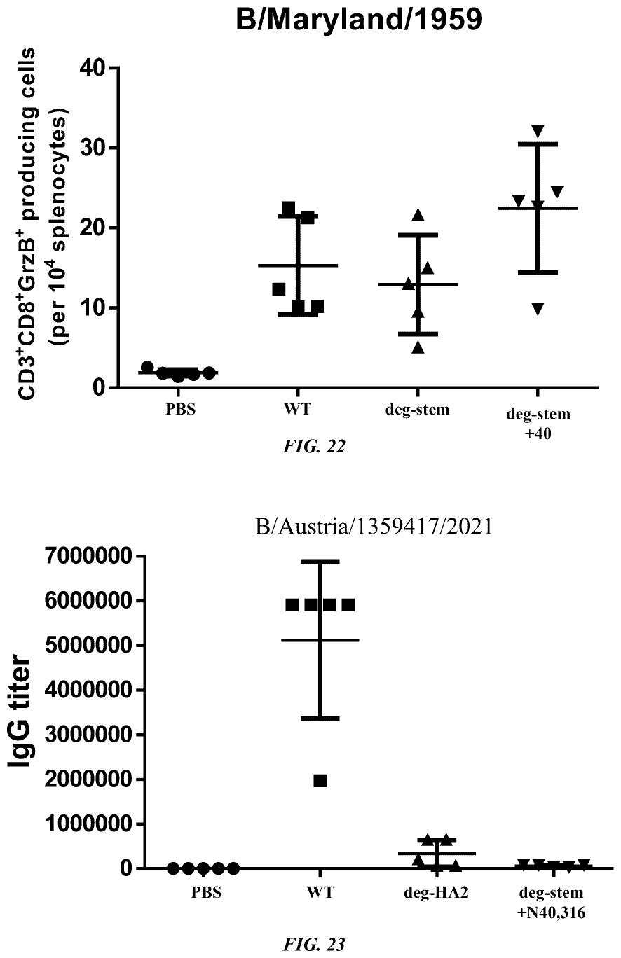

On the other hand, the T cell responses induced by the tested vaccines showed a similar trend. The wild-type vaccine and the deg-stem+N40 vaccine induced similar T cell response against Yamagata B/Phuket/3073/2013 (FIG. 20), but the deg-stem+N40 vaccine performed much better against Colorado/06/2017 (FIG. 21) and Maryland/1959 strain (FIG. 22), both are Victoria lineages. Together, the result shows that the removal of glycosylation sites in the stem region can increase the cross-activity of the vaccine. If the proper protein folding is concerned, the glycosylation sites in the N-terminal region of the HA1 subunit ((particularly, the N40 residue of the HA) can be kept, and the resulting vaccine maintains a better cross-activity than the vaccine encoding a wild-type HA.

TABLE
ELISA assay results showing IgG titer

| | WT | deg-stem | deg-stem + 40 | PBS |
|---|---|---|---|---|
| | (B/Yamagata) | (B/Yamagata) | (B/Yamagata) | (B/Yamagata) |
| B/Austria/1359417/2021 | 14580 | 1500 | 37260 | 100 |
| (B/Victoria lineage) | | | | |
| B/Phuket/3073/2013 | 393600 | 24480 | 92340 | 100 |
| (B/Yamagata lineage) | | | | |
| Percentage of IgG with | 3.7% | 6.1% | 40.3% | |
| cross-activities | | | | |

### Example A2: Low-Sugar IAV mRNA Vaccine

This experiment prepared an exemplary mRNA vaccine comprising a nucleic acid encoding a low-sugar IAV HA and tested its cross-activities. The encoded IAV HA was derived from a wild-type A/Victoria/4897/2022 (H1N1) strain and was designed to have no glycosylation site in the stem region or only one glycosylation site at N27/28 residue.

The mRNA synthesis, mRNA-LNP preparation, Animals and Immunization, and ELISA performed in this experiment followed the same protocols as described in Example 1, except that the wild-type HA proteins used in these ELISA were HAs respectively from A/Victoria/4897/2022 (H1N1), A/California/06/2009 (H1N1), A/Pavia/65/2016 (H1N1), and A/Cambodia/NPH230032/2023 (H5N1). The California H1N1 is a homologous strain to the Victoria H1N1; the Pavia H1N1 is heterologous to the Victoria H1N1; and the Cambodia H5N1 is a heterosubtype to the Victoria H1N1. As a result, exemplary mRNA vaccines encoding the following HAs were prepared.

T cell response. Splenocytes obtained from BALB/c mice immunized with either indicated influenza mRNA vaccine or PBS were collected and restimulated with strain-specific HA peptide pools, including H1N1 (A/Victoria/4897/2022, A/California/07/2009) and H5N1 (A/Cambodia/NPH230032/2023) for 48 hours. Protein transport inhibitor (Brefeldin A) was added at the last 4 hours of the culture. Subsequently, the cells were harvested, fixed, permeabilized and analyzed for T cell surface markers (CD3 and CD8) and intracellular granzyme B (GrzB) staining by flow. Table: Exemplary mRNA vaccines of Example A2

| Name | HA |
|---|---|
| H1N1 Wilde type | A/Victoria/4897/2022 HA (Control) |
| deg-stem IAV | N27Q/N28Q/N40Q/N293Q/N304Q/N498Qvariant HA |
| deg-stem + N27/28 IAV | N40Q/N293Q/N304Q/N498Q variant HA (SEQ ID NO: 6) |

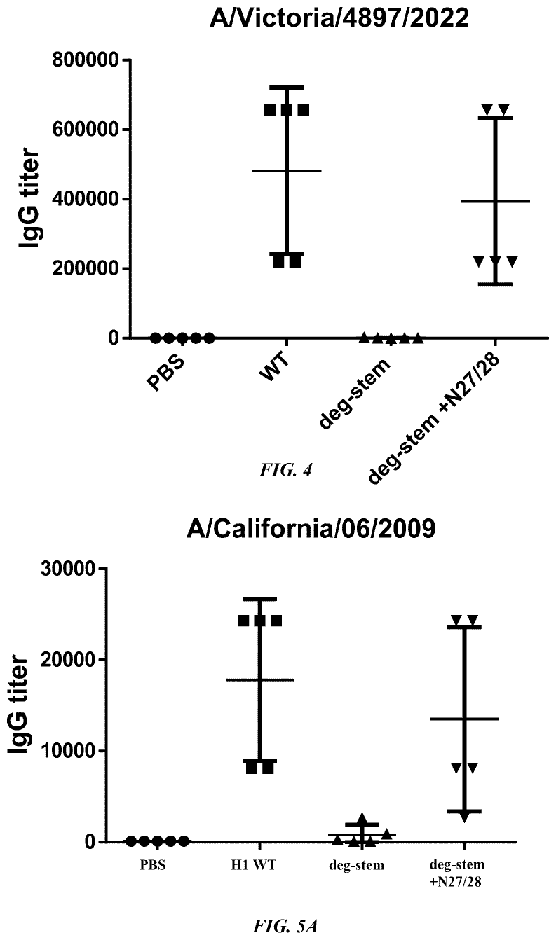

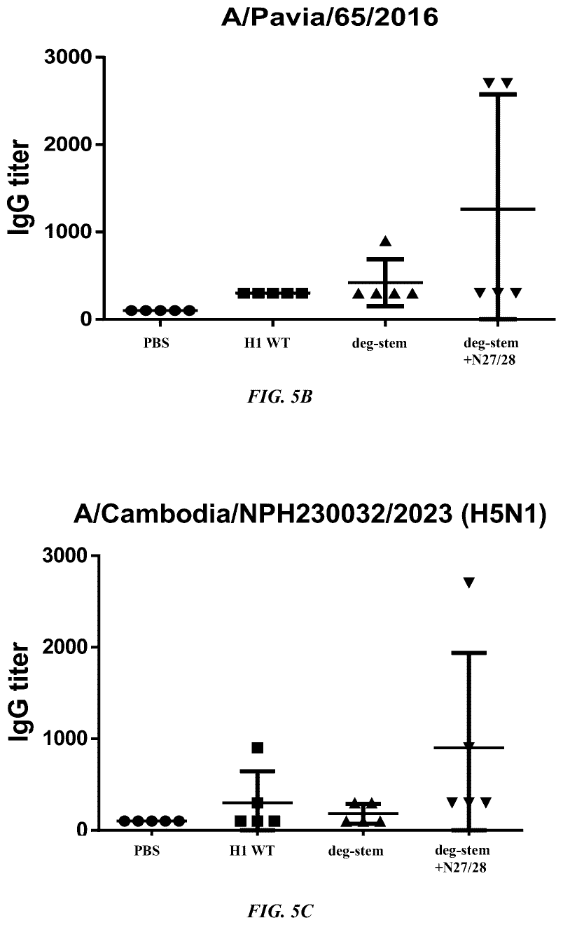

Results. FIG. 4 shows the IgG titers (presented as OD values) of the serum samples against a wild-type Victoria H1N1 strain. As expected, the mRNA vaccine encoding wild-type HA (“LNP-H1 WT”) induced an immune response in host animals against the Victoria H1N1 strain. The IgG titer induced by the mRNA vaccine encoding the HA with no glycosylation sites in the stem region thereof (“LNP-deg-stem”) was noticeably lower than that of the LNP-H1 WT vaccine. Nevertheless, as the IgG titer of the LNP-deg-stem vaccine was still higher than the negative control (PBS), this shows that the LNP-deg-stem vaccine still can induce the immune response against the Victoria H1N1 strain. The LNP-deg-stem vaccine also induced an immune response against the A/California/06/2009 (H1N1), a homologous strain to the Victoria H1N1 (FIG. 5A) Besides, compared with the wild-type vaccine, the LNP-deg-stem vaccine induced a similar level of immune response against the A/Pavia/65/2016 (H1N1), a heterologous strain to the Victoria H1N1 (FIG. 5B) and a better immune response against the A/Cambodia/NPH230032/2023 (H5N1), a heterosubtype strain to the Victoria H1N1 (FIG. 5C). In other words, despite that the removal of all glycosylation sites of the stem region inevitably impacted the protein folding, it exposed the hydrophobic pockets of the stem region, thereby obtaining better cross-activities.

The decrease in IgG titer observed in the LNP-deg-stem vaccine was restored if the N27 residue was kept as shown by the LNP-deg-stem+N27 vaccine (FIG. 4). This observation shows that the N27 residue, located in the N-terminal region of the HA1 subunit forming the stem region, played a bigger role in protein folding than other glycosylation sites, and keeping it can assure proper protein folding. Besides, compared with the wild-type vaccine, although the LNP-deg-stem+N27 vaccine induced slightly less IgG titer against the homologous California strain (FIG. 5A), it induced a much better IgG response against the heterologous Pavia strain (FIG. 5B; about 4.2 times better) and the heterosubtype Cambodia strain (FIG. 5C; about 3 times better).

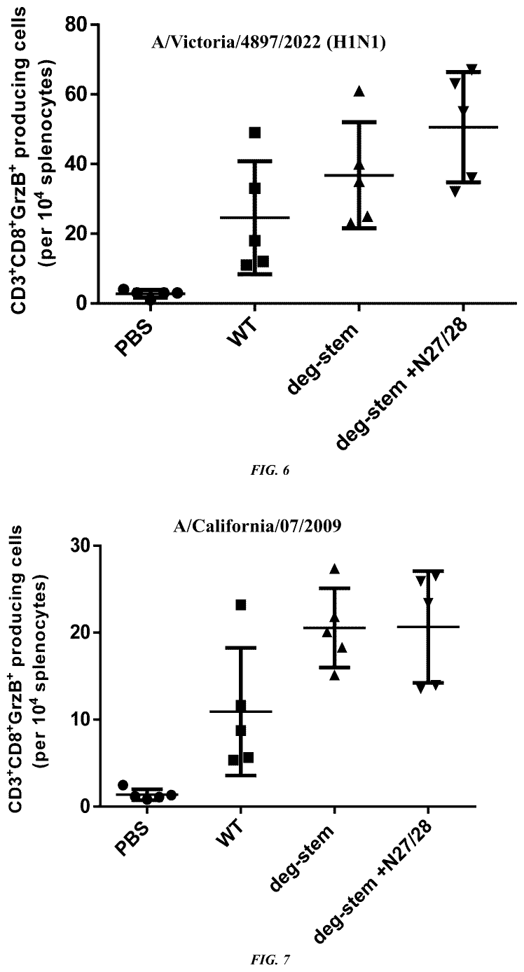

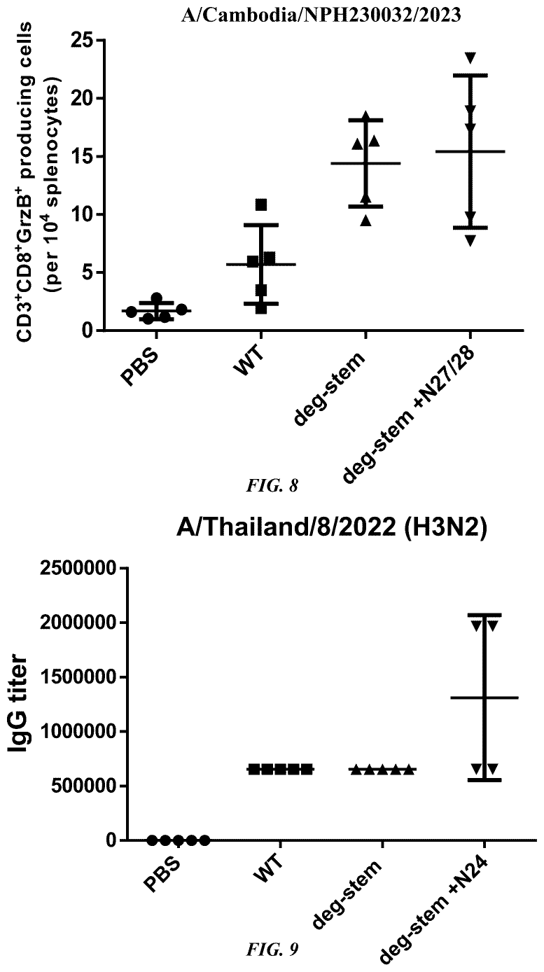

On the other hand, both LNP-deg-stem vaccine and LNP-deg-stem+N27 vaccine exhibited stronger T cell response than the wild-type vaccine, and LNP-deg-stem+N27 vaccine was the best in the group (FIG. 6). Moreover, while the two deglycosylation vaccines performed similarly against the California (H1N1) strain (FIG. 7), the LNP-deg-stem+N27 vaccine exhibited much better T cell response against the heterosubtype Cambodia strain (H5N1) (FIG. 8).

Accordingly, the results show that while deglycosylation vaccines provided better immune responses overall, N27 was identified to be the key N-glycosylation site to maintain a proper protein structure and exert better neutralization and effector function across different subtypes of the IAV. The observation also suggests that by keeping the glycosylation site in the N-terminal region of the HA1 subunit while removing other glycosylation sites in the stem region, the glycan shield covering the stem region was removed sufficiently to expose the hydrophobic pockets of the stem region for better cross-activities.

### Example A3: Low-Sugar IAV mRNA Vaccine

Similar to Example A2, this example demonstrated the low-sugar strategy in IAV H3 strain. The mRNA vaccines preparation, the IgG assay, and the T cell response assay were all performed as described in Example A2.

Exemplary mRNA vaccines in this example were prepared based on IAV A/Thailand/8/2022 (H3N2) strain, encoding HAs of different deglycosylation statuses. See Table below. The LNP-deg-stem vaccine encoded the HA of the A/Thailand/8/2022 strain with all glycosylation sites within the stem region removed. The deg stem IAV vaccine encoded the H3N2 HA with all glycosylation sites in stem region removed, and the deg-stem+N24 IAV vaccine encoded the stem-region deglycosylation HA but with the N24 site remained. Additionally, this experiment also tested several other deglycosylations. The H3N2 HA encoded by the deg-stem-N54-61-79 IAV vaccine had all glycosylation sites in stem region removed and also had N54, N61, and N79 sites removed. Similarly, the deg-stem-N54-61-79-301 IAV vaccine, the deg-stem-N61-301 IAV, and the deg-stem-N262-301 IAV all had additional glycosylation sites removed as indicated by the amino acid numbers.

TABLE
Exemplary mRNA vaccines of Example A3

| Name | HA |
|---|---|
| H3N2 Wilde type | A/Thailand/8/2022 (H3N2) HA (Control)(SEQ ID NO: 7) |
| deg-stem | N24Q/N38Q/N499Q variant HA |
| deg-stem + N24 | N38Q/N499Q variant HA (SEQ ID NO: 9) |
| deg-stem − N54-61-79 | N24Q/N38Q/N54Q/N61Q/N79Q/N499Q variant HA (SEQ ID NO: 10) |
| deg-stem − N54-61-79-301 | N24Q/N38Q/N54Q/N61Q/N79Q/N301Q/N499Q variant HA (SEQ ID |
| | NO: 11) |
| deg-stem − N61-301 | N24Q/N38Q/N61Q/N301Q/N499Q variant HA (SEQ ID NO: 12) |
| deg-stem − N262-301 | N24Q/N38Q/N262Q/N301Q/N499Q variant HA (SEQ ID NO: 13) |

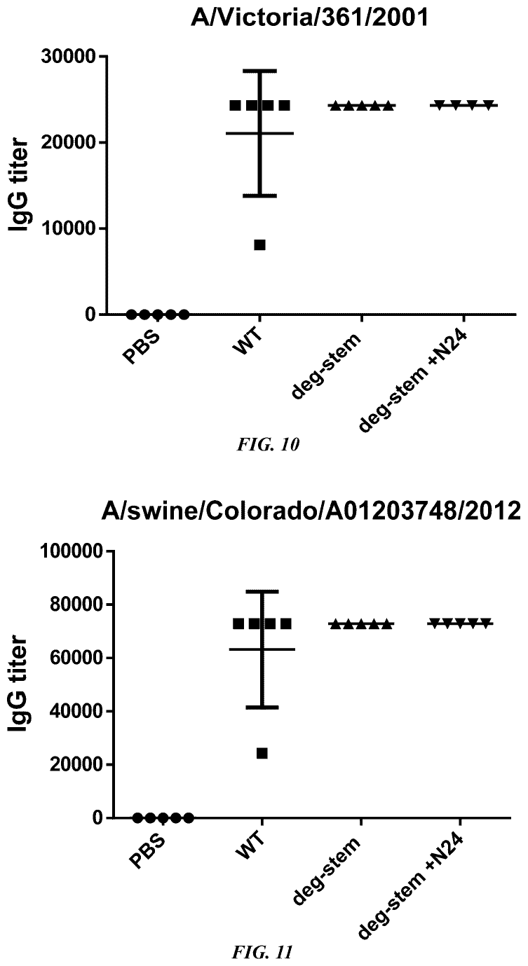

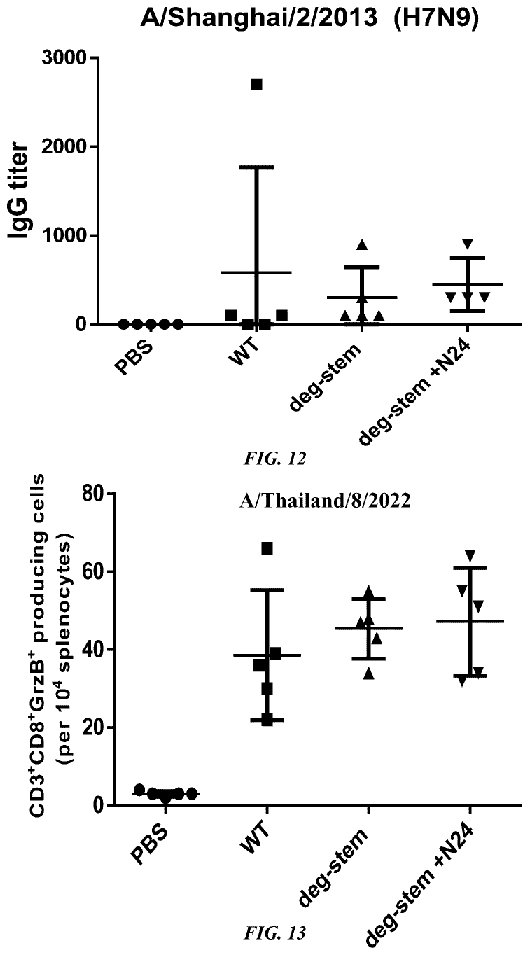

Result—Stem Region Deglycosylation. It was observed that the vaccine encoding a HA with the deglycosylated stem region did not exhibit noticeably better IgG response against the A/Thailand/8/2022 strain than the wide-type vaccine. However, the deg-stem+N24 vaccine triggered a much stronger IgG response (FIG. 9) than the wide-type vaccine and the deg-stem vaccine. Furthermore, both of the deg-stem vaccine and the deg-stem+N24 vaccine were slightly better than the wide-type vaccine in triggering IgG responses against A/Victoria/361/2001 (FIG. 10) and A/Swine/Colorado/A01203748/2012 (FIG. 11), which are different H3N2 strains, and their HA are 93% and 86% identical to that of the A/Thailand/8/2022 strain. On the other hands, the wild-type vaccine, the deg-stem vaccine, and the deg-stem+N24 vaccine performed similarly well against a H7N9 strain, A/Shanghai/2/2013 (FIG. 12).

From the T cell response assay (GrzB staining), we observed that both of the deg-stem vaccine and the deg-stem+N24 vaccine showed enhanced T cell response than the wild-type vaccine against A/Thailand/8/2022 strain (FIG. 13), A/Victoria/361/2001 (FIG. 14), and A/Swine/Colorado/A01203748/2012 (FIG. 15). Moreover, the deg-stem+N24 vaccine was better than the deg-stem vaccine. Together with the results observed in the IgG responses, these experiments confirmed that deglycosylation in the stem region was able to increase both the neutralization and effector function of the vaccines. Most importantly, keeping the N24 glycosite did not adversely affect the benefit of stem-region deglycosylation; instead, that increased the immunogenicity of the vaccine.

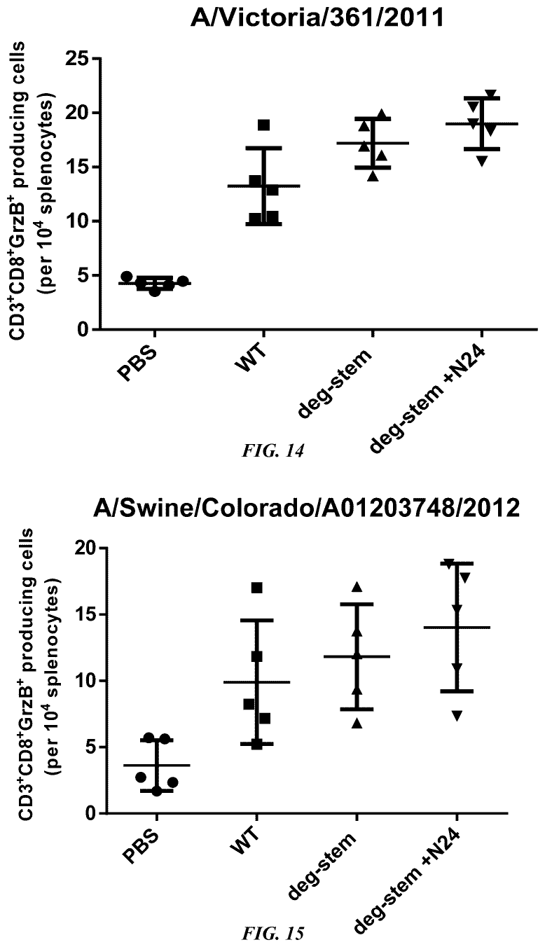

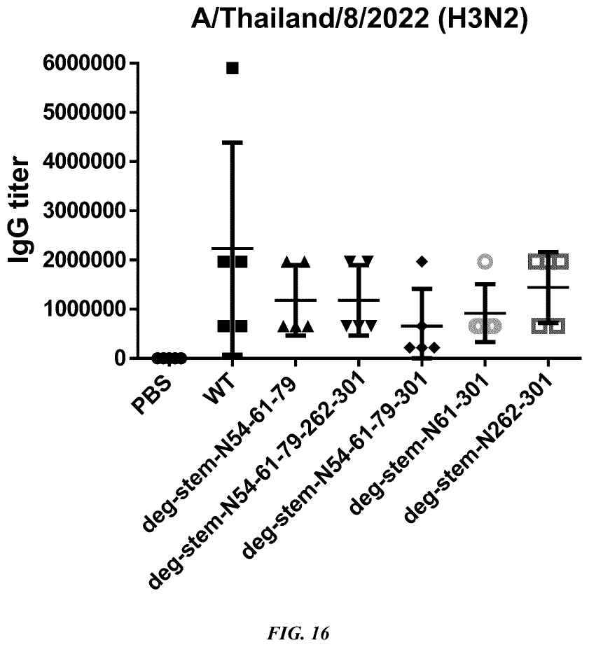

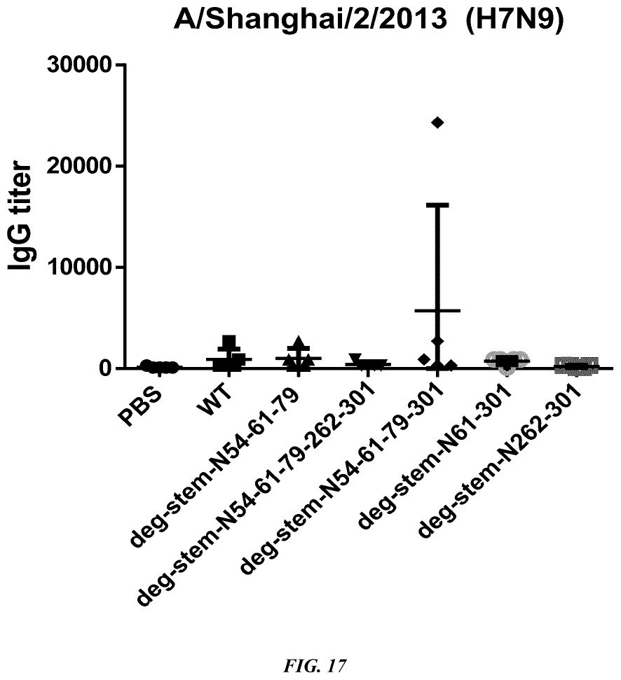

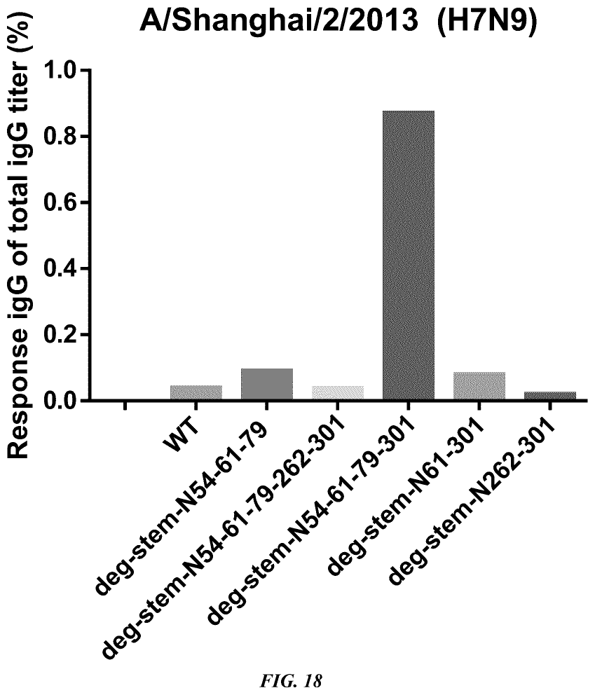

Result—Stem Region and Additional Deglycosylation. Additional deglycosylations were tested to see whether other glycosylation sites also contribute to better immunogenicity. The results demonstrated that all the tested deglycosylation vaccines in this particular experiment did not trigger a higher IgG titer than the wild-type vaccine against A/Thailand/8/2022 strain (FIG. 16). Most of the deglycosylation vaccines did not show advantages against A/Shanghai/2/2013 (H7N9) either, except for the deg-stem-N54-61-79-301 vaccine (FIG. 17). The deg-stem-N54-61-79-301 vaccine triggered a 6-time higher IgG response than the wild-type vaccine (FIG. 17). Moreover, the deg-stem-N54-61-79-301 vaccine also exhibited a better T cell response—22 times higher—than the wild-type vaccine (FIG. 18). This experiment demonstrated that in addition to the deglycosylation in the stem region, additionally removing the glycosylation sites at N54, N61, N79, and N301 further improved immunogenicity in both neutralization and effector function aspects.

### Example A4: Low-Sugar IBV Victoria mRNA Vaccine

This example tested the present disclosure's deglycosylation concept in IBV Victoria lineage. The mRNA vaccines preparation, the IgG assay, and the T cell response assay were all performed as described in Example A1 and other examples described herein. Exempary mRNA vaccines in this example were prepared based on IBV Victoria B/Austria/1359417/2021 strain, encoding HAs of different deglycosylation statuses. See Table below. The deg-stem vaccine encoded a B/Austria/1359417/2021 HA with all glycosylation sites in the stem region removed. One should note that the glycosylation sites removed included N316. Although N316 technically is not within the stem region, but the glycosylation site is considered within the stem region. This is because the sequon of N316 comprises the 316^{th} amino acid, the 317^{th} amino acid, and the 318^{th} amino acid—the latter two amino acids are both within the stem region. Therefore, it is believed that the glycan conjugated to this glycosylation site will shield the stem region. Hence, N316 was removed in the deg-stem vaccine.

Besides, and also described in the table, the deg-stem+N40 vaccine encoded a B/Austria/1359417/2021 HA with all glycosylation sites, except for the N40 one, in the stem region removed. The deg-stem+N40,316 vaccine encoded a B/Austria/1359417/2021 HA with all glycosylation sites, except for N40 and N316, in the stem region removed. The deg-stem+N40, 316, 345 vaccine encoded a B/Austria/1359417/2021 HA with all glycosylation sites, except for N40, N316, and N345, in the stem region removed.

The B/Austria/1359417/2021 HA comprises glycosylation sites including N40, N74, N160, N178, N245, N316, N345, N504, N530, N543, and N575. Among them, N40, N316, N345, N504, N530, and N543 are within the stem region, and N40, N316, are N345 belong to the HA1 domain, and N504, N530, and N543 belong to the HA2 domain. N504, N530, and N543 happen to be the only glycosylation sites in the HA2 domain. Therefore, the deg-stem+N40, 316, 345 vaccine is also viewed as a deg-HA2 vaccine, encoding a B/Austria/1359417/2021 HA with all glycosylation sites of the HA2 domain removed.

TABLE
Exemplary mRNA vaccines of Example A4

| Name | HA |
|---|---|
| IBV Victoria Wild type | B/Austria/1359417/2021 HA (Control)(SEQ ID NO: 14) |
| deg-stem | N40Q/N316Q/N345Q/N504Q/N530Q/N543Q variant HA |
| deg-stem + N40 | N316Q/N345Q/N504Q/N530Q/N543Q variant HA (SEQ ID NO: |
| | 16) |
| deg-stem + N40, 316 | N345Q/N504Q/N530Q/N543Q variant HA (SEQ ID NO: 17) |
| deg-stem + N40, 316, 345 (deg- | N504Q/N530Q/N543Q variant HA (SEQ ID NO: 18) |
| HA2) | |

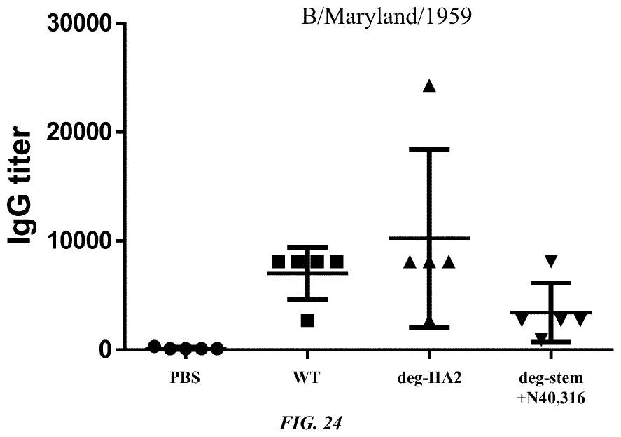

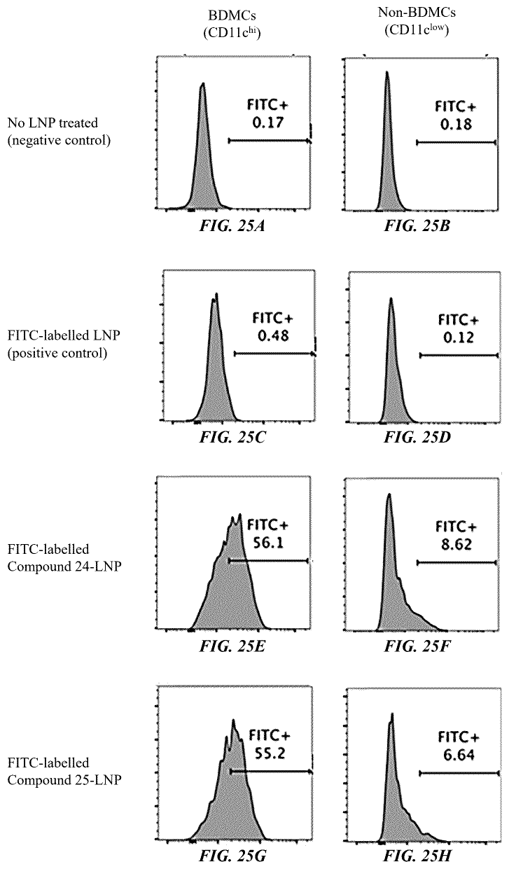

Result. It was observed that removing all glycosylation sites of the stem region diminished significantly the immunogenicity of the vaccine, and maintaining the glycosylation site of N40 did not make much difference (data not shown). Therefore, the experiments then focused on testing the deg-stem+N40,316 and the deg-stem+N40,316,345 vaccines. These two deglycosylation vaccines did not induce more IgG response against the vaccine strain than the wild-type vaccines (FIG. 23). They induced IgG response against B/Maryland/1959 strain at a similar level as the wild-type vaccine did (FIG. 24). However, data showed that among the IgG titers induced by the two deglycosylation vaccines, a higher percentage of them were able to neutralize the B/Maryland/1959 strain. See Table below. Among the IgG titer induced by the deg-HA2 vaccine, around 3.1% of them can neutralize the non-vaccine strain (B/Maryland/1959). For the deg-stem+N40,316 vaccine, around 6.4% of the IgG titer induced can neutralize the non-vaccine strain. These were about 20-time and 40-time better than that of the wild-type vaccine, which only had around 0.14% of the induced IgG titer capable of neutralizing the non-vaccine strain. These results again verified the value of the deglycosylation concept and how maintaining the glycosylation sites in certain region can significantly affect and improve immunogenicity.

TABLE
ELISA assay results showing IgG titer

| | WT | | |
|---|---|---|---|
| | (B/Austria/ | | |
| | 1359417/ | | deg- |
| | 2021) | deg-HA2 | stem + N40, 316 |
| B/Austria/ | 5118000 | 335340 | 53100 |
| 1359417/2021 | | | |
| (B/Victoria lineage; | | | |
| vaccine strain) | | | |
| B/Maryland/1959 | 7020 | 10260 | 3420 |
| (B/Victoria lineage) | | | |
| Percentage of IgG with | ~0.14% | ~3.1% | ~6.4% |
| cross-activities | | | |

### Example B1: Synthesis of Exemplary Compounds of the Present Disclosure

### Chemical Materials and Methods

For chemical synthesis, all starting materials and commercially obtained reagents were purchased from SIGMAR-Aldrich and used as received unless otherwise noted. All reactions were performed in oven-dried glassware under a nitrogen atmosphere using dry solvents. ¹H and ¹³C NMR spectra were recorded on Brucker AV-600 spectrometer, and were referenced to the solvent used (CDCl₃ at δ 7.24 and 77.23, CD₃OD at δ 3.31 and 49.2, and D₂O at δ 4.80, and DMSO-d₆ at δ 2.5 and 39.51 for ¹H and ¹³C, respectively). Chemical shifts (δ) are reported in ppm using the following convention: chemical shift, multiplicity (s=singlet, d=doublet, t=triplet, q=quartet, m=multiplet), integration, and coupling constants (J), with J reported in Hz. High-resolution mass spectra were recorded under ESI-TOF mass spectroscopy conditions. Silica gel (E, Merck) was used for flash chromatography. IMPACT™ system (Intein Mediated Purification with Affinity Chitinbinding Tag) was purchased from New England Biolabs. His-tag purification resin was purchased from Roche. HiTrap IMAC column (5 mL) was purchased from GE Healthcare Life Sciences. Gel permeation chromatography (GPC) equipped with Ultimate 3000 liquid chromatography associated with a 101 refractive index detector and Shodex columns was used to analyze the polymeric products using THE as the eluent at 30° C. with 1 mL min⁻¹ flow rate. The calibration was based on the narrow linear poly(styrene) Shodex standard (SM-105). The Mw and dispersity of the polymeric products were calculated using DIONEX chromeleon software. Transmission electron microscopy (TEM) images were obtained by a FEI Tecnai G2 F20 S-Twin.

The chemical materials and methods described herein apply to all examples described in the present disclosure.

### Synthesis and Results

The exemplary compounds described here were synthesized according to the synthesis Scheme 1, Scheme 2, and Scheme 3 below. The detailed synthesis procedures are described below.

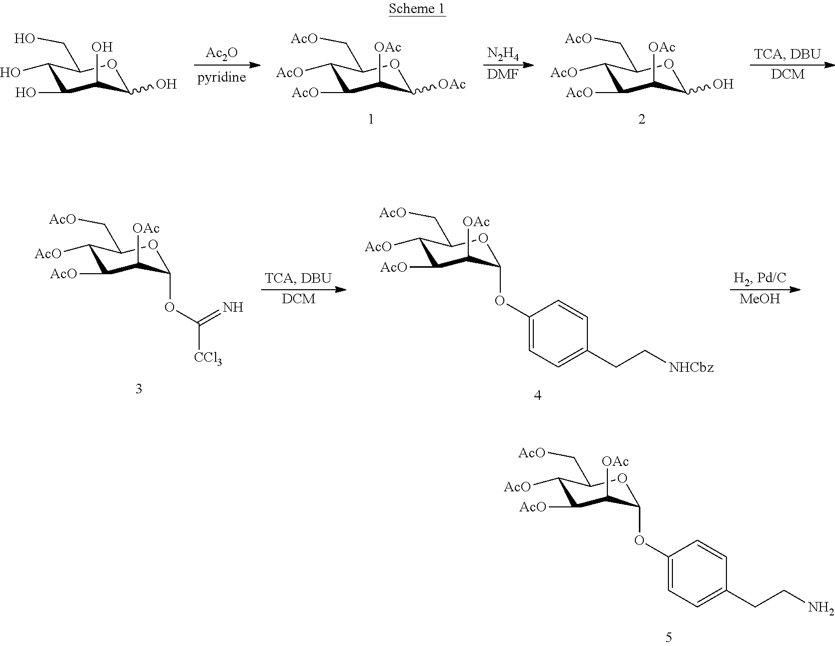

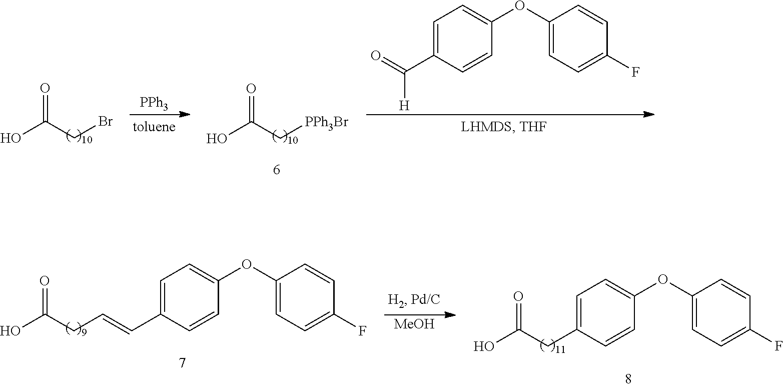

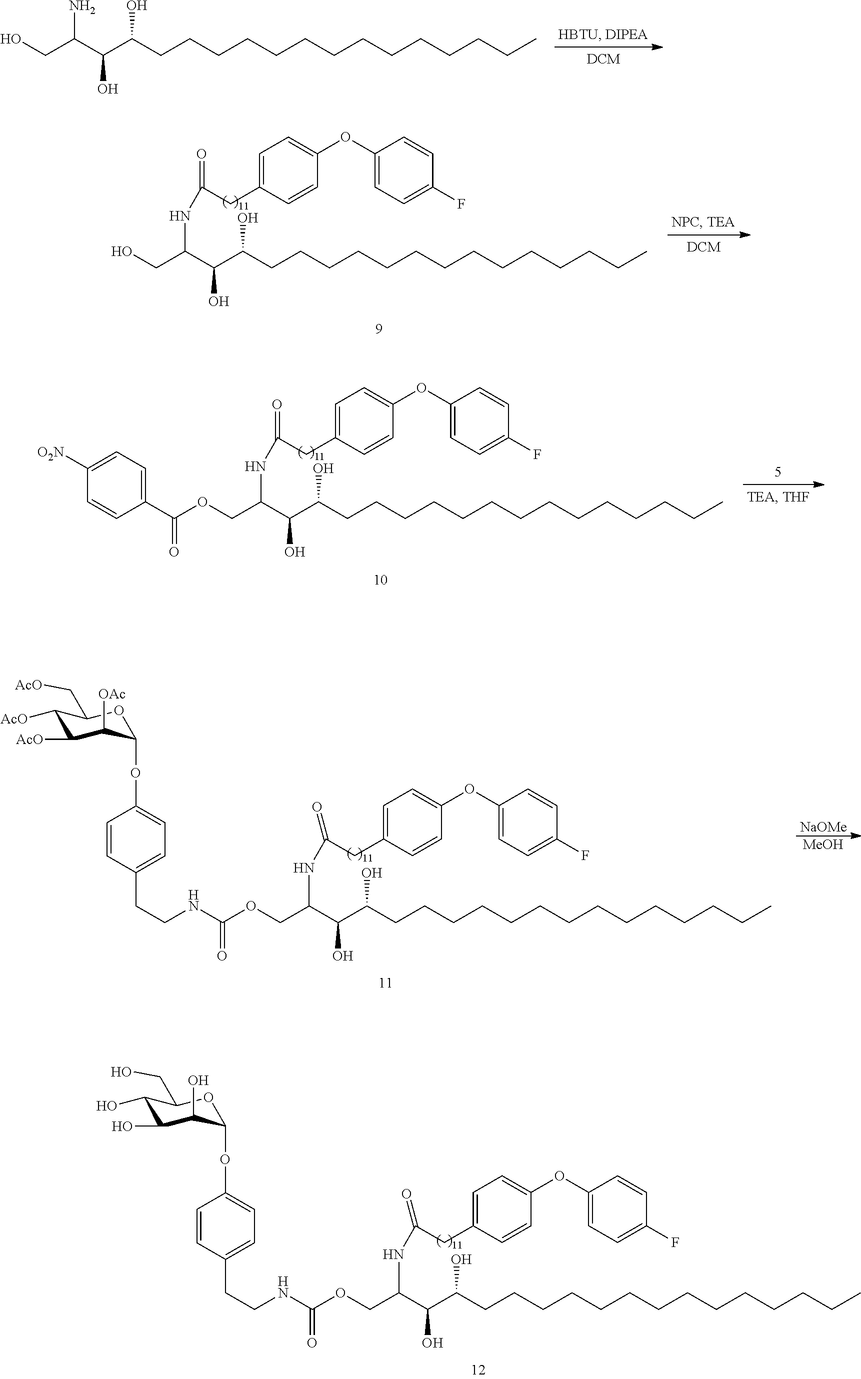

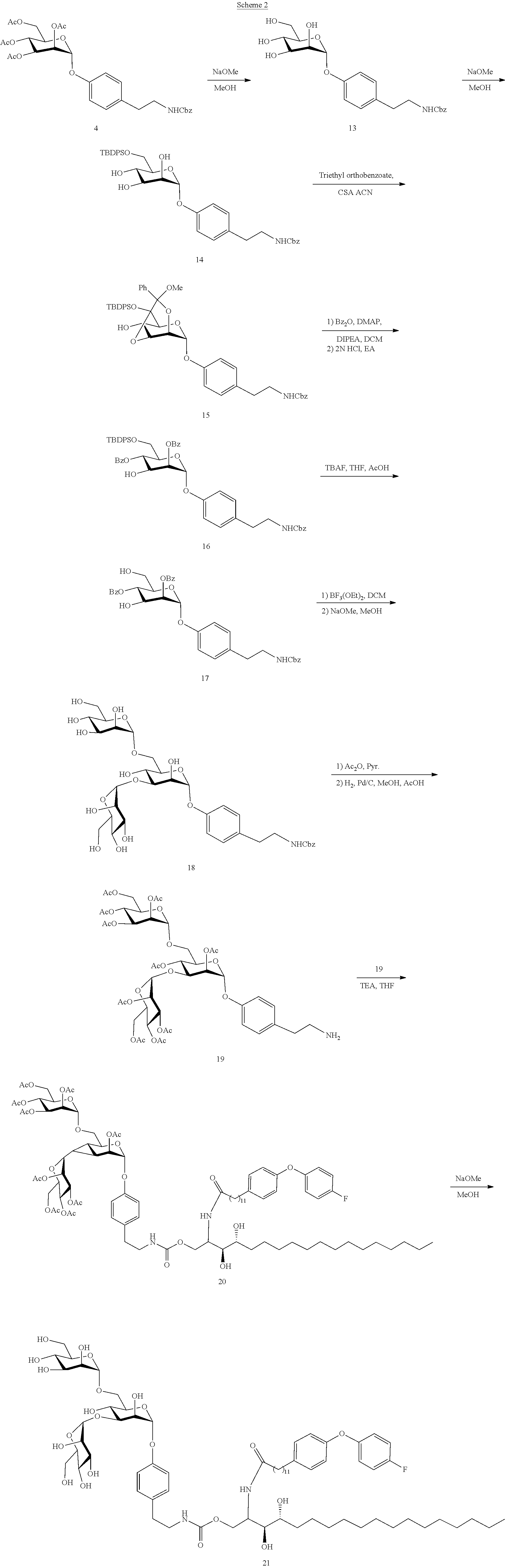

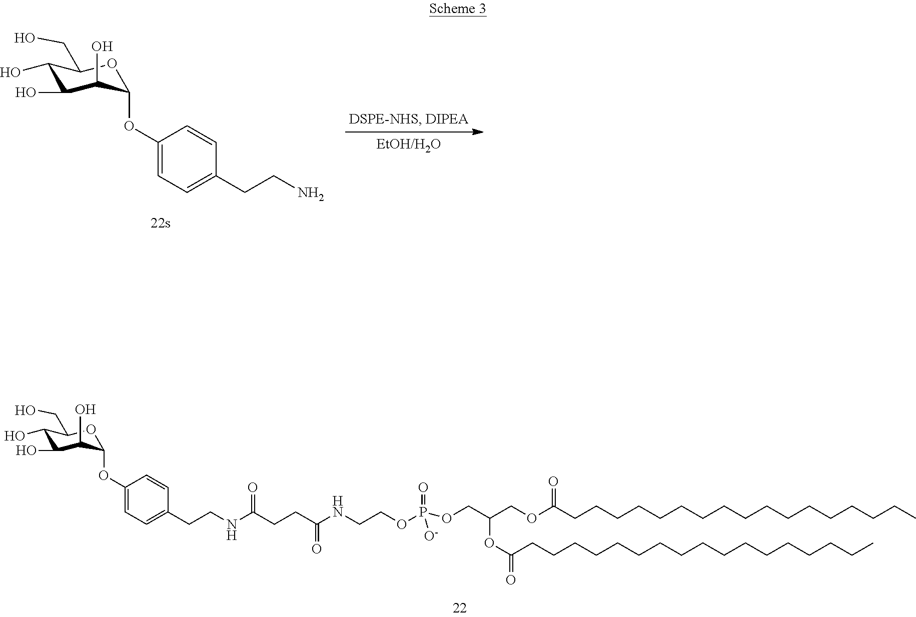

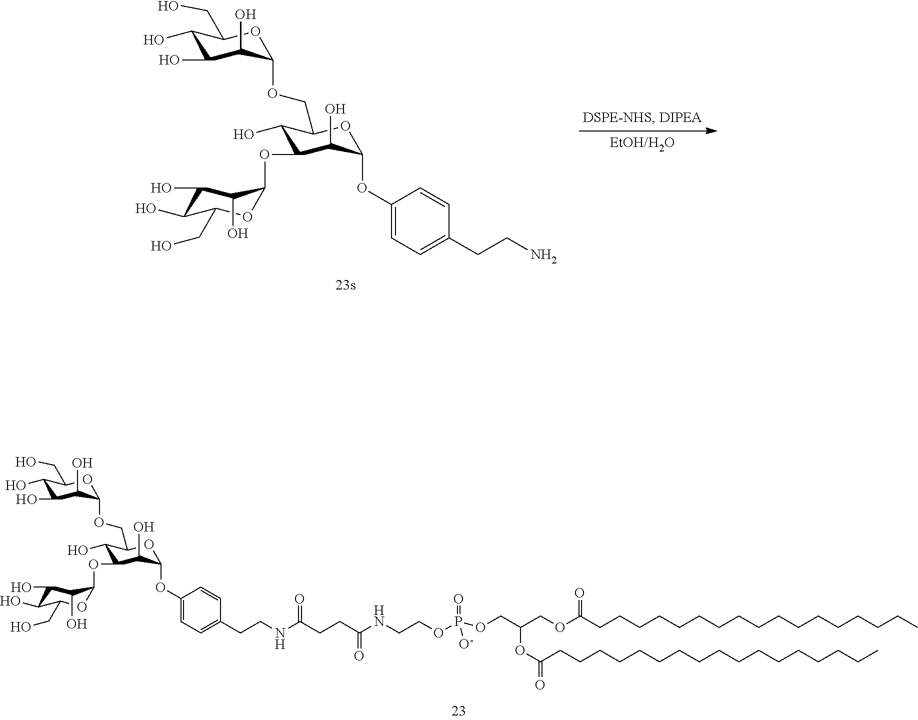

### Compounds 1 to 5

Compounds 1-5 were synthesized and characterized according to a published protocol (ACS Nano 2021, 15, 309-321).

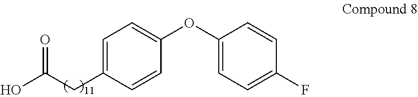

Compound (11-Carboxynonyl)triphenylphosphonium bromide 6 (2.5 g, 10 mmol) was prepared by refluxing triphenylphosphine (10 mmol) and 11-bromoundecanoic acid (10 mmol). It was then dissolved in 50 mL of tetrahydrofuran (THF) and cooled to 0° C. lithium bis(trimethylsilyl)amide (LHMDS; 1 M in THF, 20 mmol) was added to the solution to produce an orange ylide. After that, 4-(4-Fluorophenoxy)benzaldehyde (12 mmol) in 20 mL of THF was added dropwise to the solution, which was stirred for 4 h at room temperature. The reaction was quenched with methanol and concentrated. The residue was extracted with EA and brine and then dried over MgSO₄. After removal of the solvent, the mixture was chromatographed on silica gel (EA-Hex=1:2) to give the unsaturated fatty acid 7. The saturated fatty acid was prepared by catalytic hydrogenation in 50 ml of methanol containing 10 mol % of 10% palladium on charcoal (Pd/C). The reaction mixture was stirred under H₂ at room temperature overnight. The hydrogenated product was filtered through Celite, and the resulting solution was concentrated and chromatographed on silica gel (EA-Hex=1:2) to give the product as a yellow solid (66%).

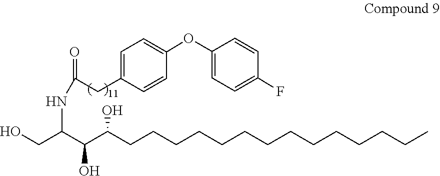

Compound 9. Compound 8 (1 mmol) in THF (10 mL) was added EDC (1.5 mmol), HOBt (1.5 mmol), DMAP (0.1 mmol), trimethylamine (2 mmol), and phytosphingosine (1.2 mmol), and the resulting solution was stirred under nitrogen at rt for 12 h. The solvent was then removed by evaporation, followed by extraction with EA/H₂O. The collected organic layer was washed with saturated NaHCO₃(aq), water and brine, and dried over MgSO₄. The crude product was purified by column chromatography on silica gel (EA/Hex 1:1) to yield Compound 9 (74%).

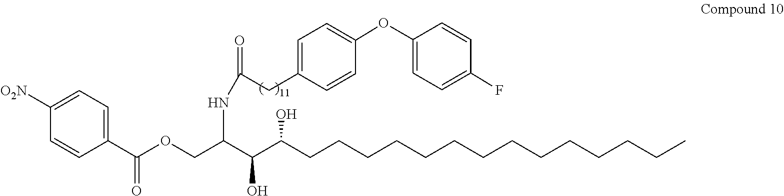

Compound 9 (1 mmol) in THF (10 mL) was added 4-nitrophenylchloroformate (2 mmol), trimethylamine (2 mmol), and the resulting solution was stirred under nitrogen at rt for 12 h. The solvent was then removed by evaporation, and the crude compound was directly used for the next step without further purification.

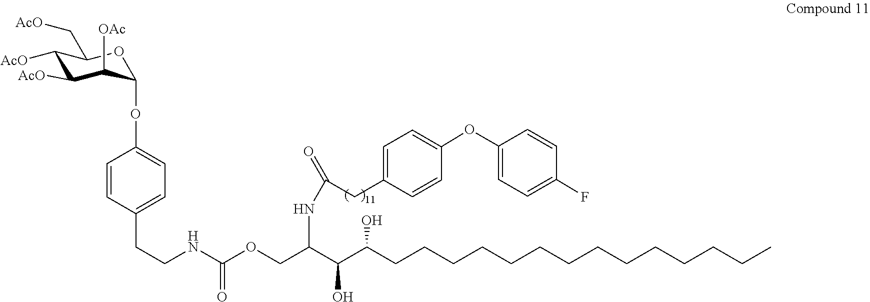

Compound 5 (1 mmol) in THF (10 mL) was added to Compound 10 (1 mmol) and trimethylamine (2 mmol), and the resulting solution was stirred under nitrogen at rt for 2 h. The solvent was then removed by evaporation, followed by extraction with EA/H₂O. The collected organic layer was washed with saturated NaHCO₃(aq), water and brine, and dried over MgSO₄. The crude product was purified by column chromatography on silica gel (EA/Hex 1:1+10% MeOH) to yield Compound 11 (59%).

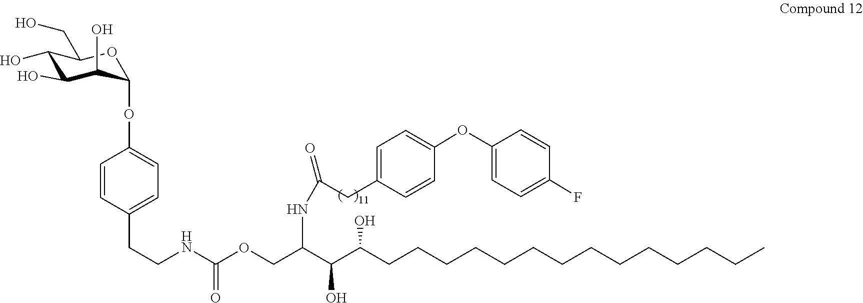

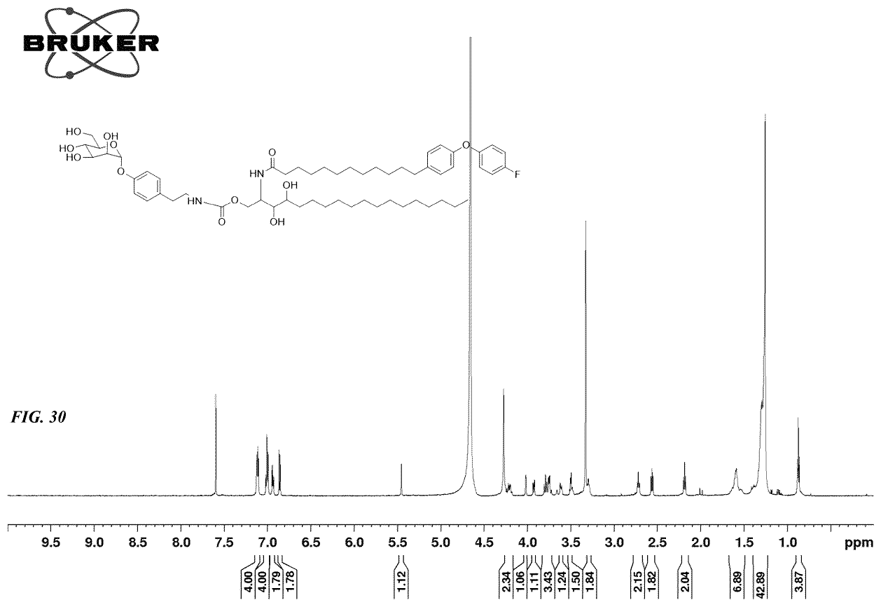

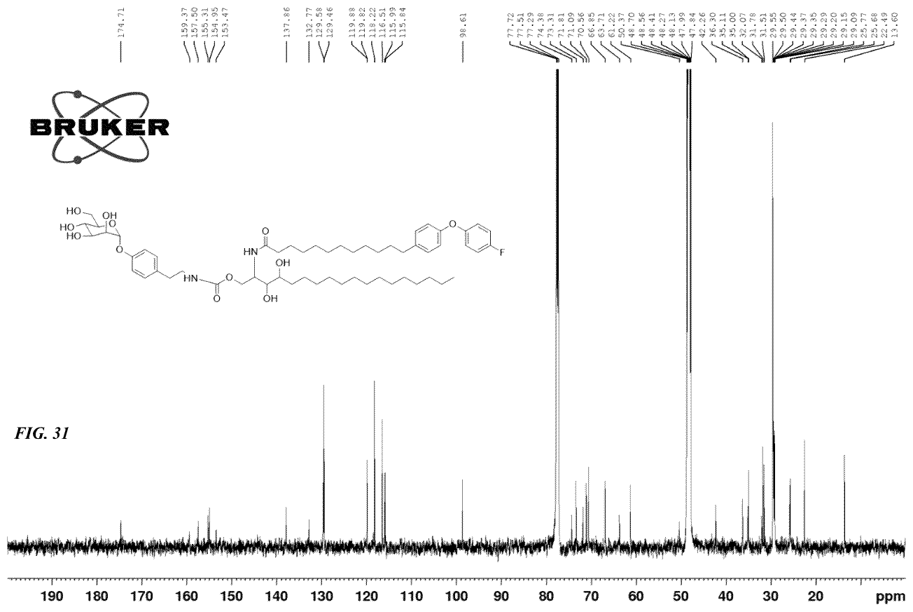

Compound 11 in MeOH was added NaOMe (0.2 eq), and the resulting solution was stirred under nitrogen at room temperature for 2 hours. The mixture was neutralized with IR-120 and then filtered and concentrated to dryness in vacuo to give Compound 12 (quant.) (FIG. 30 and FIG. 31).

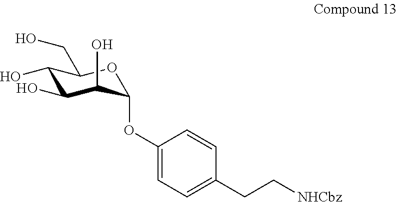

Compound 4 in MeOH was added NaOMe (0.2 eq), and the resulting solution was stirred under nitrogen at rt for 2 h. The mixture was neutralized with IR-120 and then filtered and concentrated to dryness in vacuo to give compound 13 (quant.).

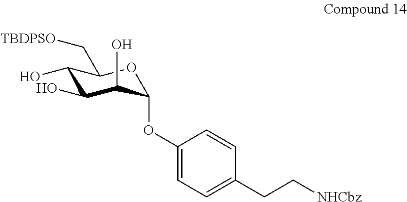

Compound 13 (1 mmol) in MeOH was added NaOMe (0.2 eq), and the resulting solution was stirred under nitrogen at rt for 2 h. The mixture was neutralized by IR-120 and then filtered and concentrated to dryness in vacuo. It was then dissolved in anhydrous DCM (10 mL) and treated with imidazole (1.5 mmol) at 0° C., followed by the addition of TBDPSCl (1.2 mmol). The mixture was stirred at room temperature for 2.5 h under a nitrogen atmosphere. The reaction was quenched by adding MeOH. After stirring at room temperature for 10 min, the solvent was removed under reduced pressure to give a dry residue that was purified by column chromatography with MeOH/DCM (1/10) to give Compound 14 (82%).

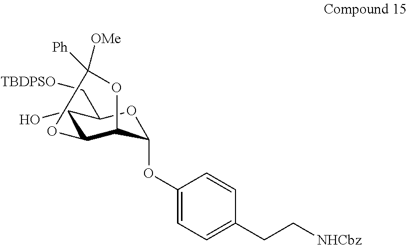

To a solution of Compound 14 (1 mmol) and a catalytic amount of CSA (0.1 mmol) in CH₃CN (20 mL), trimethyl orthobenzoate (3 mmol) was added at room temperature under an atmospheric pressure of nitrogen. After stirring for 30 min, Et₃N was added to quench the reaction, and the resulting mixture was dried under reduced pressure. The residue was purified by column chromatography with EA/Hex (1/2) to give Compound 15 (79%).

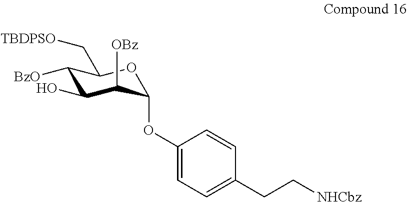

Compound 15 (1 mmol) was dissolved in DCM (10 mL) and sequentially mixed with DIPEA (2 mmol), benzoic anhydride (2 mmol), and DMAP (0.1 mmol). After stirring for 2 hr, the solvent was evaporated under reduced pressure to give a dry residue and then poured into EA (20 mL) and 2 N HCl (10 mL) with vigorous stirring for 30 min. The solvent was then removed by evaporation, followed by extraction with EA/H₂O. The collected organic layer was washed with ice-cold saturated NaHCO₃(aq), water and brine, and dried over MgSO₄. The dry residue was purified by column chromatography with EA/Hex (1/2) to give Compound 16 (71%).

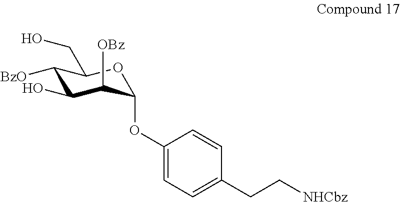

Compound 16 (1 mmol) was added AcOH (4 mmol) and 1 M TBAF (2.4 mmol in THF) at 0° C. The resulting mixture was warmed up to room temperature gradually, stirred for another 2 h, and then diluted with EA. The organic layer was washed with saturated NaHCO₃(aq), water, and brine, dried with anhydrous MgSO₄, and concentrated under reduced pressure. The dry residue was purified by column chromatography with EA/Hex (1/2) to give Compound 47 (88%).

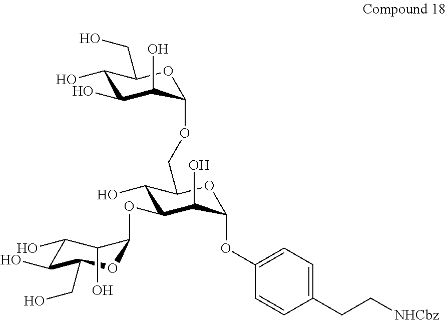

To a stirred solution of Compound 17 (1 mmol) and 4 Å molecular sieve (0.1 g) in anhydrous DCM (10 mL) was cooled to −40° C. and then BF₃(OEt)₂ (0.1 mmol) was added dropwise to the solution. A solution of 3 in anhydrous DCM was added dropwise to the above mixture and stirred for 1 h at −40° C. After that, the reaction was gradually warmed to room temperature and stirred for another 1 h. The solution was quenched by adding triethylamine, then filtered and added saturated. NaHCO₃ aq, and extracted with DCM. The organic layer was dried with MgSO₄ and evaporated to dryness. The residue was purified by flash column chromatography on silica gel to give a trisaccharide product. The product was then dissolved in MeOH, and NaOMe (0.2 eq) was added, and the resulting solution was stirred at room temperature for 2 h. The mixture was neutralized by IR-120 and then filtered and concentrated to dryness in vacuo. The deacetylated mixture was purified using Bio-Gel P-2 Gel (Bio-Rad) with H2O as the eluent to obtain a pure trisaccharide. The compound was lyophilized to dryness to give Compound 18 (39%).

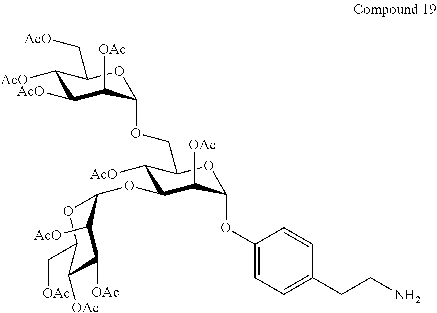

Compound 13 (1 mmol) in MeOH was added NaOMe (0.2 eq), and the resulting solution was stirred under nitrogen at rt for 2 h. The mixture was neutralized by IR-120 and then filtered and concentrated to dryness in vacuo.

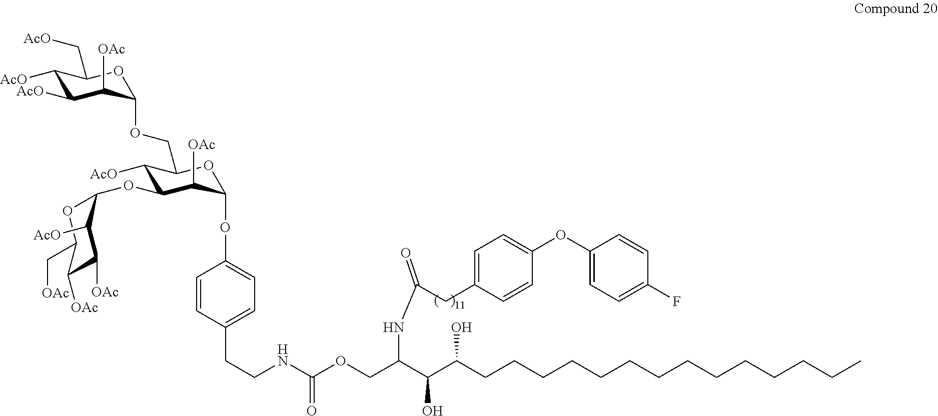

Compound 19 (1 mmol) in THF (10 mL) was added 10 (1 mmol) and trimethylamine (2 mmol), and the resulting solution was stirred under nitrogen at rt for 2 h. The solvent was then removed by evaporation, followed by extraction with EA/H₂O. The collected organic layer was washed with saturated NaHCO₃(aq), water and brine, and dried over MgSO₄. The crude product was purified by column chromatography on silica gel (EA/Hex 1:1+10% MeOH) to yield Compound 20.

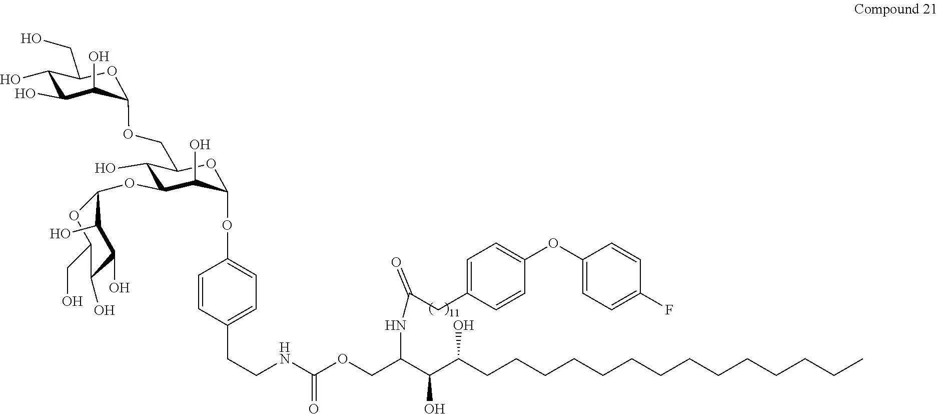

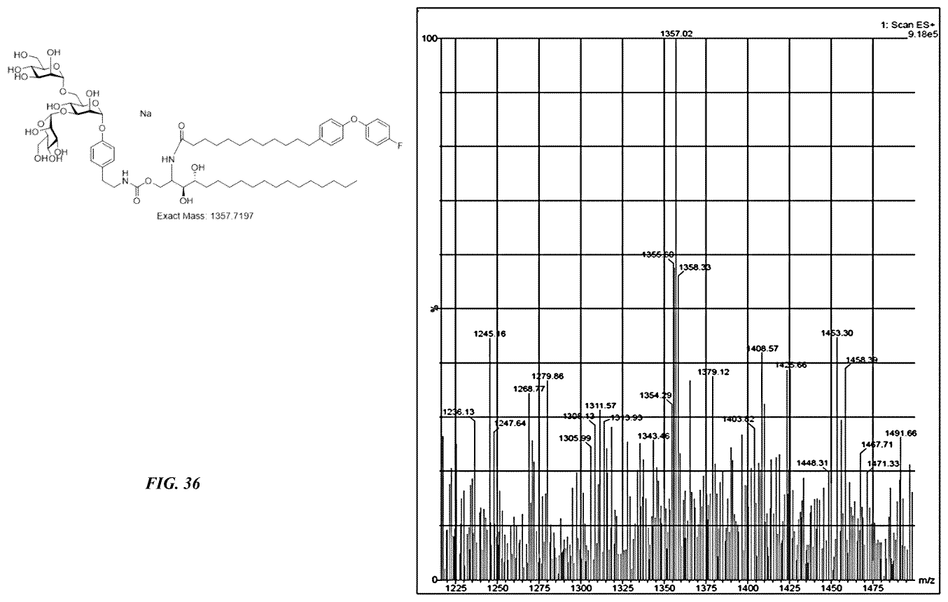

Compound 20 in MeOH was added NaOMe (0.2 eq), and the resulting solution was stirred under nitrogen at room temperature for 2 hours. The mixture was neutralized with IR-120 and then filtered and concentrated to dryness in vacuo to give Compound 21 (quant.). The resulting compound 21 was examined using an LC-MS spectrum, which shows peaks at 1236.13, 1245.16, 1247.64, 1268.77, 1279.86, 1305.99, 1308.13, 1311.57, 1313.93, 1343.46, 1354.29, 1355.60, 1358.33, 1379.12, 1403.82, 1408.57, 1425.66, 1448.31, 1453.30, 1458.39, 1467.71, 1471.33, and 1491.66 (FIG. 36).

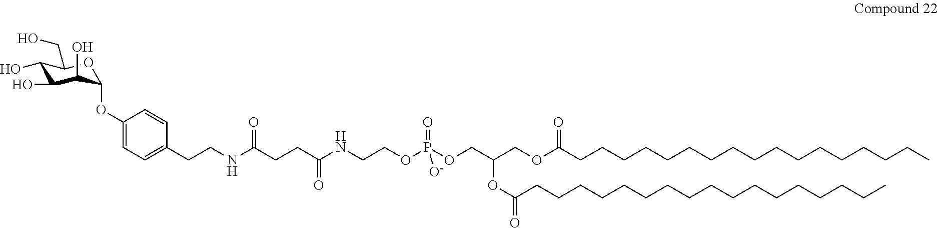

Arylmannoside 22s (0.1 mmol) in EtOH/H₂O (0.5/0.5 mL) was added to DSPE-NHS (0.1 mmol), and trimethylamine (2 mmol), and the resulting solution was stirred at rt for 12 h. The solvent was removed by evaporation and the crude product was purified Bio-Gel P-2 Gel with H₂O as eluent to yield Compound 22 (79%).

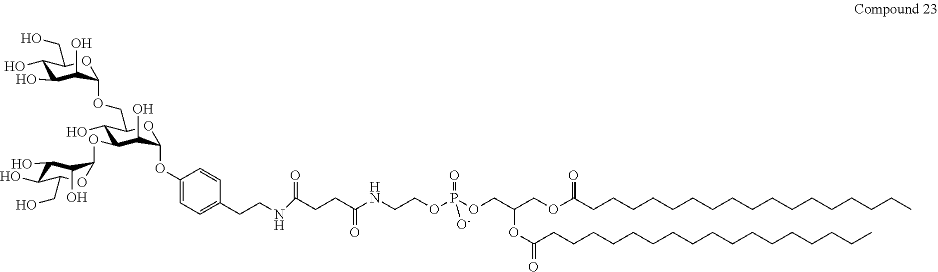

Aryltrimannoside 23s (0.1 mmol) in EtOH/H₂O (0.5/0.5 mL) was added DSPE-NHS (0.1 mmol) and trimethylamine (2 mmol), and the resulting solution was stirred at room temperature for 12 h. The solvent was removed by evaporation and the crude product was purified Bio-Gel P-2 Gel with H₂O as eluent to yield Compound 23 (76%).

### Example B2: Preparation and Characterization of the LNP of the Present Disclosure

### Preparation of LNP

A lipid mix solution in EtOH (10 mg/ml) having a molar ratio of 50% SM-102, 10% DSPC, 38.5% cholesterol, and 1.5% DMG-PEG2000 was prepared. An LNP formulation was prepared by mixing the compound of the present disclosure with the lipid mix solution (with a molar ratio of 45% SM-102, 9% DSPC, 34.5% cholesterol, 1.5% DMG-PEG2000, and 10% compound of the present disclosure). The LNP formulation was added into a 1.5 mL tube. Then, an mRNA payload, diluted with citrate buffer before use (10 mM, pH 4), was added to the tube at a final concentration of 0.18 μg/μL. The mRNA aqueous solution in the tube was then quickly added to an ethanol solution and mixed well by vortexing for 1 minute. The resulting solution was then dialyzed using a MicroFloat-A-Lyzer (8-10 kD) against PBS at 4° C. overnight to obtain the LNP for this example. The resulting LNP can be stored at 4° C. for a few days before use.

### Characterization of LNP

Size Measurement. The LNP prepared above was examined by dynamic light scattering (DLS) to determine its size. First, 5 μL of the LNP solution was transferred to a clean 1.5 mL tube and diluted with 95 μL of PBS. The mixture was then transferred to a cuvette, and the LNP particle size was measured using a Nano ZS machine. The following table shows the sizes and the Polydispersity Index (PDI) of the LNP samples prepared.

TABLE
the size and PDI measurement

| Sample | Size (nm)_Mean ± SD | PDI_Mean ± SD |
|---|---|---|
| Compound 24- | 138.5 ± 0.7074 | 0.1498 ± 0.002694 |
| LNP | | |
| Compound 25- | 161 ± 0.3246 | 0.1221 ± 0.02095 |
| LNP | | |
| Compound 12- | 177.5 ± 1.79 | 0.1381 ± 0.02234 |
| LNP | | |
| Compound 22- | 191.7 ± 1.617 | 0.1625 ± 0.0294 |
| LNP | | |
| Compound 23- | 170.7 ± 1.353 | 0.1109 ± 0.01918 |
| LNP | | |

Zeta potential and encapsulation efficiency. Next, the encapsulation efficiency of the LNP of the present disclosure was evaluated using a Quant-it Ribogreen assay. A 2000-fold dilution of the quant-it Ribogreen reagent with 1×TE (working solution) was prepared. Then, an RNA standard dilution series from 0 to 50 ng/ml (100 μL) was prepared to generate a standard curve. 5 μL of the LNP solution prepared above was transferred to a clean tube and diluted to a final volume of 100 μL. The working solution of the quant-it Ribogreen reagent (100 μL) was then added to the LNP sample. The fluorescent signal of the sample was then detected using a microplate reader (ex/em 485/535). According to the standard curve, the fluorescence signal was used to calculate the concentration of unencapsulated mRNA in solution (ng/mL). For zeta potential measurement, 0.75 mL DP-intermediate was introduced into capillary cells, and measurements were performed at 25° C. using a Malvern Zetasizer Pro.

TABLE
Zeta potential and encapsulation efficiency

| | | Encapsulation |
|---|---|---|
| Sample | Zeta potential (mV) | efficiency (%) |
| Compound 24- | 0.113 | 92.15 |
| LNP | | |
| Compound 25- | −4.382 | 86.69 |
| LNP | | |
| Compound 12- | 0.932 | 81.47 |
| LNP | | |
| Compound 22- | −0.5107 | 82.86 |
| LNP | | |
| Compound 23- | 0.3875 | 90.00 |
| LNP | | |

### Example B3: In Vitro Uptake and Transfection of mRNA-LNPs in Dendritic Cells

### Experiment 3-1

Splenic cell preparation and BMDC culture. This example tested the uptake of several exemplary LNPs (as shown in the table below) according to the embodiments of the present disclosure in bone marrow-derived dendritic cells (BMDCs) and splenic cells. To prepare splenic cells, the mouse spleen was homogenized with the frosted end of a glass slide, treated with RBC lysis buffer (SIGMA®) to deplete red blood cells (RBCs), and then passed through a cell strainer (BD BIOSCIENCES®). Bone marrow was isolated from mouse femurs and tibiae and treated with RBC lysis buffer (SIGMA®) to deplete RBCs. Cells were then cultured in RPMI-1640 containing 10% heat-inactivated FBS (THERMO FISHER SCIENTIFIC®), 1% Penicillin/Streptomycin (THERMO FISHER SCIENTIFIC®), 50 μM 2-mercaptoethanol (THERMO FISHER SCIENTIFIC®), and 20 ng/ml recombinant mouse GM-CSF (eBioscience) at a density of 2× 10⁵ cells/ml. The cells were supplemented with an equal volume of the complete culture medium (RPMI-1640, 100 U/ml Pen/Strep, 55 μM 2-mercaptoethanol, and 10% FBS) at day 3 and refreshed with one-half the volume of the medium at day 6. On day 8, the suspended cells were harvested.

Table of the exemplary LNPs tested in this experiment.

| | | | | | L5 |
|---|---|---|---|---|---|
| | | | | | The |
| | | | | | compound |
| | L1: | L2: | | | of the |
| | Ionizable | Phosphatidyl- | L3: | L4: | present |
| Example | lipid | choline | Cholesterol | PEG-Lipid | disclosure |
| 1 | 45 mol % | 9 mol % | 34.5 mol % | 1.5 mol % | Compound |
| | | | | | 24 |
| | | | | | 10 mol % |
| 2 | 45 mol % | 9 mol % | 34.5 mol % | 1.5 mol % | Compound |
| | | | | | 25 |
| | | | | | 10 mol % |
| 3 | none | none | none | none | none |
| N.C. | | | | | |
| (no LNP) | | | | | |
| 4 | 50 mol % | 10 mol % | 38.5 mol % | 1.5 mol % | none |
| P.C. | | | | | |

Treatment of LNPs to splenic cells and BMDCs. Splenic cells or BMDCs were incubated with different FITC-labeled LNP formulations in RPMI-1640 at 37° C. for 1 hour. Cells were blocked with an Fc receptor binding inhibitor (clone: 93, eBioscience) for 20 minutes. Splenocytes were stained with antibodies against CD3 (clone: 17A2, BV421-conjugated, BIOLEGEND®), CD19 (clone: 1D3, PECy7-conjugated, BD BIOSCIENCES®). BMDCs were stained with antibodies against CD11c (clone N418 APC-conjugated, BIOLEGEND®). Labeled cells were analyzed using FACSC and a Flow Cytometer (BD BIOSCIENCES®).

Flow Cytometry. After incubation with different mRNA-LNPs, BMDC cells were washed with ice-cold FACS buffer (1% FBS in 1×DPBS with 0.1% Sodium Azide), and incubated with purified anti-mouse CD16/32 antibody (BIOLEGEND®) in FACS buffer on ice for 20 min, followed by washing with FACS buffer. BMDCs were stained with APC anti-mouse CD11c antibody (BIOLEGEND®) at 4° C. for 30 min, and washed with FACS buffer. Finally, BMDCs were stained with propidium iodide (SIGMA®-Aldrich). Flow cytometry was performed on a FACS Canto™ flow cytometer (BD Bioscience).

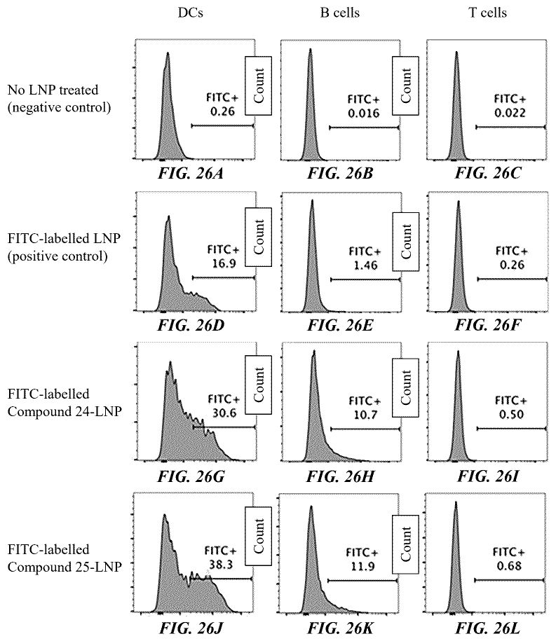

Results. The FACS results are shown in FIGS. 25A-25H and FIGS. 26A-26L and the table below. FIGS. 25A-25H shows that BMDCs specifically uptake LNPs made using compounds of the present disclosure compared with non-BMDCs. A traditional LNP (i.e., without using the compound of the present disclosure) showed slightly higher uptake by the BMDCs than the non-BDMCs, but the specificity was insignificant compared to that of the LNPs of the present disclosure (56.1/8.62 or 55.2/6.64 vs. 0.48/0.12). Similarly, in FIGS. 26A-26L, dendritic cells (DCs) showed specific uptake of the LNPs of the present disclosure at least 3 times higher than B cells (30.6/10.7 and 38.3/11.9) and at least 6 times higher than T cells (30.6/0.5 and 38.3/0.68). DCs showed higher uptake of traditional LNPs, but the inclination was less significant than that of the LNPs of the present disclosure.

Table of the uptake results (arbitrary unit of the FITC signals)

| | L5 | | | | | |
|---|---|---|---|---|---|---|
| | the compound of | FITC signal (A.U.) | | | | |
| Experiment | the present | | Non- | | | |
| 3-1 | disclosure | BMDC | BMDC | DCs | B cells | T cells |
| Example 1 | Compound 24 | 56.1 | 8.62 | 30.6 | 10.7 | 0.50 |
| Example 2 | Compound 25 | 55.2 | 6.64 | 38.3 | 11.9 | 0.68 |
| Example 3 | Negative control | 0.17 | 0.18 | 0.26 | 0.016 | 0.022 |
| Example 4 | Positive control | 0.48 | 0.12 | 16.9 | 1.46 | 0.26 |

### Experiment 3-2

Exemplary LNPs (as shown in the table below) made using different formulations according to the embodiments of the present disclosure were tested in this experiment. Both uptake and transfection were tested to assess whether the payload delivered by the LNPs of the present disclosure can be expressed properly in targeted cells. Bone marrow-derived dendritic cells (BMDCs) were isolated from murine tibia and femurs of 57BL/6 mice. Bone marrow cells were stimulated for 8 days with 20 ng/mL GM-CSF in RPMI medium (RPMI-1640, 100 U/ml Pen/Strep, 55 μM 2-mercaptoethanol, and 10% FBS). After 8 days of culture, 1×10⁶ BMDCs (centrifuge 400 g, 5 mins and replace medium with 1 ml Opti-MEM) were plated in 6-well plates, and different samples of LNPs encapsulating mRNA were diluted by 0.25 mL Opti-MEM and incubated with BMDC.

For uptake analysis, FITC-labelled LNPs encapsulating mRNA that encodes a SARS-CoV-2 Spike protein were incubated with the BMDCs at 37° C. for 2 hours. For transfection analysis, the LNPs encapsulating eGFP mRNA were incubated with the BMDCs at 37° C. for 4 hours. After 4 hours of transfection, BMDCs were supplemented with 1.25 mL of complete RPMI medium and incubated at 37° C. for 48 hours. The experiments were conducted using FACS, similar to that described above.

Table of the exemplary LNPs tested in this experiment.

| | | | | | L5 |
|---|---|---|---|---|---|
| | | | | | the |
| | | | | | compound |
| | L1: | L2: | | | of the |
| | Ionizable | Phosphatidyl- | L3: | L4: | present |
| Example | lipid | choline | Cholesterol | PEG-Lipid | disclosure |
| 1 | 47.5 mol % | 9.5 mol % | 36.5 mol % | 1.5 mol % | 5 mol % |
| | | | | | Compound |
| | | | | | 22 |
| 2 | 45 mol % | 9 mol % | 34.5 mol % | 1.5 mol % | 10 mol % |
| | | | | | Compound |
| | | | | | 22 |
| 3 | 40 mol % | 8 mol % | 30.5 mol % | 1.5 mol % | 20 mol % |
| | | | | | Compound |
| | | | | | 22 |
| 4 | 47.5 mol % | 9.5 mol % | 36.5 mol % | 1.5 mol % | 5 mol % |
| | | | | | Compound |
| | | | | | 23 |
| 5 | 45 mol % | 9 mol % | 34.5 mol % | 1.5 mol % | 10 mol % |
| | | | | | Compound |
| | | | | | 23 |
| 6 | 40 mol % | 8 mol % | 30.5 mol % | 1.5 mol % | 20 mol % |
| | | | | | Compound |
| | | | | | 23 |
| 7 | 50 mol % | 10 mol % | 38.5 mol % | 1.5 mol % | none |
| (control) | | | | | |

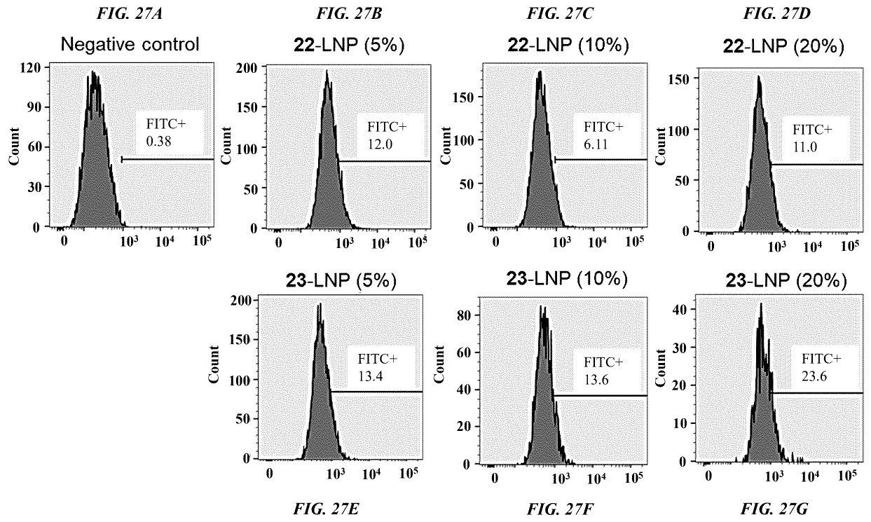

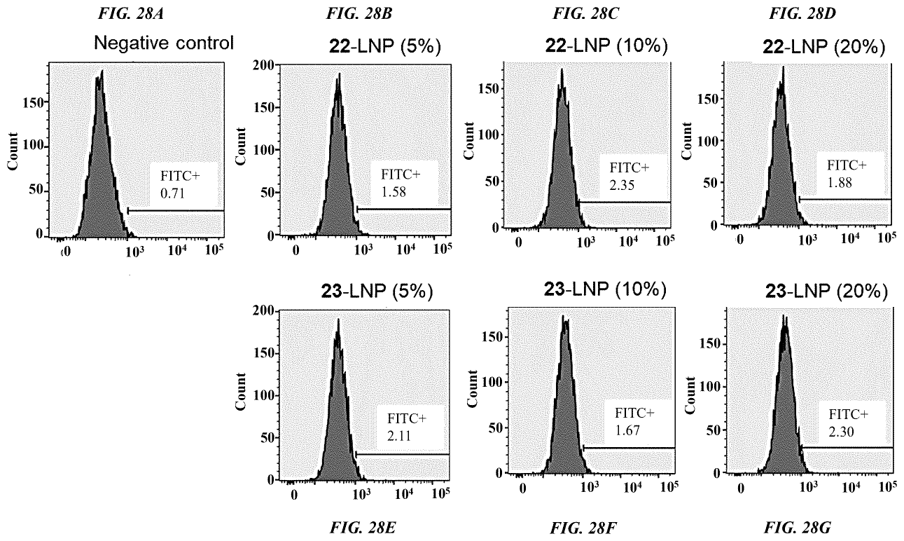

Results. The FACS results are shown in FIGS. 27A-27G, FIGS. 28A-28G, and the table below. LNPs made using the compounds of the present disclosure at different molar ratios all showed higher uptake than the negative control (“traditional” LNP without using the compound of the present disclosure). The data also confirms that the LNPs of the present disclosure not only can deliver the payload into the targeted cells but also can transfect and allow the targeted cells to express the payload. Given the higher specificity towards the targeted cells, the transfection signals detected from the groups using the LNPs of the present disclosures were also significantly higher than those detected from the traditional group. This result suggests that using the LNPs of the present disclosure allows a lower dosage of the payload for a similar outcome.

Table showing the results of uptake and transfection
(arbitrary unit of the FITC signals)

| | | | FITC signal |
|---|---|---|---|
| | L5 | FITC signal | intensity (A.U.) |
| Experiment | the compound of the present | intensity (A.U.) | derived from |
| 3-2 | disclosure | derived from uptake | transfection |
| Example 1 | 5 mol % Compound 22 | 12.0 | 1.58 |
| Example 2 | 10 mol % Compound 22 | 6.11 | 2.35 |
| Example 3 | 20 mol % Compound 22 | 11.0 | 1.88 |
| Example 4 | 5 mol % Compound 23 | 13.4 | 2.11 |
| Example 5 | 10 mol % Compound 23 | 13.6 | 1.67 |
| Example 6 | 20 mol % Compound 23 | 23.6 | 2.30 |
| Example 7 | None (traditional LNP) | 0.38 | 0.71 |
| (control) | | | |

### Experiment 3-3

To assess the binding of DC-SIGN to the LNPs of the present disclosure, ELISA plates were coated with exemplary LNPs in PBS at 4° C. overnight, respectively. The plates were incubated with diluted DC-SIGN ECD (15 to 0.075 nM in HEPES buffer containing 20 mM HEPES, 150 mM NaCl, 10 mM CaCl₂, 0.1% BSA) at pH 7.4, 6.0, and 5.0 for 1 hour at room temperature. The bound DC-SIGN ECD was detected using HRP-conjugated anti-DC-SIGN (B2) IgG antibody (Santa Cruz Biotechnology). After 1 hour of incubation at room temperature, the plates were treated with tetramethylbenzidine (TMB) for 10 min. The optical density was measured at 450 nm after adding 0.5 M sulfuric acid to the plates using a microplate reader. The apparent Kd was calculated using a nonlinear regression curve fit for total binding using GraphPad Prism.

### Example B4: In Vivo Delivery of Luciferase mRNA-LNP

This experiment tested the targeted delivery of the LNPs of the present disclosure (shown in the table below) in vivo. The LNPs tested in this experiment carried mRNA encoding luciferase. Mice were injected intravenously with the LNPs (200 μL) and maintained for one hour or six hours before In vivo Imaging System (IVIS®) measurement. For the IVIS measurement, the animals were first anesthetized using the rodent anesthesia system with isoflurane (2.5% (vol/vol) in 0.2 L/min O2 flow). Then, the animals were injected intravenously with D-luciferin solution (dissolved in 1×PBS; 150 mg/kg body weight). After 3 minutes from the injection, the animals were scanned using the IVIS imaging system (data not shown). After imaging, the animals were euthanized in a CO₂ chamber. The organs (heart, lungs, liver, spleen, kidneys, and lymph nodes) of the animals were collected, and the luminescence was detected and quantified using the IVIS system.

Table of the exemplary LNPs tested in this experiment.

| | | | | | L5 |
|---|---|---|---|---|---|
| | | | | | the |
| | | | | | compound |
| | L1: | L2: | | | of the |
| | Ionizable | Phosphatidyl- | L3: | L4: | present |
| Example | lipid | choline | Cholesterol | PEG-Lipid | disclosure |
| 1 | 45 mol % | 9 mol % | 34.5 mol % | 1.5 mol % | 10 mol % |
| | | | | | Compound |
| | | | | | 22 |
| 2 | 45 mol % | 9 mol % | 34.5 mol % | 1.5 mol % | 10 mol % |
| | | | | | Compound |
| | | | | | 12 |
| 7 | 50 mol % | 10 mol % | 38.5 mol % | 1.5 mol % | none |
| (control) | | | | | |

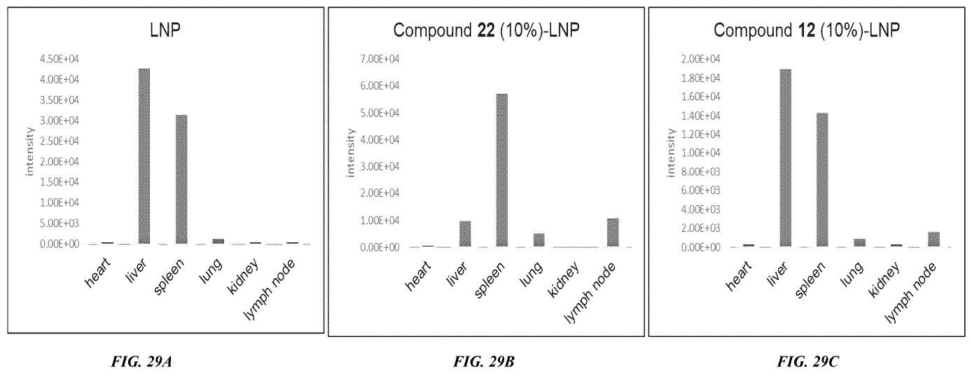

Results. The results (FIGS. 29A-29C) show that both compound 22-LNP and compound 12-LNP tend to accumulate in spleens and lymph nodes. While compound 12-LNP also accumulated in livers, compound 22-LNP showed high-level specificities targeting spleens and lymph nodes. The results demonstrate the targeting delivery functionalities of the lipid nanoparticle formulations of the present disclosure, which match the observations of the experiments above.

### Example B5: Humoral Immune Response Induced by LNPs

This experiment verified the capabilities of the LNPs of the present disclosure in delivering immunogenic cargos and inducing humoral immune responses in vivo. First, traditional LNPs (i.e., without using the compound of the present disclosure) and the LNPs using compound 24 of the present disclosure (see Sample 1 of Experiment 3-1) were prepared and carried COVID spike protein-encoding mRNA. A micelle-type mRNA nanoparticle made from compound 24 and carrying the spike protein-encoding mRNA was also prepared for this experiment. Balb/c mice were separated into groups, and each group was intravenously injected with the traditional LNPs, LNP-compound 24, and compound 24-micelles, respectively, or injected with PBS as a negative control. Then, blood samples were collected from the experimental mice at 2 hours, 24 hours, and 48 hours after injections. The sera of the blood samples were obtained using centrifugation (3000×g, 10 minutes).

Cytokine concentration in the obtained sera was then determined using BD OptEIA™ Mouse ELISA Set. Briefly, 96-well plates were coated with anti-interleukin-4 (IL-4) antibody solution or anti-interferon-γ (IFNγ) antibody solution (1 μg/ml, 100 μl/well) and incubated at 4° C. overnight. Then, the plates were washed with PBST buffer (0.05% Tween 20 in PBS) and blocked using diluent buffer (10% FBS/PBS) at room temperature for 1 hour, followed by another washing procedure. The plates were then added with biotinylated detection antibodies and SA-HRP (100 μl/well) and incubated at room temperature for 1 hour. After that, the plates were washed with PBST buffer, and a substrate solution (100 μl/well) was added. The plates were then incubated at room temperature for 30 minutes in the dark. After stopping the development by adding a stop solution (50 μl/well), the plates were observed, and signals were detected using an ELISA reader at 450 nm.

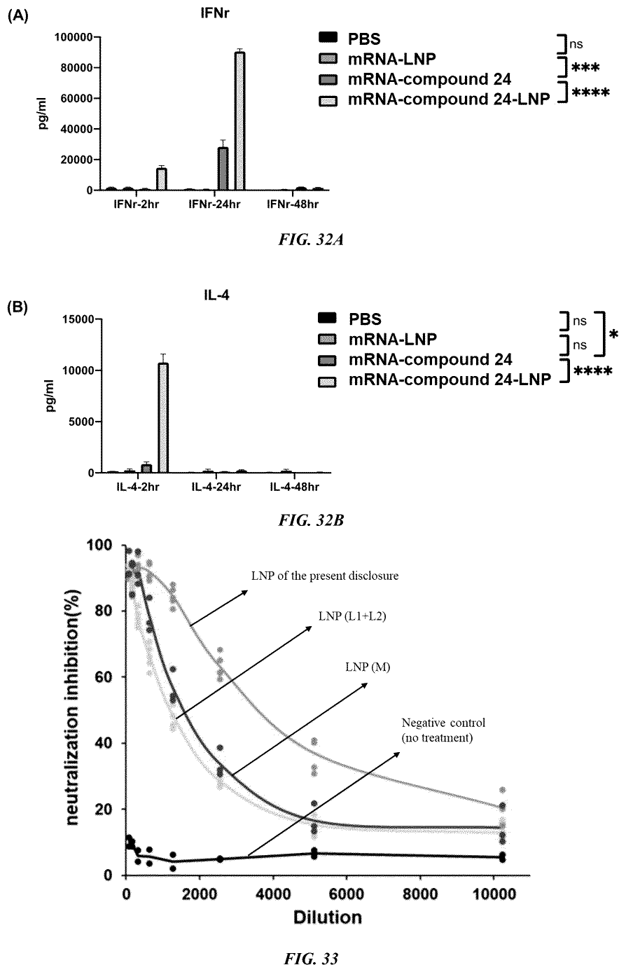

Result. The detection results are shown in FIG. 32A and FIG. 32B. The sera obtained from mice administered with the LNPs of the present disclosure contained detectably increased IFNγ and IL-4 after 2 hours of administration. This observation suggested that the LNPs of the present disclosure were able to deliver payload and induce humoral immune responses rapidly. In contrast, the traditional LNPs did not induce detectable humoral immune responses, showing that the targeted delivery capability of the present disclosure's LNPs was able to improve the efficiency of payload delivery thereby improving the desired effects.

### Example B6: Immunization

Animals. Balb/c mice (8 weeks) were purchased from the National Laboratory Animal Center, Taiwan. All the mice were maintained in a specific pathogen-free environment. Eight-week-old Balb/c mice were immunized i.m. twice at 2-week intervals. Each vaccination contains PBS (100 μl). Sera collected from immunized mice were subjected to ELISA analysis 10 days after the last immunization. The experimental protocol was approved by Academia Sinica's Institutional Animal Care and Utilization Committee (approval no. 22-08-1901).

LNPs. For neutralization assay, LNPs, according to an embodiment of the present disclosure, were prepared for this experiment. Two control LNPs were also prepared to compare the performance of the present disclosure's LNPs. The first control LNP was formed using SM-102 and DSPC (“L1+L2”) without using the compound of the present disclosure. The second control LNP was a MODERNA® product for Spikevax (“LNP (M)”). All tested LNPs carried mRNA cargo encoding SARC-CoV-2 spike protein. For IgG titer assay, LNPs of the present disclosure were prepared to carry either a mRNA encoding wild-type SARC-CoV-2 spike protein or a mRNA encoding wild-type SARC-CoV-2 spike protein with low-sugar modification.

Animal Immunizations. BALB/c mice aged 6 to 8 wk old (n=5) were immunized intramuscularly with 15 μg of LNPs in phosphate-buffered saline (PBS). Animals were immunized at week 0 and boosted with a second vaccination at week 2, and serum samples were collected from each mouse 2 weeks after the second immunization.

Pseudovirus neutralization assay. Pseudovirus was constructed by the RNAi Core Facility at Academia Sinica using a procedure similar to that described previously. Briefly, the pseudotyped lentivirus carrying SARS-CoV-2 spike protein was generated by transiently transfecting HEK-293T cells with pCMV-ΔR8.91, pLAS2w.Fluc.Ppuro. HEK-293T cells were seeded one day before transfection, and indicated plasmids were delivered into cells using TransITR-LT1 transfection reagent (Mirus). The culture medium was refreshed at 16 hours and harvested at 48 hours and 72 hours post-transfection. Cell debris was removed by centrifugation at 4,000×g for 10 min, and the supernatant was passed through a 0.45-μm syringe filter (Pall Corporation). The pseudotyped lentivirus was aliquot and then stored at −80° C. To estimate the lentiviral titer by AlarmaBlue assay (Thermo Scientific), The transduction unit (TU) of SARS-CoV-2 pseudotyped lentivirus was estimated by using cell viability assay in responded to the limited dilution of lentivirus. In brief, HEK-293T cells stably expressing the human ACE2 gene were plated on a 96-well plate one day before lentivirus transduction. For the tittering pseudotyped lentivirus, different amounts of lentivirus were added into the culture medium containing polybrene (final concentration 8 μg/ml). Spin infection was carried out at 1,100×g in a 96-well plate for 30 minutes at 37° C. After incubating cells at 37° C. for 16 hr, the culture medium containing virus and polybrene was removed and replaced with fresh complete DMEM containing 2.5 μg/ml puromycin. After treating puromycin for 48 hrs, the culture media was removed, and the cell viability was detected using 10% AlamarBlue reagents according to the manufacturer's instructions. The survival rate of uninfected cells (without puromycin treatment) was set as 100%. The virus titer (transduction units) was determined by plotting the survival cells versus the diluted viral dose. For neutralization assay, heat-inactivated sera or antibodies were serially diluted and incubated with 1,000 TU of SARS-CoV-2 pseudotyped lentivirus in DMEM for 1 h at 37° C. The mixture was then inoculated with 10,000 HEK-293T cells stably expressing the human ACE2 gene in a 96-well plate. The culture medium was replaced with fresh complete DMEM (supplemented with 10% FBS and 100 U/mL penicillin/streptomycin) at 16 h postinfection and continuously cultured for another 48 h. The expression level of the luciferase gene was determined by using the Bright-Glo Luciferase Assay System (Promega). The relative light unit (RLU) was detected by Tecan i-control (Infinite 500). The percentage of inhibition was calculated as the ratio of RLU reduction in the presence of diluted serum to the RLU value of no serum control using the formula (RLU^{control}−RLU^{Serum})/RLU control.

Measurement of serum IgG titer. ELISA was used to determine the IgG titer of the mouse serum. The wells of a 96-well ELISA plate (Greiner Bio-One) were coated with 100 ng SARS-CoV-2 spike protein (ACROBiosystems, wild-type, Delta, or Omicron, respectively) in 100 mM sodium bicarbonate pH 8.8 at 4° C. overnight. The wells were blocked with 200 μl 5% skim milk in 1×PBS at 37° C. for 1 hour and washed with 200 μl PBST (1×PBS, 0.05% Tween 20, pH 7.4) three times. Mice serum samples with 2-fold serial dilution were added into wells for incubation at 37° C. for 2 hours and washed with 200 μl PBST six times. The wells were incubated with 100 μl HRP conjugated anti-mouse secondary antibody (1:10000, in PBS) at 37° C. for 1 hour and washed with 200 μl PBST six times. 100 μl horseradish peroxidase substrate (1-Step™ Ultra TMB-ELISA Substrate Solution) (THERMO FISHER SCIENTIFIC®) was added into wells, followed by 100 μl 1M H₂SO₄. After incubation for 30 minutes, absorbance (OD 450 nm) was measured using SpectraMax M5.

Results. FIG. 33 shows that all tested LNPs carrying the mRNA cargo were able to deliver and express the mRNA in vivo, thereby invoking immune responses that resulted in neutralization inhibition. Nevertheless, the inhibitory effect of those tested LNPs differed as the dilution factor increased. Both L1+L2 LNP and LNP (M) only showed a slightly higher inhibitory effect at 1:5000 dilution compared with the negative control, but the LNP of the present disclosure maintained around 40% inhibitory effect. The data demonstrates that the LNP of the present disclosure was able to invoke immune responses at a much lower concentration than other LNPs tested in this experiment.

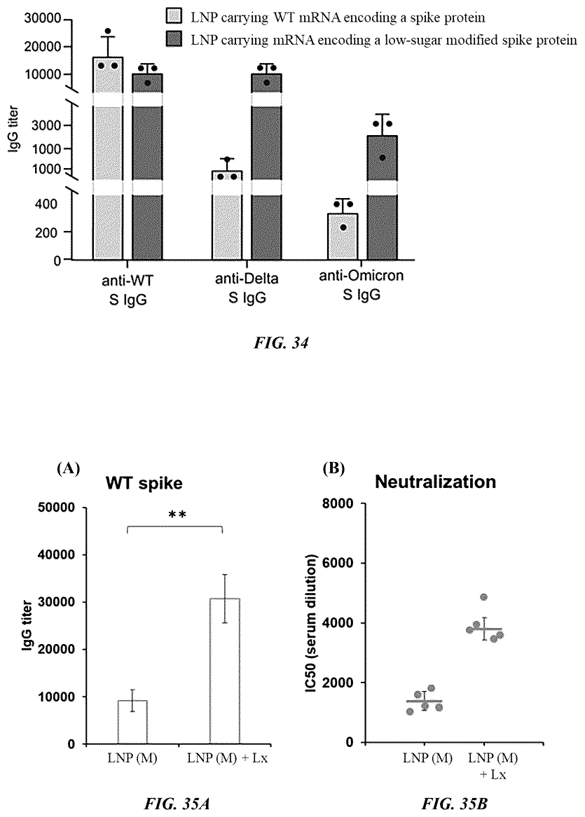

FIG. 34 verifies that the LNPs of the present disclosure were capable of inducing antigen-specific IgG in vivo. The LNP, carrying mRNA encoding wide-type spike protein, induced IgGs that were still able to recognize the spike proteins of both the Delta variant and Omicron variant at a good level. The data shows that the targeted delivery feature of the LNP can at least partially overcome the immune escape due to the spike protein variations between variants.

Furthermore, it was observed that LNP carrying wild-type SARS-CoV-2 spike protein-encoding mRNA (“WT LNP”) and LNP carrying mRNA encoding a low-sugar modified spike protein (“low-sugar LNP”) induced comparable IgG titers against wide-type viruses, the WT LNP had lower IgG titers against the Delta and Omicron strains, suggesting an immune escape. In contrast, the low-sugar LNP maintains a high level of IgG titers against the two variant strains. The results demonstrate that removing glycan shields improves the immunogenicity of the LNP formulations.

FIG. 35A and FIG. 35B show the results of an additional experiment. In this experiment, a MODERNA® LNP was prepared using MODERNA®'s proprietary formulation. In addition, LNP made using the MODERNA® formulation and adding a compound of the present disclosure was also prepared to test whether the compound of the present disclosure improves the performance of the MODERNA® formulation. Animals were administered with the LNPs, and IgG titer assay and neutralization assay were all performed as described above in this example. The results demonstrate that the compound of the present disclosure increased the spike protein-specific IgGs. The serum obtained from mice administered with the LNP of the present disclosure's compound also exhibited better neutralization. This experiment confirmed the targeted delivery feature of the compound of the present disclosure and verified that it can be applied to commercially available LNP formulations.

### Embodiments

Embodiment 1: An isolated nucleic acid, which encodes a recombinant influenza hemagglutinin (HA) comprising a signal peptide, an HA1 subunit, and an HA2 subunit, wherein the influenza HA comprises at least one glycosylation site, and the HA2 subunit is devoid of a glycosylation site.

Embodiment 2: The isolated nucleic acid of embodiment 1, wherein the HA1 subunit and the HA2 subunit are defined by a conserved proteolytic cleavage site comprising a X_{b}-X_{b}—R-G (SEQ ID NO: 33) peptide, wherein G denotes a glycine (G) residue, R denotes an arginine (R) residue, and X_{b} denotes any amino acid.

Embodiment 3: The isolated nucleic acid of embodiment 1 or embodiment 2, wherein the influenza HA comprises at least one glycosylation site in the HA1 subunit.

Embodiment 4: The isolated nucleic acid of any one of embodiments 1 to 3, wherein the influenza HA comprises a stem region, comprising (1) the HA2 subunit, (2) a HA1 N-terminal loop motif, (3) a HA1 lower helix motif, and (4) a HA1 C-terminal beta-strands motif, wherein, except for the HA1 N-terminal loop motif, the stem region is devoid of a glycosylation site.

Embodiment 5: The isolated nucleic acid of embodiment 4, wherein the HA1 N-terminal loop motif comprises the first 10 to 40 amino acids, the first 15 to 40 amino acids, or the first 20 to 40 amino acids immediately C-terminal to the signal peptide.

Embodiment 6: The isolated nucleic acid of embodiment 4 or embodiment 5, wherein the HA1 C-terminal beta-strands motif comprises the last 10 to 30 amino acids of the HA1 subunit, and the HA1 lower helix motif comprises the 5 to 15 amino acids immediately upstream of the HA1 C-terminal beta-strands motif.

Embodiment 7: The isolated nucleic acid of any one of embodiments 1 to 6, wherein the HA2 subunit and/or the stem region comprise a deglycosylation sequon of Z-Xₐ-S/T, wherein Z denotes any amino acid residue except for asparagine (N; Asn), S denotes a serine (S; Ser) residue, T denotes a threonine (T; Thr) residue, and Xₐ is any amino acid residue except for proline (P).

Embodiment 8: The isolated nucleic acid of embodiment 7, wherein, in the deglycosylation sequon, Z denotes glutamine (Q; Gln).

Embodiment 9: The isolated nucleic acid of any one of embodiments 4 to 8, wherein the HA1 N-terminal loop motif comprises a glycosylation site.

Embodiment 10: The isolated nucleic acid of any one of embodiments 4 to 8, wherein the HA1 N-terminal loop motif is devoid of a glycosylation site.

Embodiment 11: The isolated nucleic acid of any one of embodiments 1 to 10, wherein the HA2 subunit and/or the stem region is devoid of a glycosylation site, compared with a reference influenza HA, wherein the reference influenza HA comprises an amino acid sequence at least 80%, 85%, 90%, 91%, 92%, 93%, 94%, 95%, 96%, 97%, 98%, or 99%, or 100% identical to SEQ ID NO: 1, SEQ ID NO: 4, SEQ ID NO: 7, SEQ ID NO: 14, or SEQ ID NO: 19.

Embodiment 12: The isolated nucleic acid of any one of embodiments 1 to 11, wherein the recombinant influenza HA is derived from an influenza A virus or an influenza B virus.

Embodiment 13: The isolated nucleic acid of embodiment 12, wherein the influenza A virus is a H1, H2, H3, H4, H5, H6, H7, H8, H9, H10, H11, H12, H13, H14, H15, H16, H17, or H18 lineage.

Embodiment 14: The isolated nucleic acid of embodiment 12, wherein the influenza B virus is a Yamagata or Victoria lineage.

Embodiment 15: The isolated nucleic acid of any one of embodiments 1 to 8, wherein the influenza HA comprises an amino acid sequence at least 80%, 85%, 90%, 91%, 92%, 93%, 94%, 95%, 96%, 97%, 98%, or 99%, or 100% identical to SEQ ID NO 5:

MKAILVVMLY TFTTANADTL CIGYHAXXST DTVDTVLEKX
VTVTHSVNLL EDKHNGKLCK LRGVAPLHLG QCNIAGWILG
NPECESLSTA RSWSYIVETS NSDNGTCYPG DFINYEELRE
QLSSVSSFER FEIFPKTSSW PNHDSDNGVT AACSHAGARS
FYKNLIWLVK KGKSYPKINQ TYINDKGKEV LVLWGIHHPP
TITDQESLYQ NADAYVFVGT SRYSKKFKPE IAARPKVRDR
AGRMNYYWTL VEPGDKITFE ATGNLVAPRY AFTMEKEAGS
GIIISDTPVH DCXATCQTPE GAIXTSLPFQ NVHPITIGKC
PKYVRSTKLR LATGLRNVPS IQSRGLFGAI AGFIEGGWTG
MVDGWYGYHH QNDQGSGYAA DLKSTQNAID KITNKVNSVI
EKMNTQFTAV GKEFNHLEKR IENLNKKVDD GFLDVWTYNA
ELLVLLENER TLDYHDSNVK NLYEKVRHQL KNNAKEIGNG
CFEFYHKCDN TCMESVKXGT YDYPKYSEEA KLNREKIDGV
KLDSTRIYQI LAIYSTVASS LVLVVSLGAI SFWMCSNGSL
QCRICI,

wherein X denotes any amino acid, provided that (1) X40, X293, X304, and X498 are not asparagine (N; Asn), and both X27 and X28 are Asn; or (2) X27, X40, X293, X304, and X498 are not Asn.

Embodiment 16: The isolated nucleic acid of embodiment 15, wherein X40, X293, X304, and/or X498 are glutamine (Q; Gln).

Embodiment 17: The isolated nucleic acid of embodiment 15 or embodiment 16, wherein the influenza HA comprises SEQ ID NO: 6.

Embodiment 18: The isolated nucleic acid of any one of embodiments 1 to 8, wherein the influenza HA comprises an amino acid sequence at least 80%, 85%, 90%, 91%, 92%, 93%, 94%, 95%, 96%, 97%, 98%, or 99%, or 100% identical to SEQ ID NO: 8:

MKAIIALSNI LCLVFAQKIP GNDXSTATLC LGHHAVPXGT
IVKTITNDRI EVTNATELVQ NSSIGKICNS PHQILDGGNC
TLIDALLGDP QCDGFQNKEW DLFVERSRAN SSCYPYDVPD
YASLRSLVAS SGTLEFKNES FNWTGVKQNG TSSACKRGSS
SSFFSRLNWL ISLNNIYPAQ NVTMPNKEQF DKLYIWGVHH
PDTDKNQFSL FAQSSGRITV STKRSQQAVI PNIGSRPRVR
DIPSRISIYW TIVKPGDILL INSTGNLIAP RGYFKIRSGK
SSIMRSDAPI GKCKSECITP NGSIPNDKPF QNVNRITYGA
CPRYVKQSTL KLATGMRNVP EKQTRGIFGA IAGFIENGWE
GMVDGWYGFR HQNSEGRGQA ADLKSTQAAI DQISGKLNRL
IGKTNEKFHQ IEKEFSEVEG RVQDLEKYVE DTKIDLWSYN
AELLVALENQ HTIDLTDSEM NKLFEKTKKQ LRENAEDMGN
GCFKIYHKCD NACIGSIRXE TYDHNVYRDE ALNNRFQIKG
VELKSGYKDW ILWISFAMSC FLLCIALLGF IMWACQKGNI
RCNICI,

and
wherein X denotes any amino acid, provided that (1) X38 and X499 are not asparagine (N; Asn), and N24 is Asn, or (2) X24, X38 and X499 are not Asn.

Embodiment 19: The isolated nucleic acid of embodiment 18, wherein X38 and/or X499 are glutamine (Q; Gln).

Embodiment 20: The isolated nucleic acid of embodiment 18 or embodiment 19, wherein the influenza HA comprises SEQ ID NO: 9.

Embodiment 21: The isolated nucleic acid of any one of embodiments 1 to 8, wherein the influenza HA comprises an amino acid sequence at least 80%, 85%, 90%, 91%, 92%, 93%, 94%, 95%, 96%, 97%, 98%, or 99%, or 100% identical to SEQ ID NO: 8:

MKAIIALSNI LCLVFAQKIP GNDXSTATLC LGHHAVPXGT
IVKTIINDRI EVTNATELVQ NSSIGKICNS PHQILDGGNC
TLIDALLGDP QCDGFQNKEW DLFVERSRAN SSCYPYDVPD
YASLRSLVAS SGTLEFKNES FNWTGVKQNG TSSACKRGSS
SSFFSRLNWL TSLNNIYPAQ NVTMPNKEQF DKLYIWGVHH
PDTDKNQFSL FAQSSGRITV STKRSQQAVI PNIGSRPRVR
DIPSRISIYW TIVKPGDILL INSTGNLIAP RGYFKIRSGK
SSIMRSDAPI GKCKSECITP NGSIPNDKPF QNVNRITYGA
CPRYVKQSTL KLATGMRNVP EKQTRGIFGA IAGFIENGWE
GMVDGWYGFR HQNSEGRGQA ADLKSTQAAI DQISGKLNRL
IGKTNEKFHQ IEKEFSEVEG RVQDLEKYVE DTKIDLWSYN
AELLVALENQ HTIDLTDSEM NKLFEKTKKQ LRENAEDMGN
GCFKIYHKCD NACIGSIRXE TYDHNVYRDE ALNNRFQIKG
VELKSGYKDW ILWISFAMSC FLLCIALLGF IMWACQKGNI
RCNICI,

and
wherein X denotes any amino acid, provided that X24, X38, and X499 are not asparagine (N; Asn), and N54, N61, N79, and N301 are replaced with an amino acid other than Asn.

Embodiment 22: The isolated nucleic acid of embodiment 21, wherein X24, X38, and/or X499 are glutamine (Q; Gln).

Embodiment 23: The isolated nucleic acid of embodiment 21 or embodiment 22, wherein N54, N61, N79, and N301 are replaced with glutamine (Q; Gln).

Embodiment 24: The isolated nucleic acid of any one of embodiments 21 to 23, wherein the influenza HA comprises SEQ ID NO: 11.

Embodiment 25: The isolated nucleic acid of any one of embodiments 1 to 8, wherein the influenza HA comprises an amino acid sequence at least 80%, 85%, 90%, 91%, 92%, 93%, 94%, 95%, 96%, 97%, 98%, or 99%, or 100% identical to SEQ ID NO 2:

MKAIIVLLMV VTSNADRICT GITSSNSPHV VKTATQGEVX
VTGVIPLTTT PTKSYFANLK GTRTRGKLCP DCLNCTDLDV
ALGRPMCVGT TPSAKASILH EVRPVTSGCF PIMHDRTKIR
QLPNLLRGYE KIRLSTQNVI DAEKAPGGPY RLGTSGSCPN
ATSKIGFFAT MAWAVPKDNY KNATNPLTVE VPYICTEGED
QITVWGFHSD DKTQMKSLYG DSNPQKFTSS ANGVTTHYVS
QIGDFPDQTE DGGLPQSGRI VVDYMMQKPG KTGTIVYQRG
VLLPQKVWCA SGRSKVIKGS LPLIGEADCL HEEYGGLXKS
KPYYTGKHAK AIGNCPIWVK TPLKLAXGTK YRPPAKLLKE
RGFFGAIAGF LEGGWEGMIA GWHGYTSHGA HGVAVAADLK
STQEAINKIT KNLNSLSELE VKNLQRLSGA MDELHNEILE
LDEKVDDLRA DTISSQIELA VLLSNEGIIN SEDEHLLALE
RKLKKMLGPS AVDIGNGCFE TKHKCXQTCL DRIAAGTFNA
GEFSLPTFDS LXITAASLND DGLDXHTILL YYSTAASSLA
VTLMLAIFIV YMVSRDNVSC SICL,

wherein X denotes any amino acid, provided that (1) X318, X347, X506, X532, and X545 are not asparagine (N; Asn), and X40 is Asn or (2) X40, X318, X347, X506, X532, and X545 are not Asn.

Embodiment 26: The isolated nucleic acid of embodiment 25, wherein X318, X347, X506, X532, and/or X545 are glutamine (Q; Gln).

Embodiment 27: The isolated nucleic acid of embodiment 25 or embodiment 27, wherein the influenza HA comprises SEQ ID NO: 3.

Embodiment 28: The isolated nucleic acid of any one of embodiments 1 to 8, wherein the influenza HA comprises an amino acid sequence at least 80%, 85%, 90%, 91%, 92%, 93%, 94%, 95%, 96%, 97%, 98%, or 99%, or 100% identical to SEQ ID NO: 15:

MKAIIVLLMV VTSNADRICT GITSSNSPHV VKTATQGEVX
VTGVIPLTTT PTKSHFANLK GTETRGKLCP KCLNCTDLDV
ALGRPKCTGK IPSARVSILH EVRPVTSGCF PIMHDRTKIR
QLPNLLRGYE HVRLSTHNVI NTEDAPGGPY EIGTSGSCLN
ITNGKGFFAT MAWAVPKNKT ATNPLTIEVP YICTEEEDQI
TVWGFHSDDE TQMARLYGDS KPQKFTSSAN GVTTHYVSQI
GGFPNQTEDG GLPQSGRIVV DYMVQKSGKT GTITYQRGIL
LPQKVWCASG KSKVIKGSLP LIGEADCLHE KYGGLXKSKP
YYTGEHAKAI GNCPIWVKTP LKLAXGTKYR PPAKLLKERG
FFGAIAGFLE GGWEGMIAGW HGYTSHGAHG VAVAADLKST
QEAINKITKN LNSLSELEVK NLQRLSGAMD ELHNEILELD
EKVDDLRADT ISSQIELAVL LSNEGIINSE DEHLLALERK
LKKMLGPSAV EIGNGCFETK HKCXQTCLDR IAAGTFDAGE
FSLPTFDSLX ITAASLNDDG LDXHTILLYY STAASSLAVT
LMIAIFVVYM VSRDNVSCSI CL,

wherein X denotes any amino acid, provided that (1) X316, X345, X504, X530, and X543 are not asparagine (N; Asn), and X40 is Asn; (2) X504, X530, and X543 are not Asn, and X40, X316, and X345 are Asn; or (3) X40, X316, X345, X504, X530, and X543 are not Asn.

Embodiment 29: The isolated nucleic acid of embodiment 28, wherein (1) when X316, X345, X504, X530, and X543 are not Asn, X316, X345, X504, X530, and/or X543 are glutamine (Q; Gln), (2) when X504, X530, and X543 are not Asn, X504, X530, and/or X543 are Gln, and (3) when X40, X316, X345, X504, X530, and X543 are not Asn, X40, X316, X345, X504, X530, and/or X543 are Gln.

Embodiment 30: The isolated nucleic acid of embodiment 28 or embodiment 29, wherein the influenza HA comprises SEQ ID NO: 16 or SEQ ID NO: 18.

Embodiment 31: An isolated nucleic acid, encoding a recombinant influenza hemagglutinin (HA), wherein the influenza hemagglutinin (HA) comprises an amino acid sequence at least 80%, 85%, 90%, 91%, 92%, 93%, 94%, 95%, 96%, 97%, 98%, or 99%, or 100% identical to SEQ ID NO: 3.

Embodiment 32: The isolated nucleic acid of embodiment 31, comprising a nucleotide sequence at least 80%, 85%, 90%, 91%, 92%, 93%, 94%, 95%, 96%, 97%, 98%, or 99%, or 100% identical to SEQ ID NO: 20.

Embodiment 33: An isolated nucleic acid, encoding a recombinant influenza hemagglutinin (HA), wherein the influenza hemagglutinin (HA) comprises an amino acid sequence at least 80%, 85%, 90%, 91%, 92%, 93%, 94%, 95%, 96%, 97%, 98%, or 99%, or 100% identical to SEQ ID NO: 6.

Embodiment 34: The isolated nucleic acid of embodiment 33, comprising a nucleotide sequence at least 80%, 85%, 90%, 91%, 92%, 93%, 94%, 95%, 96%, 97%, 98%, or 99%, or 100% identical to SEQ ID NO: 21.

Embodiment 35: An isolated nucleic acid, encoding a recombinant influenza hemagglutinin (HA), wherein the influenza hemagglutinin (HA) comprises an amino acid sequence at least 80%, 85%, 90%, 91%, 92%, 93%, 94%, 95%, 96%, 97%, 98%, or 99%, or 100% identical to SEQ ID NO: 9.

Embodiment 36: The isolated nucleic acid of embodiment 35, comprising a nucleotide sequence at least 80%, 85%, 90%, 91%, 92%, 93%, 94%, 95%, 96%, 97%, 98%, or 99%, or 100% identical to SEQ ID NO: 22.

Embodiment 37: An isolated nucleic acid, encoding a recombinant influenza hemagglutinin (HA), wherein the influenza hemagglutinin (HA) comprises an amino acid sequence at least 80%, 85%, 90%, 91%, 92%, 93%, 94%, 95%, 96%, 97%, 98%, or 99%, or 100% identical to SEQ ID NO: 11.

Embodiment 38: The isolated nucleic acid of embodiment 37, comprising a nucleotide sequence at least 80%, 85%, 90%, 91%, 92%, 93%, 94%, 95%, 96%, 97%, 98%, or 99%, or 100% identical to SEQ ID NO: 23.

Embodiment 39: An isolated nucleic acid, encoding a recombinant influenza hemagglutinin (HA), wherein the influenza hemagglutinin (HA) comprises an amino acid sequence at least 80%, 85%, 90%, 91%, 92%, 93%, 94%, 95%, 96%, 97%, 98%, or 99%, or 100% identical to SEQ ID NO: 16.

Embodiment 40: The isolated nucleic acid of embodiment 39, comprising a nucleotide sequence at least 80%, 85%, 90%, 91%, 92%, 93%, 94%, 95%, 96%, 97%, 98%, or 99%, or 100% identical to SEQ ID NO: 24.

Embodiment 41: An isolated nucleic acid, encoding a recombinant influenza hemagglutinin (HA), wherein the influenza hemagglutinin (HA) comprises an amino acid sequence at least 80%, 85%, 90%, 91%, 92%, 93%, 94%, 95%, 96%, 97%, 98%, or 99%, or 100% identical to SEQ ID NO: 18.

Embodiment 42: The isolated nucleic acid of embodiment 41, comprising a nucleotide sequence at least 80%, 85%, 90%, 91%, 92%, 93%, 94%, 95%, 96%, 97%, 98%, or 99%, or 100% identical to SEQ ID NO: 25.

Embodiment 43: An expression vector, comprising an expression cassette, which comprises the isolated nucleic acid of any one of embodiments 1 to 42.

Embodiment 44: The expression vector of embodiment 43, wherein the expression cassette further comprises a promoter, a 5′ untranslated region (5′UTR), a 3′ untranslated region (3′UTR), a 5′ cap, a poly-A tail, or a combination thereof, operably linked to the isolated nucleic acid.

Embodiment 45: The expression vector of embodiment 43 or embodiment 44, wherein the expression vector is a lipid nanoparticle, a liposome, a polymersome, a viral particle, a plasmid, or a bead.

Embodiment 46: The expression vector of embodiment 45, wherein the expression vector is a lipid nanoparticle, and the lipid nanoparticle comprises a membrane defining an inner space, and wherein the membrane encompasses the isolated nucleic acid, and the membrane is formed with a plurality of lipid components comprising a bi-functional compound, and the bi-functional compound comprises:

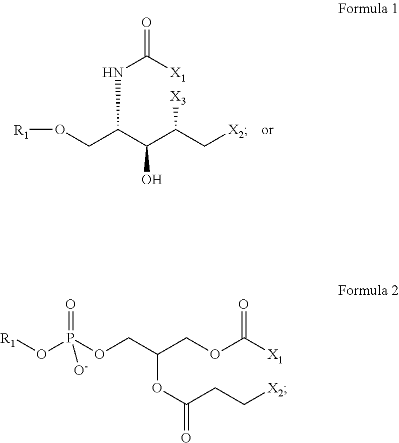

wherein R₁ comprises a substituted or non-substituted glycosyl group;
wherein X₁ and X₂ are each independently hydrogen, C₁₋₃₀ alkyl, C₁₋₃₀ alkenyl, C₁₋₃₀ alkynyl, aryl, aryloxy, or a substituted version thereof, or
   —(CH₂)nX₄, n is 0 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof; and
wherein X₃ is hydrogen, C₁₋₆ alkyl, or hydroxyl.

Embodiment 47: The expression vector of embodiment 46, wherein R₁ comprises a formula of R₂—R_{A}—, wherein R_{A} is an attachment group and R₂ is the substituted or non-substituted glycosyl group, and wherein the attachment group comprises an aryl, an alkyl, an amide, an alkylamide, a substituted version thereof, a combination thereof, or a covalent bond.

Embodiment 48: The expression vector of embodiment 47, wherein R_{A} comprises the aryl having 0 to 3 substituents, wherein the substituent is C₁₋₆ alkyl, halide, or C₁₋₆ alkyl halide.

Embodiment 49: The expression vector of embodiment 48, wherein R_{A} further comprises a polyethylene glycol (PEG) moiety having 2 to 72 (OCH₂CH₂) subunits.

Embodiment 50: The expression vector of any one of embodiments 46 to 49, wherein the glycosyl group comprises mannoside, fucoside, or a combination thereof.

Embodiment 51: The expression vector of any one of embodiments 46 to 50, wherein the glycosyl group comprises a terminal mannoside, a terminal fucoside, or both.

Embodiment 52: The expression vector of any one of embodiments 46 to 51, wherein the glycosyl group comprises a mono-mannoside, a di-mannoside, or a tri-mannoside.

Embodiment 53: The expression vector of embodiment 52, wherein the tri-mannoside is a linear or branched tri-mannoside.

Embodiment 54: The expression vector of embodiment 53, wherein the branched tri-mannoside is a α-1,3-α-1,6-trimannoside.

Embodiment 55: The expression vector of any one of embodiments 46 to 54, wherein R₁ is a substituted glycosyl group.

Embodiment 56: The expression vector of embodiment 55, wherein the glycosyl group comprises 1 to 6 substituents, wherein the substituent is C₁₋₆ alkyl, C₁₋₆ alkenyl, halogen, C₁₋₆ alkyl halide, C₁₋₆ alkoxy, amine, nitro, C₁₋₆ alkyl amine, amide, azido, aryl, cycloalkyl, heterocycloalkyl, sulfite, or a substituted version thereof, or a combination thereof.

Embodiment 57: The expression vector of embodiment 56, wherein the substituent of the glycosyl group is selected from the group consisting of aryl, 5-membered cycloalkyl, 6-membered cycloalkyl, 5-membered heterocycloalkyl, and 6-membered heterocycloalkyl, and a substituted version thereof, which comprises 1 to 6 substituents selected from the group consisting of C₁₋₆ alkyl, halogen, C₁₋₆ alkyl halide, C₁₋₆ alkoxy, amine, nitro, C₁₋₆ alkyl amine, amide, azido, carboxyl, hydroxyl, aryl, cycloalkyl, heterocycloalkyl, or a substituted version thereof, or a combination thereof.

Embodiment 58: The expression vector of embodiment 56 or embodiment 57, wherein the substituent of the glycosyl group is a substituted or non-substituted aryl, optionally the substituent of the glycosyl group is a phenyl substituted with OH, CH₃, NH₂, CF₃, OCH₃, F, Br, Cl, NO₂, N₃, or a combination thereof.

Embodiment 59: The expression vector of any one of embodiments 56 to 58, wherein the heterocycloalkyl comprises 1 to 3 heteroatoms, selected from the group consisting of O, S, and N.

Embodiment 60: The expression vector of any one of embodiments 46 to 59, wherein R₁ is selected from the group consisting of:

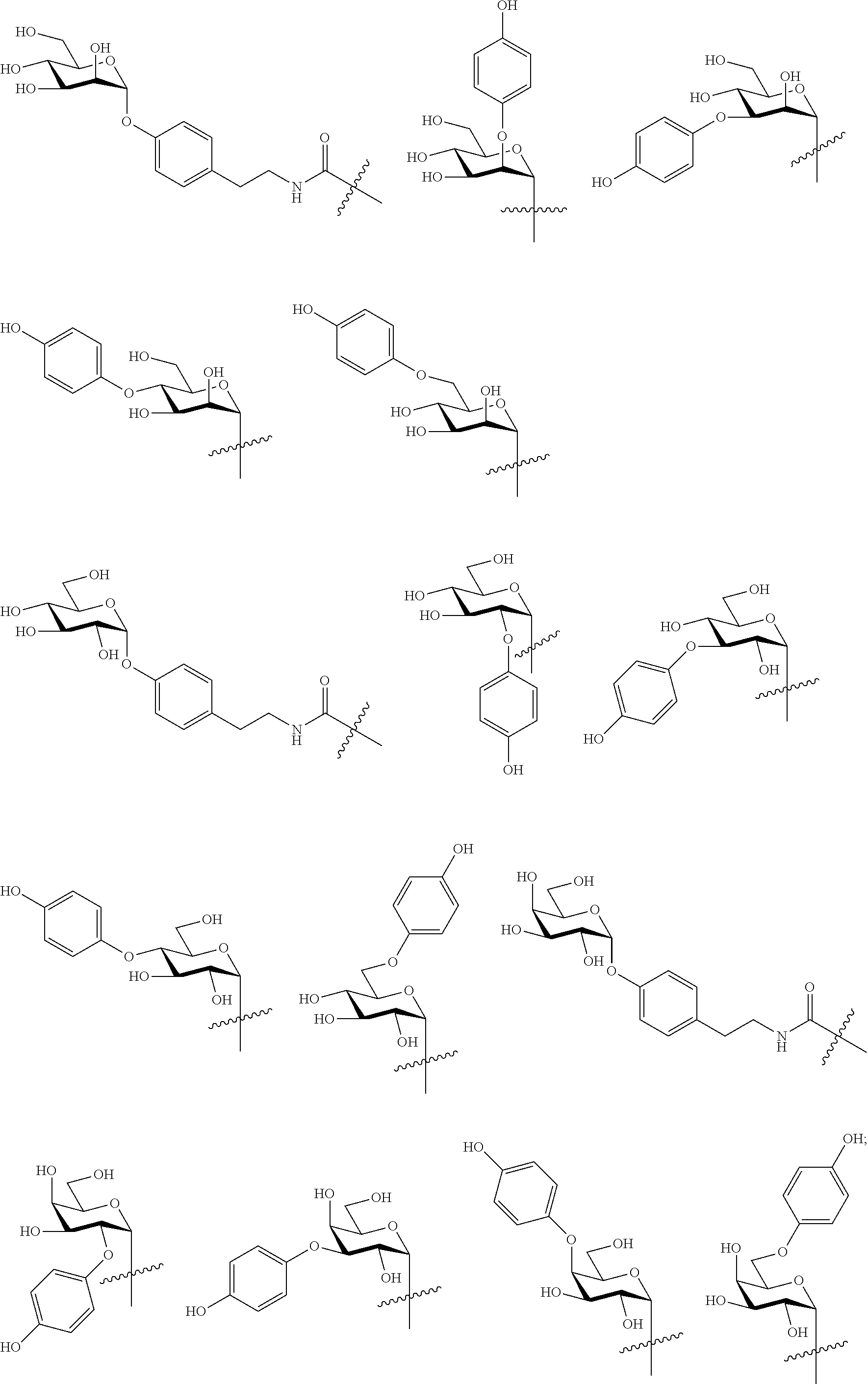

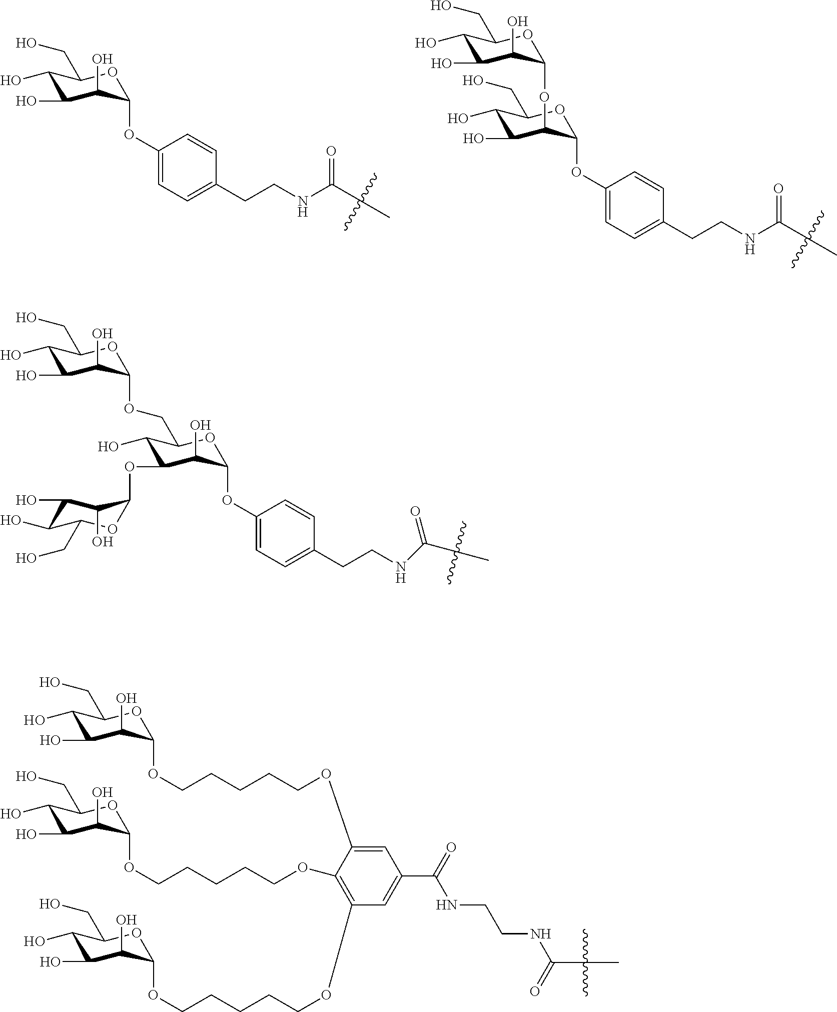

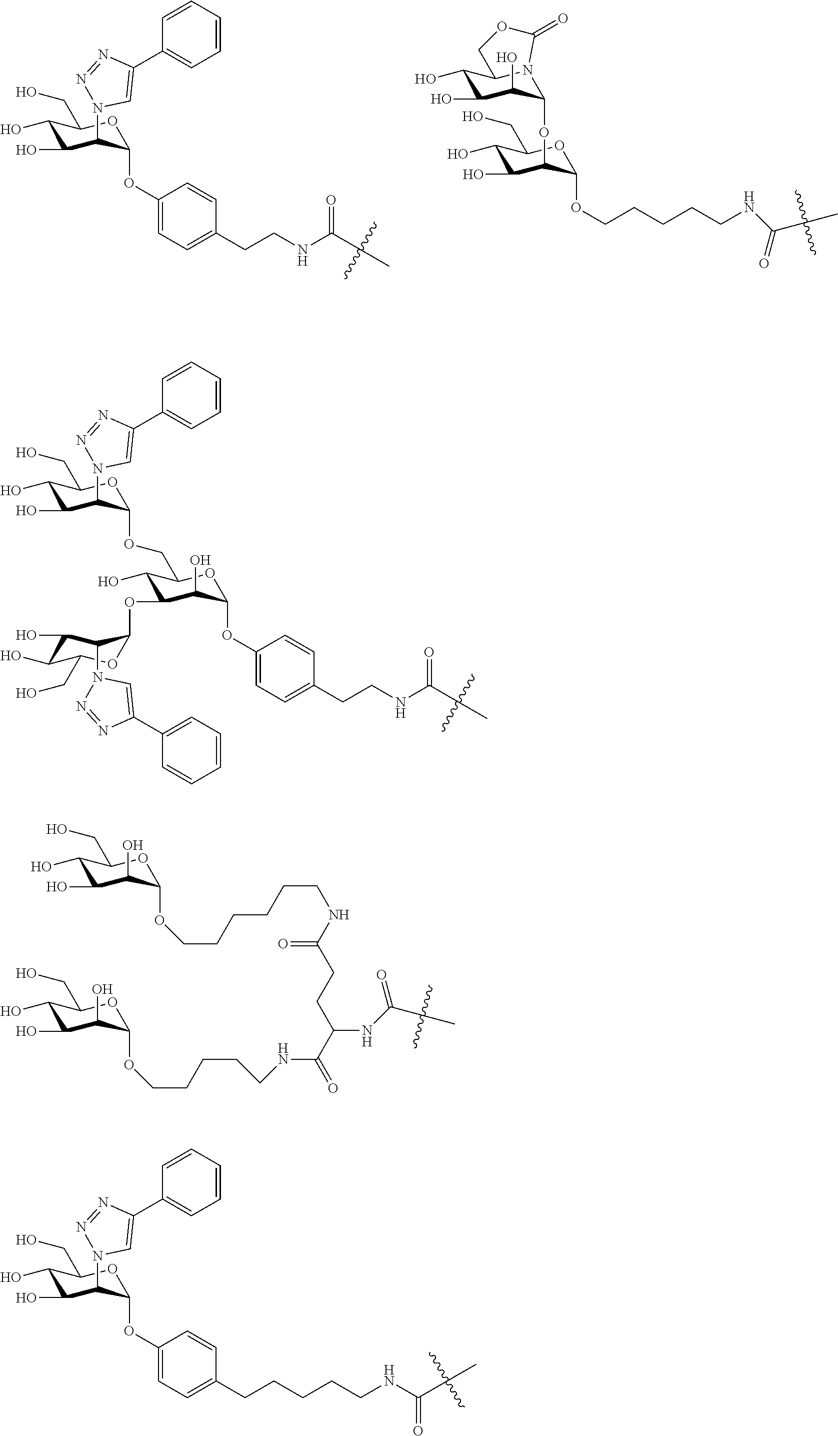

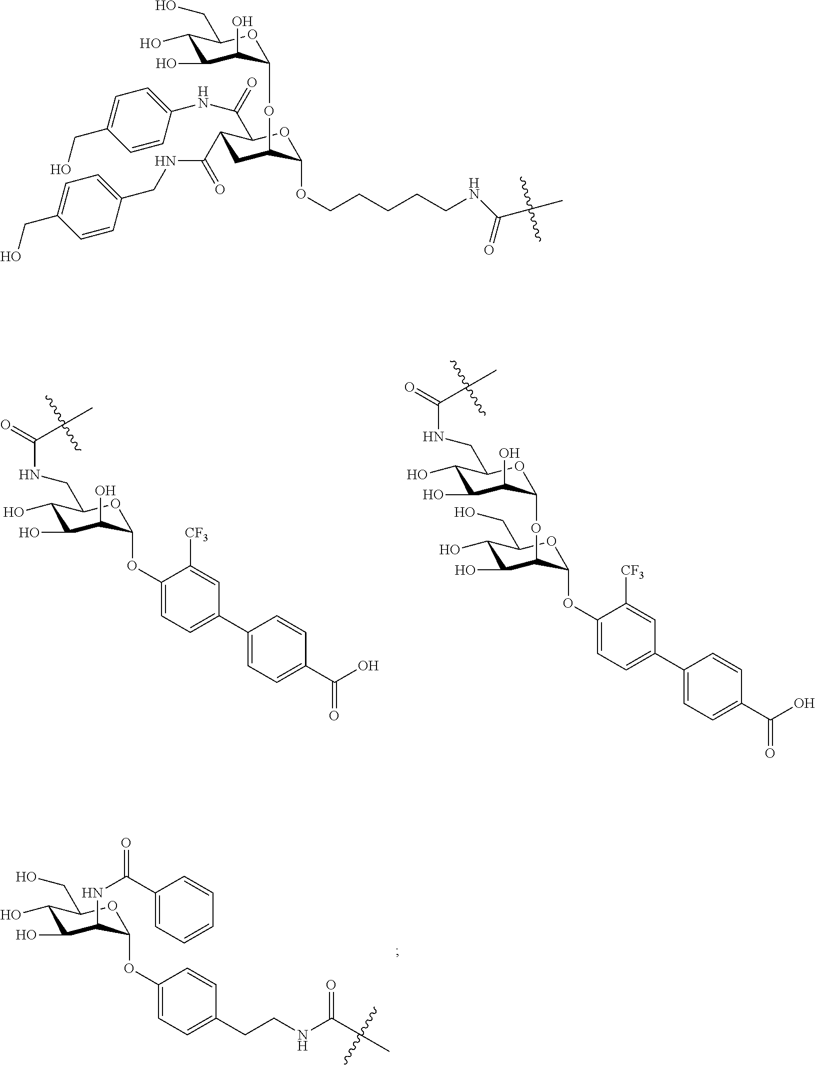

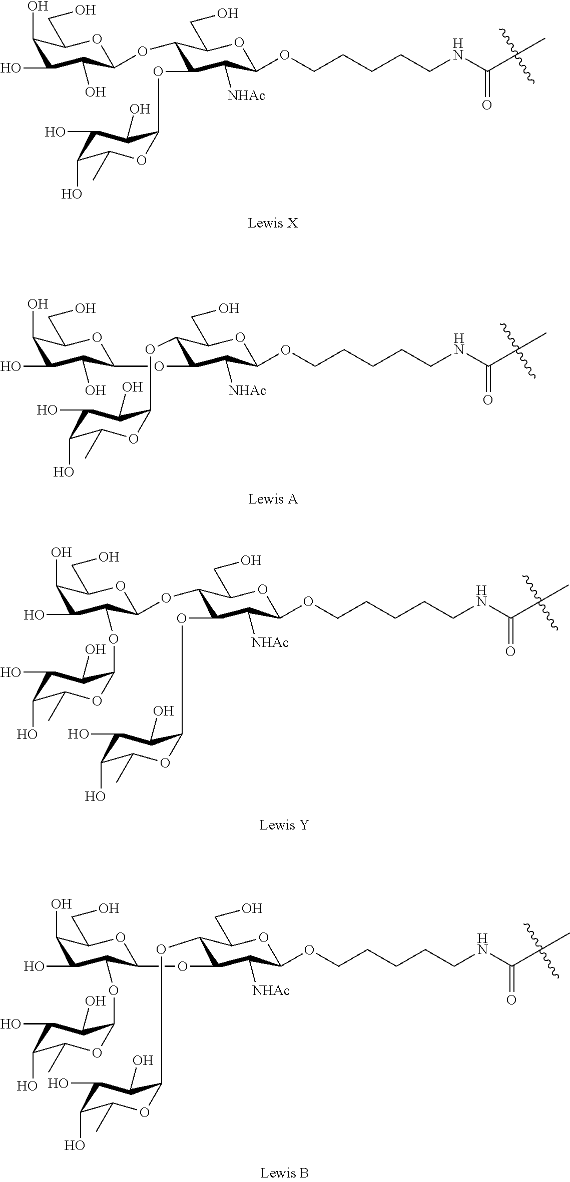

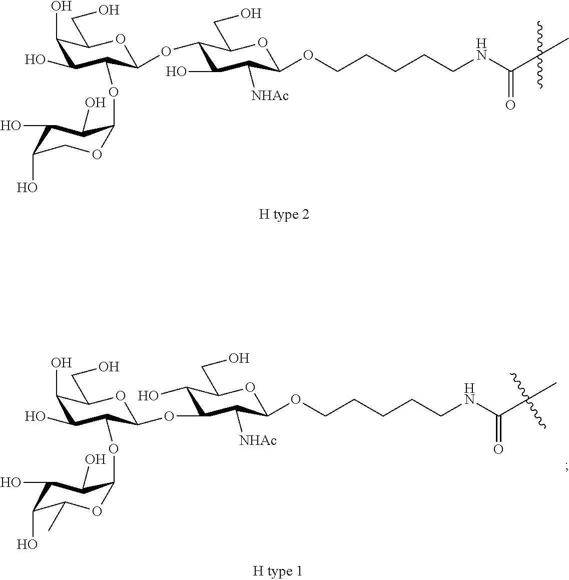

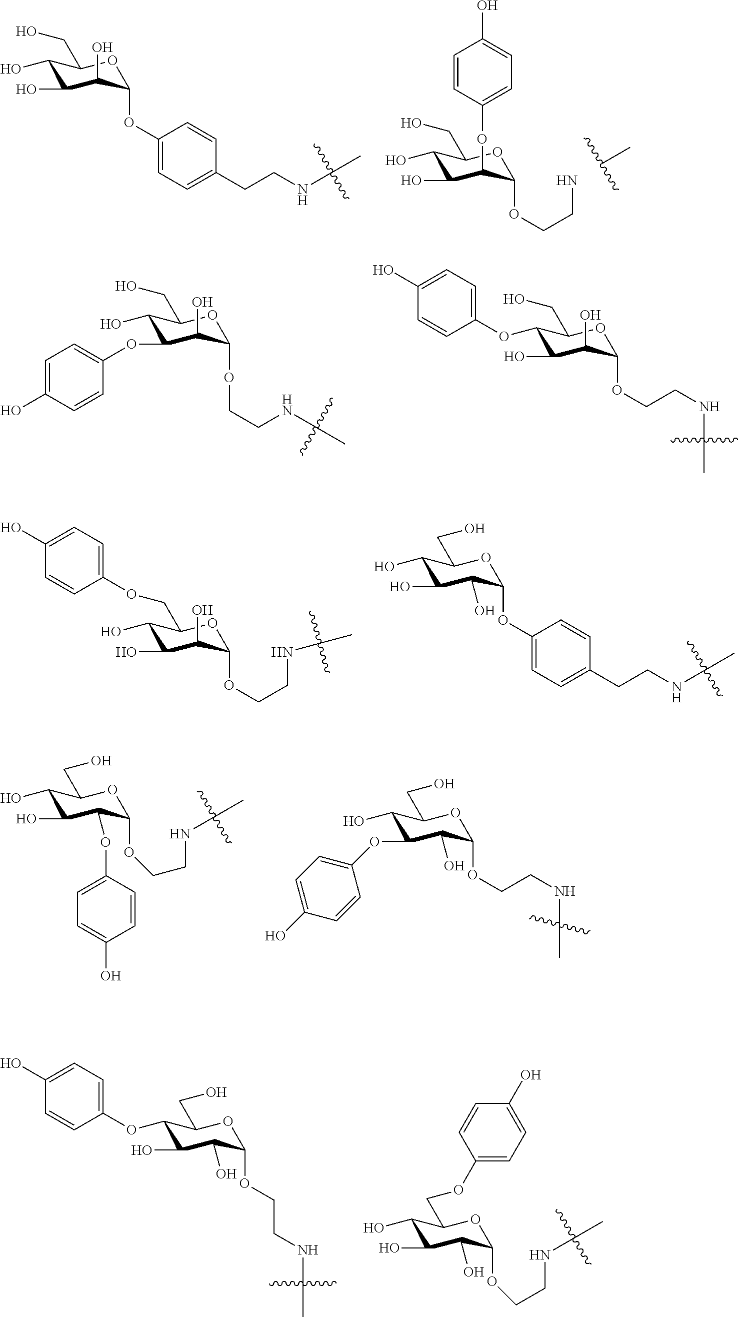

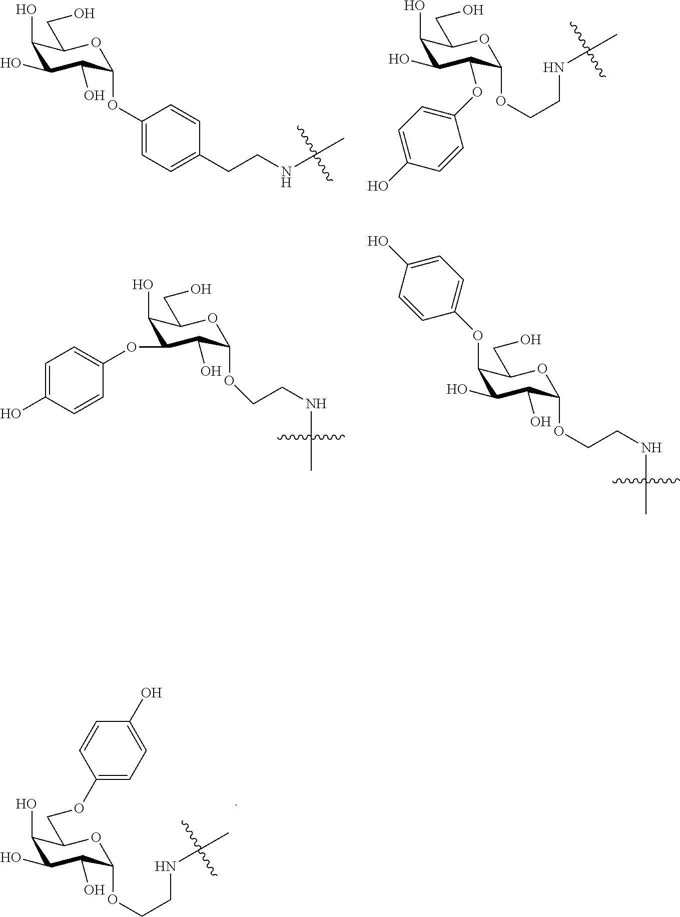

Embodiment 61: The expression vector of any one of embodiments 46 to 60, wherein the compound is of Formula 1.

Embodiment 62: The expression vector of any one of embodiments 46 to 61, wherein the compound is of Formula 2.

Embodiment 63: The expression vector of embodiment 62, wherein the compound is of Formula 3:

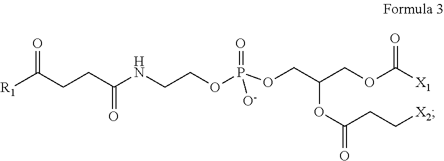

and
wherein R₁ is selected from the group consisting of:

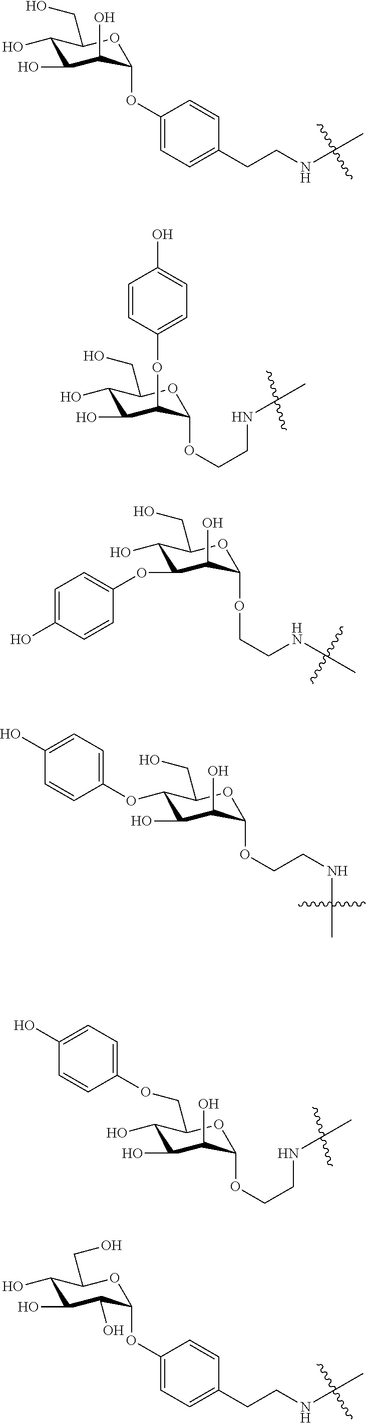

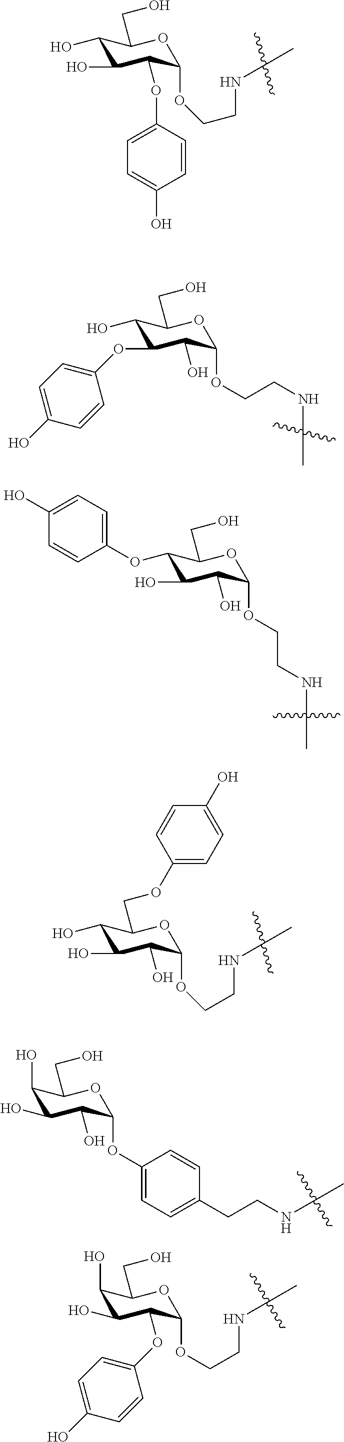

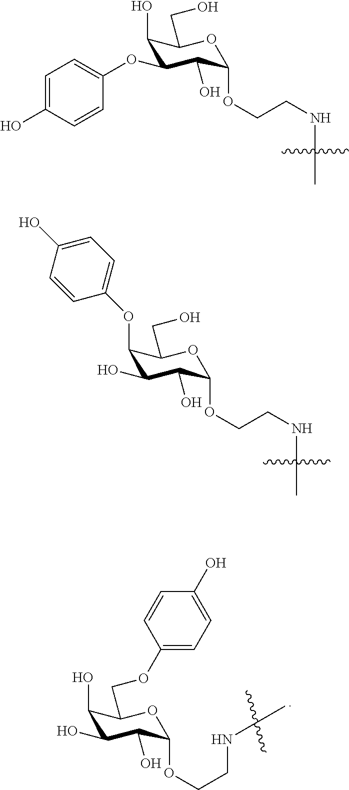

Embodiment 64: The expression vector of any one of embodiments 46 to 63, wherein at least one of X₁ and X₂ comprises a saturated hydrocarbon chain, comprising at least 2, 3, 4, 5, 6, 7, 8, 9, 10, 11, 12, 13, 14, 15, 16, 17, 18, 19, 20, 22, 24, 26, 28, or 30 carbons.

Embodiment 65: The expression vector of any one of embodiments 46 to 64, wherein X₁ and X₂ are each independently hydrogen, C₄₋₃₀ alkyl, C₄₋₃₀ alkenyl, C₄₋₃₀ alkynyl, aryl, aryloxy, or a substituted version thereof, or
—(CH₂)nX₄, n is 4 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof.

Embodiment 66: The expression vector of embodiment 65, wherein X₁ and X₂ are each independently hydrogen, C₈₋₃₀ alkyl, C₈₋₃₀ alkenyl, C₈₋₃₀ alkynyl, aryl, aryloxy, or a substituted version thereof, or
—(CH₂)nX₄, n is 8 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof.

Embodiment 67: The expression vector of any one of embodiments 46 to 66, provided that when one of X₁ and X₂ is hydrogen, the other one is not hydrogen.

Embodiment 68: The expression vector of any one of embodiments 46 to 67, wherein X₄ is an aryl, aryloxy, heterocyclic group, cycloalkyl, heterocycloalkyl, or a combination thereof, and wherein X₄ comprises 0 to 6 substituents, selected from the group consisting of C₁₋₆ alkyl, halogen, C₁₋₆ alkyl halogen, and C₁₋₆ alkoxy.

Embodiment 69: The expression vector of embodiment 68, wherein the substituent is CH₃, CF₃, F, or OCH₃.

Embodiment 70: The expression vector of embodiment 68 or embodiment 69, wherein X₄ comprises 1 to 3 substituents.

Embodiment 71: The expression vector of any one of embodiments 68 to 70, wherein X₄ is —R₃—O—R₄, wherein R₃ and R₄ are each independently aryl, heterocyclic group, cycloalkyl, heterocycloalkyl, each comprising 0 to 6 substituents selected from the group consisting of C₁₋₆ alkyl, halogen, C₁₋₆ alkyl halogen, and C₁₋₆ alkoxy.

Embodiment 72: The expression vector of any one of embodiments 46 to 71, wherein one of X₁ and X₂ is C₁₅₋₃₀ alkyl, and the other one is —(CH₂)nX₄.

Embodiment 73: The expression vector of any one of embodiments 46 to 72, wherein X₄ is selected from the group consisting of:

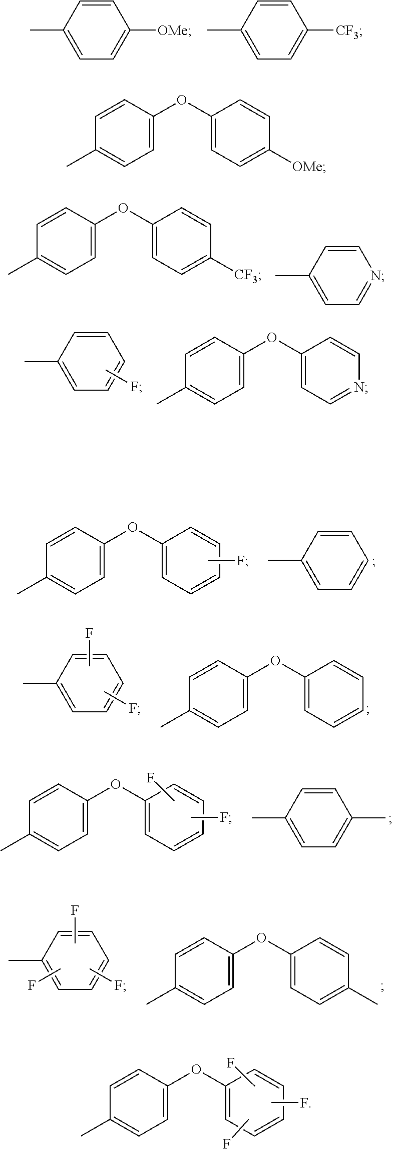

Embodiment 74: The expression vector of any one of embodiments 46 to 73, wherein the compound is selected from the group consisting of:

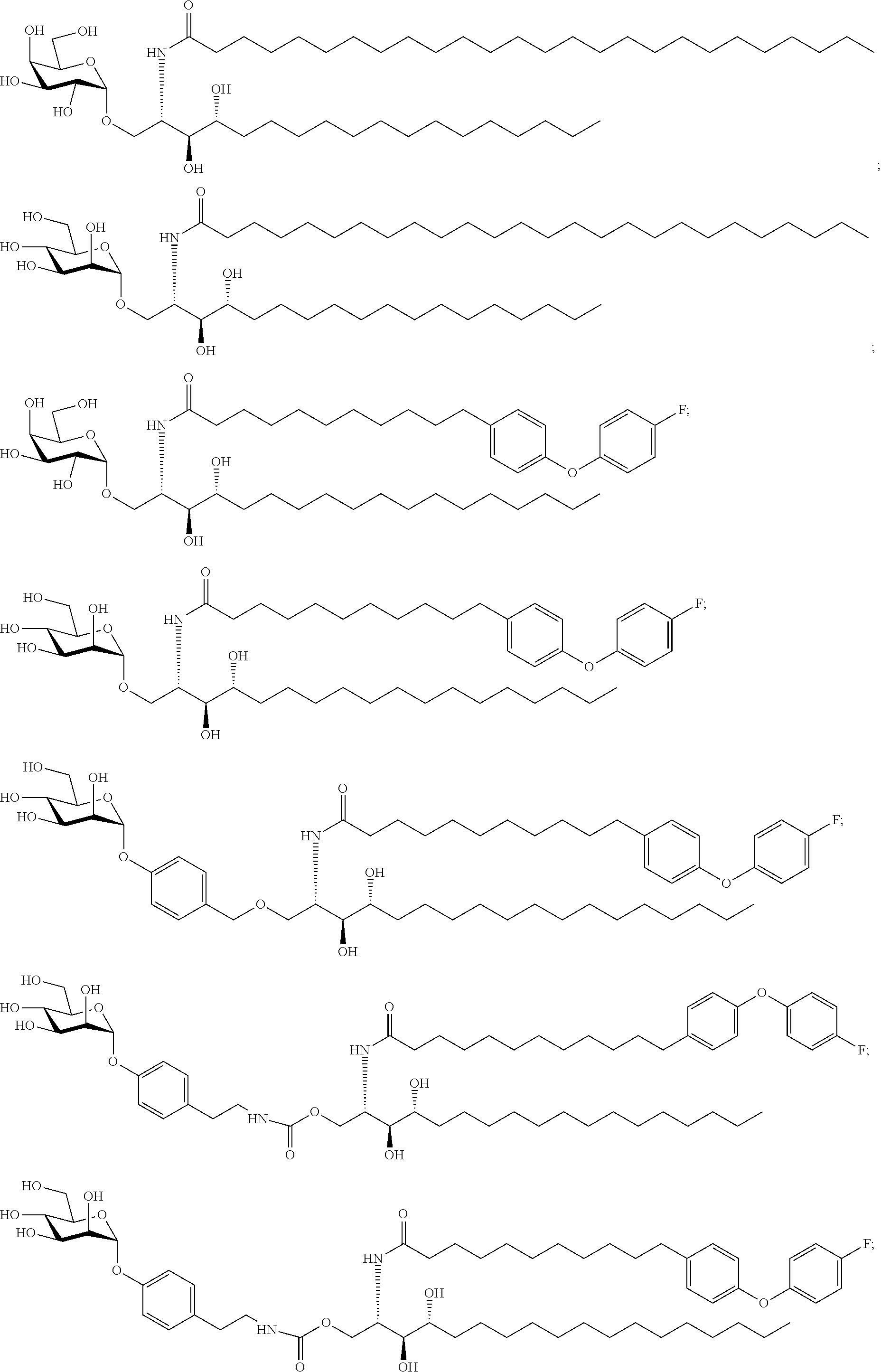

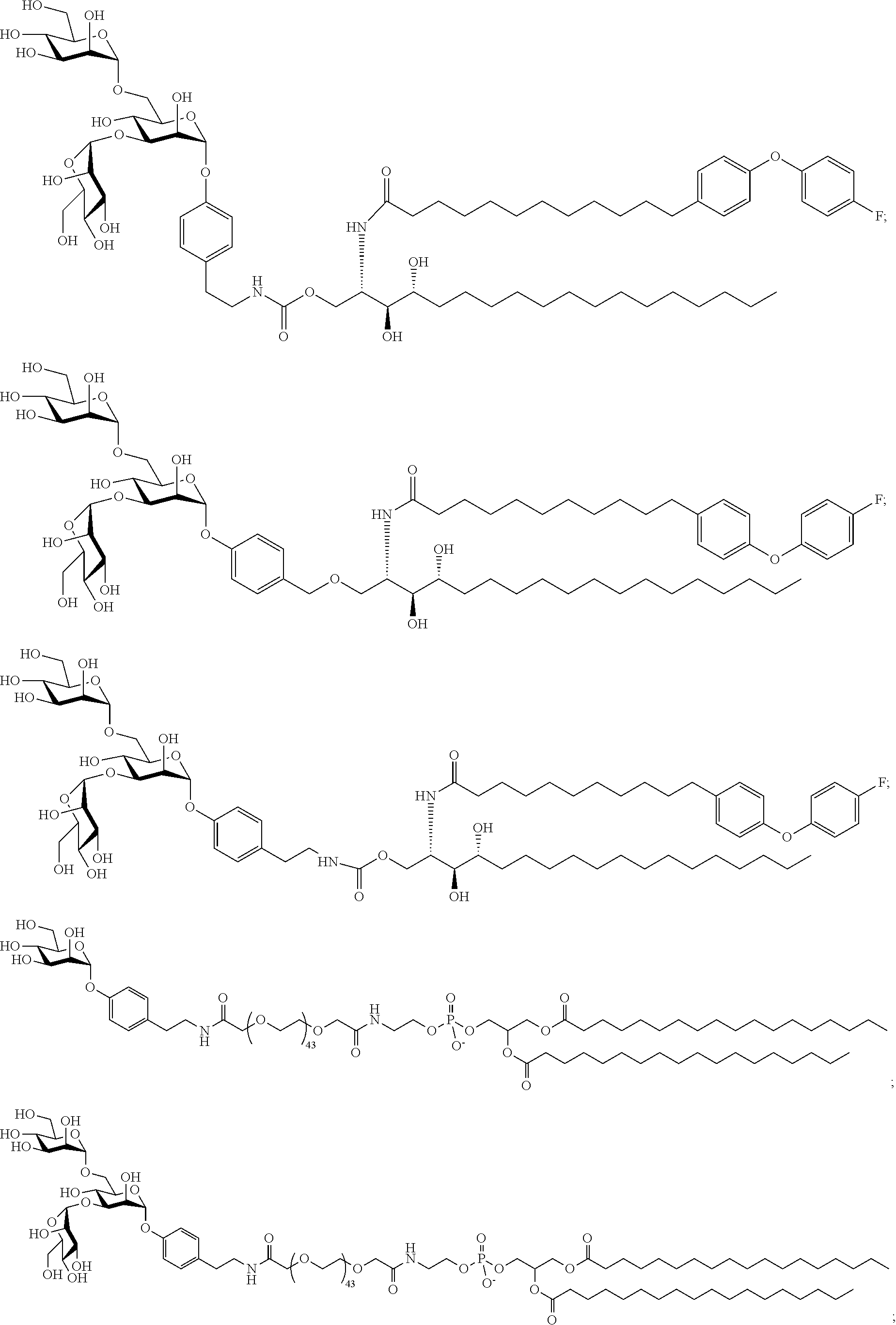

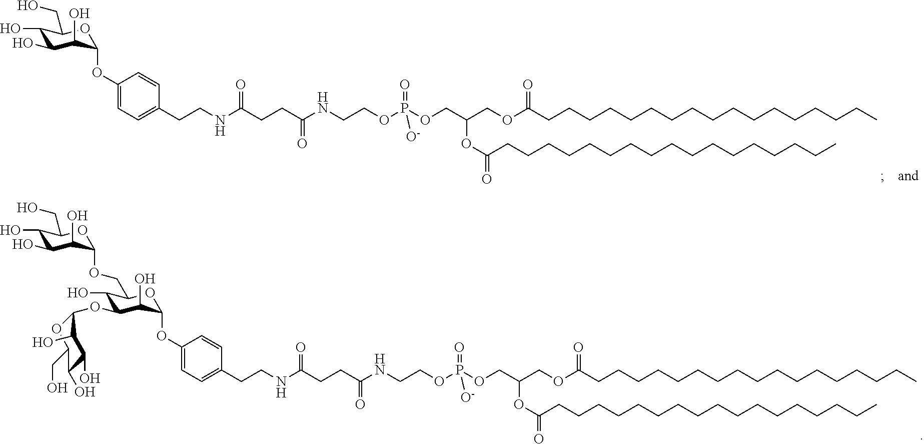

Embodiment 75: The expression vector of any one of embodiments 46 to 74, wherein the component is not glycolipid C₃₄ or α-galactosylceramide.

Embodiment 76: The expression vector of any one of embodiments 46 to 75, wherein the plurality of the lipid components further comprises an ionizable lipid, a helper lipid, or a combination thereof.

Embodiment 77: The expression vector of embodiment 76, wherein the ionizable lipid comprises heptadecan-9-yl 8-[2-hydroxyethyl-(6-oxo-6-undecoxyhexyl)amino]octanoate (SM-102™), (4-hydroxybutyl) azanediyl)bis(hexane-6,1-diyl)bis(2-hexyldecanoate) (ALC-0315™, Pfizer), or a combination thereof.

Embodiment 78: The expression vector of embodiment 76 or embodiment 77, wherein the helper lipid comprises a phosphatidylcholine, a cholesterol or a derivative thereof, a polyethylene glycol-lipid (PEG-lipid), or a mixture thereof.

Embodiment 79: The expression vector of embodiment 78, wherein the phosphatidylcholine comprises distearoylphosphatidylcholine (DSPC), dioleoylphosphatidylethanolamine (DPOE), or a mixture thereof.

Embodiment 80: The expression vector of embodiment 78 or embodiment 79, wherein the cholesterol or a derivative thereof is a cholesterol, campesterol, beta-sitosterol, brassicasterol, ergosterol, dehydroergosterol, stigmasterol, fucosterol, DC-cholesterol HCl, OH-Chol, HAPC-Chol, MHAPC-Chol, DMHAPC-Chol, DMPAC-Chol, cholesteryl chloroformate, GL67, cholesteryl myristate, cholesteryl oleate, cholesteryl nervonate, LC10, cholesteryl hemisuccinate, (3β,5β)-3-hydroxycholan-24-oic acid, alkyne cholesterol, 27-alkyne cholesterol, E-cholesterol alkyne, trifluoroacetate salt (Dios-Arg, 2H-Cho-Arg, or Cho-Arg), or a mixture thereof.

Embodiment 81: The expression vector of any one of embodiments 78 to 80, wherein the PEG-lipid is DMG-PEG, DSG-PEG, mPEG-DPPE, DOPE-PEG, mPEG-DMPE, mPEG-DOPE, DSPE-PEG-amine, DSPE-PEG, mPEG-DSPE, PEG PE, m-PEG-Pentacosadiynoic acid, bromoacetamido-PEG, amine-PEG, azide-PEG, or a mixture thereof.

Embodiment 82: An immunogenic composition, comprising an expression vector of any one of embodiments 43 to 81.

Embodiment 83: The immunogenic composition of embodiment 82, comprising at least about 0.01, 0.05, 0.1, 0.5, 1, 2, 3, 4, 5, 10, 15, 20, 25, 30, 40, 50, 60, 70, 80, 90, 95% (w/w) the expression vector.

Embodiment 84: The immunogenic composition of embodiment 82 or embodiment 83, further comprising pharmaceutically acceptable excipient, adjuvant, or a combination thereof.

Embodiment 85: The immunogenic composition of embodiment 84, wherein the pharmaceutically acceptable excipient comprises a solvent, dispersion media, diluent, dispersion, suspension aid, surface active agent, isotonic agent, thickening or emulsifying agent, preservative, polymer, peptide, protein, cell, hyaluronidase, or mixtures thereof.

Embodiment 86: The immunogenic composition of embodiment 84 or embodiment 85, wherein the adjuvant comprises C34, Gluco-C34, 7DW8-5, C17, C23, C30, α-galactosylceramide (α-GalCer), Aluminum salt (e.g., aluminum hydroxide, aluminum phosphate, alum (potassium aluminum sulfate), mixed aluminum salts), Squalene, MF59, QS-21, Freund's complete adjuvant, Freund's incomplete adjuvant, AS03 (GlaxoSmithKline), MF59 (Seqirus), CpG 1018 (Dynavax), or a mixture thereof.

Embodiment 87: An immunogenic composition, comprising
a first isolated nucleic acid, encoding a first recombinant influenza hemagglutinin (HA), which comprises an amino acid sequence at least 80%, 85%, 90%, 91%, 92%, 93%, 94%, 95%, 96%, 97%, 98%, or 99%, or 100% identical to SEQ ID NO: 6, provided that each of the 40^{th}, the 293^{rd}, the 304^{th}, and the 498^{th} amino acids thereof does not form a glycosylation site, and the 27^{th} amino acid thereof forms a glycosylation site;
a second isolated nucleic acid, encoding a second recombinant influenza hemagglutinin (HA), which comprises an amino acid sequence at least 80%, 85%, 90%, 91%, 92%, 93%, 94%, 95%, 96%, 97%, 98%, or 99%, or 100% identical to SEQ ID NO: 11, provided that each of the 24^{th}, the 38^{th}, the 54^{th}, the 61^{st}, the 79^{th}, the 301^{st}, and the 499^{th} amino acids thereof does not form a glycosylation site;
a third isolated nucleic acid, encoding a third recombinant influenza hemagglutinin (HA), which comprises an amino acid sequence at least 80%, 85%, 90%, 91%, 92%, 93%, 94%, 95%, 96%, 97%, 98%, or 99%, or 100% identical to SEQ ID NO: 3, provided that each of the 318^{th}, the 347^{th}, the 506^{th}, the 532^{nd}, and the 545^{th} amino acids does not form a glycosylation site, and the 40^{th} amino acid forms a glycosylation site; and
a fourth isolated nucleic acid, encoding a fourth recombinant influenza hemagglutinin (HA), which comprises an amino acid sequence at least 80%, 85%, 90%, 91%, 92%, 93%, 94%, 95%, 96%, 97%, 98%, or 99%, or 100% identical to SEQ ID NO: 18, provided that each of the 504^{th}, the 530^{th}, and the 543^{rd} amino acid does not form a glycosylation site, and each of the 40^{th}, the 316^{th}, and the 345^{th}, amino acids forms a glycosylation site.

Embodiment 88: The immunogenic composition of embodiment 87, wherein the first isolated nucleic acid is carried by a first expression vector, the second isolated nucleic acid is carried by a second expression vector, the third expression vector is carried by a third expression vector, and a fourth expression vector is carried by a fourth expression vector, and further wherein the first expression vector, the second expression vector, the third expression vector, and the fourth expression vector are independently a lipid nanoparticle, a liposome, a polymersome, a viral particle, a plasmid, or a bead.

Embodiment 89: The immunogenic composition of embodiment 87 or embodiment 88, further comprising pharmaceutically acceptable excipient, adjuvant, or a combination thereof.

Embodiment 90: The immunogenic composition of embodiment 89, wherein the pharmaceutically acceptable excipient comprises a solvent, dispersion media, diluent, dispersion, suspension aid, surface active agent, isotonic agent, thickening or emulsifying agent, preservative, polymer, peptide, protein, cell, hyaluronidase, or mixtures thereof.

Embodiment 91: The immunogenic composition of embodiment 89 or embodiment 90, wherein the adjuvant comprises C34, Gluco-C34, 7DW8-5, C17, C23, C30, α-galactosylceramide (α-GalCer), Aluminum salt (e.g., aluminum hydroxide, aluminum phosphate, alum (potassium aluminum sulfate), mixed aluminum salts), Squalene, MF59, QS-21, Freund's complete adjuvant, Freund's incomplete adjuvant, AS03 (GlaxoSmithKline), MF59 (Seqirus), CpG 1018 (Dynavax), or a mixture thereof.

Embodiment 92: A method for generating an immune response against influenza virus infection, comprising administering the isolated nucleic acid of any one of embodiments 1 to 42, the expression vector of any one of embodiments 43 to 81, or the immunogenic composition of any one of embodiments 82 to 91 to a subject in need at an effective amount.

Embodiment 93: The method of embodiment 92, wherein the administering is performed via oral, nasal, mucosal, submucosal, intravenous, intramuscular, intraperitoneal, subcutaneous, intradermal, transdermal, or buccal route.

Embodiment 94: The method of embodiment 92 or embodiment 93, wherein administering is performed 1, 2, 3, 4, 5, 6, 7, 8, 9, or 10 times.

Embodiment 95: The method of embodiment 94, wherein an interval of each administration to the next administration is about 1, 2, 3, 4, 5, 6, 7 days, about 1, 2, 3, 4, 5, 6, 7, 8, 9, or 10 weeks, or about 1, 2, 3, 4, 5, 6, 7, 8, 9, 10, 11, or 12 months.

Embodiment 96: The method of any one of embodiments 92 to 95, wherein the influenza virus is an influenza A virus or an influenza B virus.

Embodiment 97: The method of embodiment 96, wherein the influenza A virus is a H1, H2, H3, H4, H5, H6, H7, H8, H9, H10, H11, H12, H13, H14, H15, H16, H17, or H18 lineage.

Embodiment 98: The method of embodiment 96, wherein the influenza B virus is a Yamagata or Victoria lineage.

Embodiment 99: A recombinant influenza hemagglutinin, comprising an amino acid sequence at least 80%, 85%, 90%, 91%, 92%, 93%, 94%, 95%, 96%, 97%, 98%, or 99%, or 100% identical to SEQ ID NO: 3, SEQ ID NO: 6, SEQ ID NO: 9, SEQ ID NO: 11, SEQ ID NO: 16, or SEQ ID NO: 18.

### SEQUENCE LISTING

| | |
|---|---|
| SEQ ID NO: 1 | MKAIIVLLMV VTSNADRICT GITSSNSPHV VKTATQGEVN VTGVIPLTTT |
| IBV Yamagata | PTKSYFANLK GTRTRGKLCP DCLNCTDLDV ALGRPMCVGT TPSAKASILH |
| B/Phuket/3073/2013 | EVRPVTSGCF PIMHDRTKIR QLPNLLRGYE KIRLSTQNVI DAEKAPGGPY |
| WT | RLGTSGSCPN ATSKIGFFAT MAWAVPKDNY KNATNPLTVE VPYICTEGED |
| Synthetic | QITVWGFHSD DKTQMKSLYG DSNPQKFTSS ANGVTTHYVS QIGDFPDQTE |
| Amino acids in the stem | DGGLPQSGRI VVDYMMQKPG KTGTIVYQRG VLLPQKVWCA SGRSKVIKGS |
| region are in bold; Amino | LPLIGEADCL HEEYGGLNKS KPYYTGKHAK AIGNCPIWVK TPLKLANGTK |
| acids in the HA2 domain are bordered. | 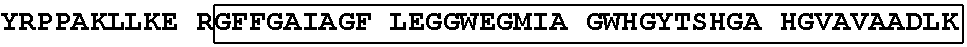 |
| | 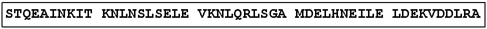 |
| | 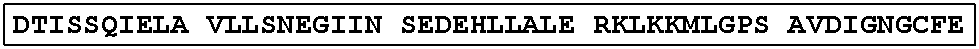 |
| | 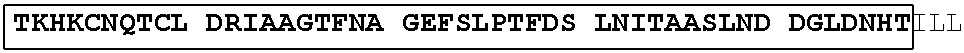 |
| | YYSTAASSLA VTLMLAIFIV YMVSRDNVSC SICL |
| SEQ ID NO: 2 | MKAIIVLLMV VTSNADRICT GITSSNSPHV VKTATQGEVX VTGVIPLTTT |
| IBV Yamagata | PTKSYFANLK GTRTRGKLCP DCLNCTDLDV ALGRPMCVGT TPSAKASILH |
| B/Phuket/3073/2013 (Stem | EVRPVTSGCF PIMHDRTKIR QLPNLLRGYE KIRLSTQNVI DAEKAPGGPY |
| region - X) | RLGTSGSCPN ATSKIGFFAT MAWAVPKDNY KNATNPLIVE VPYICTEGED |
| Synthetic | QITVWGFHSD DKTQMKSLYG DSNPQKFTSS ANGVTTHYVS QIGDFPDQTE |
| Amino acids in the stem | DGGLPQSGRI VVDYMMQKPG KTGTIVYQRG VLLPQKVWCA SGRSKVIKGS |
| region are in bold; Amino | LPLIGEADCL HEEYGGLXKS KPYYTGKHAK AIGNCPIWVK TPLKLAXGTK |
| acids in the HA2 domain are bordered. | 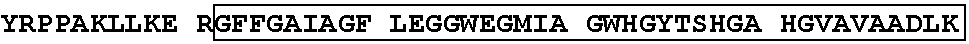 |
| | 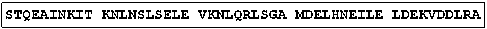 |
| | 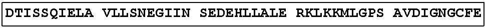 |
| | 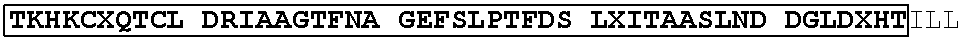 |
| | YYSTAASSLA VTLMLAIFIV YMVSRDNVSC SICL |
| SEQ ID NO: 3 | MKAIIVLLMV VTSNADRICT GITSSNSPHV VKTATQGEVN VTGVIPLTTT |
| IBV Yamagata | PTKSYFANLK GTRTRGKLCP DCLNCTDLDV ALGRPMCVGT TPSAKASILH |
| B/Phuket/3073/2013 | EVRPVTSGCF PIMHDRTKIR QLPNLLRGYE KIRLSTQNVI DAEKAPGGPY |
| Synthetic | RLGTSGSCPN ATSKIGFFAT MAWAVPKDNY KNATNPLTVE VPYICTEGED |
| Stem region | QITVWGFHSD DKTQMKSLYG DSNPQKFTSS ANGVTTHYVS QIGDFPDQTE |
| deglycosylation except for | DGGLPQSGRI VVDYMMQKPG KTGTIVYQRG VLLPQKVWCA SGRSKVIKGS |
| N40 | LPLIGEADCL HEEYGGLQKS KPYYTGKHAK AIGNCPIWVK TPLKLAQGTK |
| Amino acids in the stem region are in bold; Amino | 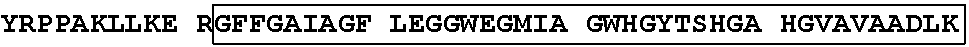 |
| acids in the HA2 domain are bordered. | 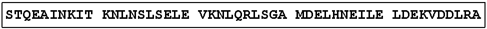 |
| | 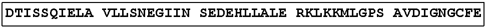 |
| | 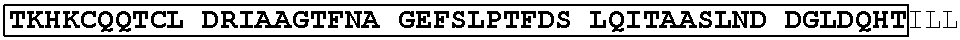 |
| | YYSTAASSLA VTLMLAIFIV YMVSRDNVSC SICL |
| SEQ ID NO: 4 | MKAILVVMLY TFTTANADTL CIGYHANNST DTVDTVLEKN VTVTHSVNLL |
| IAV Victoria | EDKHNGKLCK LRGVAPLHLG QCNIAGWILG NPECESLSTA RSWSYIVETS |
| A/Victoria/4897/2022 | NSDNGTCYPG DFINYEELRE QLSSVSSFER FEIFPKTSSW PNHDSDNGVT |
| (H1N1) | AACSHAGARS FYKNLIWLVK KGKSYPKINQ TYINDKGKEV LVLWGIHHPP |
| WT | TITDQESLYQ NADAYVFVGT SRYSKKFKPE IAARPKVRDR AGRMNYYWTL |
| Synthetic | VEPGDKITFE ATGNLVAPRY AFTMEKEAGS GIIISDTPVH DCNATCQTPE |
| Amino acids in the stem region are in bold; Amino | 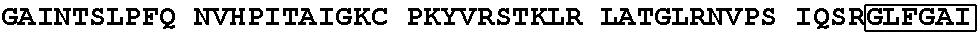 |
| acids in the HA2 domain are bordered. | 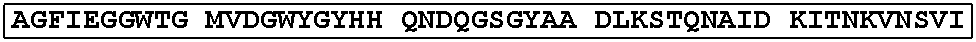 |
| | 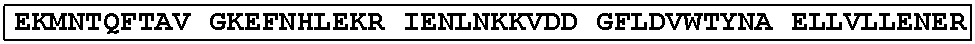 |
| | 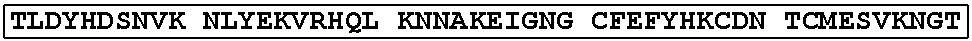 |
| | 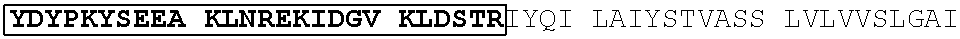 |
| | SFWMCSNGSL QCRICI |
| SEQ ID NO: 5 | MKAILVVMLY TFTTANADTL CIGYHAXXST DTVDTVLEKX VTVTHSVNLL |
| IAV Victoria | EDKHNGKLCK LRGVAPLHLG QCNIAGWILG NPECESLSTA RSWSYIVETS |
| A/Victoria/4897/2022 | NSDNGTCYPG DFINYEELRE QLSSVSSFER FEIFPKTSSW PNHDSDNGVT |
| (H1N1) (Stem region - X) | AACSHAGARS FYKNLIWLVK KGKSYPKINQ TYINDKGKEV LVLWGIHHPP |
| Synthetic | TITDQESLYQ NADAYVFVGT SRYSKKFKPE IAARPKVRDR AGRMNYYWTL |
| Amino acids in the stem | VEPGDKITFE ATGNLVAPRY AFTMEKEAGS GIIISDTPVH DCXATCQTPE |
| region are in bold; Amino acids in the HA2 domain | 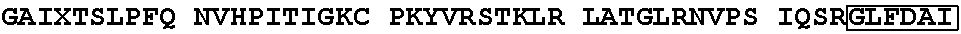 |
| are bordered. | 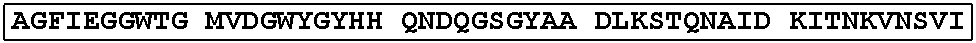 |
| | 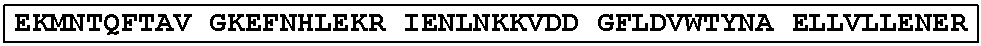 |
| | 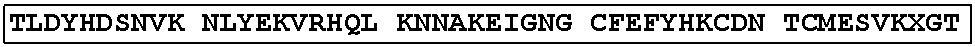 |
| | 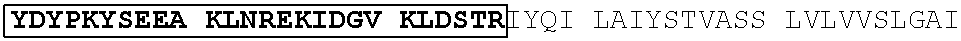 |
| | SFWMCSNGSL QCRICI |
| SEQ ID NO: 6 | MKAILVVMLY TFTTANADTL CIGYHANNST DTVDTVLEKQ VTVTHSVNLL |
| IAV Victoria | EDKHNGKLCK LRGVAPLHLG QCNIAGWILG NPECESLSTA RSWSYIVETS |
| A/Victoria/4897/2022 | NSDNGTCYPG DFINYEELRE QLSSVSSFER FEIFPKTSSW PNHDSDNGVT |
| (H1N1) | AACSHAGARS FYKNLIWLVK KGKSYPKINQ TYINDKGKEV LVLWGIHHPP |
| Synthetic | TITDQESLYQ NADAYVFVGT SRYSKKFKPE IAARPKVRDR AGRMNYYWTL |
| Stem region | VEPGDKITFE ATGNLVAPRY AFTMEKEAGS GIIISDTPVH DCQATCQTPE |
| deglycosylation except for N27/28 | 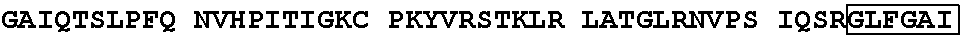 |
| Amino acids in the stem region are in bold; Amino | 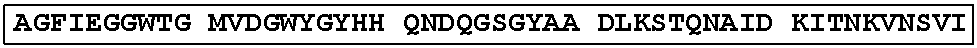 |
| acids in the HA2 domain are bordered. | 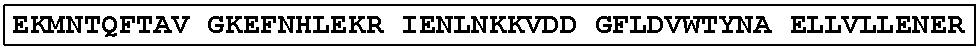 |
| | 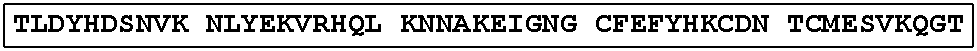 |
| | 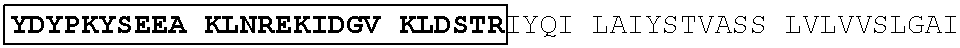 |
| | SFWMCSNGSL QCRICI |
| SEQ ID NO: 7 | MKAIIALSNI LCLVFAQKIP GNDNSTATLC LGHHAVPNGT IVKTIINDRI |
| IAV A/Thailand/8/2022 | EVTNATELVQ NSSIGKICNS PHQILDGGNC TLIDALLGDP QCDGFQNKEW |
| (H3N2) | DLFVERSRAN SSCYPYDVPD YASLRSLVAS SGTLEFKNES FNWTGVKQNG |
| WT | TSSACKRGSS SSFFSRLNWL TSLNNIYPAQ NVTMPNKEQF DKLYIWGVHH |
| Synthetic | PDTDKNQFSL FAQSSGRITV STKRSQQAVI PNIGSRPRVR DIPSRISIYW |
| Amino acids in the stem | TIVKPGDILL INSTGNLIAP RGYFKIRSGK SSIMRSDAPI GKCKSECITP |
| region are in bold; Amino acids in the HA2 domain | 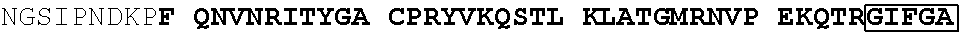 |
| are bordered. | 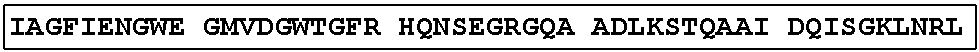 |
| | 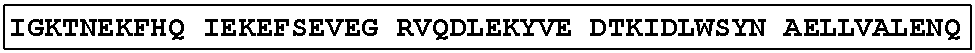 |
| | 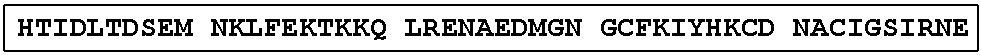 |
| | 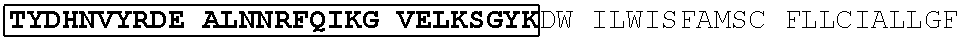 |
| | IMWACQKGNI RCNICI |
| SEQ ID NO: 8 | MKAIIALSNI LCLVFAQKIP GNDXSTATLC LGHHAVPXGT IVKTITNDRI |
| IAV A/Thailand/8/2022 | EVTNATELVQ NSSIGKICNS PHQILDGGNC TLIDALLGDP QCDGFQNKEW |
| (H3N2) (Stem-X) | DLFVERSRAN SSCYPYDVPD YASLRSLVAS SGTLEFKNES FNWTGVKQNG |
| Synthetic | TSSACKRGSS SSFFSRLNWL TSLNNIYPAQ NVTMPNKEQF DKLYIWGVHH |
| Amino acids in the stem | PDTDKNQFSL FAQSSGRITV STKRSQQAVI PNIGSRPRVR DIPSRISIYW |
| region are in bold; Amino | TIVKPGDILL INSTGNLIAP RGYFKIRSGK SSIMRSDAPI GKCKSECITP |
| acids in the HA2 domain are bordered. | 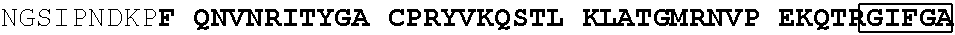 |
| | 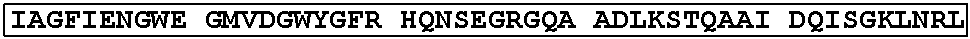 |
| | 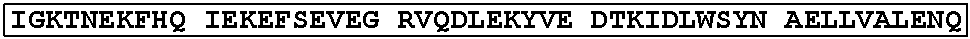 |
| | 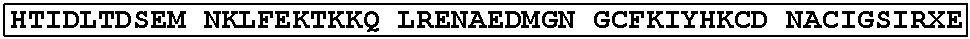 |
| | 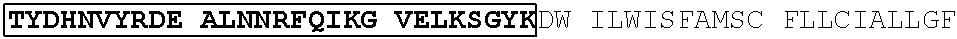 |
| | IMWACQKGNI RCNICI |
| SEQ ID NO: 9 | MKAIIALSNI LCLVFAQKIP GNDNSTATLC LGHHAVPQGT IVKTITNDRI |
| IAV A/Thailand/8/2022 | EVTNATELVQ NSSIGKICNS PHQILDGGNC TLIDALLGDP QCDGFQNKEW |
| (H3N2) | DLFVERSRAN SSCYPYDVPD YASLRSLVAS SGTLEFKNES FNWTGVKQNG |
| Synthetic | TSSACKRGSS SSFFSRLNWL TSLNNIYPAQ NVTMPNKEQF DKLYIWGVHH |
| Stem region | PDTDKNQFSL FAQSSGRITV STKRSQQAVI PNIGSRPRVR DIPSRISIYW |
| deglycosylation except for | TIVKPGDILL INSTGNLIAP RGYFKIRSGK SSIMRSDAPI GKCKSECITP |
| N24 Amino acids in the stem | 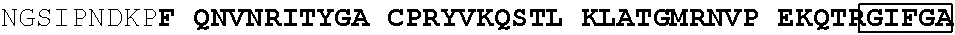 |
| region are in bold; Amino acids in the HA2 domain | 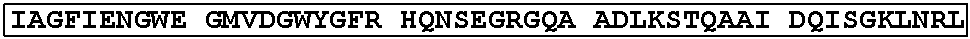 |
| are bordered. | 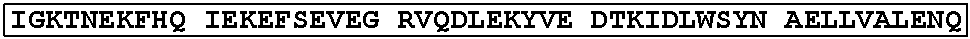 |
| | 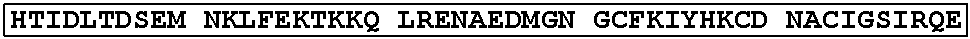 |
| | 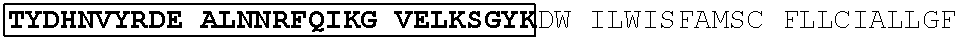 |
| | IMWACQKGNI RCNICI |
| SEQ ID NO: 10 | MKAIIALSNI LCLVFAQKIP GNDQSTATLC LGHHAVPQGT IVKTITNDRI |
| IAV A/Thailand/8/2022 | EVTQATELVQ QSSIGKICNS PHQILDGGQC TLIDALLGDP QCDGFQNKEW |
| (H3N2) | DLFVERSRAN SSCYPYDVPD YASLRSLVAS SGTLEFKNES FNWTGVKQNG |
| Synthetic | TSSACKRGSS SSFFSRLNWL TSLNNIYPAQ NVTMPNKEQF DKLYIWGVHH |
| Stem region | PDTDKNQFSL FAQSSGRITV STKRSQQAVI PNIGSRPRVR DIPSRISIYW |
| deglycosylation + | TIVKPGDILL INSTGNLIAP RGYFKIRSGK SSIMRSDAPI GKCKSECITP |
| additional N54, N61, N79 deglycosylation in HA1. | 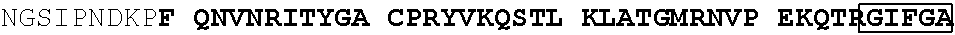 |
| Amino acids in the stem region are in bold; Amino | 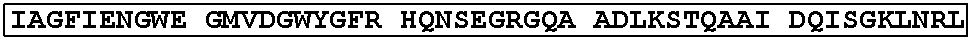 |
| acids in the HA2 domain are bordered. | 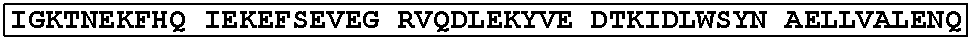 |
| | 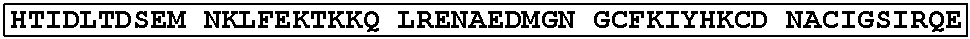 |
| | 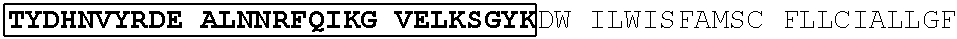 |
| | IMWACQKGNI RCNICI |
| SEQ ID NO: 11 | MKAIIALSNI LCLVFAQKIP GNDQSTATLC LGHHAVPQGT IVKTITNDRI |
| IAV A/Thailand/8/2022 | EVTQATELVQ QSSIGKICNS PHQILDGGQC TLIDALLGDP QCDGFQNKEW |
| (H3N2) | DLFVERSRAN SSCYPYDVPD YASLRSLVAS SGTLEFKNES FNWTGVKQNG |
| Synthetic | TSSACKRGSS SSFFSRLNWL TSLNNIYPAQ NVTMPNKEQF DKLYIWGVHH |
| Stem region | PDTDKNQFSL FAQSSGRITV STKRSQQAVI PNIGSRPRVR DIPSRISIYW |
| deglycosylation + | TIVKPGDILL INSTGNLIAP RGYFKIRSGK SSIMRSDAPI GKCKSECITP |
| additional N54, N61, N79, N301 deglycosylation in | 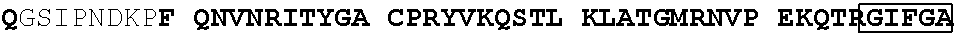 |
| HA1. Amino acids in the stem | 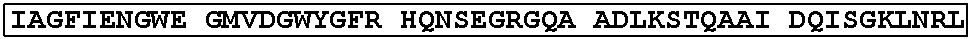 |
| region are in bold; Amino acids in the HA2 domain | 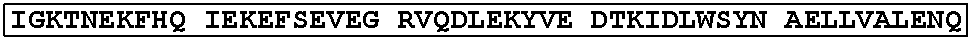 |
| are bordered. | 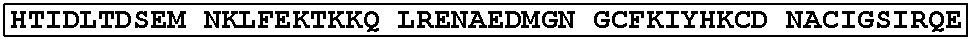 |
| | 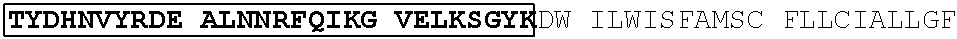 |
| | IMWACQKGNI RCNICI |
| SEQ ID NO: 12 | MKAIIALSNI LCLVFAQKIP GNDQSTATLC LGHHAVPQGT IVKTITNDRI |
| IAV A/Thailand/8/2022 | EVTNATELVQ QSSIGKICNS PHQILDGGNC TLIDALLGDP QCDGFQNKEW |
| (H3N2) | DLFVERSRAN SSCYPYDVPD YASLRSLVAS SGTLEFKNES FNWTGVKQNG |
| Synthetic | TSSACKRGSS SSFFSRLNWL TSLNNIYPAQ NVTMPNKEQF DKLYIWGVHH |
| Stem region | PDTDKNQFSL FAQSSGRITV STKRSQQAVI PNIGSRPRVR DIPSRISIYW |
| deglycosylation + | TIVKPGDILL INSTGNLIAP RGYFKIRSGK SSIMRSDAPI GKCKSECITP |
| additional N61, N301 deglycosylation in HA1. | 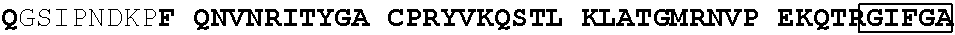 |
| Amino acids in the stem region are in bold; Amino | 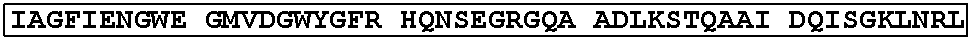 |
| acids in the HA2 domain are bordered. | 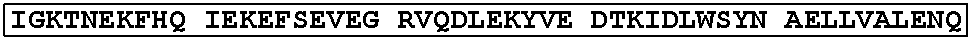 |
| | 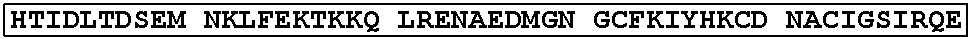 |
| | 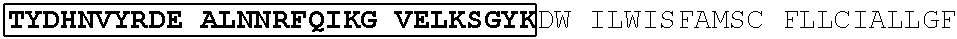 |
| | IMWACQKGNI RCNICI |
| SEQ ID NO: 13 | MKAIIALSNI LCLVFAQKIP GNDQSTATLC LGHHAVPQGT IVKTITNDRI |
| IAV A/Thailand/8/2022 | EVTNATELVQ NSSIGKICNS PHQILDGGNC TLIDALLGDP QCDGFQNKEW |
| (H3N2) | DLFVERSRAN SSCYPYDVPD YASLRSLVAS SGTLEFKNES FNWTGVKQNG |
| Synthetic | TSSACKRGSS SSFFSRLNWL TSLNNIYPAQ NVTMPNKEQF DKLYIWGVHH |
| Stem region | PDTDKNQFSL FAQSSGRITV STKRSQQAVI PNIGSRPRVR DIPSRISIYW |
| deglycosylation + | TIVKPGDILL IQSTGNLIAP RGYFKIRSGK SSIMRSDAPI GKCKSECITP |
| additional N262, N301 deglycosylation in HA1. | 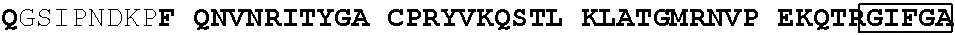 |
| Amino acids in the stem region are in bold; Amino | 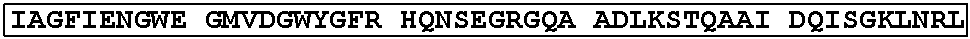 |
| acids in the HA2 domain are bordered. | 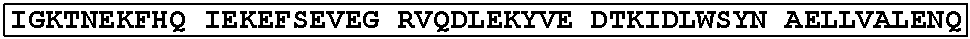 |
| | 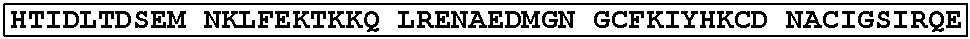 |
| | 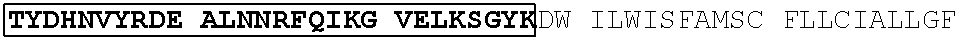 |
| | IMWACQKGNI RCNICI |
| SEQ ID NO: 14 | MKAIIVLLMV VTSNADRICT GITSSNSPHV VKTATQGEVN VTGVIPLTTT |
| IBV Victoria | PTKSHFANLK GTETRGKLCP KCLNCTDLDV ALGRPKCTGK IPSARVSILH |
| B/Austria/1359417/2021 | EVRPVTSGCF PIMHDRTKIR QLPNLLRGYE HVRLSTHNVI NTEDAPGGPY |
| WT | EIGTSGSCLN ITNGKGFFAT MAWAVPKNKT ATNPLTIEVP YICTEEEDQI |
| Synthetic | TVWGFHSDDE TQMARLYGDS KPQKFTSSAN GVTTHYVSQI GGFPNQTEDG |
| Amino acids in the stem | GLPQSGRIVV DYMVQKSGKT GTITYQRGIL LPQKVWCASG KSKVIKGSLP |
| region are in bold; Amino | LIGEADCLHE KYGGLNKSKP YYTGEHAKAI GNCPIWVKTP LKLANGTKYR |
| acids in the HA2 domain are bordered. | 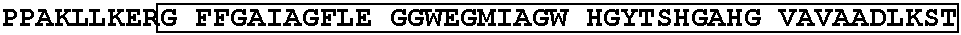 |
| | 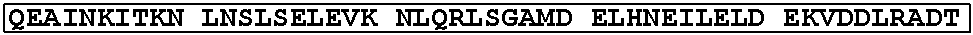 |
| | 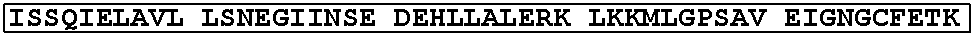 |
| | 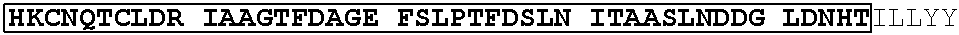 |
| | STAASSLAVT LMIAIFVVYM VSRDNVSCSI CL |
| SEQ ID NO: 15 | MKAIIVLLMV VTSNADRICT GITSSNSPHV VKTATQGEVX VTGVIPLTTT |
| IBV Victoria | PTKSHFANLK GTETRGKLCP KCLNCTDLDV ALGRPKCTGK IPSARVSILH |
| B/Austria/1359417/2021 | EVRPVTSGCF PIMHDRTKIR QLPNLLRGYE HVRLSTHNVI NTEDAPGGPY |
| (Stem-X) | EIGTSGSCLN ITNGKGFFAT MAWAVPKNKT ATNPLTIEVP YICTEEEDQI |
| Synthetic | TVWGFHSDDE TQMARLYGDS KPQKFTSSAN GVTTHYVSQI GGFPNQTEDG |
| Amino acids in the stem | GLPQSGRIVV DYMVQKSGKT GTITYQRGIL LPQKVWCASG KSKVIKGSLP |
| region are in bold; Amino | LIGEADCLHE KYGGLXKSKP YYTGEHAKAI GNCPIWVKTP LKLAXGTKYR |
| acids in the HA2 domain are bordered. | 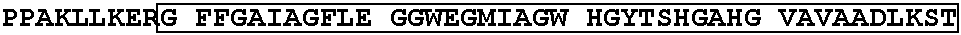 |
| | 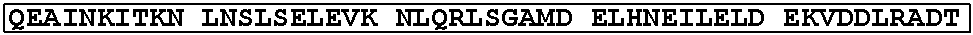 |
| | 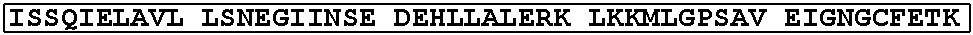 |
| | 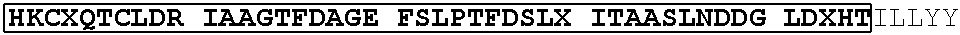 |
| | STAASSLAVT LMIAIFVVYM VSRDNVSCSI CL |
| SEQ ID NO: 16 | MKAIIVLLMV VTSNADRICT GITSSNSPHV VKTATQGEVN VTGVIPLTTT |
| IBV Victoria | PTKSHFANLK GTETRGKLCP KCLNCTDLDV ALGRPKCTGK IPSARVSILH |
| B/Austria/1359417/2021 | EVRPVTSGCF PIMHDRTKIR QLPNLLRGYE HVRLSTHNVI NTEDAPGGPY |
| Synthetic | EIGTSGSCLN ITNGKGFFAT MAWAVPKNKT ATNPLTIEVP YICTEEEDQI |
| Stem region | TVWGFHSDDE TQMARLYGDS KPQKFTSSAN GVTTHYVSQI GGFPNQTEDG |
| deglycosylation except for | GLPQSGRIVV DYMVQKSGKT GTITYQRGIL LPQKVWCASG KSKVIKGSLP |
| N40 | LIGEADCLHE KYGGLQKSKP YYTGEHAKAI GNCPIWVKTP LKLAQGTKYR |
| Amino acids in the stem region are in bold; Amino | 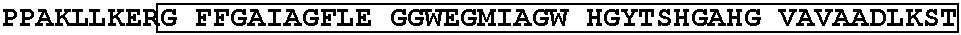 |
| acids in the HA2 domain are bordered. | 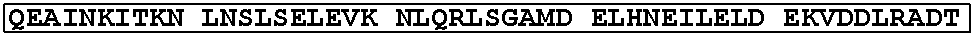 |
| | 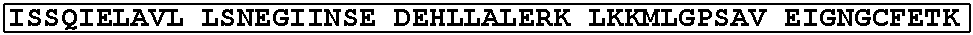 |
| | 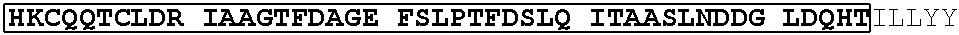 |
| | STAASSLAVT LMIAIFVVYM VSRDNVSCSI CL |
| SEQ ID NO: 17 | MKAIIVLLMV VTSNADRICT GITSSNSPHV VKTATQGEVN VTGVIPLTTT |
| IBV Victoria | PTKSHFANLK GTETRGKLCP KCLNCTDLDV ALGRPKCTGK IPSARVSILH |
| B/Austria/1359417/2021 | EVRPVTSGCF PIMHDRTKIR QLPNLLRGYE HVRLSTHNVI NTEDAPGGPY |
| Synthetic | EIGTSGSCLN ITNGKGFFAT MAWAVPKNKT ATNPLTIEVP YICTEEEDQI |
| Stem region | TVWGFHSDDE TQMARLYGDS KPQKFTSSAN GVTTHYVSQI GGFPNQTEDG |
| deglycosylation except for | GLPQSGRIVV DYMVQKSGKT GTITYQRGIL LPQKVWCASG KSKVIKGSLP |
| N40, N316 | LIGEADCLHE KYGGLNKSKP YYTGEHAKAI GNCPIWVKTP LKLAQGTKYR |
| Amino acids in the stem region are in bold; Amino | 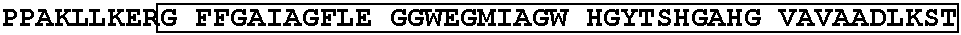 |
| acids in the HA2 domain are bordered. | 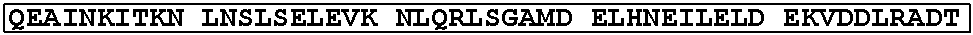 |
| | 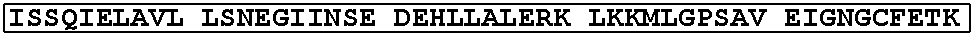 |
| | 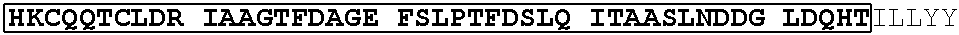 |
| | STAASSLAVT LMIAIFVVYM VSRDNVSCSI CL |
| SEQ ID NO: 18 | MKAIIVLLMV VTSNADRICT GITSSNSPHV VKTATQGEVN VTGVIPLTTT |
| IBV Victoria | PTKSHFANLK GTETRGKLCP KCLNCTDLDV ALGRPKCTGK IPSARVSILH |
| B/Austria/1359417/2021 | EVRPVTSGCF PIMHDRTKIR QLPNLLRGYE HVRLSTHNVI NTEDAPGGPY |
| Synthetic | EIGTSGSCLN ITNGKGFFAT MAWAVPKNKT ATNPLTIEVP YICTEEEDQI |
| Stem region | TVWGFHSDDE TQMARLYGDS KPQKFTSSAN GVTTHYVSQI GGFPNQTEDG |
| deglycosylation except for | GLPQSGRIVV DYMVQKSGKT GTITYQRGIL LPQKVWCASG KSKVIKGSLP |
| N40, N316, N345 (deg- | LIGEADCLHE KYGGLNKSKP YYTGEHAKAI GNCPIWVKTP LKLANGTKYR |
| HA2) Amino acids in the stem | 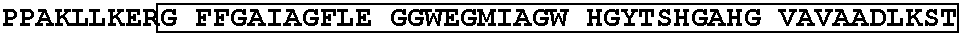 |
| region are in bold; Amino acids in the HA2 domain | 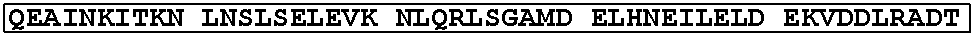 |
| are bordered. | 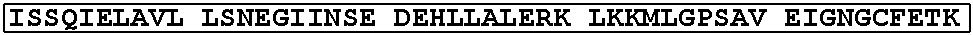 |
| | 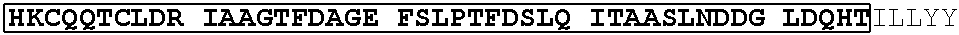 |
| | STAASSLAVT LMIAIFVVYM VSRDNVSCSI CL |
| SEQ ID NO: 19 | MKANLLVLLC ALAAADADTI CIGYHANNST DTVDTVLEKN VTVTHSVNLL |
| A/Puerto Rico/8/1934 | EDSHNGKLCR LKGIAPLQLG KCNIAGWLLG NPECDPLLPV RSWSYIVETP |
| H1N1 | NSENGICYPG DFIDYEELRE QLSSVSSFER FEIFPKESSW PNHNINGVTA |
| Synthetic | ACSHEGKSSF YRNLLWLTEK EGSYPKLKNS YVNKKGKEVL VLWGIHHPPN |
| | SKEQQNLYQN ENAYVSVVTS NYNRRFTPEI AERPKVRDQA GRMNYYWILL |
| | KPGDTIIFEA NGNLIAPMYA FALSRGFGSG IITSNASMHE CNTKCQTPLG |
| | AINSSLPYQN IHPVTIGECP KYVRSAKLRM VTGLRNIPSI QSRGLFGAIA |
| | GFIEGGWTGM IDGWYGYHHQ NEQGSGYAAD QKSTQNAING ITNKVNTVIE |
| | KMNIQFTAVG KEFNKLEKRM ENLNKKVDDG FLDIWTYNAE LLVLLENERT |
| | LDFHDSNVKN LYEKVKSQLK NNAKEIGNGC FEFYHKCDNE CMESVRNGTY |
| | DYPKYSEESK LNREKVDGVK LESMGIYQIL AIYSTVASSL VLLVSLGAIS |
| | FWMCSNGSLQ CRICI |
| SEQ ID NO: 20 | atgaaggccatcatcgtgctgctgatggtggtgacctccaacgccgatagaatct |
| Nucleotide sequences | gcaccggcatcacatcctccaattcccctcacgtggtgaagaccgccacacaggg |
| encoding SEQ ID NO: 3 | cgaggtgaacgtgacaggcgtgatccctctgaccacaacacctaccaagtcctac |
| Synthetic | ttcgccaacctgaagggcaccaggacaaggggcaagctgtgccctgattgtctg |
| | aactgtaccgacctggatgtggccctgggcaggcctatgtgtgtgggcaccacac |
| | ctagcgccaaggccagcatcctgcacgaggtgaggcctgtgacaagcggctgct |
| | ttcccatcatgcacgatagaacaaagatcagacagctgcccaatctgctgagggg |
| | ctacgagaagatcaggctgtccacccagaacgtgatcgatgccgagaaggcccc |
| | cggcggcccttacagactgggcacaagcggcagctgccccaacgccaccagcaag |
| | atcggctttttcgccaccatggcctgggccgtgcctaaggataactacaagaat |
| | gccaccaaccccctgacagtggaggtgccctacatctgtaccgagggcgaggatc |
| | agatcacagtgtggggctttcactccgatgataagacacagatgaagtccctgta |
| | cggcgactccaatcctcagaagttcacatcctccgccaatggcgtgacaacccac |
| | tacgtgagccagatcggcgactttcctgaccagaccgaggacggcggcctgccac |
| | agagcggaaggatcgtggtggactacatgatgcagaagcctggcaagacaggcac |
| | aatcgtgtaccagaggggcgtgctgctgccccagaaggtgtggtgcgcctccg |
| | gcaggtccaaggtcattaagggcagcctgcccctgatcggcgaggccgattgtct |
| | gcacgaggagtacggcggcctgaataagtccaagccctactacaccggcaagca |
| | cgccaaggccatcggcaattgtcctatctgggtgaagacccctctgaagctggcc |
| | aacggcaccaagtacaggcctcctgccaagctgctgaaggagaggggcttctttg |
| | gcgccatcgccggctttctggagggcggatgggagggcatgatcgccggctggca |
| | cggctacaccagccacggagcccacggcgtggccgttgctgctgatctgaagag |
| | cacacaggaggccatcaacaagatcacaaagaacctgaactccctgtccgagctg |
| | gaggtgaagaacctgcagaggctgagcggcgccatggacgagctgcacaatga |
| | gatcctggagctggatgagaaggtggacgacctgagagccgatacaatctcctcc |
| | cagatcgagctggccgtgctgctgtccaatgagggcatcatcaacagcgaggacg |
| | agcacctgctggccctggagaggaagctgaagaagatgctgggccccagcgccgt |
| | ggacatcggcaatggctgcttcgagacaaagcacaagtgcaaccagacatgcct |
| | ggacagaatcgccgccggcaccttcaatgccggcgagttctccctgcccacattt |
| | gactccctgaacatcaccgccgcctccctgaacgacgatggcctggataaccacac |
| | catcctgctgtactacagcacagccgccagcagcctggccgtgaccctgatgctg |
| | gccatctttatcgtgtacatggtgtccagggataatgtgagctgctccatctgtc |
| | tgtgatagtaa |
| SEQ ID NO: 21 | atgaaggccatcatcgtgctgctgatggtggtgacatccaatgccgacagaatct |
| Nucleotide sequences | gcacaggcatcaccagctccaacagcccccacgtggtgaagacagccacacaggg |
| encoding SEQ ID NO: 6 | cgaggtgaacgtgaccggcgtgatccctctgacaaccacacctaccaagtcccac |
| Synthetic | ttcgccaatctgaagggcaccgagacaaggggcaagctgtgccctaagtgtctga |
| | actgtaccgatctggatgtggccctgggcaggcctaagtgtaccggcaagatccc |
| | tagcgccagggtgagcatcctgcacgaggtgagacctgtgacaagcggctgcttcc |
| | ccatcatgcacgacagaaccaagatcaggcagctgcccaatctgctgagaggcta |
| | cgagcacgtgagactgtccacccacaacgtgatcaataccgaggatgcccctggc |
| | ggcccttacgagatcggcaccagcggctcctgtctgaatatcacaaatggcaagg |
| | gcttctttgccaccatggcctgggccgtgcctaagaacaagaccgccaccaaccct |
| | ctgaccatcgaggtgccctacatctgtacagaggaggaggatcagatcaccgtgt |
| | ggggctttcactccgatgacgagacccagatggccagactgtacggcgacagcaa |
| | gccccagaagttcacctcctccgccaacggcgtgaccacacactacgtgtcccag |
| | atcggcggcttccctaaccagaccgaggacggcggcctgcctcagagcggaagga |
| | tcgtggtggattacatggtgcagaagtccggcaagacaggcaccatcacatacca |
| | gaggggcatcctgctgcctcagaaggtgtggtgcgcctccggcaagagcaaggtc |
| | attaagggcagcctgcctctgatcggcgaggccgactgcctgcacgagaagtacg |
| | gcggcctgaataagagcaagccctactacaccggcgagcacgccaaggccatcg |
| | gcaactgtcccatctgggtgaagacacctctgaagctggccaatggcaccaagta |
| | cagaccccccgccaagctgctgaaggagagaggctttttcggcgccatcgccggct |
| | tcctggagggcggatgggagggaatgatcgccggctggcacggctacaccagcca |
| | cggagcccacggcgtggccgttgctgctgacctgaagagcacccaggaggccat |
| | caacaagatcaccaagaatctgaatagcctgtccgagctggaggtgaagaatctg |
| | cagagactgtccggcgccatggatgagctgcacaatgagatcctggagctggatg |
| | agaaggtggacgacctgagagccgacacaatctccagccagatcgagctggccgt |
| | gctgctgagcaacgagggcatcatcaatagcgaggatgagcacctgctggccctg |
| | gagagaaagctgaagaagatgctgggcccttccgccgtggagatcggcaacggct |
| | gttttgagaccaagcacaagtgcaaccagacatgtctggataggatcgccgccgg |
| | cacctttgacgccggcgagttctccctgcccacattcgatagcctgaatatcacc |
| | gccgcctccctgaacgatgacggcctggataatcacaccatcctgctgtactaca |
| | gcaccgccgccagcagcctggccgtgacactgatgatcgccatcttcgtggtgta |
| | catggtgagcagggataatgtgtcctgttccatctgtctgtgatagtaa |
| SEQ ID NO: 22 | atgaagacaatcatcgccctgagcaacatcctgtgcctggtgttcgcccagaaga |
| Nucleotide sequences | tccctggcaatgataacagcaccgccaccctgtgtctgggccaccacgccgtgcc |
| encoding SEQ ID NO: 9 | tcagggcacaatcgtgaagacaatcacaaacgacagaatcgaggtgacaaacgcc |
| Synthetic | accgagctggtgcagaattcctccatcggcgagatctgtgacagcccccaccaga |
| | tcctggacggcggcaattgtaccctgatcgacgccctgctgggcgacccccagtg |
| | tgatggcttccagaacaaggagtgggatctgtttgtggagaggtccagagccaac |
| | tccaactgctaccettacgatgtgcctgactacgccagcctgaggagcctggtgg |
| | ccagctccggcaccctggagtttaagaacgagtccttcaactggaccggcgtgaa |
| | gcagaatggcacctccagcgcctgtatcagaggcagctccagcagcttctttagc |
| | aggctgaactggctgacaagcctgaataacatctaccctgcccagaacgtgacca |
| | tgcccaacaaggagcagtttgataagctgtacatctggggcgtgcaccaccccaa |
| | caccgataagaatcagatctccctgttcgcccagtcctccggcagaatcacagtg |
| | agcacaaagagaagccagcaggccgtgatccccaacatcggctccagacccagaa |
| | tcagaggcatccctagcagaatctccatctactggaccatcgtgaagcctggcga |
| | tatcctgctgatcaactccaccggcaacctgatcgcccctaggggctacttcaag |
| | atcagatccggcaagagcagcatcatgaggagcgacgcccctatcggcaagtgta |
| | agtccgagtgcatcacacccaatggctccatccctaatgataagccctttcagaa |
| | tgtgaacagaatcacctacggcgcctgccccaggtacgtgaagcagagcaccctg |
| | aagctggccacaggcatgagaaatgtgcccgagaagcagaccaggggcatcttcg |
| | gcgccatcgccggcttcatcgagaatggctgggagggcatggtggatggctggta |
| | cggcttcagacaccagaactccgagggcaggggccaggccgctgatctgaagagc |
| | acccaggccgccatcgaccagatcaacggcaagctgaacaggctgatcggcaaga |
| | caaatgagaagttccaccagatcgagaaggagttctccgaggtggagggcagagt |
| | gcaggacctggagaagtacgtggaggataccaagatcgacctgtggagctacaac |
| | gccgagctgctggtggccctggagaatcagcacaccatcgacctgaccgattccg |
| | agatgaataagctgttcgagaagaccaagaagcagctgagggagaatgccgagga |
| | tatgggcaatggctgctttaagatctaccacaagtgcgataacgcctgcatcggc |
| | agcatcagaaatgagacctacgaccaccaggtgtacagagatgaggccctgaata |
| | ataggtttcagatcaagggcgtggagctgaagagcggctacaaggactggatcct |
| | gtggatctcctttgccatgtcctgctttctgctgtgcatcgccctgctgggattc |
| | attatgtgggcctgccagaagggcaatatcagatgtaacatctgtatctgatagt |
| | aa |
| SEQ ID NO: 23 | atgaaggccatcatcgccctgagcaacatcctgtgtctggtgttcgcccagaaga |
| Nucleotide sequences | tccctggcaacgatcaaagcaccgccaccctgtgtctgggccaccacgccgtgcc |
| encoding SEQ ID NO: 11 | ccaaggcacaatcgtgaagaccatcaccaatgacaggatcgaggtgacacaagcc |
| Synthetic | acagagctggtgcagcaatccagcatcggcaagatctgcaatagcccccaccaga |
| | tcctggacggcggccaatgcaccctgatcgacgccctgctgggcgatccccagtg |
| | cgatggctttcagaataaggagtgggatctgttcgtggagaggtccagagccaat |
| | tcctcctgctacccctacgacgtgcctgactacgcctccctgagaagcctggtgg |
| | ccagcagcggcaccctggagtttaagaatgagagcttcaactggacaggcgtgaa |
| | gcagaacggcacatccagcgcctgtaagaggggcagctccagcagcttcttctcc |
| | agactgaattggctgacctccctgaacaacatctaccccgcccagaatgtgacaa |
| | tgcccaacaaggagcagttcgacaagctgtacatctggggcgtgcaccaccccga |
| | tacagacaagaaccagttctccctgtttgcccagtccagcggcagaatcacagtg |
| | tccaccaagagatcccagcaggccgtgatccccaacatcggcagcagacccagag |
| | tgagggatatccccagcaggatctccatctactggacaatcgtgaaacctggcga |
| | catcctgctgatcaacagcaccggcaatctgatcgcccccaggggctactttaag |
| | atcaggagcggcaagagctccatcatgaggagcgacgcccccatcggcaagtgca |
| | agtccgagtgtatcacccctaacggcagcatccccaacgataagccttttcagaa |
| | tgtgaataggatcacctacggcgcctgtcctagatacgtgaagcagagcaccctg |
| | aagctggccaccggcatgagaaatgtgcctgagaagcagacaagaggcatcttcg |
| | gcgccatcgccggctttatcgagaacggctgggagggcatggtggatggctggta |
| | cggcttcaggcaccagaattccgagggcagaggccaggccgccgatctgaagagc |
| | acccaggccgccatcgatcagatctccggcaagctgaacagactgatcggcaaga |
| | ccaacgagaagttccaccagatcgagaaggagttcagcgaggtggagggcagggt |
| | gcaggatctggagaagtacgtggaggacacaaagatcgacctgtggagctacaat |
| | gccgagctgctggtggccctggagaaccagcacacaatcgacctgacagatagcg |
| | agatgaataagctgtttgagaagacaaagaagcagctgagggagaacgccgagga |
| | tatgggcaatggctgttttaagatctaccacaagtgcgacaatgcctgtatcggc |
| | agcatcagacaggagacatacgaccacaacgtgtacagggatgaggccctgaaca |
| | atagattccagatcaagggcgtggagctgaagtccggctacaaggactggatcct |
| | gtggatcagctttgccatgtcctgtttcctgctgtgcatcgccctgctgggattt |
| | atcatgtgggcctgtcagaagggcaatatcagatgcaacatctgtatctgatagt |
| | aa |
| SEQ ID NO: 24 | atgaaggccatcatcgtgctgctgatggtggtgaccagcaatgccgataggatct |
| Nucleotide sequences | gcacaggcatcacctccagcaatagcccccacgtggtgaagacagccacccaggg |
| encoding SEQ ID NO: 16 | cgaggtgaacgtgaccggcgtgatccccctgacaaccacacctacaaagtcccac |
| Synthetic | ttcgccaacctgaagggcacagagacaagaggcaagctgtgtcccaagtgcctga |
| | actgcacagacctggatgtggccctgggcagacccaagtgtaccggcaagatccc |
| | cagcgccagggtgtccatcctgcacgaggtgagacctgtgacatccggctgtttc |
| | cccatcatgcacgataggaccaagatcaggcagctgcctaacctgctgagaggct |
| | acgagcacgtgaggctgagcacacacaacgtgatcaatacagaggacgcccccgg |
| | cggcccctacgaaatcggaacaagcggcagctgtctgaacatcaccaacggcaag |
| | ggctttttcgccaccatggcctgggccgtgcctaagaataagaccgccaccaatc |
| | ccctgaccatcgaggtgccctacatctgcaccgaggaggaggatcagatcaccgt |
| | gtggggctttcacagcgacgatgagacccagatggccaggctgtacggcgactcc |
| | aagcctcagaagtttacaagctccgccaacggcgtgaccacccactacgtgtccc |
| | agatcggcggctttcccaatcagacagaggacggcggcctgcctcagtccggcag |
| | aatcgtggtggattacatggtgcagaagtccggcaagacaggcaccatcacctac |
| | cagagaggcatcctgctgccccagaaggtgtggtgcgccagcggcaagtccaagg |
| | tcattaagggctccctgcccctgatcggcgaggccgattgcctgcacgagaagta |
| | cggcggcctgcagaagagcaagccttactacaccggcgagcacgccaaggccatc |
| | ggcaactgtcccatctgggtgaagacacctctgaagctggcccagggcacaaagt |
| | acagacctcccgccaagctgctgaaggagagaggctttttcggcgccatcgccgg |
| | ctttctggagggcggatgggagggcatgatcgccggctggcacggctacaccagc |
| | cacggagcccacggcgtggccgttgctgctgatctgaagagcacccaggaggcca |
| | tcaataagatcacaaagaacctgaactccctgagcgagctggaggtgaagaatct |
| | gcagaggctgagcggcgccatggatgagctgcacaacgagatcctggagctggac |
| | gagaaggtggacgacctgagggccgacacaatcagcagccagatcgagctggccg |
| | tgctgctgagcaacgagggcatcatcaatagcgaggatgagcacctgctggccct |
| | ggagaggaagctgaagaagatgctgggccccagcgccgtggagatcggcaacgga |
| | tgttttgagacaaagcacaagtgtcagcagacatgcctggacaggatcgccgccg |
| | gcacatttgatgccggcgagttttccctgcccacattcgatagcctgcagatcac |
| | cgccgccagcctgaacgatgacggcctggatcagcacacaatcctgctgtactac |
| | tccaccgccgcctccagcctggccgtgacactgatgatcgccatcttcgtggtgt |
| | acatggtgtccagggataatgtgagctgttccatctgcctgtgatagtaa |
| SEQ ID NO: 25 | atgaaggccatcatcgtgctgctgatggtggtgacatccaatgccgacagaatct |
| Nucleotide sequences | gcacaggcatcaccagctccaacagcccccacgtggtgaagacagccacacaggg |
| encoding SEQ ID NO: 18 | cgaggtgaacgtgaccggcgtgatccctctgacaaccacacctaccaagtcccac |
| Synthetic | ttcgccaatctgaagggcaccgagacaaggggcaagctgtgccctaagtgtctga |
| | actgtaccgatctggatgtggccctgggcaggcctaagtgtaccggcaagatccc |
| | tagcgccagggtgagcatcctgcacgaggtgagacctgtgacaagcggctgcttc |
| | cccatcatgcacgacagaaccaagatcaggcagctgcccaatctgctgagaggct |
| | acgagcacgtgagactgtccacccacaacgtgatcaataccgaggatgcccctgg |
| | cggcccttacgagatcggcaccagcggctcctgtctgaatatcacaaatggcaag |
| | ggcttctttgccaccatggcctgggccgtgcctaagaacaagaccgccaccaacc |
| | ctctgaccatcgaggtgccctacatctgtacagaggaggaggatcagatcaccgt |
| | gtggggctttcactccgatgacgagacccagatggccagactgtacggcgacagc |
| | aagccccagaagttcacctcctccgccaacggcgtgaccacacactacgtgtccc |
| | agatcggcggcttccctaaccagaccgaggacggcggcctgcctcagagcggaag |
| | gatcgtggtggattacatggtgcagaagtccggcaagacaggcaccatcacatac |
| | cagaggggcatcctgctgcctcagaaggtgtggtgcgcctccggcaagagcaagg |
| | tcattaagggcagcctgcctctgatcggcgaggccgactgcctgcacgagaagta |
| | cggcggcctgaataagagcaagccctactacaccggcgagcacgccaaggccatc |
| | ggcaactgtcccatctgggtgaagacacctctgaagctggccaatggcaccaagt |
| | acagaccccccgccaagctgctgaaggagagaggctttttcggcgccatcgccgg |
| | cttcctggagggcggatgggagggaatgatcgccggctggcacggctacaccagc |
| | cacggagcccacggcgtggccgttgctgctgacctgaagagcacccaggaggcca |
| | tcaacaagatcaccaagaatctgaatagcctgtccgagctggaggtgaagaatct |
| | gcagagactgtccggcgccatggatgagctgcacaatgagatcctggagctggat |
| | gagaaggtggacgacctgagagccgacacaatctccagccagatcgagctggccg |
| | tgctgctgagcaacgagggcatcatcaatagcgaggatgagcacctgctggccct |
| | ggagagaaagctgaagaagatgctgggcccttccgccgtggagatcggcaacggc |
| | tgttttgagaccaagcacaagtgccaacagacatgtctggataggategccgccg |
| | gcacctttgacgccggcgagttctccctgcccacattcgatagcctgcaaatcac |
| | cgccgcctccctgaacgatgacggcctggatcaacacaccatcctgctgtactac |
| | agcaccgccgccagcagcctggccgtgacactgatgatcgccatcttcgtggtgt |
| | acatggtgagcagggataatgtgtcctgttccatctgtctgtgatagtaa |
| SEQ ID NO: 26 | QSRG |
| Synthetic | |
| SEQ ID NO: 27 | QTRG |
| Synthetic | |
| SEQ ID NO: 28 | RRKKRGLF |
| Synthetic | |
| SEQ ID NO: 29 | KERG |
| Synthetic | |
| SEQ ID NO: 30 | GLFGAIAGFIEGGW |
| Synthetic | |
| SEQ ID NO: 31 | GIFGAIAGFIEGGW |
| Synthetic | |
| SEQ ID NO: 32 | GLFGAIAGFIEGGWTGM |
| Synthetic | |

What is claimed is:

1. An isolated nucleic acid, which encodes a recombinant influenza hemagglutinin (HA) comprising a signal peptide, an HA1 subunit, and an HA2 subunit, wherein the influenza HA comprises at least one glycosylation site, and the HA2 subunit is devoid of a glycosylation site.

2. The isolated nucleic acid of claim 1, wherein the HA1 subunit and the HA2 subunit are defined by a conserved proteolytic cleavage site comprising a X_{b}-X_{b}—R-G (SEQ ID NO: 33) peptide, wherein G denotes a glycine (G) residue, R denotes an arginine (R) residue, and X_{b} denotes any amino acid.

3. The isolated nucleic acid of claim 1, wherein the influenza HA comprises at least one glycosylation site in the HA1 subunit.

4. The isolated nucleic acid of claim 1, wherein the influenza HA comprises a stem region, comprising (1) the HA2 subunit, (2) a HA1 N-terminal loop motif, (3) a HA1 lower helix motif, and (4) a HA1 C-terminal beta-strands motif, wherein, except for the HA1 N-terminal loop motif, the stem region is devoid of a glycosylation site.

5. The isolated nucleic acid of claim 4, wherein the HA1 N-terminal loop motif comprises the first 10 to 40 amino acids, the first 15 to 40 amino acids, or the first 20 to 40 amino acids immediately C-terminal to the signal peptide; and further wherein the HA1 C-terminal beta-strands motif comprises the last 10 to 30 amino acids of the HA1 subunit, and the HA1 lower helix motif comprises the 5 to 15 amino acids immediately upstream of the HA1 C-terminal beta-strands motif.

6. The isolated nucleic acid of claim 1, wherein the HA2 subunit comprise a deglycosylation sequon of Z-Xₐ-S/T, wherein Z denotes any amino acid residue except for asparagine (N; Asn), S denotes a serine (S; Ser) residue, T denotes a threonine (T; Thr) residue, and Xₐ is any amino acid residue except for proline (P).

7. The isolated nucleic acid of claim 6, wherein, in the deglycosylation sequon, Z denotes glutamine (Q; Gln).

8. The isolated nucleic acid of claim 4, wherein the HA1 N-terminal loop motif comprises a glycosylation site.

9. The isolated nucleic acid of claim 4, wherein the HA1 N-terminal loop motif is devoid of a glycosylation site.

10. The isolated nucleic acid of claim 1, wherein the HA2 subunit is devoid of a glycosylation site, compared with a reference influenza HA, wherein the reference influenza HA comprises an amino acid sequence at least 80% identical to SEQ ID NO: 1, SEQ ID NO: 4, SEQ ID NO: 7, SEQ ID NO: 14, or SEQ ID NO: 19.

11. The isolated nucleic acid of claim 1, wherein the influenza HA comprises an amino acid sequence at least 80%, 85%, 90%, 91%, 92%, 93%, 94%, 95%, 96%, 97%, 98%, or 99%, or 100% identical to SEQ ID NO 5:
MKAILVVMLY TFTTANADTL CIGYHAXXST DTVDTVLEKX
VTVTHSVNLL EDKHNGKLCK LRGVAPLHLG QCNIAGWILG
NPECESLSTA RSWSYIVETS NSDNGTCYPG DFINYEELRE
QLSSVSSFER FEIFPKTSSW PNHDSDNGVT AACSHAGARS
FYKNLIWLVK KGKSYPKINQ TYINDKGKEV LVLWGIHHPP
TITDQESLYQ NADAYVFVGT SRYSKKFKPE IAARPKVRDR
AGRMNYYWTL VEPGDKITFE ATGNLVAPRY AFTMEKEAGS
GIIISDTPVH DCXATCQTPE GAIXTSLPFQ NVHPITIGKC
PKYVRSTKLR LATGLRNVPS IQSRGLFGAI AGFIEGGWTG
MVDGWYGYHH QNDQGSGYAA DLKSTQNAID KITNKVNSVI
EKMNTQFTAV GKEFNHLEKR IENLNKKVDD GFLDVWTYNA
ELLVLLENER TLDYHDSNVK NLYEKVRHQL KNNAKEIGNG
CFEFYHKCDN TCMESVKXGT YDYPKYSEEA KLNREKIDGV
KLDSTRIYQI LAIYSTVASS LVLVVSLGAI SFWMCSNGSL
QCRICI,
wherein X denotes any amino acid, provided that (1) X40, X293, X304, and X498 are not asparagine (N; Asn), and both X27 and X28 are Asn; or (2) X27, X40, X293, X304, and X498 are not Asn.

12. The isolated nucleic acid of claim 1, wherein the influenza HA comprises an amino acid sequence at least 80% identical to SEQ ID NO: 8:
MKAIIALSNI LCLVFAQKIP GNDXSTATLC LGHHAVPXGT
IVKTITNDRI EVTNATELVQ NSSIGKICNS PHQILDGGNC
TLIDALLGDP QCDGFQNKEW DLFVERSRAN SSCYPYDVPD
YASLRSLVAS SGTLEFKNES FNWTGVKQNG TSSACKRGSS
SSFFSRLNWL TSLNNIYPAQ NVTMPNKEQF DKLYIWGVHH
PDTDKNQFSL FAQSSGRITV STKRSQQAVI PNIGSRPRVR
DIPSRISIYW TIVKPGDILL INSTGNLIAP RGYFKIRSGK
SSIMRSDAPI GKCKSECITP NGSIPNDKPF QNVNRITYGA
CPRYVKQSTL KLATGMRNVP EKQTRGIFGA IAGFIENGWE
GMVDGWYGFR HQNSEGRGQA ADLKSTQAAI DQISGKLNRL
IGKTNEKFHQ IEKEFSEVEG RVQDLEKYVE DTKIDLWSYN
AELLVALENQ HTIDLTDSEM NKLFEKTKKQ LRENAEDMGN
GCFKIYHKCD NACIGSIRXE TYDHNVYRDE ALNNRFQIKG
VELKSGYKDW ILWISFAMSC FLLCIALLGF IMWACQKGNI
RCNICI,
and
wherein X denotes any amino acid, provided that (1) X38 and X499 are not asparagine (N; Asn), and N24 is Asn, or (2) X24, X38 and X499 are not Asn.

13. The isolated nucleic acid of claim 1, wherein the influenza HA comprises an amino acid sequence at least 80% identical to SEQ ID NO: 8:
MKAIIALSNI LCLVFAQKIP GNDXSTATLC LGHHAVPXGT
IVKTITNDRI EVTNATELVQ NSSIGKICNS PHQILDGGNC
TLIDALLGDP QCDGFQNKEW DLFVERSRAN SSCYPYDVPD
YASLRSLVAS SGTLEFKNES FNWTGVKQNG TSSACKRGSS
SSFFSRLNWL TSLNNIYPAQ NVTMPNKEQF DKLYIWGVHH
PDTDKNQFSL FAQSSGRITV STKRSQQAVI PNIGSRPRVR
DIPSRISIYW TIVKPGDILL INSTGNLIAP RGYFKIRSGK
SSIMRSDAPI GKCKSECITP NGSIPNDKPF QNVNRITYGA
CPRYVKQSTL KLATGMRNVP EKQTRGIFGA IAGFIENGWE
GMVDGWYGFR HQNSEGRGQA ADLKSTQAAI DQISGKLNRL
IGKTNEKFHQ IEKEFSEVEG RVQDLEKYVE DTKIDLWSYN
AELLVALENQ HTIDLTDSEM NKLFEKTKKQ LRENAEDMGN
GCFKIYHKCD NACIGSIRXE TYDHNVYRDE ALNNRFQIKG
VELKSGYKDW ILWISFAMSC FLLCIALLGF IMWACQKGNI
RCNICI,
and
wherein X denotes any amino acid, provided that X24, X38, and X499 are not asparagine (N; Asn), and N54, N61, N79, and N301 are replaced with an amino acid other than Asn.

14. The isolated nucleic acid of claim 1, wherein the influenza HA comprises an amino acid sequence at least 80% identical to SEQ ID NO 2:
MKAIIVLLMV VTSNADRICT GITSSNSPHV VKTATQGEVX
VTGVIPLTTT PTKSYFANLK GTRTRGKLCP DCLNCTDLDV
ALGRPMCVGT TPSAKASILH EVRPVTSGCF PIMHDRTKIR
QLPNLLRGYE KIRLSTQNVI DAEKAPGGPY RLGTSGSCPN
ATSKIGFFAT MAWAVPKDNY KNATNPLTVE VPYICTEGED
QITVWGFHSD DKTQMKSLYG DSNPQKFTSS ANGVTTHYVS
QIGDFPDQTE DGGLPQSGRI VVDYMMQKPG KTGTIVYQRG
VLLPQKVWCA SGRSKVIKGS LPLIGEADCL HEEYGGLXKS
KPYYTGKHAK AIGNCPIWVK TPLKLAXGTK YRPPAKLLKE
RGFFGAIAGF LEGGWEGMIA GWHGYTSHGA HGVAVAADLK
STQEAINKIT KNLNSLSELE VKNLQRLSGA MDELHNEILE
LDEKVDDLRA DTISSQIELA VLLSNEGIIN SEDEHLLALE
RKLKKMLGPS AVDIGNGCFE TKHKCXQTCL DRIAAGTFNA
GEFSLPTFDS LXITAASLND DGLDXHTILL YYSTAASSLA
VTLMLAIFIV YMVSRDNVSC SICL,
wherein X denotes any amino acid, provided that (1) X318, X347, X506, X532, and X545 are not asparagine (N; Asn), and X40 is Asn or (2) X40, X318, X347, X506, X532, and X545 are not Asn.

15. The isolated nucleic acid of claim 1, wherein the influenza HA comprises an amino acid sequence at least 80%, 85%, 90%, 91%, 92%, 93%, 94%, 95%, 96%, 97%, 98%, or 99%, or 100% identical to SEQ ID NO: 15:
MKAIIVLLMV VTSNADRICT GITSSNSPHV VKTATQGEVX
VTGVIPLTTT PTKSHFANLK GTETRGKLCP KCLNCTDLDV
ALGRPKCTGK IPSARVSILH EVRPVTSGCF PIMHDRTKIR
QLPNLLRGYE HVRLSTHNVI NTEDAPGGPY ITNGKGFFAT
MAWAVPKNKT ATNPLTIEVP YICTEEEDQI TVWGFHSDDE
TQMARLYGDS KPQKFTSSAN GVTTHYVSQI GGFPNQTEDG
GLPQSGRIVV DYMVQKSGKT GTITYQRGIL LPQKVWCASG
KSKVIKGSLP LIGEADCLHE KYGGLXKSKP YYTGEHAKAI
GNCPIWVKTP LKLAXGTKYR PPAKLLKERG FFGAIAGFLE
GGWEGMIAGW HGYTSHGAHG VAVAADLKST QEAINKITKN
LNSLSELEVK NLQRLSGAMD ELHNEILELD EKVDDLRADT
ISSQIELAVL LSNEGIINSE DEHLLALERK LKKMLGPSAV
EIGNGCFETK HKCXQTCLDR IAAGTFDAGE FSLPTFDSLX
ITAASLNDDG LDXHTILLYY STAASSLAVT LMIAIFVVYM
VSRDNVSCSI CL,
wherein X denotes any amino acid, provided that (1) X316, X345, X504, X530, and X543 are not asparagine (N; Asn), and X40 is Asn; (2) X504, X530, and X543 are not Asn, and X40, X316, and X345 are Asn; or (3) X40, X316, X345, X504, X530, and X543 are not Asn.

16. An expression vector, comprising an expression cassette, and wherein the expression cassette comprises the isolated nucleic acid of claim 1.

17. The expression vector of claim 16, wherein the expression cassette further comprises a promoter, a 5′ untranslated region (5′UTR), a 3′ untranslated region (3′UTR), a 5′ cap, a poly-A tail, or a combination thereof, operably linked to the isolated nucleic acid.

18. The expression vector of claim 16, wherein the expression vector is a lipid nanoparticle, and the lipid nanoparticle comprises a membrane defining an inner space, and wherein the membrane encompasses the isolated nucleic acid, and the membrane is formed with a plurality of lipid components comprising a bi-functional compound, and the bi-functional compound comprises: 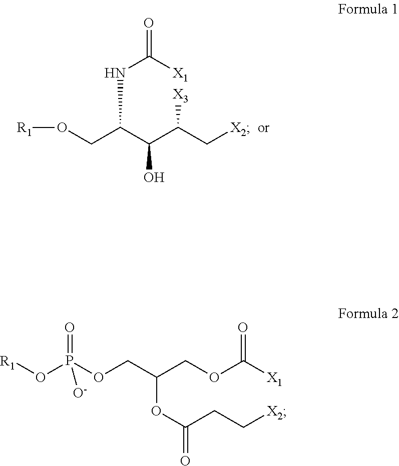
wherein R₁ comprises a substituted or non-substituted glycosyl group;
wherein X₁ and X₂ are each independently hydrogen, C₁₋₃₀ alkyl, C₁₋₃₀ alkenyl, C₁₋₃₀ alkynyl, aryl, aryloxy, or a substituted version thereof, or
—(CH₂)nX₄, n is 0 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof; and
wherein X₃ is hydrogen, C₁₋₆ alkyl, or hydroxyl.

19. The expression vector of claim 18, wherein R₁ comprises a formula of R₂—R_{A}—, wherein R_{A} is an attachment group and R₂ is the substituted or non-substituted glycosyl group, and wherein the attachment group comprises an aryl, an alkyl, an amide, an alkylamide, a substituted version thereof, a combination thereof, or a covalent bond, and further wherein R_{A} comprises the aryl having 0 to 3 substituents, wherein the substituent is C₁₋₆ alkyl, halide, or C₁₋₆ alkyl halide.

20. The expression vector of claim 18, wherein the glycosyl group comprises a terminal mannoside, a terminal fucoside, or both.

21. The expression vector of claim 18, wherein R₁ is a substituted glycosyl group.

22. The expression vector of claim 18, wherein R₁ is selected from the group consisting of:

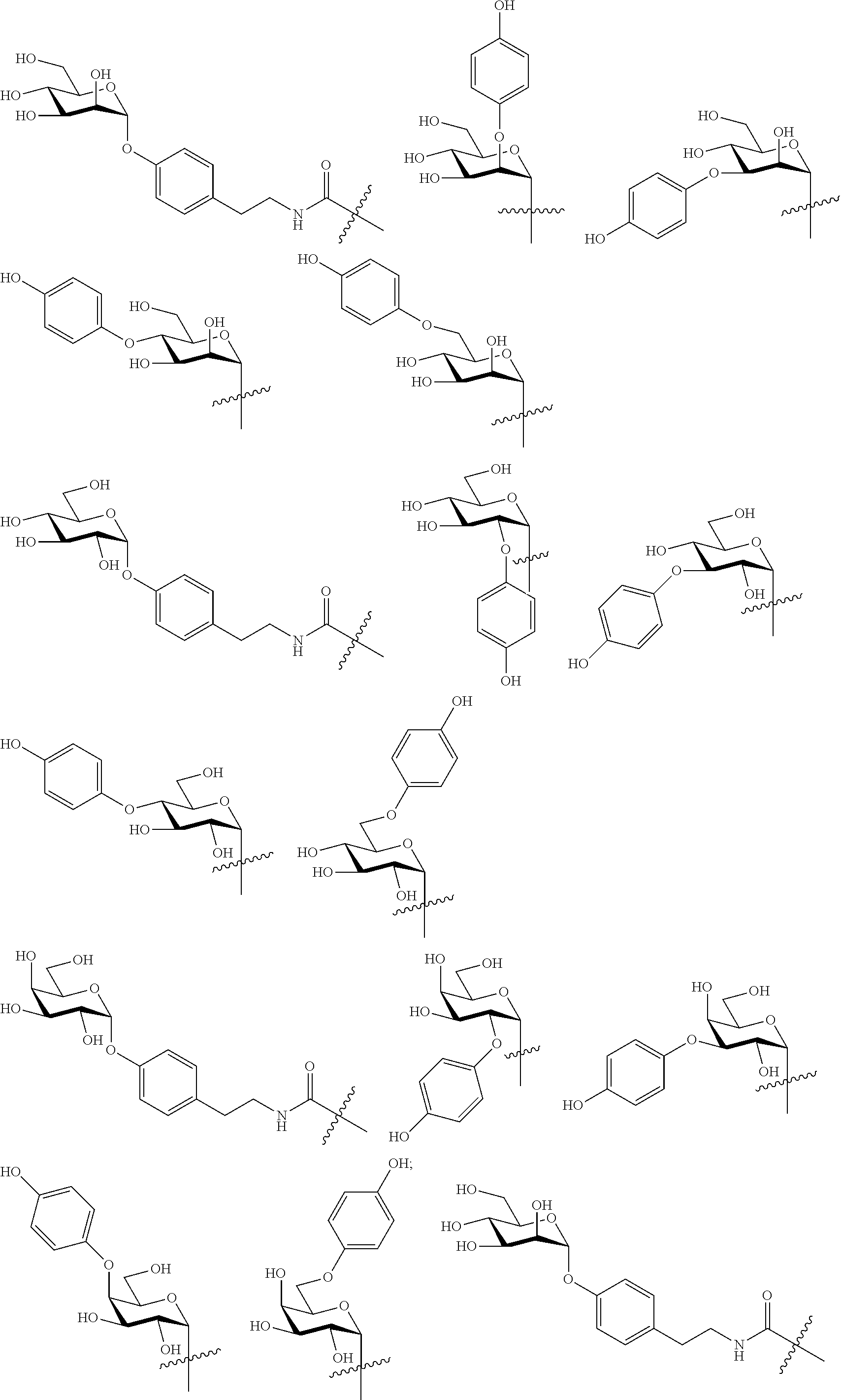

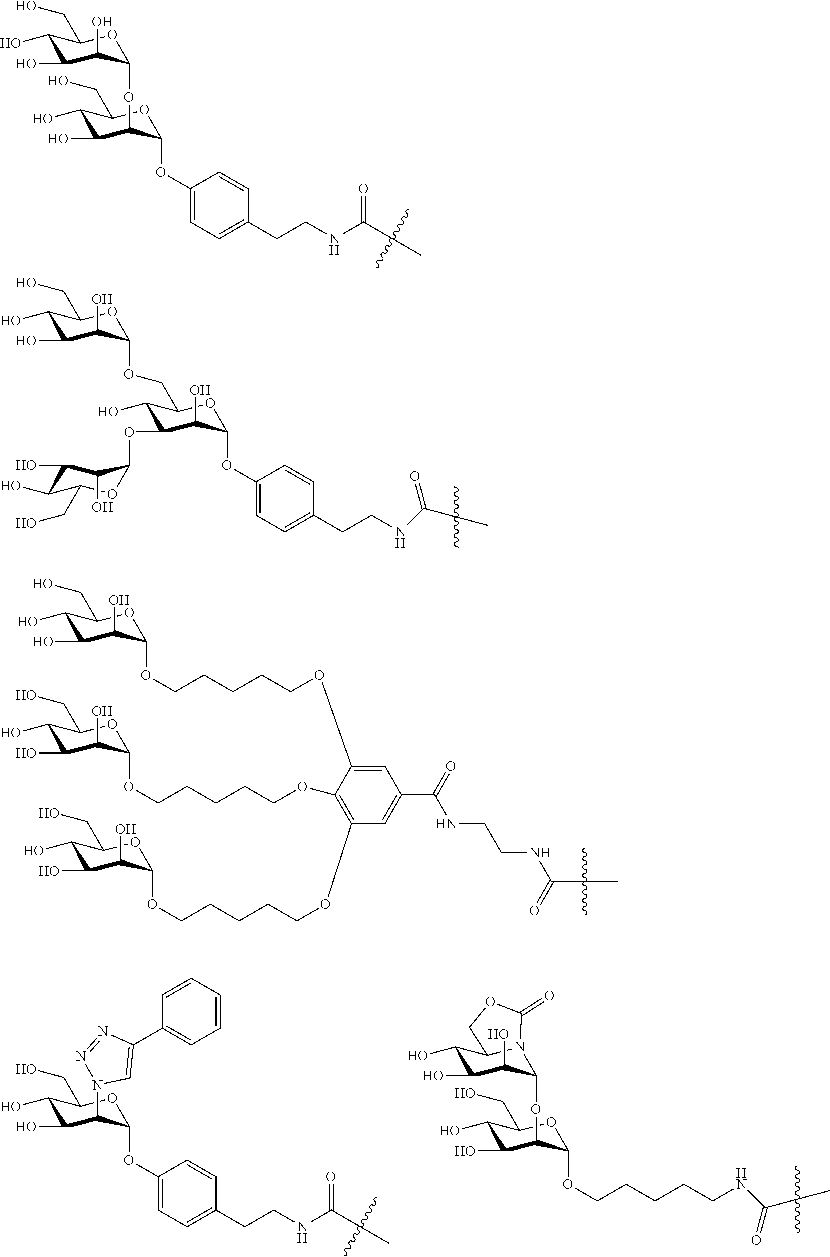

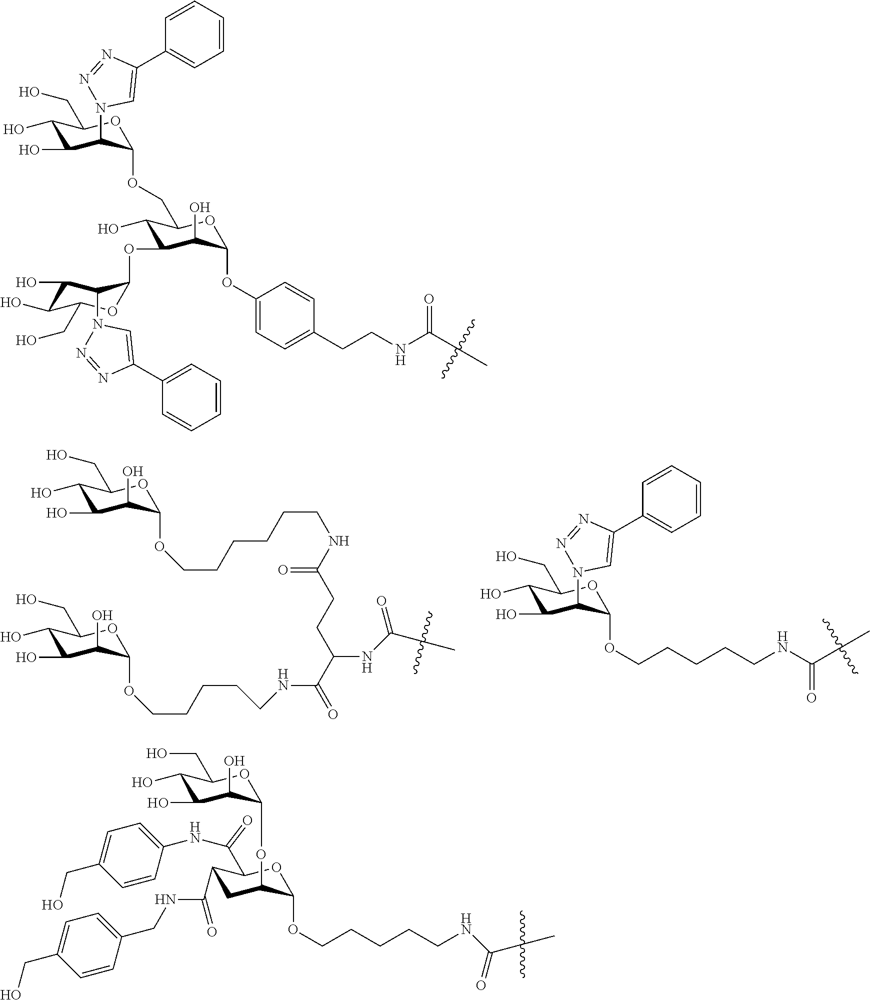

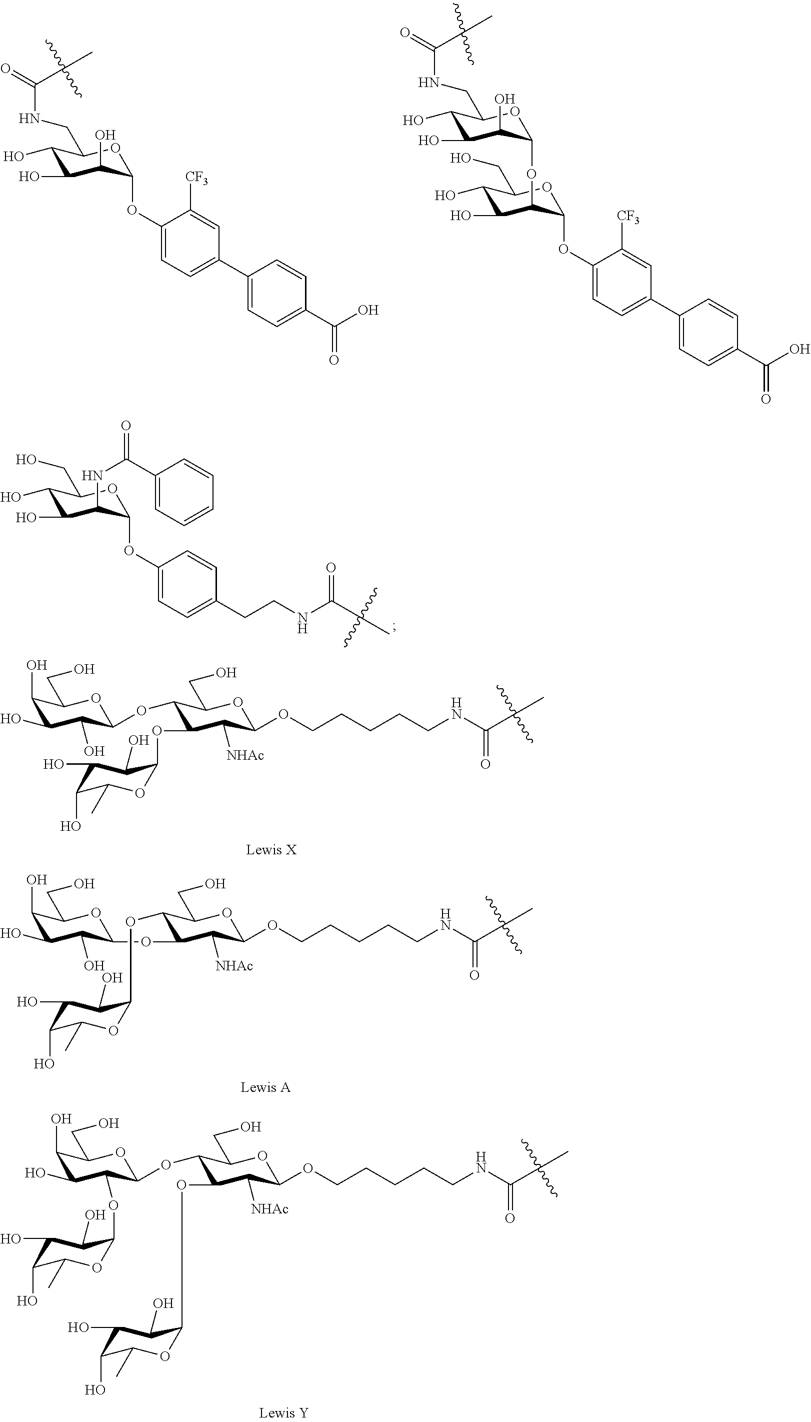

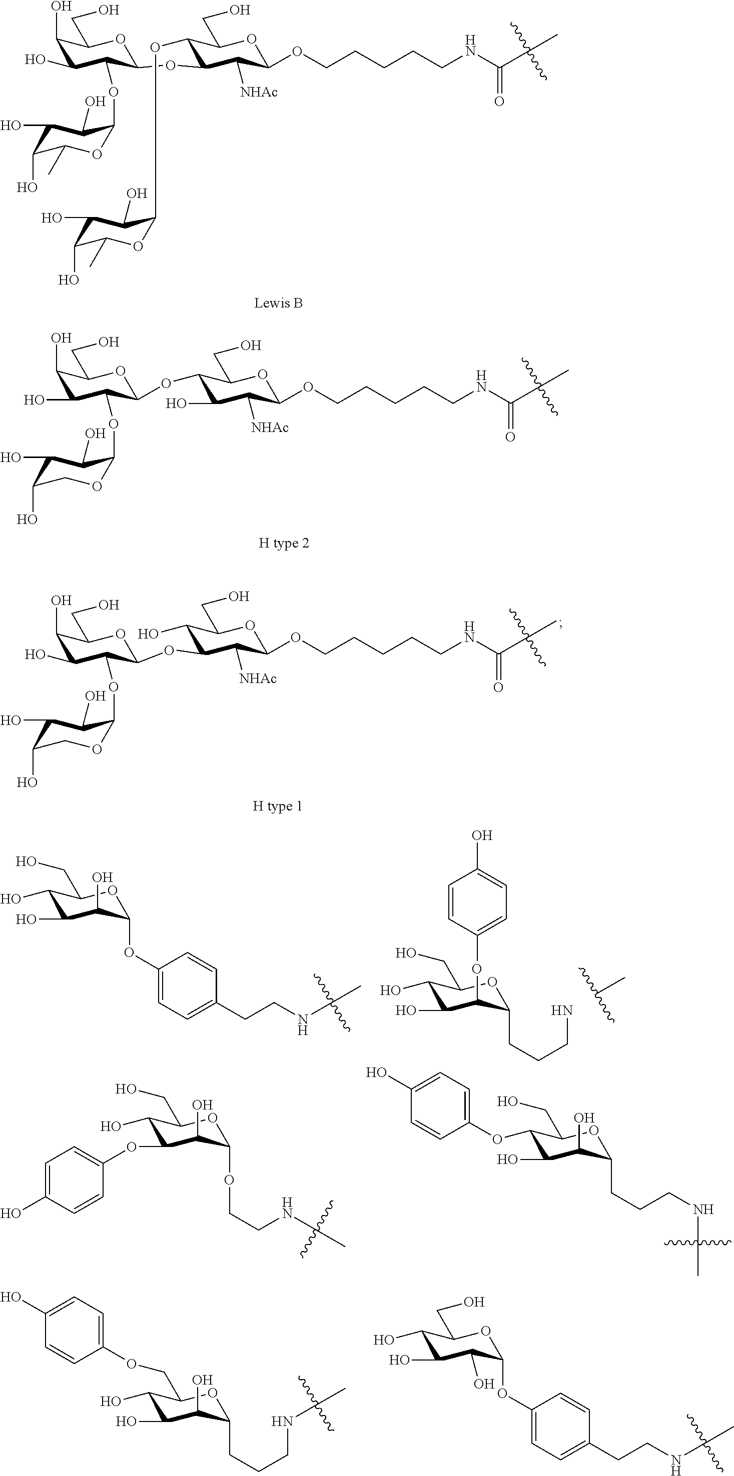

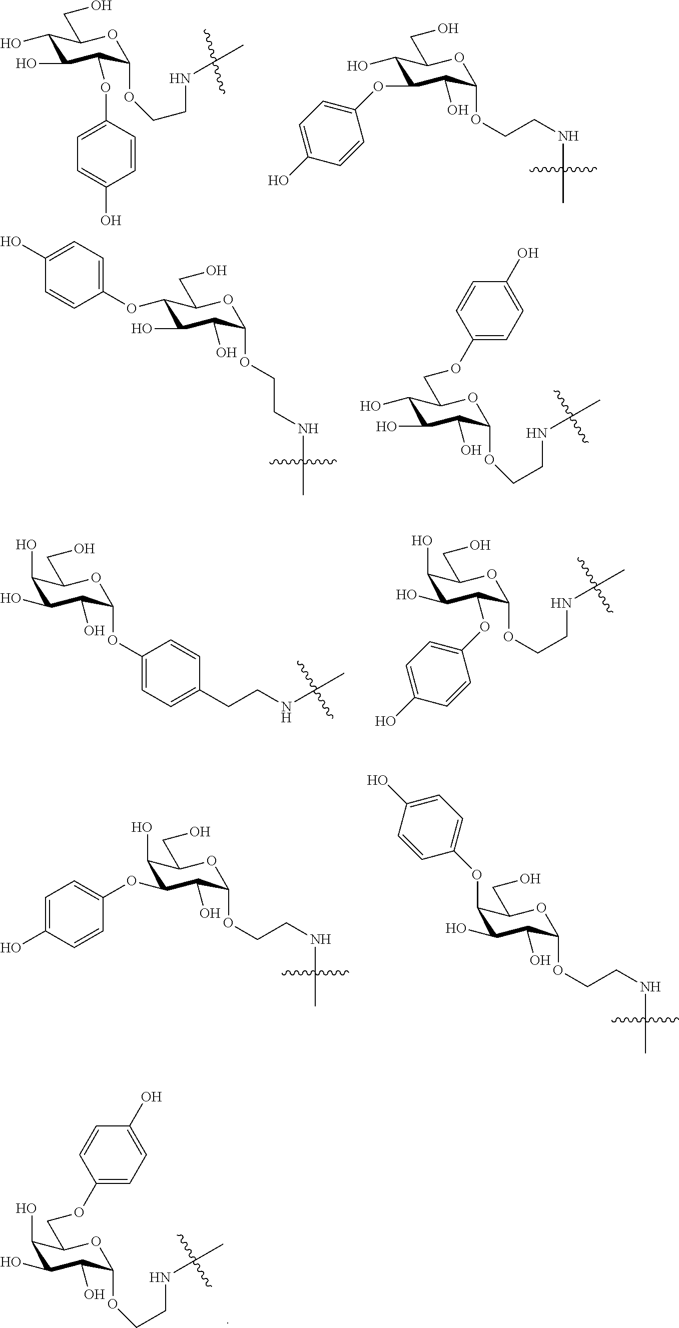

23. The expression vector of claim 18, wherein the bi-functional compound is of Formula 3: 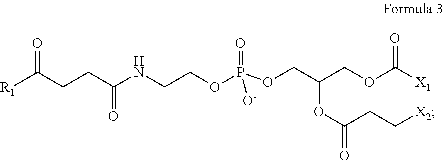
and
wherein R₁ is selected from the group consisting of:

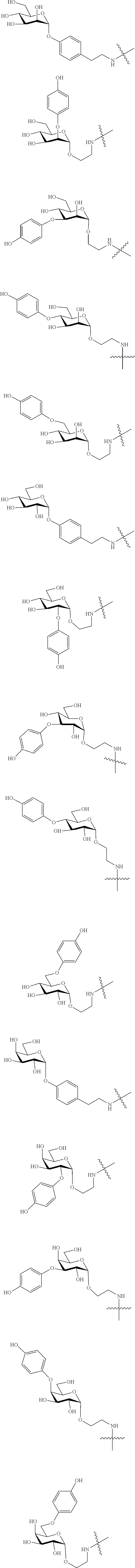

24. The expression vector of claim 18, wherein X₁ and X₂ are each independently hydrogen, C₄₋₃₀ alkyl, C₄₋₃₀ alkenyl, C₄₋₃₀ alkynyl, aryl, aryloxy, or a substituted version thereof, or
—(CH₂)nX₄, n is 4 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof.

25. The expression vector of claim 18, wherein the compound is selected from the group consisting of:

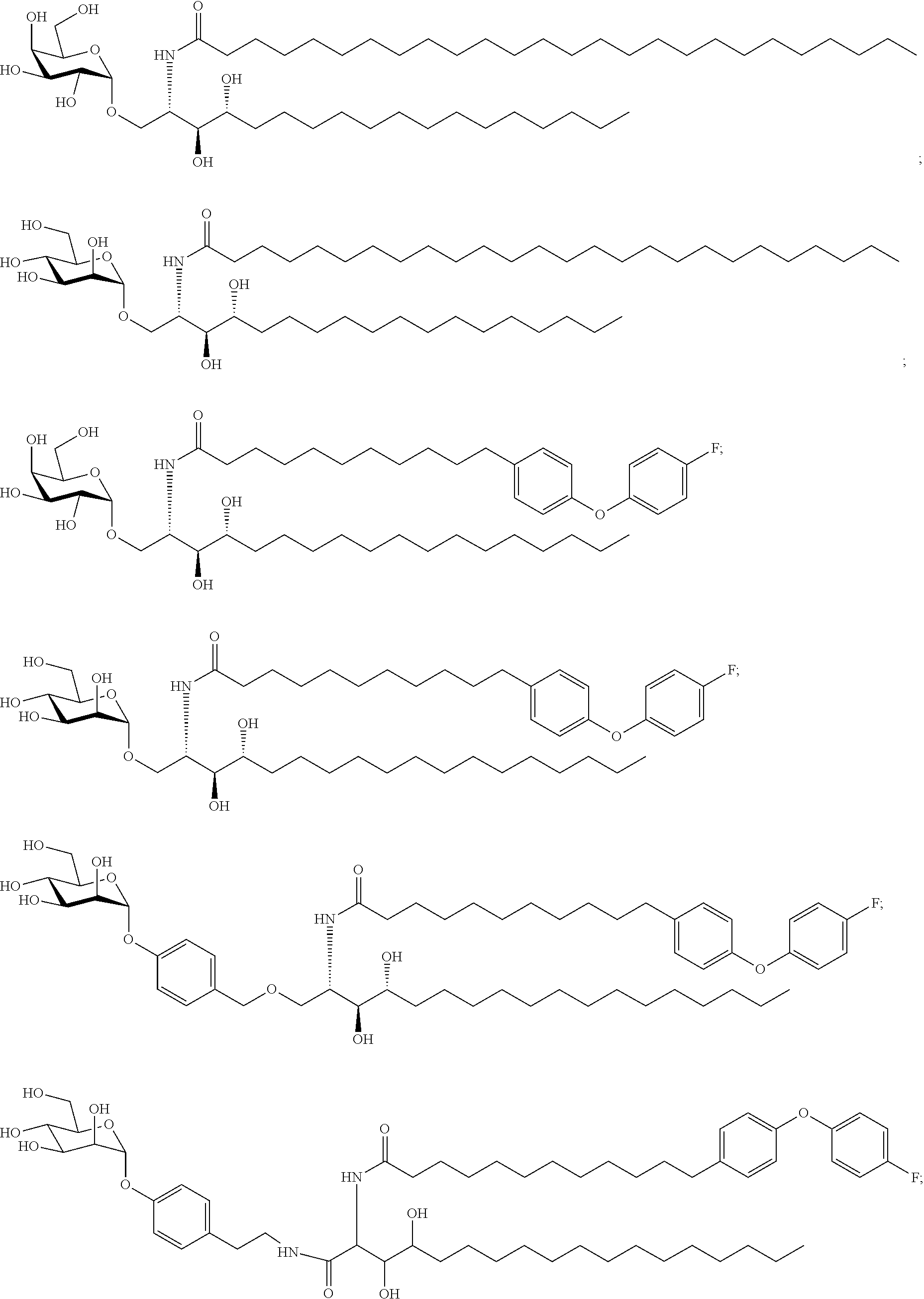

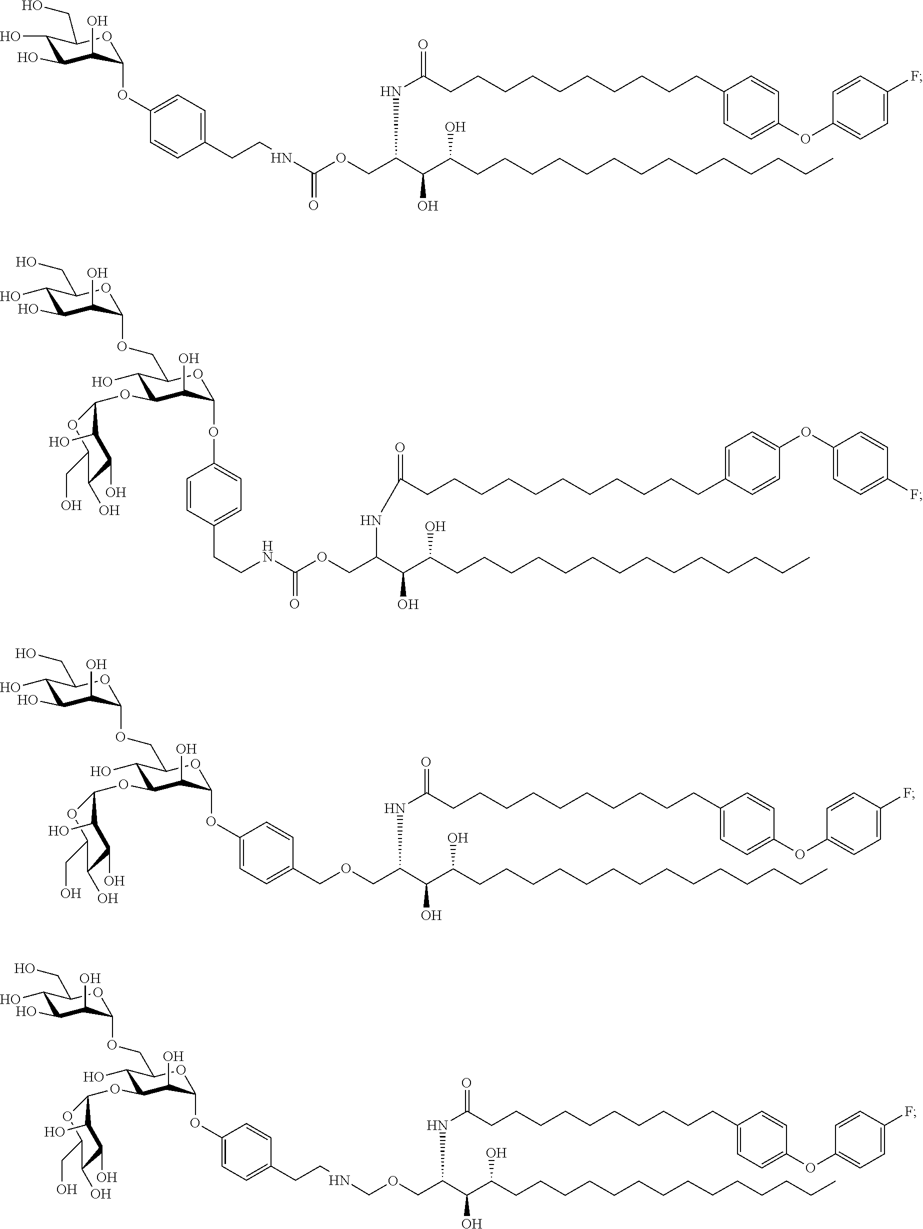

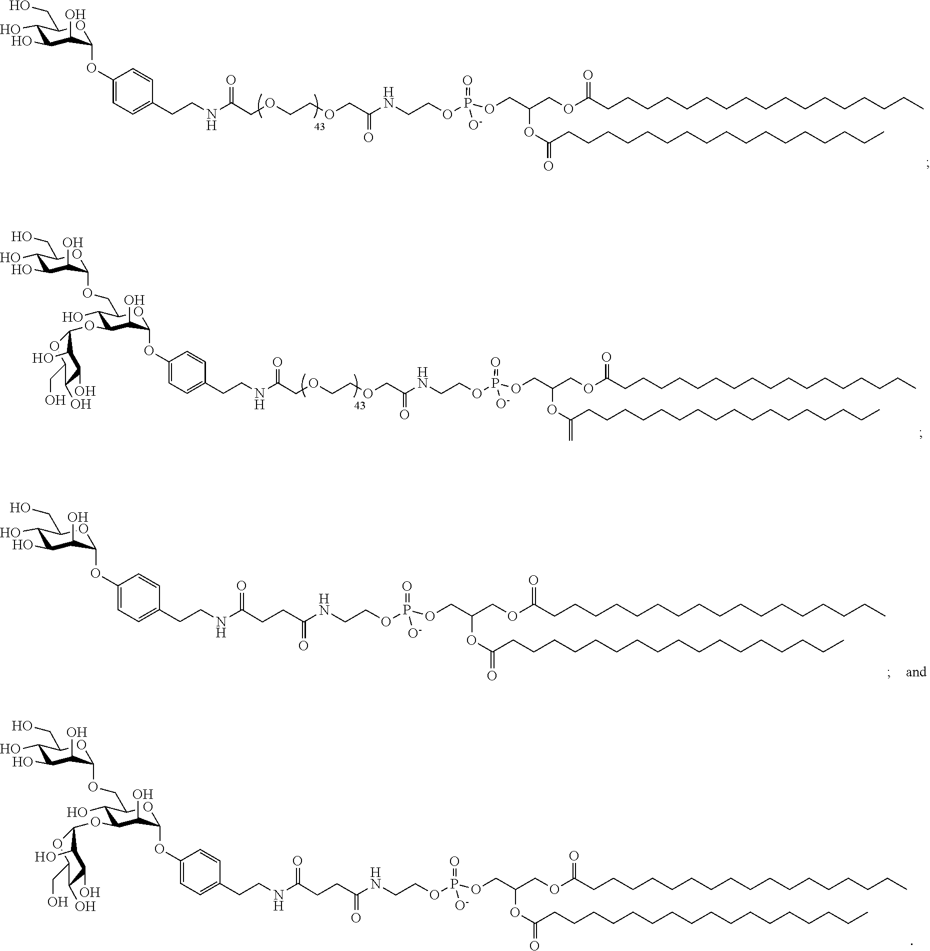

26. The expression vector of claim 18, wherein the plurality of the lipid components further comprises an ionizable lipid, a helper lipid, or a combination thereof.

27. An immunogenic composition, comprising an expression vector of claim 16.

28. An immunogenic composition, comprising
a first isolated nucleic acid, encoding a first recombinant influenza hemagglutinin (HA), which comprises an amino acid sequence at least 80%, 85%, 90%, 91%, 92%, 93%, 94%, 95%, 96%, 97%, 98%, or 99%, or 100% identical to SEQ ID NO: 6, provided that each of the 40^{th}, the 293^{rd}, the 304^{th}, and the 498^{th} amino acids thereof does not form a glycosylation site, and the 27^{th} amino acid thereof forms a glycosylation site;
a second isolated nucleic acid, encoding a second recombinant influenza hemagglutinin (HA), which comprises an amino acid sequence at least 80%, 85%, 90%, 91%, 92%, 93%, 94%, 95%, 96%, 97%, 98%, or 99%, or 100% identical to SEQ ID NO: 11, provided that each of the 24^{th}, the 38^{th}, the 54^{th}, the 61^{st}, the 79^{th}, the 301^{st}, and the 499^{th} amino acids thereof does not form a glycosylation site;
a third isolated nucleic acid, encoding a third recombinant influenza hemagglutinin (HA), which comprises an amino acid sequence at least 80%, 85%, 90%, 91%, 92%, 93%, 94%, 95%, 96%, 97%, 98%, or 99%, or 100% identical to SEQ ID NO: 3, provided that each of the 318^{th}, the 347^{th}, the 506^{th}, the 532^{nd}, and the 545^{th} amino acids does not form a glycosylation site, and the 40^{th} amino acid forms a glycosylation site; and
a fourth isolated nucleic acid, encoding a fourth recombinant influenza hemagglutinin (HA), which comprises an amino acid sequence at least 80%, 85%, 90%, 91%, 92%, 93%, 94%, 95%, 96%, 97%, 98%, or 99%, or 100% identical to SEQ ID NO: 18, provided that each of the 504^{th}, the 530^{th}, and the 543^{rd} amino acid does not form a glycosylation site, and each of the 40^{th}, the 316^{th}, and the 345^{th}, amino acids forms a glycosylation site.

29. A method for generating an immune response against influenza virus infection, comprising administering the isolated nucleic acid of claim 1 to a subject in need at an effective amount.

30. A recombinant influenza hemagglutinin, comprising an amino acid sequence at least 80%, 85%, 90%, 91%, 92%, 93%, 94%, 95%, 96%, 97%, 98%, or 99%, or 100% identical to SEQ ID NO: 3, SEQ ID NO: 6, SEQ ID NO: 9, SEQ ID NO: 11, SEQ ID NO: 16, or SEQ ID NO: 18.
